(19) United States
(12) Patent Application Publication
(10) Pub. No.: US 2015/0307562 A1
(43) Pub. Date: Oct. 29, 2015

(54) ENGINEERED SECRETED PROTEINS AND METHODS

(71) Applicant: PRONUTRIA BIOSCIENCES, INC., Cambridge, MA (US)

(72) Inventors: Subhayu Basu, Chestnut Hill, MA (US); Katherine G. Gora, Boston, MA (US); Ying-Ja Chen, Cambridge, MA (US); David M. Young, Portsmouth, NH (US); Nathaniel W. Silver, Cambridge, MA (US); Michael J. Hamill, Wellesley, MA (US); David A. Berry, Brookline, MA (US)

(21) Appl. No.: 14/443,773

(22) PCT Filed: Nov. 20, 2013
(86) PCT No.: PCT/US13/71091

§ 371 Date: May 19, 2015

Related U.S. Application Data
(60) Provisional application No. 61/728,427, filed on Nov. 20, 2012.

(51) Int. Cl. C07K 14/32; A23L 1/305; C07K 14/195

(52) U.S. Cl. CPC C07K 14/32; C07K 14/195; A23L 1/3053; A23V 2002/00

(57) ABSTRACT

Nutritive proteins are provided herein. Also provided are various other embodiments including nucleic acids encoding the proteins, recombinant microorganisms that make the proteins, vectors for expressing the proteins, methods of making the proteins using recombinant microorganisms, compositions that comprise the proteins, and methods of using the proteins. Nutritive proteins include engineered proteins, wherein the engineered proteins comprise a sequence of at least 20 amino acids that comprise an altered amino acid sequence compared to the amino acid sequence of a reference secreted protein and a ratio of essential amino acids to total amino acids present in the engineered protein higher than the ratio of essential amino acids to total amino acids present in the reference secreted protein. In some embodiments, the engineered protein comprises at least one essential amino acid residue substitution of a non-essential amino acid residue in the reference secreted protein.

### CROSS REFERENCE TO RELATED APPLICATIONS

This application claims priority to U.S. Ser. No. 61/728,427, filed Nov. 20, 2012, and is related to PCT/US2013/032232, filed Mar. 15, 2013, PCT/US2013/032180, filed Mar. 15, 2013, PCT/US2013/032225, filed Mar. 15, 2013, PCT/US2013/032218, filed Mar. 15, 2013, PCT/US2013/032212, filed Mar. 15, 2013, PCT/US2013/032206, filed Mar. 15, 2013, and PCT/US2013/038682, filed Apr. 29, 2013; the entire disclosures of which are hereby incorporated by reference in their entirety for all purposes.

### SEQUENCE LISTING

The instant application contains a Sequence Listing which has been submitted electronically in ASCII format and is hereby incorporated by reference in its entirety. Said ASCII copy, created on Mar. 26, 2014, is named 25045PCT_CRF_SequenceListing.txt and is 83,758,406 bytes in size.

### INTRODUCTION

Naturally occurring proteins are made from the twenty different types of amino acids, namely alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine (S), threonine (T), tryptophan (W), tyrosine (Y), and valine (V). During digestion, ingested protein is broken down into amino acids. Protein is an important component of the human diet, because most mammals cannot synthesize all the amino acids they need; essential amino acids must be obtained from food. The amino acids considered essential are Histidine (H), Isoleucine (I), Leucine (L), Lysine (K), Methionine (M), Phenylalanine (F), Threonine (T), Tryptophan (W), and Valine (V).

The World Health Organization recommends that dietary protein should contribute approximately 10 to 15% of energy intake when in energy balance and weight stable. Average daily protein intakes in various countries indicate that these recommendations are consistent with the amount of protein being consumed worldwide. Meals with an average of 20 to 30% of energy from protein are representative of high-protein diets when consumed in energy balance.

Both plant and animal foods contain protein. Proteins that provide all the essential amino acids are referred to as “high quality” proteins. Animal foods such as meat, fish, poultry, eggs, and dairy products are all high quality protein sources. These foods provide a good balance of essential amino acids. Proteins that do not provide a good balance of essential amino acids are referred to as “low quality” proteins. Most fruits and vegetables are poor sources of protein. Some plants foods including beans, peas, lentils, nuts and grains such as wheat are better sources of protein.

Casein, whey, and soy are major sources of protein. Casein is commonly found in mammalian milk, making up 80% of the proteins in cow milk and between 20% and 40% of the proteins in human milk. Casein is also a major component of cheese. Whey is the liquid remaining after milk has been curdled and strained and is also a byproduct of the manufacture of cheese or casein. Soy is a vegetable protein manufactured from soybeans. While most vegetable proteins are considered to be low quality proteins, soy protein is considered by some to be a high quality protein, and it is comparable to many animal/milk based proteins.

Studies of the acute effects of consuming high amounts of protein in humans have shown that inclusion of, and in some cases increasing, protein content in the diet can have beneficial effects. For example, studies have shown that protein can induce postprandial satiety (including by suppressing hunger), that protein diets induce thermogenesis and that glycemic response is reduced by protein diets.

Studies of the chronic use of high protein diets for weight loss have shown that protein positively affects energy expenditure and lean body mass, that overeating produces significantly less weight gain in diets containing at least 5% of energy from protein, and that a high-protein diet decreases energy intake.

Clinical studies provide evidence that protein prevents muscle protein loss due to aging or bed rest. In particular, muscle fractional synthetic rate (FSR) increases after protein supplementation during prolonged bed rest, protein supplementation maintains leg mass and strength during prolonged bed rest, protein supplementation increases lean body mass, protein supplementation improves functional measures of gait and balance, and essential amino acid supplementation may serve as a viable intervention for individuals at risk of sarcopenia due to immobility or prolonged bed rest.

Studies on increasing muscle protein anabolism in athletes have shown that protein provided following exercise promotes muscle hypertrophy to a greater extent than that achieved by exercise alone. It has also been shown that protein provided following exercise supports protein synthesis without any increase in protein breakdown, resulting in a net positive protein balance and muscle mass accretion. While muscle protein synthesis appears to respond in a dose-response fashion to essential amino acid supplementation, not all proteins are equal in building muscle. For example, milk proteins appear to be superior to soy in supporting muscle mass accretion with resistance training, while both are superior to carbohydrate alone. The amino acid leucine is an important factor in stimulating muscle protein synthesis.

Whole proteins commonly found in foods do not necessarily provide an amino acid composition that meets the amino acid requirements of a mammal, such as a human, in an efficient manner. The result is that, in order to attain the minimal requirements of each essential amino acid, a larger amount of total protein must be consumed in the diet than would be required if the quality of the dietary protein were higher. By increasing the quality of the protein in the diet it is possible to reduce the total amount of protein that must be consumed compared to diets that include lower quality proteins.

In general, proteins that have higher protein quality are considered more beneficial in a mammalian diet than other proteins that do not. Such proteins are useful, for example, as components of a mammalian diet. Under certain circumstances such proteins promote maintenance of muscle mass, a healthy body mass index, and glycemic balance, among other things. Accordingly, there is a need for sources of proteins that have high protein quality.

In general, proteins that have higher protein quality are considered more beneficial in a mammalian diet than other proteins that do not. Such proteins are useful, for example, as components of a mammalian diet. Under certain circumstances such proteins promote maintenance of muscle mass, a healthy body mass index, and glycemic balance, among other things. Accordingly, there is a need for sources of proteins that have high protein quality.

In theory polypeptides comprising a high proportion of at least one of branch chain amino acids, and essential amino acids could be designed entirely in silico. Nucleic acids encoding the synthetic proteins could then be synthesized and recombinant microbes comprising the nucleic acids produced for production of recombinant proteins. This approach has several potential drawbacks, however. For example, skilled artisans are aware that obtaining high levels of production of soluble versions of such synthetic sequences is very challenging.

### SUMMARY

In one aspect, provided are nutritive polypeptides and formulations comprising nutritive polypeptides. For example, provided is an isolated nutritive polypeptide, wherein the nutritive polypeptide comprises a ratio of one or more essential amino acids to total amino acids that is higher than the ratio of one or more essential amino acids to total amino acids in a reference secreted protein at least 50 amino acids in length, wherein the nutritive polypeptide is present in the formulation in a nutritional amount, and wherein the formulation is substantially free of non-comestible products. In an embodiment, the one or more essential amino acids are present in the formulation in a nutritional amount. In another embodiment, the nutritive polypeptide comprises a ratio of total essential amino acids to total amino acids that is higher than the ratio of total essential amino acids to total amino acids in the reference secreted protein. In another embodiment, the nutritive polypeptide comprises a ratio of a single essential amino acid to total amino acids that is higher than the ratio of a single essential amino acid to total amino acids in the reference secreted protein. In another embodiment, the nutritive polypeptide comprises a ratio of two essential amino acids to total amino acids that is higher than the ratio of two essential amino acids to total amino acids in the reference secreted protein. In another embodiment, the reference secreted protein comprises a secreted enzyme polypeptide. For example, the isolated nutritive polypeptide is capable of a decreased level of the primary enzymatic activity of the secreted enzyme polypeptide. In another embodiment, the isolated nutritive polypeptide is substantially purified from a host cell. In another embodiment, the solubility of the nutritive polypeptide exceeds about 10 g/l at pH 7. In another embodiment, the solubility of the nutritive polypeptide exceeds the solubility of the reference secreted protein. In another embodiment, the digestibility of the nutritive polypeptide has a simulated gastric digestion half-life of less than sixty minutes. In another embodiment, the digestibility of the nutritive polypeptide exceeds the digestibility of the reference secreted protein. In another embodiment, the thermostability of the nutritive polypeptide exceeds the thermostability of the reference secreted protein. In another embodiment, the nutritive polypeptide has a calculated solvation score of −20 or less. In another embodiment, the nutritive polypeptide has a calculated aggregation score of 0.75 or less. In another embodiment, the solubility and digestibility of the nutritive polypeptide exceeds the solubility and digestibility of the reference secreted protein. In another embodiment, the nutritive polypeptide has less than about 50% homology to a known allergen. Exemplary formulations contain at least 1.0 g of nutritive polypeptide at a concentration of at least 100 g per 1 kg of formulation. In some embodiments, the formulation is present as a liquid, semi-liquid or gel in a volume not greater than about 500 ml or as a solid or semi-solid in a mass not greater than about 200 g. In another embodiment, the nutritive polypeptide is produced in a recombinant organism. In another embodiment, the nutritive polypeptide is produced by a unicellular organism comprising a recombinant nucleic acid sequence encoding the nutritive polypeptide. In another embodiment, the formulation provides a nutritional benefit of at least about 2% of a reference daily intake value of protein or is otherwise present in an amount sufficient to provide a feeling of satiety when consumed by a human subject. In another embodiment, the formulation provides a nutritional benefit of at least about 2% of a reference daily intake value of one or more essential amino acids. In another embodiment, the formulation provides a nutritional benefit of at least about 2% of a reference daily intake value of total essential amino acids. In another embodiment, the formulation provides at least 10 grams of nutritive polypeptide. Formulations are preferably formulated for enteral administration. In another embodiment, i) the nutritive polypeptide comprises at least about 98%, or 99%, or 99.5% or 99.9% overall sequence identity to the reference secreted protein over the full-length of the nutritive polypeptide or the reference secreted protein, or ii) the nutritive polypeptide comprises an ortholog of the reference secreted protein, wherein the ortholog comprises at least about 70% overall sequence identity to the reference secreted protein over the full-length of the nutritive polypeptide or the reference secreted protein. Also provided are food products comprising at least about 1 gram of the formulations provided herein. In another embodiment, the formulation provides a nutritional benefit per 100 g equivalent to or greater than at least about 2% of a reference daily intake value of protein. In another embodiment, the effective amount of the nutritive polypeptide is lower than the effective amount of the reference secreted protein when administered to a human subject. Preferred formulations are substantially free of a surfactant, a polyvinyl alcohol, a propylene glycol, a polyvinyl acetate, a polyvinylpyrrolidone, a non-comestible polyacid or polyol, a fatty alcohol, an alkylbenzyl sulfonate, an alkyl glucoside, or a methyl paraben. In some embodiments the formulations also comprise a tastant, a vitamin, a mineral, or a combination thereof, or a flavorant or non-nutritive polyol, or a nutritive carbohydrate and/or a nutritive lipid.

In another aspect, provided are recombinant unicellular organisms that individually comprise a recombinant nucleic acid sequence encoding an isolated nutritive polypeptide, wherein the nutritive polypeptide comprises a ratio of one or more essential amino acids to total amino acids that is higher than the ratio of one or more essential amino acids to total amino acids in a reference secreted protein at least 50 amino acids in length. In some embodiments, the nutritive polypeptide is secreted from the unicellular organism.

Also provided are methods of formulating a nutritive product, comprising the steps of providing a composition comprising an effective amount of an isolated nutritive polypeptide, wherein the nutritive polypeptide comprises a ratio of one or more essential amino acids to total amino acids that is higher than the ratio of one or more essential amino acids to total amino acids in a reference secreted protein at least 50 amino acids in length, wherein the nutritive polypeptide is present in the composition at a concentration of at least 1 mg of nutritive polypeptide per gram of the composition, and combining the composition with at least one food component, thereby formulating the nutritive product. For example, the food component comprises a flavorant, a tastant, an agriculturally-derived food product, a vitamin, a mineral, a nutritive carbohydrate, a nutritive lipid, a binder, a filler or a combination thereof, wherein the nutritive product is comestible, and wherein the nutritive product comprises at least 1.0 g of nutritive polypeptide at a concentration of at least 100 g per 1 kg of nutritive product, and wherein the nutritive product is present as a liquid, semi-liquid or gel in a volume not greater than about 500 ml or as a solid or semi-solid in a mass not greater than about 200 g.

Also provided are methods of selecting a nutritive composition for administration to a human subject who can benefit from same, the method comprising: identifying a minimal essential amino acid nutritive need in the subject; calculating an essential amino acid content score required to meet the minimal essential amino acid nutritive need; and providing a nutritive composition comprising an effective amount of a nutritive polypeptide, wherein the nutritive composition has at least the required essential amino acid content score.

Further provided are methods of selecting a nutritive composition for administration to a human subject who can benefit from same, the method comprising: identifying a maximal essential amino acid nutritive need in the subject; calculating an essential amino acid content score required to not exceed the maximal essential amino acid nutritive need; and providing a nutritive composition comprising an effective amount of a nutritive polypeptide, wherein the nutritive composition has no greater than the required essential amino acid content score.

In another aspect, provided are methods of treating a disease, disorder or condition characterized or exacerbated by protein malnourishment in a human subject in need thereof, comprising the step of administering to the human subject a nutritive formulation in an amount sufficient to treat such disease, disorder or condition, wherein the nutritive formulation comprises a nutritive polypeptide and an agriculturally-derived food product, wherein the nutritive polypeptide comprises a ratio of one or more essential amino acids to total amino acids that is higher than the ratio of one or more essential amino acids to total amino acids in a reference secreted protein at least 50 amino acids in length. In one embodiment, the human subject is an elderly subject. In another embodiment, the human subject is a child under 18 years old. In another embodiment, the human subject is a pregnant subject or lactating female subject. In another embodiment, the human subject is an adult between 18 years old and about 65 years old. In another embodiment, the human subject is an adult suffering from or at risk of developing obesity, diabetes, or cardiovascular disease.

Also provided are methods of improving the nutritional status of a human subject, comprising administering to the subject an effective amount of a nutritive formulation comprising an agriculturally-derived food product and an isolated nutritive polypeptide, wherein the nutritive polypeptide comprises a ratio of one or more essential amino acids to total amino acids that is higher than the ratio of one or more essential amino acids to total amino acids in a reference secreted protein at least 50 amino acids in length.

In another aspect, provided are nutrient polypeptides comprising engineered proteins. In some embodiments the engineered protein comprises a sequence of at least 20 amino acids that comprise an altered amino acid sequence compared to the amino acid sequence of a reference secreted protein and a ratio of essential amino acids to total amino acids present in the engineered protein higher than the ratio of essential amino acids to total amino acids present in the reference secreted protein.

In some embodiments, the engineered protein comprises at least one essential amino acid residue substitution of a non-essential amino acid residue in the reference secreted protein. In some embodiments, the engineered protein comprises at least one branch chain amino acid residue substitution of a non-branch chain amino acid residue in the reference secreted protein. In some embodiments, the engineered protein comprises at least one Arginine (Arg) or Glutamine (Glu) amino acid residue substitution of a non-Arginine (Arg) or non-Glutamine (Glu) amino acid residue in the reference secreted protein.

In some embodiments, the engineered protein comprises at least one leucine (Leu) amino acid residue substitution of a non-Leu amino acid residue in the reference secreted protein. In some embodiments the Leu amino acid residue substitution is at an amino acid position with a Leu frequency score greater than 0. In some embodiments the Leu amino acid residue substitution is at an amino acid position with a Leu frequency score of at least 0.1. In some embodiments the Leu amino acid residue substitution is at an amino acid position with a branch chain amino acid frequency score of greater than 0. In some embodiments the Leu amino acid residue substitution is at an amino acid position with a branch chain amino acid frequency score of at least 0.1. In some embodiments the Leu amino acid residue substitution is at an amino acid position with a hydrophobic amino acid frequency score of greater than 0. In some embodiments the Leu amino acid residue substitution is at an amino acid position with a hydrophobic amino acid frequency score of at least 0.1. In some embodiments the Leu amino acid residue substitution is at an amino acid position with a per amino acid position entropy of at least 1.5. In some embodiments the difference in total folding free energy between the reference secreted protein and the engineered protein is less than or equal to 0.5.

In some embodiments of the engineered protein, at least two non-leucine (Leu) amino acid residues in the reference secreted protein are substituted by a Leu amino acid residue in the engineered protein, wherein the difference in total folding free energy between the reference secreted protein and the engineered protein is less than or equal to 0.5, and wherein the major energetic component of the total folding free energies for each amino acid substitution is different.

In some embodiments the engineered protein comprises at least one Leu amino acid residue substitution of a non-Leu amino acid residue in a reference secreted protein at a position with a position entropy of at least 1.5. In some embodiments the difference in total folding free energy between the reference secreted protein and the engineered protein is less than or equal to 0.5. In some embodiments the engineered protein comprises at least two Leu amino acid residue substitutions of non-Leu amino acid residues in the reference secreted protein, wherein the contribution to the difference in total folding free energy between the reference secreted protein and the engineered protein from each of the Leu amino acid residue substitutions considered independently is less than or equal to 0.5 and the major energetic component of the total folding free energies for each amino acid substitution is different.

In some embodiments the engineered protein comprises at least one Leu amino acid residue substitution of a non-Leu amino acid residue in a reference secreted protein at a position at which the total free folding energy that results from the Leu substitution is less than or equal to 0.5. In some embodiments the engineered protein comprises at least two Leu amino acid residue substitutions of non-Leu amino acid residues in the reference secreted protein, wherein the contribution to the difference in total folding free energy between the reference secreted protein and the engineered protein from each of the Leu amino acid residue substitutions considered independently is less than or equal to 0.5 and the major energetic component of the total folding free energies for each amino acid substitution is different.

In some embodiments, the engineered protein comprises at least one valine (Val) amino acid residue substitution of a non-Val amino acid residue in the reference secreted protein. In some embodiments the Val amino acid residue substitution is at an amino acid position with a Val frequency score greater than 0. In some embodiments the Val amino acid residue substitution is at an amino acid position with a Val frequency score of at least 0.1. In some embodiments the Val amino acid residue substitution is at an amino acid position with a branch chain amino acid frequency score of greater than 0. In some embodiments the Val amino acid residue substitution is at an amino acid position with a branch chain amino acid frequency score of at least 0.1. In some embodiments the Val amino acid residue substitution is at an amino acid position with a hydrophobic amino acid frequency score of greater than 0. In some embodiments the Val amino acid residue substitution is at an amino acid position with a hydrophobic amino acid frequency score of at least 0.1. In some embodiments the Val amino acid residue substitution is at an amino acid position with a per amino acid position entropy of at least 1.5. In some embodiments the difference in total folding free energy between the reference secreted protein and the engineered protein is less than or equal to 0.5.

In some embodiments of the engineered protein, at least two non-valine (Val) amino acid residues in the reference secreted protein are substituted by a Val amino acid residue in the engineered protein, wherein the difference in total folding free energy between the reference secreted protein and the engineered protein is less than or equal to 0.5, and wherein the major energetic component of the total folding free energies for each amino acid substitution is different.

In some embodiments the engineered protein comprises at least one Val amino acid residue substitution of a non-Val amino acid residue in a reference secreted protein at a position with a position entropy of at least 1.5. In some embodiments the difference in total folding free energy between the reference secreted protein and the engineered protein is less than or equal to 0.5. In some embodiments the engineered protein comprises at least two Val amino acid residue substitutions of non-Val amino acid residues in the reference secreted protein, wherein the contribution to the difference in total folding free energy between the reference secreted protein and the engineered protein from each of the Val amino acid residue substitutions considered independently is less than or equal to 0.5 and the major energetic component of the total folding free energies for each amino acid substitution is different.

In some embodiments the engineered protein comprises at least one Val amino acid residue substitution of a non-Val amino acid residue in a reference secreted protein at a position at which the total free folding energy that results from the Val substitution is less than or equal to 0.5. In some embodiments the engineered protein comprises at least two Val amino acid residue substitutions of non-Val amino acid residues in the reference secreted protein, wherein the contribution to the difference in total folding free energy between the reference secreted protein and the engineered protein from each of the Val amino acid residue substitutions considered independently is less than or equal to 0.5 and the major energetic component of the total folding free energies for each amino acid substitution is different.

In some embodiments, the engineered protein comprises at least one isoleucine (Ile) amino acid residue substitution of a non-Ile amino acid residue in the reference secreted protein. In some embodiments the Ile amino acid residue substitution is at an amino acid position with a Ile frequency score greater than 0. In some embodiments the Ile amino acid residue substitution is at an amino acid position with a Ile frequency score of at least 0.1. In some embodiments the Ile amino acid residue substitution is at an amino acid position with a branch chain amino acid frequency score of greater than 0. In some embodiments the Ile amino acid residue substitution is at an amino acid position with a branch chain amino acid frequency score of at least 0.1. In some embodiments the Ile amino acid residue substitution is at an amino acid position with a hydrophobic amino acid frequency score of greater than 0. In some embodiments the Ile amino acid residue substitution is at an amino acid position with a hydrophobic amino acid frequency score of at least 0.1. In some embodiments the Ile amino acid residue substitution is at an amino acid position with a per amino acid position entropy of at least 1.5. In some embodiments the difference in total folding free energy between the reference secreted protein and the engineered protein is less than or equal to 0.5.

In some embodiments of the engineered protein, at least two non-isoleucine (Ile) amino acid residues in the reference secreted protein are substituted by a Ile amino acid residue in the engineered protein, wherein the difference in total folding free energy between the reference secreted protein and the engineered protein is less than or equal to 0.5, and wherein the major energetic component of the total folding free energies for each amino acid substitution is different.

In some embodiments the engineered protein comprises at least one Ile amino acid residue substitution of a non-Ile amino acid residue in a reference secreted protein at a position with a position entropy of at least 1.5. In some embodiments the difference in total folding free energy between the reference secreted protein and the engineered protein is less than or equal to 0.5. In some embodiments the engineered protein comprises at least two Ile amino acid residue substitutions of non-Ile amino acid residues in the reference secreted protein, wherein the contribution to the difference in total folding free energy between the reference secreted protein and the engineered protein from each of the Ile amino acid residue substitutions considered independently is less than or equal to 0.5 and the major energetic component of the total folding free energies for each amino acid substitution is different.

In some embodiments the engineered protein comprises at least one Ile amino acid residue substitution of a non-Ile amino acid residue in a reference secreted protein at a position at which the total free folding energy that results from the Ile substitution is less than or equal to 0.5. In some embodiments the engineered protein comprises at least two Ile amino acid residue substitutions of non-Ile amino acid residues in the reference secreted protein, wherein the contribution to the difference in total folding free energy between the reference secreted protein and the engineered protein from each of the Ile amino acid residue substitutions considered independently is less than or equal to 0.5 and the major energetic component of the total folding free energies for each amino acid substitution is different.

In some embodiments the reference secreted protein is a naturally occurring protein. In some embodiments the engineered protein is secreted from a compatible microorganism when expressed therein. In some embodiments the compatible microorganism is the same genus as the microorganism that the reference secreted protein naturally occurs in. In some embodiments the microorganism is a heterotroph. In some embodiments the microorganism is photosynthetic. In some embodiments the photosynthetic microorganism is a cyanobacterium.

In some embodiments the amino acid sequence of the engineered protein is at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.5% homologous to the reference secreted protein.

In some embodiments from 5 to 50 non-essential amino acid residues in the reference secreted protein are substituted by essential amino acid residues in the engineered protein. In some embodiments from 5 to 50 non-branch chain amino acid residues in the reference secreted protein are substituted by branch chain amino acid residues in the engineered protein. In some embodiments from 5 to 50 non-Leu amino acid residues in the reference secreted protein are substituted by Leu amino acid residues in the engineered protein. In some embodiments from 5 to 50 non-Val amino acid residues in the reference secreted protein are substituted by Val amino acid residues in the engineered protein. In some embodiments from 5 to 50 non-Ile amino acid residues in the reference secreted protein are substituted by Ile amino acid residues in the engineered protein.

In some embodiments from 5 to 50% of the non-essential amino acid residues in the reference secreted protein are substituted by essential amino acid residues in the engineered protein. In some embodiments from 5 to 50% of the non-branch chain amino acid residues in the reference secreted protein are substituted by branch chain amino acid residues in the engineered protein. In some embodiments from 5 to 50% of the non-Leu amino acid residues in the reference secreted protein are substituted by Leu amino acid residues in the engineered protein. In some embodiments from 5 to 50% of the non-Val amino acid residues in the reference secreted protein are substituted by Val amino acid residues in the engineered protein. In some embodiments from 5 to 50%, e.g., 5 to 10%, 5 to 15%, 5 to 20%, 5 to 25%, 5 to 30%, 5 to 40%, 5 to 45%, 10 to 15%, 10 to 20%, 10 to 25%, 10 to 30%, 10 to 35%, 10 to 40%, 10 to 45%, 15 to 20%, 15 to 25%, 15 to 30%, 15 to 35%, 15 to 40%, 15 to 45%, 20 to 25%, 20 to 30%, 20 to 35%, 20 to 40%, 20 to 45%, 25 to 30%, 25 to 35%, 25 to 40%, 25 to 45%, 30 to 35%, 30 to 40%, 30 to 45%, 35 to 40%, 35 to 45%, or 40 to 45% of the non-Ile amino acid residues in the reference secreted protein are substituted by Ile amino acid residues in the engineered protein.

In some embodiments the engineered protein comprises of: a) a ratio of branch chain amino acid residues to total amino acid residues present in the engineered nutritional protein sequence of at least 26.3%; b.) a ratio of Leu residues to total amino acid residues present in the engineered nutritional protein sequence of at least 11.8%; and c) a ratio of essential amino acid residues to total amino acid residues present in the engineered nutritional protein sequence of at least 55.5%. In some embodiments the engineered protein comprises each essential amino acid. In some embodiments of the engineered protein, the reference secreted protein is from a member of a genus selected from Aspergillus, Trichoderma, Penicillium, Chrysosporium, Acremonium, Fusarium, Trametes, and Rhizopus. In some embodiments of the engineered protein, the reference secreted protein is from a microorganism selected from Escherichia coli, Bacillus subtilis, Saccharomyces cerevisiae, Pichia pastoris, Corynebacterium species, Synechocystis species, and Synechococcus species. In some embodiments of the engineered protein, the reference secreted protein is a protein selected from the proteins listed in Appendix A. In some embodiments of the engineered protein, the reference secreted protein is selected from SEQ ID NOS: 1-9. In some embodiments of the engineered protein, the reference secreted protein comprises a consensus sequence for a fold selected from cellulose binding domain, carbohydrate binding module, fibronectin type III domain, and hydrophobin. In some embodiments of the engineered protein, the reference secreted protein is selected from proteins identified by UniProt Accession Numbers Q4WBW4, Q99034, A1DBP9, Q8NJP6, A1CU44, B0Y8K2, Q4WM08, Q0CMT2, Q8NK02, A1DNL0, A1CCN4, B0XWL3, Q4WFK4, A2QYR9, Q0CFP1, Q5B2E8, A1DJQ7, A1C4H2, B0Y9G4, B8MXJ7, Q4WBU0, Q96WQ9, A2RSN0, Q2US83, Q0CEU4, Q5BCX8, A1DBS6, Q9HE18, O14405, P62694, Q06886, P13860, Q9P8P3, P62695, P07987, A1C8U0, B0Y9E7, B8NIV9, Q4WBS1, Q2U2I3, Q5AR04, A1DBV1, B0YEK2, B8N7Z0, A4DA70, A2R2S6, Q2UI87, Q0CVX4, Q5AX28, A1D9S3, A1CC12, B0Y2K1, Q4WW45, Q5AQZ4, Q99024, P29026, P29027, P69328, P69327, P36914, P23176, P22832, A2QHE1, A1CR85, B0XPE1, B8NRX2, Q4WJJ3, P87076, A2RAL4, Q2UUD6, D0VKF5, Q0CTD7, Q5B5S8, A1D451, B8NJF4, A2QPK4, Q2UNR0, Q5AUW5, B0Y7Q8, B8NP65, Q4WMU3, Q2UN12, Q0CI67, Q5B6C6, A1DMR8, B8NMR5, Q2U325, Q0CUC1, Q5B0F4, A1DC16, A1CUR8, B0XM94, B8NPL7, Q4WL79, Q2U9M7, Q5B6C7, A1DPG0, A1CA51, B0Y3M6, B8NDE2, Q4WU49, A2R989, Q2U8Y5, Q0CAF5, Q5BB53, A1DFA8, B0Y8M8, Q4WLY1, Q5AV15, A1DNN8, Q5BA18, B0YB65, Q4WGT3, Q0CEF3, Q5B9F2, A1DCV5, B0XPB8, B8N5S6, Q4WR62, A5ABF5, Q2UDK7, Q0C7L4, Q5AWD4, A1D122, Q5B681, Q5BG51, A1CCL9, Q0CB82, Q5ATH9, Q4AEG8, B0XP71, B8MYV0, Q4WRB0, A2QA27, O00089, Q2UR38, Q0CMH8, Q5BAS1, P29026, P29027, P48827, A1CIA7, B0Y708, P35211, B8N106, P28296, P12547, Q00208, A1CWF3, P52750, P52754, P79073, P52755, P41746, and P28346. The sequences indicated by the accession numbers provided herein are those sequences in the database as of the filing date of the instant application.

In some embodiments the engineered protein is selected from SEQ ID NOS: 10-13. In some embodiments the engineered protein further comprises a polypeptide tag for affinity purification. In some embodiments the tag for affinity purification is a polyhistidine-tag. In some embodiments the engineered protein has a net absolute per amino acid charge of at least 0.05 at pH 7. In some embodiments the engineered protein has a net absolute per amino acid charge of at least 0.10 at pH 7. In some embodiments the engineered protein has a net absolute per amino acid charge of at least 0.15 at pH 7. In some embodiments the engineered protein has a net absolute per amino acid charge of at least 0.20 at pH 7. In some embodiments the engineered protein has a net absolute per amino acid charge of at least 0.25 at pH 7. In some embodiments the engineered protein has a net positive charge at pH 7. In some embodiments the engineered protein has a net negative charge at pH 7. In some embodiments the engineered protein is digestible. In some embodiments the engineered protein comprises a protease recognition site selected from a pepsin recognition site, a trypsin recognition site, and a chymotrypsin recognition site.

In a further aspect, this disclosure provides nucleic acids, including in some embodiments isolated nucleic acids. In some embodiments the nucleic acid comprises a nucleic acid sequence that encodes an engineered protein of this disclosure. In some embodiments the nucleic acid further comprises an expression control sequence operatively linked to the nucleic acid sequence that encodes the engineered protein.

In a further aspect, this disclosure provides vectors. In some embodiments the vectors comprise a nucleic acid sequence that encodes an engineered protein of this disclosure. In some embodiments the vector further comprises an expression control sequence operatively linked to the nucleic acid sequence that encodes the engineered protein.

In a further aspect, this disclosure provides recombinant microorganisms. In some embodiments the recombinant microorganism comprises at least one of a) a nucleic acid that encodes an engineered protein of this disclosure and b) a vector comprising a nucleic acid that encodes an engineered protein of this disclosure. In some embodiments, the recombinant microorganism is a prokaryote. In some embodiments, the prokaryote is heterotrophic. In some embodiments, the prokaryote is autotrophic. In some embodiments, the prokaryote is a bacteria.

In a further aspect, this disclosure provides methods of making a recombinant engineered protein of this disclosure. In some embodiments the methods comprise culturing a recombinant microorganism of this disclosure under conditions sufficient for production of the recombinant engineered protein by the recombinant microorganism. In some embodiments the methods further comprise isolating the recombinant engineered protein from the culture. In some embodiments the recombinant protein is soluble. In some embodiments, the recombinant engineered protein is secreted by the cultured recombinant microorganism and the secreted protein is isolated from the culture medium.

In a further aspect, this disclosure provides nutritive compositions. In some embodiments the nutritive compositions comprise an engineered protein of this disclosure and at least one second component. In some embodiments the second component is selected from a protein, a polypeptide, a peptide, a free amino acid, a carbohydrate, a fat, a mineral or mineral source, a vitamin, and an excipient. In some embodiments the second component is a protein. In some embodiments the protein is an engineered protein. In some embodiments the second component is a free amino acid selected from essential amino acids. In some embodiments the second component is a free amino acid selected from branch chain amino acids. In some embodiments the second component is Leu. In some embodiments the second component is Val. In some embodiments the second component is Ile. In some embodiments the second component is an excipient. In some embodiments the excipient is selected from a buffering agent, a preservative, a stabilizer, a binder, a compaction agent, a lubricant, a dispersion enhancer, a disintegration agent, a flavoring agent, a sweetener, a coloring agent. In some embodiments the nutritive composition is formulated as a liquid solution, slurry, suspension, gel, paste, powder, or solid.

In a further aspect, this disclosure provides methods of making a nutritive composition. In some embodiments the methods comprise providing an engineered protein of this disclosure and combining the engineered protein with second component. In some embodiments the second component is selected from a protein, a polypeptide, a peptide, a free amino acid, a carbohydrate, a fat, a mineral or mineral source, a vitamin, and an excipient. In some embodiments the second component is a protein. In some embodiments the second component is a free amino acid selected from essential amino acids. In some embodiments the second component is a free amino acid selected from branch chain amino acids. In some embodiments the second component is Leu. In some embodiments the second component is Val. In some embodiments the second component is Ile. In some embodiments the second component is an excipient. In some embodiments the excipient is selected from a buffering agent, a preservative, a stabilizer, a binder, a compaction agent, a lubricant, a dispersion enhancer, a disintegration agent, a flavoring agent, a sweetener, a coloring agent. In some embodiments the nutritive composition is formulated as a liquid solution, slurry, suspension, gel, paste, powder, or solid.

In a further aspect, this disclosure provides methods of maintaining or increasing at least one of muscle mass, muscle strength, and functional performance in a subject. In some embodiments the methods comprise providing to the subject a sufficient amount of an engineered protein according to disclosure, a nutritive composition according to disclosure, or a nutritive composition made by a method according to disclosure. In some embodiments the subject is at least one of elderly, critically-medically ill, and suffering from protein-energy malnutrition. In some embodiments, the engineered protein according to disclosure, the nutritive composition according to disclosure, or the nutritive composition made by a method according to disclosure is consumed by the subject in coordination with performance of exercise. In some embodiments, the engineered protein according to disclosure, the nutritive composition according to disclosure, or the nutritive composition made by a method according to disclosure is consumed by the subject by an oral, enteral, or parenteral route.

In a further aspect, this disclosure provides methods of maintaining or achieving a desirable body mass index in a subject. In some embodiments the methods comprise providing to the subject a sufficient amount of an engineered protein of this disclosure, a nutritive composition of this disclosure, or a nutritive composition made by a method of this disclosure. In some embodiments the subject is at least one of elderly, critically-medically ill, and suffering from protein-energy malnutrition. In some embodiments, the engineered protein according to disclosure, the nutritive composition according to disclosure, or the nutritive composition made by a method according to disclosure is consumed by the subject in coordination with performance of exercise. In some embodiments, the engineered protein according to disclosure, the nutritive composition according to disclosure, or the nutritive composition made by a method according to disclosure is consumed by the subject by an oral, enteral, or parenteral route.

In a further aspect, this disclosure provides methods of providing protein to a subject with protein-energy malnutrition. In some embodiments the methods comprise providing to the subject a sufficient amount of an engineered protein of this disclosure, a nutritive composition of this disclosure, or a nutritive composition of this disclosure. In some embodiments, the engineered protein according to disclosure, the nutritive composition according to disclosure, or the nutritive composition made by a method according to disclosure is consumed by the subject by an oral, enteral, or parenteral route.

In a further aspect, this disclosure provides methods of making an engineered protein. In some embodiments the methods comprise a) providing a reference secreted protein, b) identifying a set of amino acid positions of the reference secreted protein to mutate to improve the nutritive content of the protein, and c) synthesizing the engineered protein comprising the target amino acid substitutions. In some embodiments the reference secreted protein is from a member of a genus selected from Aspergillus, Trichoderma, Penicillium, Chrysosporium, Acremonium, Fusarium, Trametes, and Rhizopus. In some embodiments the reference secreted protein is from a microorganism selected from Escherichia coli, Bacillus subtilis, Saccharomyces cerevisiae, Pichia pastoris, Corynebacterium species, Synechocystis species, and Synechococcus species. In some embodiments the reference secreted protein is a protein listed in Appendix A. In some embodiments the reference secreted protein is a protein selected from proteins identified by UniProt Accession Numbers Q4WBW4, Q99034, A1DBP9, Q8NJP6, A1CU44, B0Y8K2, Q4WM08, Q0CMT2, Q8NK02, A1DNL0, A1CCN4, B0XWL3, Q4WFK4, A2QYR9, Q0CFP1, Q5B2E8, A1DJQ7, A1C4H2, B0Y9G4, B8MXJ7, Q4WBU0, Q96WQ9, A2R5NO, Q2US83, Q0CEU4, Q5BCX8, A1DBS6, Q9HE18, O14405, P62694, Q06886, P13860, Q9P8P3, P62695, P07987, A1C8U0, B0Y9E7, B8NIV9, Q4WBS1, Q2U2I3, Q5AR04, A1DBV1, B0YEK2, B8N7Z0, A4DA70, A2R2S6, Q2UI87, Q0CVX4, Q5AX28, A1D9S3, A1CC12, B0Y2K1, Q4WW45, Q5AQZ4, Q99024, P29026, P29027, P69328, P69327, P36914, P23176, P22832, A2QHE1, A1CR85, B0XPE1, B8NRX2, Q4WJJ3, P87076, A2RAL4, Q2UUD6, D0VKF5, Q0CTD7, Q5B5S8, A1D451, B8NJF4, A2QPK4, Q2UNR0, Q5AUW5, B0Y7Q8, B8NP65, Q4WMU3, Q2UN12, Q0CI67, Q5B6C6, A1DMR8, B8NMR5, Q2U325, Q0CUC1, Q5B0F4, A1DC16, A1CUR8, B0XM94, B8NPL7, Q4WL79, Q2U9M7, Q5B6C7, A1DPG0, A1CA51, B0Y3M6, B8NDE2, Q4WU49, A2R989, Q2U8Y5, Q0CAF5, Q5BB53, A1DFA8, B0Y8M8, Q4WLY1, Q5AV15, A1DNN8, Q5BA18, B0YB65, Q4WGT3, Q0CEF3, Q5B9F2, A1DCV5, B0XPB8, B8N5S6, Q4WR62, A5ABF5, Q2UDK7, Q0C7L4, Q5AWD4, A1D122, Q5B681, Q5BG51, A1CCL9, Q0CB82, Q5ATH9, Q4AEG8, B0XP71, B8MYV0, Q4WRB0, A2QA27, O00089, Q2UR38, Q0CMH8, Q5BAS1, P29026, P29027, P48827, A1CIA7, B0Y708, P35211, B8N106, P28296, P12547, Q00208, A1CWF3, P52750, P52754, P79073, P52755, P41746, and P28346. In some embodiments the reference secreted protein is selected from SEQ ID NOS: 1-9. In some embodiments the reference secreted protein comprises a consensus sequence for a fold selected from a cellulose binding domain, carbohydrate binding mudule, fibronectin type III domain, and hydrophobin.

In some embodiments identifying the set of amino acid positions of the reference secreted protein to mutate to improve the nutritive content of the protein comprises determining at least one parameter selected from amino acid likelihood (AALike), amino acid type likelihood (AATLike), position entropy (Sₚₒₛ), amino acid type position entropy (S_{AATpos}), relative free energy of folding (ΔΔG_{fold}), and secondary structure identity (LoopID) for a plurality of amino acid positions of the reference secreted protein. In some embodiments a combination of two or more parameters is determined for a plurality of amino acid positions of the reference secreted protein, wherein the combination of parameters is selected from: (A) AAlike and ΔΔG_{fold}, (B) AATlike and ΔΔG_{fold}, (C) AAlike, AATlike, and ΔΔG_{fold}, (D) Sₚₒₛ and ΔΔG_{fold}, (E) S_{AATpos} and ΔΔG_{fold}, (F) LoopID and ΔΔG_{fold}, (G) AAlike, ΔΔG_{fold}, and LoopID, (H) AAlike, AATlike, ΔΔG_{fold}, and LoopID, (I) AATlike, ΔΔG_{fold}, and LoopID, (J) Sₚₒₛ, ΔΔG_{fold}, and LoopID, and (K) S_{AATpos}, ΔΔG_{fold}, and LoopID. In some embodiments the method further comprises ranking the plurality of amino acid positions of the reference secreted protein on the basis of the parameter and mutating the amino acids at positions having at least a threshold parameter value.

In some embodiments the engineered protein is synthesized in vivo. In some embodiments the engineered protein is synthesized in vitro.

### BRIEF DESCRIPTION OF THE DRAWINGS

FIG. 1 shows leucine replacement based on amino acid likelihood in the glucoamylase protein from A. niger (SEQ ID NO: 1). FIG. 1A shows leucine replacement based on leucine likelihood, and FIG. 1B shows a blown up view of the left end of the graph in FIG. 1A. FIG. 1C shows leucine replacement based on branch chain amino acid (BCAA) likelihood and FIG. 1D shows leucine replacement based on hydrophobic amino acid (A, M, I, L, V) likelihood.

FIG. 2 shows leucine replacement based on position entropy in the glucoamylase protein from A. niger (SEQ ID NO: 1). In FIG. 2A position entropy is calculated based on the full set of twenty amino acids, while in FIG. 2B it is calculated based on 5 groups of amino acids that have similar biophysical properties: hydrophobic [A,V,I,L,M], aromatic [F,Y,W], polar [S,T,N,Q], charged [R,H,K,D,E], other [G,P,C].

FIG. 3 shows leucine replacement mutation free folding energies relative to wild type for each amino acid position in the glucoamylase protein from A. niger (SEQ ID NO: 1).

FIG. 4 shows leucine replacement based on amino acid likelihood in the endo-beta-1,4-glucanase protein from A. niger (SEQ ID NO: 2). FIG. 4A shows leucine replacement based on leucine likelihood, and FIG. 4B shows a blown up view of the left end of the graph in FIG. 4A. FIG. 4C shows leucine replacement based on branch chain amino acid (BCAA) likelihood and FIG. 4D shows leucine replacement based on hydrophobic amino acid (A, M, I, L, V) likelihood.

FIG. 5 shows leucine replacement based on position entropy in the endo-beta-1,4-glucanase protein from A. niger (SEQ ID NO: 2). In FIG. 5A position entropy is calculated based on the full set of twenty amino acids, while in FIG. 5B it is calculated based on 5 groups of amino acids that have similar biophysical properties: hydrophobic [A,V,I,L,M], aromatic [F,Y,W], polar [S,T,N,Q], charged [R,H,K,D,E], other [G,P,C].

FIG. 6 shows leucine replacement mutation free folding energies relative to wild type for each amino acid position in the endo-beta-1,4-glucanase protein from A. niger (SEQ ID NO: 2).

FIG. 7 shows leucine replacement based on amino acid likelihood in the 1,4-beta-D-glucan cellobiohydrolase protein from A. niger (SEQ ID NO: 3). FIG. 7A shows leucine replacement based on leucine likelihood, and FIG. 7B shows a blown up view of the left end of the graph in FIG. 7A. FIG. 7C shows leucine replacement based on branch chain amino acid (BCAA) likelihood and FIG. 7D shows leucine replacement based on hydrophobic amino acid (A, M, I, L, V) likelihood.

FIG. 8 shows leucine replacement based on position entropy in the 1,4-beta-D-glucan cellobiohydrolase protein from A. niger (SEQ ID NO: 3). In FIG. 8A position entropy is calculated based on the full set of twenty amino acids, while in FIG. 8B it is calculated based on 5 groups of amino acids that have similar biophysical properties: hydrophobic [A,V,I,L,M], aromatic [F,Y,W], polar [S,T,N,Q], charged [R,H,K,D,E], other [G,P,C].

FIG. 9 shows leucine replacement mutation free folding energies relative to wild type for each amino acid position in the 1,4-beta-D-glucan cellobiohydrolase protein from A. niger (SEQ ID NO: 3).

FIG. 10 shows leucine replacement based on amino acid likelihood in the endo-1,4-beta-xylanase protein from A. niger (SEQ ID NO: 4). FIG. 10A shows leucine replacement based on leucine likelihood, and FIG. 10B shows a blown up view of the left end of the graph in FIG. 10A. FIG. 10C shows leucine replacement based on branch chain amino acid (BCAA) likelihood and FIG. 10D shows leucine replacement based on hydrophobic amino acid (A, M, I, L, V) likelihood.

FIG. 11 shows leucine replacement based on position entropy in the endo-1,4-beta-xylanase protein from A. niger (SEQ ID NO: 4). In FIG. 11A position entropy is calculated based on the full set of twenty amino acids, while in FIG. 11B it is calculated based on 5 groups of amino acids that have similar biophysical properties: hydrophobic [A,V,I,L,M], aromatic [F,Y,W], polar [S,T,N,Q], charged [R,H,K,D,E], other [G,P,C].

FIG. 12 shows leucine replacement mutation free folding energies relative to wild type for each amino acid position in the endo-1,4-beta-xylanase protein from A. niger (SEQ ID NO: 4).

FIG. 13 shows leucine replacement based on amino acid likelihood in the cellulose binding domain 1 from A. niger (SEQ ID NO: 5). FIG. 13A shows leucine replacement based on leucine likelihood, and FIG. 13B shows a blown up view of the left end of the graph in FIG. 13A. FIG. 13C shows leucine replacement based on branch chain amino acid (BCAA) likelihood and FIG. 13D shows leucine replacement based on hydrophobic amino acid (A, M, I, L, V) likelihood.

FIG. 14 shows leucine replacement based on position entropy in cellulose binding domain 1 from A. niger (SEQ ID NO: 5). In FIG. 14A position entropy is calculated based on the full set of twenty amino acids, while in FIG. 14B it is calculated based on 5 groups of amino acids that have similar biophysical properties: hydrophobic [A,V,I,L,M], aromatic [F,Y,W], polar [S,T,N,Q], charged [R,H,K,D,E], other [G,P,C].

FIG. 15 shows leucine replacement mutation free folding energies relative to wild type for each amino acid position in cellulose binding domain 1 from A. niger (SEQ ID NO: 5).

FIG. 16 shows leucine replacement based on amino acid likelihood in carbohydrate binding module 20 from A. niger (SEQ ID NO: 6). FIG. 16A shows leucine replacement based on leucine likelihood, and FIG. 16B shows a blown up view of the left end of the graph in FIG. 16A. FIG. 16C shows leucine replacement based on branch chain amino acid (BCAA) likelihood and FIG. 16D shows leucine replacement based on hydrophobic amino acid (A, M, I, L, V) likelihood.

FIG. 17 shows isoleucine replacement based on amino acid likelihood in carbohydrate binding module 20 from A. niger (SEQ ID NO: 6). FIG. 17A shows isoleucine replacement based on isoleucine likelihood, and FIG. 17B shows a blown up view of the left end of the graph in FIG. 17A. FIG. 17C shows isoleucine replacement based on branch chain amino acid (BCAA) likelihood and FIG. 17D shows isoleucine replacement based on hydrophobic amino acid (A, M, I, L, V) likelihood.

FIG. 18 shows valine replacement based on amino acid likelihood in carbohydrate binding module 20 from A. niger (SEQ ID NO: 6). FIG. 18A shows valine replacement based on valine likelihood, and FIG. 18B shows a blown up view of the left end of the graph in FIG. 18A. FIG. 18C shows valine replacement based on branch chain amino acid (BCAA) likelihood and FIG. 18D shows valine replacement based on hydrophobic amino acid (A, M, I, L, V) likelihood.

FIG. 19 shows leucine replacement based on position entropy in carbohydrate binding module 20 from A. niger (SEQ ID NO: 6). In FIG. 19A position entropy is calculated based on the full set of twenty amino acids, while in FIG. 19B it is calculated based on 5 groups of amino acids that have similar biophysical properties: hydrophobic [A,V,I,L,M], aromatic [F,Y,W], polar [S,T,N,Q], charged [R,H,K,D,E], other [G,P,C].

FIG. 20 shows leucine replacement mutation free folding energies relative to wild type for each amino acid position in carbohydrate binding module 20 from A. niger (SEQ ID NO: 6).

FIG. 21 shows isoleucine replacement mutation free folding energies relative to wild type for each amino acid position in carbohydrate binding module 20 from A. niger (SEQ ID NO: 6).

FIG. 22 shows valine replacement mutation free folding energies relative to wild type for each amino acid position in carbohydrate binding module 20 from A. niger (SEQ ID NO: 6).

FIG. 23 shows arginine replacement mutation free folding energies relative to wild type for each amino acid position in carbohydrate binding module 20 from A. niger (SEQ ID NO: 6).

FIG. 24 shows leucine replacement based on amino acid likelihood in glucosidase fibronectin type III domain from A. niger (SEQ ID NO: 7). FIG. 24A shows leucine replacement based on leucine likelihood, and FIG. 24B shows a blown up view of the left end of the graph in FIG. 24A. FIG. 24C shows leucine replacement based on branch chain amino acid (BCAA) likelihood and FIG. 24D shows leucine replacement based on hydrophobic amino acid (A, M, I, L, V) likelihood.

FIG. 25 shows leucine replacement based on position entropy in glucosidase fibronectin type III domain from A. niger (SEQ ID NO: 7). In FIG. 25A position entropy is calculated based on the full set of twenty amino acids, while in FIG. 25B it is calculated based on 5 groups of amino acids that have similar biophysical properties: hydrophobic [A,V,I,L,M], aromatic [F,Y,W], polar [S,T,N,Q], charged [R,H,K,D,E], other [G,P,C].

FIG. 26 shows leucine replacement mutation free folding energies relative to wild type for each amino acid position in glucosidase fibronectin type III domain from A. niger (SEQ ID NO: 7).

FIG. 27 shows leucine replacement based on amino acid likelihood in the hydrophobin I protein from T. Reesei (SEQ ID NO: 8). FIG. 27A shows leucine replacement based on leucine likelihood, and FIG. 27B shows a blown up view of the left end of the graph in FIG. 27A. FIG. 27C shows leucine replacement based on branch chain amino acid (BCAA) likelihood and FIG. 27D shows leucine replacement based on hydrophobic amino acid (A, M, I, L, V) likelihood.

FIG. 28 shows leucine replacement based on position entropy in the hydrophobin I protein from T. Reesei (SEQ ID NO: 8). In FIG. 28A position entropy is calculated based on the full set of twenty amino acids, while in FIG. 28B it is calculated based on 5 groups of amino acids that have similar biophysical properties: hydrophobic [A,V,I,L,M], aromatic [F,Y,W], polar [S,T,N,Q], charged [R,H,K,D,E], other [G,P,C].

FIG. 29 shows leucine replacement mutation free folding energies relative to wild type for each amino acid position in the hydrophobin I protein from T. Reesei (SEQ ID NO: 8).

FIG. 30 shows leucine replacement based on amino acid likelihood in the hydrophobin II protein from T. Reesei (SEQ ID NO: 9). FIG. 30A shows leucine replacement based on leucine likelihood, and FIG. 30B shows a blown up view of the left end of the graph in FIG. 30A. FIG. 30C shows leucine replacement based on branch chain amino acid (BCAA) likelihood and FIG. 30D shows leucine replacement based on hydrophobic amino acid (A, M, I, L, V) likelihood.

FIG. 31 shows leucine replacement based on position entropy in the hydrophobin II protein from T. Reesei (SEQ ID NO: 9). In FIG. 31A position entropy is calculated based on the full set of twenty amino acids, while in FIG. 31B it is calculated based on 5 groups of amino acids that have similar biophysical properties: hydrophobic [A,V,I,L,M], aromatic [F,Y,W], polar [S,T,N,Q], charged [R,H,K,D,E], other [G,P,C].

FIG. 32 shows leucine replacement mutation free folding energies relative to wild type for each amino acid position in the hydrophobin II protein from T. Reesei (SEQ ID NO: 9).

FIG. 33 shows a schematic illustration of a library construction strategy used for making SEQID-45001 and SEQID-45029 variants.

FIGS. 34A and 34B show the result of secretion screening using the Caliper LabChip GXII. (A) Electropherograms demonstrating a hit (protein of interest peak indicated with arrow), negative control, and protein ladder. (B) Simulated gel images generated from electropherograms demonstrating secretion of protein variants (protein of interest peak in box).

FIG. 35 shows the results of anti-FLAG dotblot analysis of Aspergillus culture supernatants. (A) Isolates transformed with expression vectors encoding specific variants of SEQID-45029. Box indicates standard curve. (B) Quantification of positive wells from (A). SEQID-45029 is a positive control for wild type secretion. (C) Isolates transformed with expression vectors encoding a library of SEQID-45029 variants. (D). Quantification of positive wells from (C) based on standard curve (box).

FIG. 36 demonstrates the sequence diversity of isolate 18 and 27 expression cassettes. Numerals following the dash indicate specific sub-clone. Boxes indicate identical sequences. Clones suggesting the presence of deletions outside of the variable regions are indicated with an asterix. Figure discloses “Pos1” sequences as SEQ ID NOS 22014-22044, “Pos2” sequences as SEQ ID NOS 22045-22075, “Pos3” sequences as SEQ ID NOS 22076-22106, and “Pos4” sequences as SEQ ID NOS 22107-22137, all respectively, in order of appearance.

### DESCRIPTION OF APPENDICES

This specification includes Appendices A-D.

Appendix A lists exemplary reference secreted proteins.

Appendix B lists representative proteins that include folds/domains selected from ankyrin repeats, Leucine rich repeats, tetratricopeptide repeats, armadillo repeats, fibronectine type III domains, lipocalin-like domains, knottins, cellulose binding domains, carbohydrate binding domains, protein Z folds, PDZ domains, SH3 domains, SH2 domains, WW domains, thioredoxins, Leucine zipper, plant homeodomain, tudor domain, and hydrophobins.

Appendix C lists proteins used in multiple sequence alignments (MSAs) to analyze amino acid likelihood.

Appendix D presents analyses of the physiochemical properties of the protein and polypeptide sequences analyzed in the examples.

### DETAILED DESCRIPTION

Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art. Certain references and other documents cited herein are expressly incorporated herein by reference. Additionally, all UniProt/SwissProt records cited herein are hereby incorporated herein by reference. In case of conflict, the present specification, including definitions, will control. The materials, methods, and examples are illustrative only and not intended to be limiting.

The methods and techniques of the present disclosure are generally performed according to conventional methods well known in the art and as described in various general and more specific references that are cited and discussed throughout the present specification unless otherwise indicated. See, e.g., Sambrook et al., Molecular Cloning: A Laboratory Manual, 3d ed., Cold Spring Harbor Laboratory Press, Cold Spring Harbor, N.Y. (2001); Ausubel et al., Current Protocols in Molecular Biology, Greene Publishing Associates (1992, and Supplements to 2002); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999). Remington's Pharmaceutical Sciences, Mack Pub. Co, Easton, Pa. (18^{th} ed.) (1990). Many molecular biology and genetic techniques applicable to cyanobacteria are described in Heidorn et al., “Synthetic Biology in Cyanobacteria: Engineering and Analyzing Novel Functions,” Methods in Enzymology, Vol. 497, Ch. 24 (2011), which is hereby incorporated herein by reference.

This disclosure refers to sequence database entries (e.g., UniProt/SwissProt records) for certain protein and gene sequences that are published on the internet, as well as other information on the internet. The skilled artisan understands that information on the internet, including sequence database entries, is updated from time to time and that, for example, the reference number used to refer to a particular sequence can change. Where reference is made to a public database of sequence information or other information on the internet, it is understood that such changes can occur and particular embodiments of information on the internet can come and go. Because the skilled artisan can find equivalent information by searching on the internet, a reference to an internet web page address or a sequence database entry evidences the availability and public dissemination of the information in question. In all cases the sequence information contained in the sequence database entries referenced herein is hereby incorporated herein by reference.

Before the present proteins, compositions, methods, and other embodiments are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

The term “comprising” as used herein is synonymous with “including” or “containing”, and is inclusive or open-ended and does not exclude additional, unrecited members, elements or method steps.

This disclosure makes reference to amino acids. The full name of the amino acids is used interchangeably with the standard three letter and one letter abbreviations for each. For the avoidance of doubt, those are: Alanine (Ala, A), Arginine (Arg, R), Asparagine (Asn, N), Aspartic acid (Asp, D), Cysteine (Cys, C), Glutamic Acid (Glu, E), Glutamine (Gln, Q), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), Valine (Val, V).

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe).

As used herein, the term “isolated” refers to a substance or entity that has been (1) separated from at least some of the components with which it was associated when initially produced (whether in nature or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

As used herein, a “branch chain amino acid” is an amino acid selected from Leucine, Isoleucine, and Valine.

As used herein, an “essential amino acid” is an amino acid selected from Histidine, Isoleucine, Leucine, Lysine, Methionine, Phenylalanine, Threonine, Tryptophan, and Valine.

The term “peptide” as used herein refers to a short polypeptide, e.g., one that typically contains less than about 50 amino acids and more typically less than about 30 amino acids. The term as used herein encompasses analogs and mimetics that mimic structural and thus biological function.

The terms “polypeptide” and “protein” can be interchanged, and these terms encompass both naturally-occurring and non-naturally occurring polypeptides, and, as provided herein or as generally known in the art, fragments, mutants, derivatives and analogs thereof. A polypeptide can be monomeric, meaning it has a single chain, or polymeric, meaning it is composed of two or more chains, which can be covalently or non-covalently associated. Further, a polypeptide may comprise a number of different domains each of which has one or more distinct activities. For the avoidance of doubt, a polypeptide can be any length greater than or equal to two amino acids.

The term “isolated polypeptide” is a polypeptide that by virtue of its origin or source of derivation (1) is not associated with naturally associated components that accompany it in any of its native states, (2) exists in a purity not found in nature, where purity can be adjudged with respect to the presence of other cellular material (e.g., is free of other polypeptides from the same species or from the host species in which the polypeptide was produced) (3) is expressed by a cell from a different species, (4) is recombinantly expressed by a cell (e.g., a polypeptide is an “isolated polypeptide” if it is produced from a recombinant nucleic acid present in a host cell and separated from the producing host cell, (5) does not occur in nature (e.g., it is a domain or other fragment of a polypeptide found in nature or it includes amino acid analogs or derivatives not found in nature or linkages other than standard peptide bonds), or (6) is otherwise produced, prepared, and/or manufactured by the hand of man. Thus, an “isolated polypeptide” includes a polypeptide that is produced in a host cell from a recombinant nucleic acid (such as a vector), regardless of whether the host cell naturally produces a polypeptide having an identical amino acid sequence. A “polypeptide” includes a polypeptide that is produced by a host cell via overexpression, e.g., homologous overexpression of the polypeptide from the host cell such as by altering the promoter of the polypeptide to increase its expression to a level above its normal expression level in the host cell in the absence of the altered promoter. A polypeptide that is chemically synthesized or synthesized in a cellular system different from a cell from which it naturally originates is “isolated” from its naturally associated components. A polypeptide may also be rendered substantially free of naturally associated components by isolation, using protein purification techniques well known in the art. As thus defined, “isolated” does not necessarily require that the protein, polypeptide, peptide or oligopeptide so described has been physically removed from a cell in which it was synthesized.

The terms “purify,” “purifying” and “purified” refer to a substance (or entity, composition, product or material) that has been separated from at least some of the components with which it was associated either when initially produced (whether in nature or in an experimental setting), or during any time after its initial production. A substance such as a nutritional polypeptide is considered purified if it is isolated at production, or at any level or stage up to and including a final product, but a final product may contain other materials up to about 10%, about 20%, about 30%, about 40%, about 50%, about 60%, about 70%, about 80%, about 90%, or above about 90% and still be considered “isolated.” Purified substances or entities can be separated from at least about 10%, about 20%, about 30%, about 40%, about 50%, about 60%, about 70%, about 80%, about 90%, or more of the other components with which they were initially associated. In some embodiments, purified substances are more than about 80%, about 85%, about 90%, about 91%, about 92%, about 93%, about 94%, about 95%, about 96%, about 97%, about 98%, about 99%, or more than about 99% pure. In the instance of polypeptides and other polypeptides provided herein, such a polypeptide can be purified from one or more other polypeptides capable of being secreted from the unicellular organism that secretes the polypeptide. As used herein, a polypeptide substance is “pure” if it is substantially free of other components or other polypeptide components.

The term “polypeptide fragment” or “protein fragment” as used herein refers to a polypeptide or domain thereof that has less amino acids compared to a reference polypeptide, e.g., a full-length polypeptide or a polypeptide domain of a naturally occurring protein. A “naturally occurring protein” or “naturally occurring polypeptide” includes a polypeptide having an amino acid sequence produced by a non-recombinant cell or organism. In an embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, or at least 12, 14, 16 or 18 amino acids long, or at least 20 amino acids long, or at least 25, 30, 35, 40 or 45, amino acids, or at least 50, 60, 70, 80, 90 or 100 amino acids long, or at least 110, 120, 130, 140, 150, 160, 170, 180, 190 or 200 amino acids long, or 225, 250, 275, 300, 325, 350, 375, 400, 425, 450, 475, 500, 525, 550, 575, 600 or greater than 600 amino acids long. A fragment can be a portion of a larger polypeptide sequence that is digested inside or outside the cell. Thus, a polypeptide that is 50 amino acids in length can be produced intracellularly, but proteolyzed inside or outside the cell to produce a polypeptide less than 50 amino acids in length. This is of particular significance for polypeptides shorter than about 25 amino acids, which can be more difficult than larger polypeptides to produce recombinantly or to purify once produced recombinantly. The term “peptide” as used herein refers to a short polypeptide or oligopeptide, e.g., one that typically contains less than about 50 amino acids and more typically less than about 30 amino acids, or more typically less than about 15 amino acids, such as less than about 10, 9, 8, 7, 6, 5, 4, or 3 amino acids. The term as used herein encompasses analogs and mimetics that mimic structural and thus biological function.

The term “fusion protein” refers to a polypeptide comprising a polypeptide or fragment coupled to heterologous amino acid sequences. Fusion proteins are useful because they can be constructed to contain two or more desired functional elements that can be from two or more different proteins. A fusion protein comprises at least 10 contiguous amino acids from a polypeptide of interest, or at least 20 or 30 amino acids, or at least 40, 50 or 60 amino acids, or at least 75, 100 or 125 amino acids. The heterologous polypeptide included within the fusion protein is usually at least 6 amino acids in length, or at least 8 amino acids in length, or at least 15, 20, or 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

A composition, formulation or product is “nutritional” or “nutritive” if it provides an appreciable amount of nourishment to its intended consumer, meaning the consumer assimilates all or a portion of the composition or formulation into a cell, organ, and/or tissue. Generally such assimilation into a cell, organ and/or tissue provides a benefit or utility to the consumer, e.g., by maintaining or improving the health and/or natural function(s) of said cell, organ, and/or tissue. A nutritional composition or formulation that is assimilated as described herein is termed “nutrition.” By way of non-limiting example, a polypeptide is nutritional if it provides an appreciable amount of polypeptide nourishment to its intended consumer, meaning the consumer assimilates all or a portion of the protein, typically in the form of single amino acids or small peptides, into a cell, organ, and/or tissue. “Nutrition” also means the process of providing to a subject, such as a human or other mammal, a nutritional composition, formulation, product or other material. A nutritional product need not be “nutritionally complete,” meaning if consumed in sufficient quantity, the product provides all carbohydrates, lipids, essential fatty acids, essential amino acids, conditionally essential amino acids, vitamins, and minerals required for health of the consumer. Additionally, a “nutritionally complete protein” contains all protein nutrition required (meaning the amount required for physiological normalcy by the organism) but does not necessarily contain micronutrients such as vitamins and minerals, carbohydrates or lipids.

In preferred embodiments, a composition or formulation is nutritional in its provision of polypeptide capable of decomposition (i.e., the breaking of a peptide bond, often termed protein digestion) to single amino acids and/or small peptides (e.g., two amino acids, three amino acids, or four amino acids, possibly up to ten amino acids) in an amount sufficient to provide a “nutritional benefit.” In addition, in certain embodiments provided are nutritional polypeptides that transit across the gastrointestinal wall and are absorbed into the bloodstream as small peptides (e.g., larger than single amino acids but smaller than about ten amino acids) or larger peptides, oligopeptides or polypeptides (e.g., >11 amino acids). A nutritional benefit in a polypeptide-containing composition can be demonstrated and, optionally, quantified, by a number of metrics. For example, a nutritional benefit is the benefit to a consuming organism equivalent to or greater than at least about 0.5% of a reference daily intake value of protein, such as about 1%, 2%, 3%, 4%, 5%, 6%, 7%, 8%, 9%, 10%, 15%, 20%, 25%, 30%, 35%, 40%, 45%, 50%, 60%, 65%, 70%, 75%, 80%, 85%, 90%, 95%, 100% or greater than about 100% of a reference daily intake value. Alternatively, a nutritional benefit is demonstrated by the feeling and/or recognition of satiety by the consumer. In other embodiments, a nutritional benefit is demonstrated by incorporation of a substantial amount of the polypeptide component of the composition or formulation into the cells, organs and/or tissues of the consumer, such incorporation generally meaning that single amino acids or short peptides are used to produce polypeptides de novo intracellularly. A “consumer” or a “consuming organism” means any animal capable of ingesting the product having the nutritional benefit. Typically, the consumer is a mammal such as a healthy human, e.g., a healthy infant, child, adult, or older adult. Alternatively, the consumer is a mammal such as a human (e.g., an infant, child, adult or older adult) at risk of developing or suffering from a disease, disorder or condition characterized by (i) the lack of adequate nutrition and/or (ii) the alleviation thereof by the nutritional products of the present invention. An “infant” is generally a human under about age 1 or 2, a “child” is generally a human under about age 18, and an “older adult” or “elderly” human is a human aged about 65 or older.

It is an aspect of the present invention that the polypeptides provided herein have functional benefits beyond provision of polypeptide capable of decomposition, including the demonstration that peptides contained within the polypeptides have unique amino acid compositions. Moreover, provided are polypeptides that have amino acid ratios not found in naturally-occurring full-length polypeptides or mixtures of polypeptides, such ratios are beneficial, both in the ability of the polypeptides to modulate the metabolic signaling that occurs via single amino acids and small peptides, as well as the ability of polypeptides (and their amino acid components) to stimulate specific metabolic responses important to the health of the consuming organism. As provided herein, a ratio of amino acids can be demonstrated by comparison of the composition in a polypeptide of a single amino acid, or two or more amino acids, either to a reference polypeptide or a reference polypeptide mixture. In some embodiments, such comparison may include the content of one amino acid in a polypeptide versus the content of the same amino acid in a reference polypeptide or a reference polypeptide mixture. In other embodiments, such comparison may include the relative content of one amino acid in a polypeptide versus the content of all other amino acids present in a reference polypeptide or a reference polypeptide mixture.

In other preferred embodiments, a composition or formulation is nutritional in its provision of carbohydrate capable of hydrolysis by the intended consumer (termed a “nutritional carbohydrate”). A nutritional benefit in a carbohydrate-containing composition can be demonstrated and, optionally, quantified, by a number of metrics. For example, a nutritional benefit is the benefit to a consuming organism equivalent to or greater than at least about 2% of a reference daily intake value of carbohydrate.

In other preferred embodiments, a composition or formulation is nutritional in its provision of lipid capable of digestion, incorporation, conversion, or other cellular uses by the intended consumer (termed a “nutritional lipid”). A nutritional benefit in a lipid-containing composition can be demonstrated and, optionally, quantified, by a number of metrics. For example, a nutritional benefit is the benefit to a consuming organism equivalent to or greater than at least about 2% of a reference daily intake value of lipid (i.e., fat).

An “agriculturally-derived food product” is a food product resulting from the cultivation of soil or rearing of animals.

As used herein, a protein has “homology” or is “homologous” to a second protein if the nucleic acid sequence that encodes the protein has a similar sequence to the nucleic acid sequence that encodes the second protein. Alternatively, a protein has homology to a second protein if the two proteins have similar amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89.

The following six groups each contain amino acids that are conservative substitutions for one another: 1) Serine, Threonine; 2) Aspartic Acid, Glutamic Acid; 3) Asparagine, Glutamine; 4) Arginine, Lysine; 5) Isoleucine, Leucine, Methionine, Alanine, Valine, and 6) Phenylalanine, Tyrosine, Tryptophan.

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

An exemplary algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Exemplary parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, or at least about 20 residues, or at least about 24 residues, or at least about 28 residues, or more than about 35 residues. When searching a database containing sequences from a large number of different organisms, it may be useful to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990). For example, percent sequence identity between amino acid sequences can be determined using FASTA with its default parameters (a word size of 2 and the PAM250 scoring matrix), as provided in GCG Version 6.1, herein incorporated by reference.

In some embodiments, polymeric molecules (e.g., a polypeptide sequence or nucleic acid sequence) are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% similar. The term “homologous” necessarily refers to a comparison between at least two sequences (nucleotides sequences or amino acid sequences). In some embodiments, two nucleotide sequences are considered to be homologous if the polypeptides they encode are at least about 50% identical, at least about 60% identical, at least about 70% identical, at least about 80% identical, or at least about 90% identical for at least one stretch of at least about 20 amino acids. In some embodiments, homologous nucleotide sequences are characterized by the ability to encode a stretch of at least 4-5 uniquely specified amino acids. Both the identity and the approximate spacing of these amino acids relative to one another must be considered for nucleotide sequences to be considered homologous. In some embodiments of nucleotide sequences less than 60 nucleotides in length, homology is determined by the ability to encode a stretch of at least 4-5 uniquely specified amino acids. In some embodiments, two protein sequences are considered to be homologous if the proteins are at least about 50% identical, at least about 60% identical, at least about 70% identical, at least about 80% identical, or at least about 90% identical for at least one stretch of at least about 20 amino acids.

As used herein, a “modified derivative” refers to polypeptides or fragments thereof that are substantially homologous in primary structural sequence to a reference polypeptide sequence but which include, e.g., in vivo or in vitro chemical and biochemical modifications or which incorporate amino acids that are not found in the reference polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands that bind to labeled antiligands (e.g., antibodies), fluorophores, chemiluminescent agents, enzymes, and antiligands that can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002).

As used herein, “polypeptide mutant” or “mutein” refers to a polypeptide whose sequence contains an insertion, duplication, deletion, rearrangement or substitution of one or more amino acids compared to the amino acid sequence of a reference protein or polypeptide, such as a native or wild-type protein. A mutein may have one or more amino acid point substitutions, in which a single amino acid at a position has been changed to another amino acid, one or more insertions and/or deletions, in which one or more amino acids are inserted or deleted, respectively, in the sequence of the reference protein, and/or truncations of the amino acid sequence at either or both the amino or carboxy termini. A mutein may have the same or a different biological activity compared to the reference protein.

In some embodiments, a mutein has, for example, at least 85% overall sequence homology to its counterpart reference protein. In some embodiments, a mutein has at least 90% overall sequence homology to the wild-type protein. In other embodiments, a mutein exhibits at least 95% sequence identity, or 98%, or 99%, or 99.5% or 99.9% overall sequence identity.

As used herein, a “polypeptide tag for affinity purification” is any polypeptide that has a binding partner that can be used to isolate or purify a second protein or polypeptide sequence of interest fused to the first “tag” polypeptide. Several examples are well known in the art and include a His-6 tag (SEQ ID NO: 22138), a FLAG epitope, a c-myc epitope, a Strep-TAGII, a biotin tag, a glutathione 5-transferase (GST), a chitin binding protein (CBP), a maltose binding protein (MBP), or a metal affinity tag.

As used herein, a “polypeptide charge” or “protein charge” is calculated for a polypeptide or protein at pH 7 using Formula 1.

Charge_{P}=−0.002−C*0.045−D*0.999−E*0.998+H*0.091+K*1.0+R*1.0−Y*−0.001 Formula 1:

Charge_{P} is the net charge of the polypeptide or protein.

C is the number cysteine residues in the polypeptide or protein.

D is the number of aspartic acid residues in the polypeptide or protein.

E is the number of glutamic acid residues in the polypeptide or protein.

H is the number of histidine residues in the polypeptide or protein.

K is the number of lysine residues in the polypeptide or protein.

R is the number of arginine residues in the polypeptide or protein.

Y is the number of tyrosine residues in the polypeptide or protein.

As used herein, a “per amino acid charge” is calculated for a polypeptide or protein at pH 7 using Formula 2.

Charge_{A}=(−0.002−C*0.045−D*0.999−E*0.998+H*0.091+K*1.0+R*1.0−Y*−0.001)/N Formula 2:

Charge_{A} is the net charge per amino acid of the polypeptide or protein.

C, D, E, H, K, R, and Y are as in Formula 1.

N is the number of amino acids in the polypeptide or protein.

As used herein, “recombinant” refers to a biomolecule, e.g., a gene or polypeptide, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. Also, “recombinant” refers to a cell or an organism, such as a unicellular organism, herein termed a “recombinant unicellular organism,” a “recombinant host” or a “recombinant cell” that contains, produces and/or secretes a biomolecule, which can be a recombinant biomolecule or a non-recombinant biomolecule. For example, a recombinant unicellular organism may contain a recombinant nucleic acid providing for enhanced production and/or secretion of a recombinant polypeptide or a non-recombinant polypeptide. A recombinant cell or organism, is also intended to refer to a cell into which a recombinant nucleic acid such as a recombinant vector has been introduced. A “recombinant unicellular organism” includes a recombinant microorganism host cell and refers not only to the particular subject cell but to the progeny of such a cell. Because certain modifications may occur in succeeding generations due to either mutation or environmental influences, such progeny may not, in fact, be identical to the parent cell, but are still included within the scope of the terms herein.

The term “polynucleotide”, “nucleic acid molecule”, “nucleic acid”, or “nucleic acid sequence” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

A “synthetic” RNA, DNA or a mixed polymer is one created outside of a cell, for example one synthesized chemically.

The term “nucleic acid fragment” as used herein refers to a nucleic acid sequence that has a deletion, e.g., a 5′-terminal or 3′-terminal deletion compared to a full-length reference nucleotide sequence. In an embodiment, the nucleic acid fragment is a contiguous sequence in which the nucleotide sequence of the fragment is identical to the corresponding positions in the naturally-occurring sequence. In some embodiments, fragments are at least 10, 15, 20, or 25 nucleotides long, or at least 20, 30, 40, 50, 60, 70, 80, 90, 100, 110, 120, 130, 140, or 150 nucleotides long. In some embodiments a fragment of a nucleic acid sequence is a fragment of an open reading frame sequence. In some embodiments such a fragment encodes a polypeptide fragment (as defined herein) of the protein encoded by the open reading frame nucleotide sequence.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome. The term “recombinant” can also be used in reference to cloned DNA isolates, chemically-synthesized polynucleotide analogs, or polynucleotide analogs that are biologically synthesized by heterologous systems, as well as polypeptides and/or mRNAs encoded by such nucleic acids. Thus, for example, a polypeptide synthesized by a microorganism is recombinant, for example, if it is produced from an mRNA transcribed from a recombinant gene or other nucleic acid sequence present in the cell.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

The term “percent sequence identity” or “identical” in the context of nucleic acid sequences refers to the residues in the two sequences which are the same when aligned for maximum correspondence. The length of sequence identity comparison may be over a stretch of at least about nine nucleotides, usually at least about 20 nucleotides, more usually at least about 24 nucleotides, typically at least about 28 nucleotides, more typically at least about 32, and even more typically at least about 36 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences. Pearson, Methods Enzymol. 183:63-98 (1990). For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference. Alternatively, sequences can be compared using the computer program, BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “substantial homology” or “substantial similarity,” when referring to a nucleic acid or fragment thereof, indicates that, when aligned with appropriate nucleotide insertions or deletions with another nucleic acid (or its complementary strand), there is nucleotide sequence identity in at least about 76%, 80%, 85%, or at least about 90%, or at least about 95%, 96%, 97%, 98% or 99% of the nucleotide bases, as measured by any well-known algorithm of sequence identity, such as FASTA, BLAST or Gap, as discussed above.

Alternatively, substantial homology or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

In general, “stringent hybridization” is performed at about 25° C. below the thermal melting point (Tm) for the specific DNA hybrid under a particular set of conditions. “Stringent washing” is performed at temperatures about 5° C. lower than the Tm for the specific DNA hybrid under a particular set of conditions. The Tm is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

As used herein, an “expression control sequence” refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences and fusion partner sequences.

As used herein, “operatively linked” or “operably linked” expression control sequences refers to a linkage in which the expression control sequence is contiguous with the gene of interest to control the gene of interest, as well as expression control sequences that act in trans or at a distance to control the gene of interest.

As used herein, a “vector” is intended to refer to a nucleic acid molecule capable of transporting another nucleic acid to which it has been linked. One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain vectors are capable of directing the expression of genes to which they are operatively linked. Such vectors are referred to herein as “recombinant expression vectors” (or simply “expression vectors”).

The term “recombinant host cell” (or simply “recombinant cell” or “host cell”), as used herein, is intended to refer to a cell into which a recombinant nucleic acid such as a recombinant vector has been introduced. In some instances the word “cell” is replaced by a name specifying a type of cell. For example, a “recombinant microorganism” is a recombinant host cell that is a microorganism host cell. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “recombinant host cell,” “recombinant cell,” and “host cell”, as used herein. A recombinant host cell may be an isolated cell or cell line grown in culture or may be a cell which resides in a living tissue or organism.

As used herein, the term “heterotrophic” refers to an organism that cannot fix carbon and uses organic carbon for growth.

As used herein, the term “autotrophic” refers to an organism that produces complex organic compounds (such as carbohydrates, fats, and proteins) from simple inorganic molecules using energy from light (by photosynthesis) or inorganic chemical reactions (chemosynthesis).

As used herein, “muscle mass” refers to the weight of muscle in a subject's body. Muscle mass includes the skeletal muscles, smooth muscles (such as cardiac and digestive muscles) and the water contained in these muscles. Muscle mass of specific muscles can be determined using dual energy x-ray absorptiometry (DEXA) (Padden-Jones et al., 2004). Total lean body mass (minus the fat), total body mass, and bone mineral content can be measured by DEXA as well. In some embodiments a change in the muscle mass of a specific muscle of a subject is determined, for example by DEXA, and the change is used as a proxy for the total change in muscle mass of the subject. Thus, for example, if a subject consumes a nutritive protein as disclosed herein and experiences an increase over a period of time in muscle mass in a particular muscle or muscle group, it can be concluded that the subject has experienced an increase in muscle mass. Changes in muscle mass can be measured in a variety of ways including protein synthesis, fractional synthetic rate, and certain key activities such mTor/mTorc. In general, “lean muscle mass” refers to the mass of muscle tissue in the absence of other tissues such as fat.

As used herein, “muscle strength” refers to the amount of force a muscle can produce with a single maximal effort. There are two types of muscle strength, static strength and dynamic strength. Static strength refers to isometric contraction of a muscle, where a muscle generates force while the muscle length remains constant and/or when there is no movement in a joint. Examples include holding or carrying an object, or pushing against a wall. Dynamic strength refers to a muscle generating force that results in movement. Dynamic strength can be isotonic contraction, where the muscle shortens under a constant load or isokinetic contraction, where the muscle contracts and shortens at a constant speed. Dynamic strength can also include isoinertial strength.

Unless specified, “muscle strength” refers to maximum dynamic muscle strength. Maximum strength is referred to as “one repetition maximum” (1RM). This is a measurement of the greatest load (in kilograms) that can be fully moved (lifted, pushed or pulled) once without failure or injury. This value can be measured directly, but doing so requires that the weight is increased until the subject fails to carry out the activity to completion. Alternatively, 1RM is estimated by counting the maximum number of exercise repetitions a subject can make using a load that is less than the maximum amount the subject can move. Leg extension and leg flexion are often measured in clinical trials (Borsheim et al., “Effect of amino acid supplementation on muscle mass, strength and physical function in elderly,” Clin Nutr 2008; 27:189-195; Paddon-Jones, et al., “Essential amino acid and carbohydrate supplementation ameliorates muscle protein loss in humans during 28 days bed rest,” J Clin Endocrinol Metab 2004; 89:4351-4358).

As used herein, “functional performance” refers to a functional test that simulates daily activities. “Functional performance” is measured by any suitable accepted test, including timed-step test (step up and down from a 4 inch bench as fast as possible 5 times), timed floor transfer test (go from a standing position to a supine position on the floor and thereafter up to a standing position again as fast as possible for one repetition), and physical performance battery test (static balance test, chair test, and a walking test) (Borsheim et al., “Effect of amino acid supplementation on muscle mass, strength and physical function in elderly,” Clin Nutr 2008; 27:189-195).

As used herein, a “body mass index” or “BMI” or “Quetelet index” is a subject's weight in kilograms divided by the square of the subject's height in meters (kg/m²).

For adults, a frequent use of the BMI is to assess how much an individual's body weight departs from what is normal or desirable for a person of his or her height. The weight excess or deficiency may, in part, be accounted for by body fat, although other factors such as muscularity also affect BMI significantly. The World Health Organization regards a BMI of less than 18.5 as underweight and may indicate malnutrition, an eating disorder, or other health problems, while a BMI greater than 25 is considered overweight and above 30 is considered obese. (World Health Organization. BMI classification.) As used herein a “desirable body mass index” is a body mass index of from about 18.5 to about 25. Thus, if a subject has a BMI below about 18.5, then an increase in the subject's BMI is an increase in the desirability of the subject's BMI. If instead a subject has a BMI above about 25, then a decrease in the subject's BMI is an increase in the desirability of the subject's BMI.

As used herein, an “elderly” mammal is one who experiences age related changes in at least one of body mass index and muscle mass (e.g., age related sarcopenia). In some embodiments an “elderly” human is at least 50 years old, at least 60 years old, at least 65 years old, at least 70 years old, at least 75 years old, at least 80 years old, at least 85 years old, at least 90 years old, at least 95 years old, or at least 100 years old. In some embodiments and an elderly animal, mammal, or human is a human who has experienced a loss of muscle mass from peak lifetime muscle mass of at least 5%, at least 10%, at least 15%, at least 20%, at least 25%, at least 30%, at least 35%, at least 40%, at least 45%, at least 50%, at least 55%, or at least 60%. Because age related changes to at least one of body mass index and muscle mass are known to correlate with increasing age, in some embodiments an elderly mammal is identified or defined simply on the basis of age. Thus, in some embodiments an “elderly” human is identified or defined simply by the fact that their age is at least 60 years old, at least 65 years old, at least 70 years old, at least 75 years old, at least 80 years old, at least 85 years old, at least 90 years old, at least 95 years old, or at least 100 years old, and without recourse to a measurement of at least one of body mass index and muscle mass.

As used herein, “sarcopenia” refers to the degenerative loss of skeletal muscle mass (typically 0.5-1% loss per year after the age of 25), quality, and strength associated with aging. Sarcopenia is a component of the frailty syndrome. The European Working Group on Sarcopenia in Older People (EWGSOP) has developed a practical clinical definition and consensus diagnostic criteria for age-related sarcopenia. For the diagnosis of sarcopenia, the working group has proposed using the presence of both low muscle mass and low muscle function (strength or performance). Sarcopenia is characterized first by a muscle atrophy (a decrease in the size of the muscle), along with a reduction in muscle tissue “quality,” caused by such factors as replacement of muscle fibres with fat, an increase in fibrosis, changes in muscle metabolism, oxidative stress, and degeneration of the neuromuscular junction. Combined, these changes lead to progressive loss of muscle function and eventually to frailty. Frailty is a common geriatric syndrome that embodies an elevated risk of catastrophic declines in health and function among older adults. Contributors to frailty can include sarcopenia, osteoporosis, and muscle weakness. Muscle weakness, also known as muscle fatigue, (or “lack of strength”) refers to the inability to exert force with one's skeletal muscles. Weakness often follows muscle atrophy and a decrease in activity, such as after a long bout of bedrest as a result of an illness. There is also a gradual onset of muscle weakness as a result of sarcopenia.

As used herein, a patient is “critically-medically ill” if the patient, because of medical illness, experiences changes in at least one of body mass index and muscle mass (e.g., sarcopenia). In some embodiments the patient is confined to bed for at least 25%, at least 50%, at least 60%, at least 70%, at least 80%, at least 90%, at least 95%, or 100% of their waking time. In some embodiments the patient is unconscious. In some embodiments the patient has been confined to bed as described in this paragraph for at least 1 day, 2 days, 3 days, 4 days, 5 days, 10 days, 2 weeks, 3 weeks, 4 weeks, 5 weeks, 10 weeks or longer.

As used herein, “protein-energy malnutrition” refers to a form of malnutrition where there is inadequate protein intake. Types include Kwashiorkor (protein malnutrition predominant), Marasmus (deficiency in both calorie and protein nutrition), and Marasmic Kwashiorkor (marked protein deficiency and marked calorie insufficiency signs present, sometimes referred to as the most severe form of malnutrition).

As used herein, “exercise” is, most broadly, any bodily activity that enhances or maintains physical fitness and overall health and wellness. Exercise is performed for various reasons including strengthening muscles and the cardiovascular system, honing athletic skills, weight loss or maintenance, as well as for the purpose of enjoyment.

As used herein, a “sufficient amount” is an amount of a protein or polypeptide disclosed herein that is sufficient to cause a desired effect. For example, if an increase in muscle mass is desired, a sufficient amount is an amount that causes an increase in muscle mass in a subject over a period of time. A sufficient amount of a protein or polypeptide fragment can be provided directly, i.e., by administering the protein or polypeptide fragment to a subject, or it can be provided as part of a composition comprising the protein or polypeptide fragment. Modes of administration are discussed elsewhere herein.

As used herein, the term “mammal” refers to any member of the taxonomic class mammalia, including placental mammals and marsupial mammals. Thus, “mammal” includes humans, primates, livestock, and laboratory mammals. Exemplary mammals include a rodent, a mouse, a rat, a rabbit, a dog, a cat, a sheep, a horse, a goat, a llama, cattle, a primate, a pig, and any other mammal. In some embodiments, the mammal is at least one of a transgenic mammal, a genetically-engineered mammal, and a cloned mammal.

As used herein, “satiation” is the act of becoming full while eating or a reduced desire to eat. This halts or diminishes eating.

As used herein, “satiety” is the act of remaining full after a meal which manifests as the period of no eating follow the meal.

As used herein, “exercise” is, most broadly, any bodily activity that enhances or maintains physical fitness and overall health and wellness. Exercise is performed for various reasons including strengthening muscles and the cardiovascular system, honing athletic skills, weight loss or maintenance, as well as for the purpose of enjoyment.

The term “ameliorating” refers to any therapeutically beneficial result in the treatment of a disease state, e.g., including prophylaxis, lessening in the severity or progression, remission, or cure thereof.

As used herein, the term “in vitro” refers to events that occur in an artificial environment, e.g., in a test tube or reaction vessel, in cell culture, in a Petri dish, etc., rather than within an organism (e.g., animal, plant, or microbe). As used herein, the term “ex vivo” refers to experimentation done in or on tissue in an environment outside the organism.

The term “in situ” refers to processes that occur in a living cell growing separate from a living organism, e.g., growing in tissue culture.

The term “in vivo” refers to processes that occur in a living organism.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

As used herein, “amino acid likelihood” (abbreviated as “AALike”) is a measure of the frequency with which a given amino acid appears at a given position of a multiple sequence alignment (MSA) generated with reference to a reference protein. The position is defined relative to the amino acid sequence of the reference protein. The reference protein can be any protein, such as a reference secreted protein. After a MSA is generated, the frequency with which each amino acid appears at each position of the protein sequences in the MSA is calculated to give the amino acid likelihood for each position. Thus, for each amino acid position of the reference protein up to 20 different amino acid likelihood values can be calculated.

For a given query protein sequence an MSA is created using homologous proteins. Homologous proteins can be identified using any of the several methods known in the art. For example, homologous proteins may be identified by performing local sequence alignments of the query with NCBI's library of non-redundant proteins. The initial local alignments may be performed using the blastp program from the NCBI toolkit v.2.2.26+(Altschul S. F., Gish W., Miller W., Myers E. W., and Lipman D. J. “Basic Local Alignment Search Tool”. J. Mol. Biol. (1990) 215: 403-410) with parameters selected from:

An e-value cutoff of 1, a gap opening penalty of −11, a gap extension penalty of −1, and the BLOSUM62 scoring matrix;

An e-value cutoff of 1, a gap opening penalty of −15, a gap extension penalty of −2, and the BLOSUM45 scoring matrix;

An e-value cutoff of 1, a gap opening penalty of −10, a gap extension penalty of −1, and the BLOSUM80 scoring matrix;

An e-value cutoff of 1, a gap opening penalty of −10, a gap extension penalty of −1, and the PAM70 scoring matrix; and

An e-value cutoff of 1, a gap opening penalty of −9, a gap extension penalty of −1, and the PAM30 scoring matrix.

The multiple sequence alignment of the resulting library was performed using the Align123 algorithm as implemented in Discovery Studio v3.1 (Accelrys Software Inc., Discovery Studio Modeling Environment, Release 3.1, San Diego: Accelrys Software Inc., 2012). Residue secondary structure was assigned using the DSC algorithm (King R. D., Sternberg M. J. E. “Identification and application of the concepts important for accurate and reliable protein secondary structure prediction”. Prot. Sci. (1996) 5: 2298-2310) with a weight of 1. Pairwise alignments were performed using the Smith and Waterman algorithm with a Gap opening penalty of −10 and gap extension penalty of −0.1, and the BLOSUM30 scoring matrix. Higher order alignments used the BLOSUM scoring matrix set, a gap opening penalty of −10, a gap extension penalty of −0.5, and an alignment delay identity cutoff (delay divergent parameter) of 40%.

All proteins with a local alignment expectation value less than 1 (from 75 to 1000 unique hits) were identified and aligned to generate a multiple sequence alignment (MSA). The proteins used for each MSA are presented in Appendix C.

As used herein, “amino acid type likelihood” (abbreviated as “AATLike”) is a measure of the frequency with which a given type of amino acid appears at a given position of a multiple sequence alignment (MSA) generated with reference to a reference protein. The amino acid type is chosen from branched chain amino acids (BCAA) (Leu, Ile, and Val), hydrophobic amino acids (Ala, Met, Ile, Leu, and Val), positively charged amino acids (Arg, Lys, His), negatively charged amino acids (Asp, Glu), charged amino acids (Arg, Lys, His, Asp Glu), and aromatic amino acids (Phe, Tyr, Trp). The position is defined relative to the amino acid sequence of the reference protein. The reference protein can be any protein, such as a reference secreted protein. After a MSA is generated, the frequency with which each type of amino acid appears at each position of the protein sequences in the MSA is calculated to give the amino acid type likelihood for each position.

As used herein, “position entropy” (abbreviated as “Sₚₒₛ”) is a measure of the spread of the amino acid distribution at a position in a MSA. An MSA is used to compute the entropy of each amino acid position in a given reference amino acid sequence using the full amino acid alphabet, AA=[A, R, N, D, C, Q, E, G, H, I, L, K, M, F, P, S, T, W, Y, V]:

S=−Σ_{jεAA}pⱼ ln pⱼ

where pⱼ is the probability of seeing the amino acid j at that position. Highly variable positions will have large entropies (the maximum entropy at a position corresponds to each amino acid being equally likely, which yields an entropy of 2.996) and highly conserved positions will have an entropy close to 0.

As used herein, “amino acid type position entropy” (abbreviated as “S_{AATpos}”) is a variation on position entropy in which, instead of using the full amino acid alphabet to calculate the position entropy, amino acids are grouped based on physiochemical properties as follows: hydrophobic [A, V, I, L, M], aromatic [F, Y, W], polar [S, T, N, Q], charged [R, H, K, D, E], and non-classified [G, P, C]. Using this physiochemical alphabet, pⱼ now corresponds to the probability of seeing each amino acid type (hydrophobic, aromatic, polar, charged, or non-classified) at position j. These amino acid type (AAType) probabilities are the sum of the probabilities of seeing each amino acid of that type. The equation for the position entropy stays the same, although the theoretical maximum is now 1.609.

A. Engineered Proteins

In some embodiments a protein comprises or consists of a derivative or mutein of a protein or fragment of a protein that naturally occurs in an edible product. Such a protein can be referred to as an “engineered protein.” In such embodiments the natural protein or fragment thereof is a “reference” protein or polypeptide and the engineered protein or a first polypeptide sequence thereof comprises at least one sequence modification relative to the amino acid sequence of the reference protein or polypeptide. For example, in some embodiments the engineered protein or first polypeptide sequence thereof is at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.5% identical to at least one reference protein amino acid sequence. Typically the ratio of at least one of branched chain amino acid residues to total amino acid residues, essential amino acid residues to total amino acid residues, and leucine residues to total amino acid residues, present in the engineered protein or a first polypeptide sequence thereof is greater than the corresponding ratio of at least one of branched chain amino acid residues to total amino acid residues, essential amino acid residues to total amino acid residues, and leucine residues to total amino acid residues present in the reference protein or polypeptide sequence.

In some aspects the nutritive polypeptide is substantially digestible upon consumption by a mammalian subject. Preferably, the nutritive polypeptide is easier to digest than at least a reference polypeptide or a reference mixture of polypeptides, or a portion of other polypeptides in the consuming subject's diet. As used herein, “substantially digestible” can be demonstrated by measuring half-life of the nutritive polypeptide upon consumption. For example, a nutritive polypeptide is easier to digest if it has a half-life in the gastrointestinal tract of a human subject of less than 60 minutes, or less than 50, 40, 30, 20, 15, 10, 5, 4, 3, 2 minutes or 1 minute. In certain embodiments the nutritive polypeptide is provided in a formulation that provides enhanced digestion; for example, the nutritive polypeptide is provided free from other polypeptides or other materials. In some embodiments, the nutritive polypeptide contains one or more recognition sites for one or more endopeptidases. In a specific embodiment, the nutritive polypeptide contains a secretion leader (or secretory leader) sequence, which is then cleaved from the nutritive polypeptide. As provided herein, a nutritive polypeptide encompasses polypeptides with or without signal peptides and/or secretory leader sequences. In some embodiments, the nutritive polypeptide is susceptible to cleavage by one or more exopeptidases.

In some aspects the nutritive polypeptide is selected to have a desired density of one or more essential amino acids (EAA). Essential amino acid deficiency can be treated or prevented with the effective administration of the one or more essential amino acids otherwise absent or present in insufficient amounts in a subject's diet. For example, EAA density is about equal to or greater than the density of essential amino acids present in a full-length reference nutritional polypeptide, e.g., EAA density in a nutritive polypeptide is at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or above 500% greater than a reference nutritional polypeptide or the polypeptide present in an agriculturally-derived food product.

In some aspects the nutritive polypeptide is selected to have a desired density of aromatic amino acids (“AAA”, including phenylalanine, tryptophan, tyrosine, histidine, and thyroxine). AAAs are useful, e.g., in neurological development and prevention of exercise-induced fatigue. For example, AAA density is about equal to or greater than the density of essential amino acids present in a full-length reference nutritional polypeptide, e.g., AAA density in a nutritive polypeptide is at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or above 500% greater than a reference nutritional polypeptide or the polypeptide present in an agriculturally-derived food product.

In some aspects the nutritive polypeptide is selected to have a desired density of branched chain amino acids (BCAA). For example, BCAA density, either individual BCAAs or total BCAA content is about equal to or greater than the density of branched chain amino acids present in a full-length reference nutritional polypeptide, e.g., BCAA density in a nutritive polypeptide is at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or above 500% greater than a reference nutritional polypeptide or the polypeptide present in an agriculturally-derived food product. BCAA density in a nutritive polypeptide can also be selected for in combination with one or more attributes such as EAA density.

In some aspects the nutritive polypeptide is selected to have a desired density of amino acids arginine, glutamine and/or leucine (RQL amino acids). For example, RQL amino acid density is about equal to or greater than the density of essential amino acids present in a full-length reference nutritional polypeptide, e.g., RQL amino acid density in a nutritive polypeptide is at least about 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500% or above 500% greater than a reference nutritional polypeptide or the polypeptide present in an agriculturally-derived food product.

In some embodiments, the engineered protein comprises at least one threonine (Thr) amino acid residue substitution of a non-Thr amino acid residue in the reference secreted protein.

In some embodiments, the engineered protein comprises at least one arginine (Arg) amino acid residue substitution of a non-Arg amino acid residue in the reference secreted protein.

In some embodiments, the engineered protein comprises at least one histidine (His) amino acid residue substitution of a non-His amino acid residue in the reference secreted protein.

In some embodiments, the engineered protein comprises at least one lysine (Lys) amino acid residue substitution of a non-Lys amino acid residue in the reference secreted protein.

In some embodiments, the engineered protein comprises at least one leucine (Leu) amino acid residue substitution of a non-Leu amino acid residue in the reference secreted protein.

In some embodiments, the engineered protein comprises at least one isoleucine (Ile) amino acid residue substitution of a non-Ile amino acid residue in the reference secreted protein.

In some embodiments, the engineered protein comprises at least one valine (Val) amino acid residue substitution of a non-Val amino acid residue in the reference secreted protein.

In another aspect, provided are nutritive polypeptides that contain amino acid sequences homologous to naturally-occurring polypeptides or variants thereof, which are engineered to be secreted from unicellular organisms and purified therefrom. Such homologous polypeptides can be 70%, 75%, 80%, 85%, 90%, 91%, 92%, 93%, 94%, 95%, 96%, 97%, 98%, 99% or greater than 99% similar, or can be 70%, 75%, 80%, 85%, 90%, 91%, 92%, 93%, 94%, 95%, 96%, 97%, 98%, 99% or greater than 99% identical to a naturally-occurring polypeptide or variant thereof. Such nutritive polypeptides can be endogenous to the host cell or exogenous, can be naturally secreted in the host cell, or both, and can be engineered for secretion.

In some embodiments herein a fragment of a naturally-occurring protein is selected and optionally isolated. In some embodiments the fragment comprises at least 25 amino acids. In some embodiments the fragment comprises at least 50 amino acids. In some embodiments the fragment consists of at least 25 amino acids. In some embodiments the fragment consists of at least 50 amino acids. In some embodiments an isolated recombinant protein is provided. In some embodiments the protein comprises a first polypeptide sequence, and the first polypeptide sequence comprises a fragment of at least 25 or at least 50 amino acids of a naturally-occurring protein. In some embodiments the proteins is isolated. In some embodiments the proteins are recombinant. In some embodiments the proteins comprise a first polypeptide sequence comprising a fragment of at least 50 amino acids of a naturally-occurring protein. In some embodiments the proteins are isolated recombinant proteins. In some embodiments the isolated recombinant proteins disclosed herein are provided in a non-isolated and/or non-recombinant form.

In some instances herein the portion of amino acid(s) of a particular type within a polypeptide, protein or a composition is quantified based on the weight ratio of the type of amino acid(s) to the total weight of amino acids present in the polypeptide, protein or composition in question. This value is calculated by dividing the weight of the particular amino acid(s) in the polypeptide, protein or a composition by the weight of all amino acids present in the polypeptide, protein or a composition.

In other instances the ratio of a particular type of amino acid(s) residues present in a polypeptide or protein to the total number of amino acids present in the polypeptide or protein in question is used. This value is calculated by dividing the number of the amino acid(s) in question that is present in each molecule of the polypeptide or protein by the total number of amino acid residues present in each molecule of the polypeptide or protein. A skilled artisan appreciates that these two methods are interchangeable and that the weight proportion of a type of amino acid(s) present in a polypeptide or protein can be converted to a ratio of the particular type of amino acid residue(s), and vice versa.

In some embodiments the protein comprises from 10 to 5,000 amino acids, from 20-2,000 amino acids, from 20-1,000 amino acids, from 20-500 amino acids, from 20-250 amino acids, from 20-200 amino acids, from 20-150 amino acids, from 20-100 amino acids, from 20-40 amino acids, from 30-50 amino acids, from 40-60 amino acids, from 50-70 amino acids, from 60-80 amino acids, from 70-90 amino acids, from 80-100 amino acids, at least 10 amino acids, at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, at least 24 amino acids, at least 25 amino acids, at least 30 amino acids, at least 35 amino acids, at least 40 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, at least 105 amino acids, at least 110 amino acids, at least 115 amino acids, at least 120 amino acids, at least 125 amino acids, at least 130 amino acids, at least 135 amino acids, at least 140 amino acids, at least 145 amino acids, at least 150 amino acids, at least 155 amino acids, at least 160 amino acids, at least 165 amino acids, at least 170 amino acids, at least 175 amino acids, at least 180 amino acids, at least 185 amino acids, at least 190 amino acids, at least 195 amino acids, at least 200 amino acids, at least 205 amino acids, at least 210 amino acids, at least 215 amino acids, at least 220 amino acids, at least 225 amino acids, at least 230 amino acids, at least 235 amino acids, at least 240 amino acids, at least 245 amino acids, or at least 250 amino acids. In some embodiments the protein consists of from 20 to 5,000 amino acids, from 20-2,000 amino acids, from 20-1,000 amino acids, from 20-500 amino acids, from 20-250 amino acids, from 20-200 amino acids, from 20-150 amino acids, from 20-100 amino acids, from 20-40 amino acids, from 30-50 amino acids, from 40-60 amino acids, from 50-70 amino acids, from 60-80 amino acids, from 70-90 amino acids, from 80-100 amino acids, at least 25 amino acids, at least 30 amino acids, at least 35 amino acids, at least 40 amino acids, at least 2455 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, at least 105 amino acids, at least 110 amino acids, at least 115 amino acids, at least 120 amino acids, at least 125 amino acids, at least 130 amino acids, at least 135 amino acids, at least 140 amino acids, at least 145 amino acids, at least 150 amino acids, at least 155 amino acids, at least 160 amino acids, at least 165 amino acids, at least 170 amino acids, at least 175 amino acids, at least 180 amino acids, at least 185 amino acids, at least 190 amino acids, at least 195 amino acids, at least 200 amino acids, at least 205 amino acids, at least 210 amino acids, at least 215 amino acids, at least 220 amino acids, at least 225 amino acids, at least 230 amino acids, at least 235 amino acids, at least 240 amino acids, at least 245 amino acids, or at least 250 amino acids. In some aspects, a protein or fragment thereof includes at least two domains: a first domain and a second domain. One of the two domains can include a tag domain, which can be removed if desired. Each domain can be 1, 2, 3, 4, 5, 6, 7, 8, 9, 10, 11, 12, 13, 14, 15, 16, 17, 18, 19, 20, 21, 22, 23, 24, 25, or greater than 25 amino acids in length. For example, the first domain can be a polypeptide of interest that is 18 amino acids in length and the second domain can be a tag domain that is 7 amino acids in length. As another example, the first domain can be a polypeptide of interest that is 17 amino acids in length and the second domain can be a tag domain that is 8 amino acids in length.

In some embodiments herein a fragment of a naturally-occurring protein is selected and optionally isolated. In some embodiments the fragment comprises at least 25 amino acids. In some embodiments the fragment comprises at least 50 amino acids. In some embodiments the fragment consists of at least 25 amino acids. In some embodiments the fragment consists of at least 50 amino acids. In some embodiments an isolated recombinant protein is provided. In some embodiments the protein comprises a first polypeptide sequence, and the first polypeptide sequence comprises a fragment of at least 25 or at least 50 amino acids of a naturally-occurring protein. In some embodiments the proteins is isolated. In some embodiments the proteins are recombinant. In some embodiments the proteins comprise a first polypeptide sequence comprising a fragment of at least 50 amino acids of a naturally-occurring protein. In some embodiments the proteins are isolated recombinant proteins. In some embodiments the isolated recombinant proteins disclosed herein are provided in a non-isolated and/or non-recombinant form.

This disclosure provides engineered proteins comprising a sequence of at least 20 amino acids that comprise an altered amino acid sequence compared to the amino acid sequence of a reference secreted protein. In some embodiments the engineered protein comprises a sequence of at least 25 amino acids, at least 30 amino acids, at least 35 amino acids, at least 40 amino acids, at least 45 amino acids, at least 50 amino acids, at least 60 amino acids, at least 70 amino acids, at least 80 amino acids, at least 85 amino acids, at least 90 amino acids, at least 95 amino acids, or at least 100 amino acids that comprises an altered amino acid sequence compared to the amino acid sequence of a reference secreted protein. In some embodiments the engineered protein comprises a sequence of at least 20 to 30 amino acids, at least 20 to 40 amino acids, at least 25 to 50 amino acids, ar at least 50 to 100 amino acids that comprises an altered amino acid sequence compared to the amino acid sequence of a reference secreted protein. As used herein, a “reference secreted protein” is a protein that is secreted from a compatible microorganism when expressed therein. A “compatible microorganism” is one that comprises the necessary machinery to synthesize and process the protein for secretion. The reference secreted protein may be a naturally occurring protein (i.e., a protein that naturally occurs in an organism) or a non-naturally occurring protein (i.e., a protein that does not naturally occur in the an organism). The compatible microorganisms for a particular reference secreted protein that is naturally occurring will necessarily include the microorganism that the reference secreted protein naturally occurs in.

The alterations between the sequence of the reference secreted protein and the engineered protein may be defined by performing a sequence alignment between the reference secreted protein and the engineered protein and identifying amino acid positions that differ. In some embodiments the sequence of at least 20 amino acids that comprises an altered amino acid sequence in the engineered protein is at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.5% homologous to a homologous sequence is the reference secreted protein. In some embodiments the amino acid sequence of the engineered protein is at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.5% homologous to the reference secreted protein.

In some embodiments the engineered protein comprises a ratio of essential amino acids to total amino acids present in the engineered protein higher than the ratio of essential amino acids to total amino acids present in the reference secreted protein. In some embodiments the engineered protein comprises at least one essential amino acid residue substitution of a non-essential amino acid residue in the reference secreted protein. In some embodiments the engineered protein comprises at least one branch chain amino acid residue substitution of a non-branch chain amino acid residue in the reference secreted protein. In some embodiments the engineered protein comprises at least one Arginine (Arg) or Glutamine (Glu) amino acid residue substitution of a non-Arginine (Arg) or non-Glutamine (Glu) amino acid residue in the reference secreted protein.

In some embodiments, the engineered protein comprises at least one leucine (Leu) amino acid residue substitution of a non-Leu amino acid residue in the reference secreted protein. In some embodiments the Leu amino acid residue substitution is at an amino acid position with a Leu frequency score greater than 0. In some embodiments the Leu amino acid residue substitution is at an amino acid position with a Leu frequency score of at least 0.1. In some embodiments the Leu amino acid residue substitution is at an amino acid position with a branch chain amino acid frequency score of greater than 0. In some embodiments the Leu amino acid residue substitution is at an amino acid position with a branch chain amino acid frequency score of at least 0.1. In some embodiments the Leu amino acid residue substitution is at an amino acid position with a hydrophobic amino acid frequency score of greater than 0. In some embodiments the Leu amino acid residue substitution is at an amino acid position with a hydrophobic amino acid frequency score of at least 0.1. In some embodiments the Leu amino acid residue substitution is at an amino acid position with a per amino acid position entropy of at least 1.5. In some embodiments the difference in total folding free energy between the reference secreted protein and the engineered protein is less than or equal to 0.5.

In some embodiments of the engineered protein, at least two non-leucine (Leu) amino acid residues in the reference secreted protein are substituted by a Leu amino acid residue in the engineered protein, wherein the difference in total folding free energy between the reference secreted protein and the engineered protein is less than or equal to 0.5, and wherein the major energetic component of the total folding free energies for each amino acid substitution is different.

In some embodiments the engineered protein comprises at least one Leu amino acid residue substitution of a non-Leu amino acid residue in a reference secreted protein at a position with a position entropy of at least 1.5. In some embodiments the difference in total folding free energy between the reference secreted protein and the engineered protein is less than or equal to 0.5. In some embodiments the engineered protein comprises at least two Leu amino acid residue substitutions of non-Leu amino acid residues in the reference secreted protein, wherein the contribution to the difference in total folding free energy between the reference secreted protein and the engineered protein from each of the Leu amino acid residue substitutions considered independently is less than or equal to 0.5 and the major energetic component of the total folding free energies for each amino acid substitution is different.

In some embodiments the engineered protein comprises at least one Leu amino acid residue substitution of a non-Leu amino acid residue in a reference secreted protein at a position at which the total free folding energy that results from the Leu substitution is less than or equal to 0.5. In some embodiments the engineered protein comprises at least two Leu amino acid residue substitutions of non-Leu amino acid residues in the reference secreted protein, wherein the contribution to the difference in total folding free energy between the reference secreted protein and the engineered protein from each of the Leu amino acid residue substitutions considered independently is less than or equal to 0.5 and the major energetic component of the total folding free energies for each amino acid substitution is different.

In some embodiments, the engineered protein comprises at least one valine (Val) amino acid residue substitution of a non-Val amino acid residue in the reference secreted protein. In some embodiments the Val amino acid residue substitution is at an amino acid position with a Val frequency score greater than 0. In some embodiments the Val amino acid residue substitution is at an amino acid position with a Val frequency score of at least 0.1. In some embodiments the Val amino acid residue substitution is at an amino acid position with a branch chain amino acid frequency score of greater than 0. In some embodiments the Val amino acid residue substitution is at an amino acid position with a branch chain amino acid frequency score of at least 0.1. In some embodiments the Val amino acid residue substitution is at an amino acid position with a hydrophobic amino acid frequency score of greater than 0. In some embodiments the Val amino acid residue substitution is at an amino acid position with a hydrophobic amino acid frequency score of at least 0.1. In some embodiments the Val amino acid residue substitution is at an amino acid position with a per amino acid position entropy of at least 1.5. In some embodiments the difference in total folding free energy between the reference secreted protein and the engineered protein is less than or equal to 0.5.

In some embodiments of the engineered protein, at least two non-valine (Val) amino acid residues in the reference secreted protein are substituted by a Val amino acid residue in the engineered protein, wherein the difference in total folding free energy between the reference secreted protein and the engineered protein is less than or equal to 0.5, and wherein the major energetic component of the total folding free energies for each amino acid substitution is different.

In some embodiments the engineered protein comprises at least one Val amino acid residue substitution of a non-Val amino acid residue in a reference secreted protein at a position with a position entropy of at least 1.5. In some embodiments the difference in total folding free energy between the reference secreted protein and the engineered protein is less than or equal to 0.5. In some embodiments the engineered protein comprises at least two Val amino acid residue substitutions of non-Val amino acid residues in the reference secreted protein, wherein the contribution to the difference in total folding free energy between the reference secreted protein and the engineered protein from each of the Val amino acid residue substitutions considered independently is less than or equal to 0.5 and the major energetic component of the total folding free energies for each amino acid substitution is different.

In some embodiments the engineered protein comprises at least one Val amino acid residue substitution of a non-Val amino acid residue in a reference secreted protein at a position at which the total free folding energy that results from the Val substitution is less than or equal to 0.5. In some embodiments the engineered protein comprises at least two Val amino acid residue substitutions of non-Val amino acid residues in the reference secreted protein, wherein the contribution to the difference in total folding free energy between the reference secreted protein and the engineered protein from each of the Val amino acid residue substitutions considered independently is less than or equal to 0.5 and the major energetic component of the total folding free energies for each amino acid substitution is different.

In some embodiments, the engineered protein comprises at least one isoleucine (Ile) amino acid residue substitution of a non-Ile amino acid residue in the reference secreted protein. In some embodiments the Ile amino acid residue substitution is at an amino acid position with a Ile frequency score greater than 0. In some embodiments the Ile amino acid residue substitution is at an amino acid position with a Ile frequency score of at least 0.1. In some embodiments the Ile amino acid residue substitution is at an amino acid position with a branch chain amino acid frequency score of greater than 0. In some embodiments the Ile amino acid residue substitution is at an amino acid position with a branch chain amino acid frequency score of at least 0.1. In some embodiments the Ile amino acid residue substitution is at an amino acid position with a hydrophobic amino acid frequency score of greater than 0. In some embodiments the Ile amino acid residue substitution is at an amino acid position with a hydrophobic amino acid frequency score of at least 0.1. In some embodiments the Ile amino acid residue substitution is at an amino acid position with a per amino acid position entropy of at least 1.5. In some embodiments the difference in total folding free energy between the reference secreted protein and the engineered protein is less than or equal to 0.5.

In some embodiments of the engineered protein, at least two non-isoleucine (Ile) amino acid residues in the reference secreted protein are substituted by a Ile amino acid residue in the engineered protein, wherein the difference in total folding free energy between the reference secreted protein and the engineered protein is less than or equal to 0.5, and wherein the major energetic component of the total folding free energies for each amino acid substitution is different.

In some embodiments the engineered protein comprises at least one Ile amino acid residue substitution of a non-Ile amino acid residue in a reference secreted protein at a position with a position entropy of at least 1.5. In some embodiments the difference in total folding free energy between the reference secreted protein and the engineered protein is less than or equal to 0.5. In some embodiments the engineered protein comprises at least two Ile amino acid residue substitutions of non-Ile amino acid residues in the reference secreted protein, wherein the contribution to the difference in total folding free energy between the reference secreted protein and the engineered protein from each of the Ile amino acid residue substitutions considered independently is less than or equal to 0.5 and the major energetic component of the total folding free energies for each amino acid substitution is different.

In some embodiments the engineered protein comprises at least one Ile amino acid residue substitution of a non-Ile amino acid residue in a reference secreted protein at a position at which the total free folding energy that results from the Ile substitution is less than or equal to 0.5. In some embodiments the engineered protein comprises at least two Ile amino acid residue substitutions of non-Ile amino acid residues in the reference secreted protein, wherein the contribution to the difference in total folding free energy between the reference secreted protein and the engineered protein from each of the Ile amino acid residue substitutions considered independently is less than or equal to 0.5 and the major energetic component of the total folding free energies for each amino acid substitution is different.

As used herein, an “amino acid frequency score,” such as a “Leu frequency score” is a measure of the frequency with which a particular amino acid or type of amino acid occurs at a homologous position across the naturally occurring sequences of homologous proteins. Thus, for a reference secreted protein, if a set of homologous sequences are identified using a multiple sequence alignment (MSA) and the sequences are aligned, the frequency with which each amino acid appears at each position across all of the sequences in the MSA may be determined and a frequency score assigned to each amino acid at each position. Alternatively, amino acids may be grouped by type, such as branch chain amino acids, essential amino acids, or hydrophobic amino acids, and frequency scores may be calculated based on the occurrence of any member of each type at each position (referred to herein as “amino acid type frequency score”). The amino acid frequency scores and amino acid type frequency scores may be used to identify amino acid positions in a reference secreted protein sequence that are tolerant of substitution by a different amino acid than the amino acid appearing at that position in the reference secreted protein sequence. For example, positions in a reference sequence that have an amino acid other than Leu, but that have a relatively high Leu frequency score may be substituted by Leu to make an engineered protein with an increased Leu content.

In some embodiments the engineered protein comprises at least one amino acid N substitution (wherein “N” stands for any amino acid) at a position with an N amino acid frequency score greater than 0. In some embodiments the engineered protein comprises at least one amino acid N substitution at a position with an N amino acid frequency score of at least 0.01. In some embodiments the engineered protein comprises at least one amino acid N substitution at a position with an N amino acid frequency score of at least 0.02. In some embodiments the engineered protein comprises at least one amino acid N substitution at a position with an N amino acid frequency score of at least 0.03. In some embodiments the engineered protein comprises at least one amino acid N substitution at a position with an N amino acid frequency score of at least 0.04. In some embodiments the engineered protein comprises at least one amino acid N substitution at a position with an N amino acid frequency score of at least 0.05. In some embodiments the engineered protein comprises at least one amino acid N substitution at a position with an N amino acid frequency score of at least 0.06. In some embodiments the engineered protein comprises at least one amino acid N substitution at a position with an N amino acid frequency score of at least 0.07. In some embodiments the engineered protein comprises at least one amino acid N substitution at a position with an N amino acid frequency score of at least 0.08. In some embodiments the engineered protein comprises at least one amino acid N substitution at a position with an N amino acid frequency score of at least 0.09. In some embodiments the engineered protein comprises at least one amino acid N substitution at a position with an N amino acid frequency score of at least 0.10. In some embodiments the engineered protein comprises at least one amino acid N substitution at a position with an N amino acid frequency score of at least 0.11. In some embodiments the engineered protein comprises at least one amino acid N substitution at a position with an N amino acid frequency score of at least 0.12. In some embodiments the engineered protein comprises at least one amino acid N substitution at a position with an N amino acid frequency score of at least 0.13. In some embodiments the engineered protein comprises at least one amino acid N substitution at a position with an N amino acid frequency score of at least 0.14. In some embodiments the engineered protein comprises at least one amino acid N substitution at a position with an N amino acid frequency score of at least 0.15. In some embodiments the engineered protein comprises at least one amino acid N substitution at a position with an N amino acid frequency score of at least 0.16. In some embodiments the engineered protein comprises at least one amino acid N substitution at a position with an N amino acid frequency score of at least 0.17. In some embodiments the engineered protein comprises at least one amino acid N substitution at a position with an N amino acid frequency score of at least 0.18. In some embodiments the engineered protein comprises at least one amino acid N substitution at a position with an N amino acid frequency score of at least 0.19. In some embodiments the engineered protein comprises at least one amino acid N substitution at a position with an N amino acid frequency score of at least 0.20. In some embodiments the engineered protein comprises at least one amino acid N substitution at a position with an N amino acid frequency score of at least 0.25. In some embodiments the engineered protein comprises at least one amino acid N substitution at a position with an N amino acid frequency score of at least 0.30. In some embodiments the engineered protein comprises at least one amino acid N substitution at a position with an N amino acid frequency score of at least 0.35. In some embodiments the engineered protein comprises at least one amino acid N substitution at a position with an N amino acid frequency score of at least 0.40. In some embodiments the engineered protein comprises at least one amino acid N substitution at a position with an N amino acid frequency score of at least 0.45. In some embodiments the engineered protein comprises at least one amino acid N substitution at a position with an N amino acid frequency score of at least 0.50. In some embodiments the amino acid N is selected from Leu, Ile, and Val. In some embodiments the amino acid N is selected from Arg and Glu. In some embodiments the amino acid N is selected from essential amino acids. In some embodiments the amino acid N is selected from hydrophobic amino acids.

In some embodiments the engineered protein comprises at least one amino acid N substitution (wherein “N” stands for any amino acid) at a position with a branch chain amino acid frequency score greater than 0. In some embodiments the engineered protein comprises at least one amino acid N substitution at a position with a branch chain amino acid frequency score of at least 0.01. In some embodiments the engineered protein comprises at least one amino acid N substitution at a position with a branch chain amino acid frequency score of at least 0.02. In some embodiments the engineered protein comprises at least one amino acid N substitution at a position with a branch chain amino acid frequency score of at least 0.03. In some embodiments the engineered protein comprises at least one amino acid N substitution at a position with a branch chain amino acid frequency score of at least 0.04. In some embodiments the engineered protein comprises at least one amino acid N substitution at a position with a branch chain amino acid frequency score of at least 0.05. In some embodiments the engineered protein comprises at least one amino acid N substitution at a position with a branch chain amino acid frequency score of at least 0.06. In some embodiments the engineered protein comprises at least one amino acid N substitution at a position with a branch chain amino acid frequency score of at least 0.07. In some embodiments the engineered protein comprises at least one amino acid N substitution at a position with a branch chain amino acid frequency score of at least 0.08. In some embodiments the engineered protein comprises at least one amino acid N substitution at a position with a branch chain amino acid frequency score of at least 0.09. In some embodiments the engineered protein comprises at least one amino acid N substitution at a position with a branch chain amino acid frequency score of at least 0.10. In some embodiments the engineered protein comprises at least one amino acid N substitution at a position with a branch chain amino acid frequency score of at least 0.11. In some embodiments the engineered protein comprises at least one amino acid N substitution at a position with a branch chain amino acid frequency score of at least 0.12. In some embodiments the engineered protein comprises at least one amino acid N substitution at a position with a branch chain amino acid frequency score of at least 0.13. In some embodiments the engineered protein comprises at least one amino acid N substitution at a position with a branch chain amino acid frequency score of at least 0.14. In some embodiments the engineered protein comprises at least one amino acid N substitution at a position with a branch chain amino acid frequency score of at least 0.15. In some embodiments the engineered protein comprises at least one amino acid N substitution at a position with a branch chain amino acid frequency score of at least 0.16. In some embodiments the engineered protein comprises at least one amino acid N substitution at a position with a branch chain amino acid frequency score of at least 0.17. In some embodiments the engineered protein comprises at least one amino acid N substitution at a position with a branch chain amino acid frequency score of at least 0.18. In some embodiments the engineered protein comprises at least one amino acid N substitution at a position with a branch chain amino acid frequency score of at least 0.19. In some embodiments the engineered protein comprises at least one amino acid N substitution at a position with a branch chain amino acid frequency score of at least 0.20. In some embodiments the engineered protein comprises at least one amino acid N substitution at a position with a branch chain amino acid frequency score of at least 0.25. In some embodiments the engineered protein comprises at least one amino acid N substitution at a position with a branch chain amino acid frequency score of at least 0.30. In some embodiments the engineered protein comprises at least one amino acid N substitution at a position with a branch chain amino acid frequency score of at least 0.35. In some embodiments the engineered protein comprises at least one amino acid N substitution at a position with a branch chain amino acid frequency score of at least 0.40. In some embodiments the engineered protein comprises at least one amino acid N substitution at a position with a branch chain amino acid frequency score of at least 0.45. In some embodiments the engineered protein comprises at least one amino acid N substitution at a position with a branch chain amino acid frequency score of at least 0.50. In some embodiments the amino acid N is selected from Leu, Ile, and Val. In some embodiments the amino acid N is selected from essential amino acids. In some embodiments the amino acid N is selected from hydrophobic amino acids.

In some embodiments the engineered protein comprises at least one amino acid N substitution (wherein “N” stands for any amino acid) at a position with an essential amino acid frequency score greater than 0. In some embodiments the engineered protein comprises at least one amino acid N substitution at a position with an essential amino acid frequency score of at least 0.01. In some embodiments the engineered protein comprises at least one amino acid N substitution at a position with an essential amino acid frequency score of at least 0.02. In some embodiments the engineered protein comprises at least one amino acid N substitution at a position with an essential amino acid frequency score of at least 0.03. In some embodiments the engineered protein comprises at least one amino acid N substitution at a position with an essential amino acid frequency score of at least 0.04. In some embodiments the engineered protein comprises at least one amino acid N substitution at a position with an essential amino acid frequency score of at least 0.05. In some embodiments the engineered protein comprises at least one amino acid N substitution at a position with an essential amino acid frequency score of at least 0.06. In some embodiments the engineered protein comprises at least one amino acid N substitution at a position with an essential amino acid frequency score of at least 0.07. In some embodiments the engineered protein comprises at least one amino acid N substitution at a position with an essential amino acid frequency score of at least 0.08. In some embodiments the engineered protein comprises at least one amino acid N substitution at a position with an essential amino acid frequency score of at least 0.09. In some embodiments the engineered protein comprises at least one amino acid N substitution at a position with an essential amino acid frequency score of at least 0.10. In some embodiments the engineered protein comprises at least one amino acid N substitution at a position with an essential amino acid frequency score of at least 0.11. In some embodiments the engineered protein comprises at least one amino acid N substitution at a position with an essential amino acid frequency score of at least 0.12. In some embodiments the engineered protein comprises at least one amino acid N substitution at a position with an essential amino acid frequency score of at least 0.13. In some embodiments the engineered protein comprises at least one amino acid N substitution at a position with an essential amino acid frequency score of at least 0.14. In some embodiments the engineered protein comprises at least one amino acid N substitution at a position with an essential amino acid frequency score of at least 0.15. In some embodiments the engineered protein comprises at least one amino acid N substitution at a position with an essential amino acid frequency score of at least 0.16. In some embodiments the engineered protein comprises at least one amino acid N substitution at a position with an essential amino acid frequency score of at least 0.17. In some embodiments the engineered protein comprises at least one amino acid N substitution at a position with a essential amino acid frequency score of at least 0.18. In some embodiments the engineered protein comprises at least one amino acid N substitution at a position with an essential amino acid frequency score of at least 0.19. In some embodiments the engineered protein comprises at least one amino acid N substitution at a position with an essential amino acid frequency score of at least 0.20. In some embodiments the engineered protein comprises at least one amino acid N substitution at a position with an essential amino acid frequency score of at least 0.25. In some embodiments the engineered protein comprises at least one amino acid N substitution at a position with an essential amino acid frequency score of at least 0.30. In some embodiments the engineered protein comprises at least one amino acid N substitution at a position with an essential amino acid frequency score of at least 0.35. In some embodiments the engineered protein comprises at least one amino acid N substitution at a position with an essential amino acid frequency score of at least 0.40. In some embodiments the engineered protein comprises at least one amino acid N substitution at a position with an essential amino acid frequency score of at least 0.45. In some embodiments the engineered protein comprises at least one amino acid N substitution at a position with an essential amino acid frequency score of at least 0.50. In some embodiments the amino acid N is selected from Leu, Ile, and Val. In some embodiments the amino acid N is selected from essential amino acids. In some embodiments the amino acid N is selected from hydrophobic amino acids.

In some embodiments the amino acid substitution(s) made to the reference secreted protein are selected so that for at least one of the substitutions the difference in total folding free energy between the reference secreted protein (without the substitution) and the engineered protein is less than or equal to −0.5, −0.4, −0.3, −0.0.2, −0.1, 0, 0.1, 0.2, 0.3, 0.4, or 0.5. In some embodiments the amino acid substitutions made to the reference secreted protein are selected so that the difference in total folding free energy between the reference secreted protein and the engineered protein is less than or equal to −0.5, −0.4, −0.3, −0.0.2, −0.1, 0, 0.1, 0.2, 0.3, 0.4, or 0.5.

In some embodiments the amino acid substitution(s) made to the reference secreted protein are selected so that for at least one of the substitutions the position entropy is at least 1.5, at least 1.6, at least 1.7, at least 1.8, at least 1.9, at least 2.0, at least 2.1, at least 2.2, at least 2.3, at least 2.4, at least 2.5, at least 2.6, at least 2.7, at least 2.8, at least 2.9, or at least 3.0.

In some embodiments from 5 to 50 non-essential amino acid residues in the reference secreted protein are substituted by essential amino acid residues in the engineered protein. In some embodiments from 10 to 50 non-essential amino acid residues in the reference secreted protein are substituted by essential amino acid residues in the engineered protein. In some embodiments from 25 to 50 non-essential amino acid residues in the reference secreted protein are substituted by essential amino acid residues in the engineered protein. In some embodiments at least 1, 2, 3, 4, 5, 6, 7, 8, 9, 10, 15, 20, 25, 30, 35, 40, 45, or 50 non-essential amino acid residues in the reference secreted protein are substituted by essential amino acid residues in the engineered protein.

In some embodiments from 5 to 50 non-branch chain amino acid residues in the reference secreted protein are substituted by branch chain amino acid residues in the engineered protein. In some embodiments from 10 to 50 non-branch chain amino acid residues in the reference secreted protein are substituted by branch chain amino acid residues in the engineered protein. In some embodiments from 25 to 50 non-branch chain amino acid residues in the reference secreted protein are substituted by branch chain amino acid residues in the engineered protein. In some embodiments at least 1, 2, 3, 4, 5, 6, 7, 8, 9, 10, 15, 20, 25, 30, 35, 40, 45, or 50 non-branch chain amino acid residues in the reference secreted protein are substituted by branch chain amino acid residues in the engineered protein.

In some embodiments from 5 to 50 non-Leu amino acid residues in the reference secreted protein are substituted by Leu amino acid residues in the engineered protein. In some embodiments from 10 to 50 non-Leu amino acid residues in the reference secreted protein are substituted by Leu amino acid residues in the engineered protein. In some embodiments from 25 to 50 non-Leu amino acid residues in the reference secreted protein are substituted Leu amino acid residues in the engineered protein. In some embodiments at least 1, 2, 3, 4, 5, 6, 7, 8, 9, 10, 15, 20, 25, 30, 35, 40, 45, or 50 non-Leu amino acid residues in the reference secreted protein are substituted by Leu amino acid residues in the engineered protein.

In some embodiments from 5 to 50 non-Val amino acid residues in the reference secreted protein are substituted by Val amino acid residues in the engineered protein. In some embodiments from 10 to 50 non-Val amino acid residues in the reference secreted protein are substituted by Val amino acid residues in the engineered protein. In some embodiments from 25 to 50 non-Val amino acid residues in the reference secreted protein are substituted Val amino acid residues in the engineered protein. In some embodiments at least 1, 2, 3, 4, 5, 6, 7, 8, 9, 10, 15, 20, 25, 30, 35, 40, 45, or 50 non-Val amino acid residues in the reference secreted protein are substituted by Val amino acid residues in the engineered protein.

In some embodiments from 5 to 50 non-Ile amino acid residues in the reference secreted protein are substituted by Ile amino acid residues in the engineered protein. In some embodiments from 10 to 50 non-Ile amino acid residues in the reference secreted protein are substituted by Ile amino acid residues in the engineered protein. In some embodiments from 25 to 50 non-Ile amino acid residues in the reference secreted protein are substituted Ile amino acid residues in the engineered protein. In some embodiments at least 1, 2, 3, 4, 5, 6, 7, 8, 9, 10, 15, 20, 25, 30, 35, 40, 45, or 50 non-Ile amino acid residues in the reference secreted protein are substituted by Ile amino acid residues in the engineered protein.

In some embodiments from 5 to 50% of non-essential amino acid residues in the reference secreted protein are substituted by essential amino acid residues in the engineered protein. In some embodiments from 10 to 50% of non-essential amino acid residues in the reference secreted protein are substituted by essential amino acid residues in the engineered protein. In some embodiments from 25 to 50% of non-essential amino acid residues in the reference secreted protein are substituted by essential amino acid residues in the engineered protein. In some embodiments at least 1, 2, 3, 4, 5, 6, 7, 8, 9, 10, 15, 20, 25, 30, 35, 40, 45, or 50% of non-essential amino acid residues in the reference secreted protein are substituted by essential amino acid residues in the engineered protein.

In some embodiments from 5 to 50% of non-branch chain amino acid residues in the reference secreted protein are substituted by branch chain amino acid residues in the engineered protein. In some embodiments from 10 to 50% of non-branch chain amino acid residues in the reference secreted protein are substituted by branch chain amino acid residues in the engineered protein. In some embodiments from 25 to 50% of non-branch chain amino acid residues in the reference secreted protein are substituted by branch chain amino acid residues in the engineered protein. In some embodiments at least 1, 2, 3, 4, 5, 6, 7, 8, 9, 10, 15, 20, 25, 30, 35, 40, 45, or 50% of non-branch chain amino acid residues in the reference secreted protein are substituted by branch chain amino acid residues in the engineered protein.

In some embodiments from 5 to 50% of non-Leu amino acid residues in the reference secreted protein are substituted by Leu amino acid residues in the engineered protein. In some embodiments from 10 to 50% of non-Leu amino acid residues in the reference secreted protein are substituted by Leu amino acid residues in the engineered protein. In some embodiments from 25 to 50% of non-Leu amino acid residues in the reference secreted protein are substituted Leu amino acid residues in the engineered protein. In some embodiments at least 1, 2, 3, 4, 5, 6, 7, 8, 9, 10, 15, 20, 25, 30, 35, 40, 45, or 50% of non-Leu amino acid residues in the reference secreted protein are substituted by Leu amino acid residues in the engineered protein.

In some embodiments from 5 to 50% of non-Val amino acid residues in the reference secreted protein are substituted by Val amino acid residues in the engineered protein. In some embodiments from 10 to 50% of non-Val amino acid residues in the reference secreted protein are substituted by Val amino acid residues in the engineered protein. In some embodiments from 25 to 50% of non-Val amino acid residues in the reference secreted protein are substituted Val amino acid residues in the engineered protein. In some embodiments at least 1, 2, 3, 4, 5, 6, 7, 8, 9, 10, 15, 20, 25, 30, 35, 40, 45, or 50% of non-Val amino acid residues in the reference secreted protein are substituted by Val amino acid residues in the engineered protein.

In some embodiments from 5 to 50% of non-Ile amino acid residues in the reference secreted protein are substituted by Ile amino acid residues in the engineered protein. In some embodiments from 10 to 50% of non-Ile amino acid residues in the reference secreted protein are substituted by Ile amino acid residues in the engineered protein. In some embodiments from 25 to 50% of non-Ile amino acid residues in the reference secreted protein are substituted Ile amino acid residues in the engineered protein. In some embodiments at least 1, 2, 3, 4, 5, 6, 7, 8, 9, 10, 15, 20, 25, 30, 35, 40, 45, or 50% of non-Ile amino acid residues in the reference secreted protein are substituted by Ile amino acid residues in the engineered protein.

In some embodiments from 5 to 50% of non-Arg amino acid residues in the reference secreted protein are substituted by Arg amino acid residues in the engineered protein. In some embodiments from 10 to 50% of non-Arg amino acid residues in the reference secreted protein are substituted by Arg amino acid residues in the engineered protein. In some embodiments from 25 to 50% of non-Arg amino acid residues in the reference secreted protein are substituted Arg amino acid residues in the engineered protein. In some embodiments at least 1, 2, 3, 4, 5, 6, 7, 8, 9, 10, 15, 20, 25, 30, 35, 40, 45, or 50% of non-Arg amino acid residues in the reference secreted protein are substituted by Arg amino acid residues in the engineered protein.

In some embodiments the engineered protein comprises at least one amino acid sequence, comprising an insertion of at least 5, at least 10, at least 15, at least 20, at least 25, or at least 50 amino acid residues. In some embodiments the at least one amino acid insertion comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% essential amino acids. In some embodiments the at least one amino acid insertion comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% branch chain amino acids. In some embodiments the at least one amino acid insertion comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% hydrophobic amino acids. In some embodiments the at least one amino acid insertion comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% Leu. In some embodiments the at least one amino acid insertion comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% Ile. In some embodiments the at least one amino acid insertion comprises at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% Val.

In some embodiments the at least one amino acid sequence insertion is located at a terminus of the engineered protein.

Phenylketonuria (PKU) is an autosomal recessive metabolic genetic disorder characterized by a mutation in the gene for the hepatic enzyme phenylalanine hydroxylase (PAH), rendering it nonfunctional. This enzyme is necessary to metabolize phenylalanine to tyrosine. When PAH activity is reduced, phenylalanine accumulates and is converted into phenylpyruvate (also known as phenylketone), which is detected in the urine. Untreated children are normal at birth, but fail to attain early developmental milestones, develop microcephaly, and demonstrate progressive impairment of cerebral function. Hyperactivity, EEG abnormalities and seizures, and severe learning disabilities are major clinical problems later in life. A characteristic odor of skin, hair, sweat and urine (due to phenylacetate accumulation); and a tendency to hypopigmentation and eczema are also observed. All PKU patients must adhere to a special diet low in Phe. Accordingly, engineered proteins intended for use by PKU patients should comprise a low number or no Phe residues. This can be done by selecting reference secreted proteins that have few or no Phe residues. Alternatively, the reference secreted protein may contain one or more Phe residues and such Phe residues may be replaced by non-Phe residues in the engineered protein. In some embodiments Phe residues present in reference secreted protein sequences are replaced by non-Phe residues such as Tyr. In some embodiments the reference secreted protein and/or engineered protein comprises a ratio of Phe residues to total amino acid residues equal to or lower than 5%, 4%, 3%, 2%, or 1%. In some embodiments the reference secreted protein and/or engineered protein comprises 10 or fewer Phe residues, 9 or fewer Phe residues, 8 or fewer Phe residues, 7 or fewer Phe residues, 6 or fewer Phe residues, 5 or fewer Phe residues, 4 or fewer Phe residues, 3 or fewer Phe residues, 2 or fewer Phe residues, 1 Phe residue, or no Phe residues.

Arginine is a conditionally nonessential amino acid, meaning most of the time it can be manufactured by the human body, and does not need to be obtained directly through the diet. Individuals who have poor nutrition, the elderly, or people with certain physical conditions (e.g., sepsis) may not produce sufficient amounts of arginine and therefore need to increase their intake of foods containing arginine. Arginine is believed to have beneficial health properties, including reducing healing time of injuries (particularly bone), and decreasing blood pressure, particularly high blood pressure during high risk pregnancies (pre-eclampsia). In addition, studies have shown that dietary supplementation with L-arginine is beneficial for enhancing the reproductive performance of pigs with naturally occurring intrauterine growth retardation, enhancing protein deposition and postnatal growth of milk-fed piglets, normalizing plasma glucose levels in streptozotocin-induced diabetic rats, reducing fat mass in obese Zucker diabetic fatty (ZDF) rats, and improving vascular function in diabetic rats. In some embodiments the engineered proteins disclosed herein comprise a ratio of Arginine residues to total amino acid residues in the engineered protein of equal to or greater than 3%, equal to or greater than 4%, equal to or greater than 5%, equal to or greater than 6%, equal to or greater than 7%, equal to or greater than 8%, equal to or greater than 9%, equal to or greater than 10%, equal to or greater than 11%, or equal to or greater than 12%.

Digestibility is a parameter relevant to the nutritive benefits and utility of engineered proteins. In some embodiments engineered proteins disclosed herein are screened to assess their digestibility. Digestibility of proteins can be assessed by any suitable method known in the art. In some embodiments the in vitro gastric and duodenal digestion assay using the physiologically relevant two-phase system described by Moreno et al. is used for this purpose. Moreno, et al., “Stability of the major allergen Brazil nut 2S albumin (Ber e 1) to physiologically relevant in vitro gastrointestinal digestion.” FEBS Journal, 341-352 (2005). Briefly, experimental proteins are sequentially exposed to a simulated gastric fluid (SGF) for 120 minutes and then transferred to a simulated duodenal fluid (SDF) to digest for an additional 120 minutes. Protein samples at different stages of the digestion (e.g., 2, 5, 15, 30, 60 and 120 min) are analyzed for digestion by sodium dodecyl sulfate polyacrylamide gel electrophoresis (SDS-PAGE). Each sample (20 μL) is added to 10 μL of ultrapure water and 10 μL of 4× NuPAGE LDS Sample buffer and heated at 95° C. for 10 min. The samples are loaded (10 μL) on a 15-lane 12% polyacrylamide NuPAGE Novex Bis-Tris gel and run for 35 min at 200 V then stained using SimplyBlue Safe Stain. The disappearance of protein over time indicates the rate at which the protein is digested in the assay. This assay can be used to assess comparative digestibility or to assess absolute digestibility. In some embodiments the digestibility of an engineered protein disclosed herein is higher (i.e., it digests to below the detection limit of the assay sooner) than whey protein. In some embodiments the engineered protein is not detectable in the assay by 2 minutes, 5 minutes, 15 minutes, 30 minutes, 60 minutes, or 120 minutes.

In some embodiments digestibility of an engineered protein is assessed by identification and quantification of digestive protease recognition sites in the protein amino acid sequence. In some embodiments the engineered protein comprises at least one protease recognition site selected from a pepsin recognition site, a trypsin recognition site, and a chymotrypsin recognition site. In some embodiments at least one amino acid mutation is made to the reference secreted protein amino acid sequence to add at least one protease recognition site to the engineered protein.

As used herein, a “pepsin recognition site” is any site in a polypeptide sequence that is experimentally shown to be cleaved by pepsin. In some embodiments it is a peptide bond after (i.e., downstream of) an amino acid residue selected from Phe, Trp, Tyr, Leu, Ala, Glu, and Gln, provided that the following residue is not an amino acid residue selected from Ala, Gly, and Val.

As used herein, a “trypsin recognition site” is any site in a polypeptide sequence that is experimentally shown to be cleaved by trypsin. In some embodiments it is a peptide bond after an amino acid residue selected from Lys or Arg, provided that the following residue is not a proline.

As used herein, a “chymotrypsin recognition site” is any site in a polypeptide sequence that is experimentally shown to be cleaved by chymotrypsin. In some embodiments it is a peptide bond after an amino acid residue selected from Phe, Trp, Tyr, and Leu.

Disulfide bonded cysteine residues in a protein tend to reduce the rate of digestion of the protein compared to what it would be in the absence of the disulfide bond. Accordingly, digestibility of a protein with fewer disulfide bonds tends to be higher than for a comparable protein with a greater number of disulfide bonds. Accordingly, in some embodiments an engineered protein disclosed herein is screened to identify the number of cysteine residues present and to allow selection of an engineered protein comprising a relatively low number of cysteine residues. In some embodiments at least one amino acid replacement is made to the reference secreted protein amino acid sequence to remove at least one protease recognition site in the engineered protein. In some embodiments the engineered protein comprises a ratio of Cys residues to total amino acid residues equal to or lower than 5%, 4%, 3%, 2%, or 1%. In some embodiments the engineered protein comprises 10 or fewer Cys residues, 9 or fewer Cys residues, 8 or fewer Cys residues, 7 or fewer Cys residues, 6 or fewer Cys residues, 5 or fewer Cys residues, 4 or fewer Cys residues, 3 or fewer Cys residues, 2 or fewer Cys residues, 1 Cys residue, or no Cys residues.

In some embodiments the engineered protein is soluble. Solubility can be measured by any method known in the art. In some embodiments solubility is examined by centrifuge concentration followed by protein concentration assays. Samples of proteins in 20 mM HEPES pH 7.5 are tested for protein concentration according to protocols using two methods, Coomassie Plus (Bradford) Protein Assay (Thermo Scientific) and Bicinchoninic Acid (BCA) Protein Assay (Sigma-Aldrich). Based on these measurements 10 mg of protein is added to an Amicon Ultra 3 kDa centrifugal filter (Millipore). Samples are concentrated by centrifugation at 10,000×g for 30 minutes. The final, now concentrated, samples are examined for precipitated protein and then tested for protein concentration as above using two methods, Bradford and BCA.

In some embodiments the engineered proteins have a final solubility limit of at least 5 g/L, 10 g/L, 20 g/L, 30 g/L, 40 g/L, 50 g/L, or 100 g/L at physiological pH. In some embodiments the engineered proteins are greater than 50%, greater than 60%, greater than 70%, greater than 80%, greater than 90%, greater than 95%, greater than 96%, greater than 97%, greater than 98%, greater than 99%, or greater than 99.5% soluble with no precipitated protein observed at a concentration of greater than 5 g/L, or 10 g/L, or 20 g/L, or 30 g/L, or 40 g/L, or 50 g/L, or 100 g/L at physiological pH. In some embodiments, the solubility of the engineered protein is higher than those typically reported in studies examining the solubility limits of whey (12.5 g/L; Pelegrine et al., Lebensm.-Wiss. U.-Technol. 38 (2005) 77-80) and soy (10 g/L; Lee et al., JAOCS 80(1) (2003) 85-90).

In some embodiments, the engineered protein exhibits enhanced stability. As used herein, a “stable” protein is one that resists changes (e.g., unfolding, oxidation, aggregation, hydrolysis, etc.) that alter the biophysical (e.g., solubility), biological (e.g., digestibility), or compositional (e.g. proportion of Leucine amino acids) traits of the protein of interest.

Protein stability can be measured using various assays known in the art and engineered proteins disclosed herein may have a stability above a threshold. In some embodiments a protein is selected that displays thermal stability that is comparable to or better than that of whey protein. In some embodiments the stability of engineered protein samples is determined by monitoring aggregation formation using size exclusion chromatography (SEC) after exposure to extreme temperatures. Samples of proteins to be tested are prepared at 10 g/L protein in water and mixed thoroughly. Protein solutions are placed in a heating block at 90° C. and samples are taken after 0, 1, 5, 10, 30 and 60 min for SEC analysis.

For example, SEC analysis can run on a Superdex 75 5/150 GL column (GE Healthcare) using an Agilent 1100 HPLC with a mobile phase of 20 mM Na₂PO₄ and 130 mM NaCl at pH 7. After heating, samples are diluted to 2 g/L for 10 μl injection onto the column. Protein is detected by monitoring absorbance at 214 nm, aggregates are characterized as peaks larger in size (eluting faster) than the protein of interest. No overall change in peak area indicates no precipitation of protein during the heat treatment. Whey protein rapidly forms approximately 80% aggregates when exposed to 90° C. in this assay. In some embodiments an engineered protein of this disclosure shows resistance to aggregation, exhibiting, for example, less than 80% aggregation, less than 10% aggregation, or no detectable aggregation.

For most embodiments it is preferred that the engineered protein not exhibit inappropriately high allergenicity. Accordingly, in some embodiments the potential allergenicy of the engineered protein is assessed. This can be done by any suitable method known in the art. In some embodiments an allergenicity score is calculated. The allergenicity score is a primary sequence based metric based on WHO recommendations (See, for example, www.fao.org/ag/agn/food/pdf/allergygm.pdf) for assessing how similar a protein is to any known allergen, the primary hypothesis being that high percent identity between a target and a known allergen is likely indicative of cross reactivity. For a given protein, the allergenicity score is found by examining all possible contiguous 80 amino acid fragments and locally aligning each fragment against a database of known allergen sequences using the FASTA algorithm with the BLOSUM50 substitution matrix, a gap open penalty of 10, and a gap extension penalty of 2. The highest percent identity of any 80 amino acid window with any allergen is taken as the final score for the protein of interest. The WHO guidelines suggest using a 35% identity cutoff. In some embodiments the engineered protein has an allergenicity score less than 35%. In some embodiments a cutoff of less than 35% identity is used. In some embodiments a cutoff of from 30% to 35% identity is used. In some embodiments a cutoff of from 25% to 30% identity is used. In some embodiments a cutoff of from 20% to 25% identity is used. In some embodiments a cutoff of from 15% to 20% identity is used. In some embodiments a cutoff of from 10% to 15% identity is used. In some embodiments a cutoff of from 5% to 10% identity is used. In some embodiments a cutoff of from 0% to 5% identity is used. In some embodiments a cutoff of greater than 35% identity is used. In some embodiments a cutoff of from 35% to 40% identity is used. In some embodiments a cutoff of from 40% to 45% identity is used. In some embodiments a cutoff of from 45% to 50% identity is used. In some embodiments a cutoff of from 50% to 55% identity is used. In some embodiments a cutoff of from 55% to 60% identity is used. In some embodiments a cutoff of from 65% to 70% identity is used. In some embodiments a cutoff of from 70% to 75% identity is used. In some embodiments a cutoff of from 75% to 80% identity is used.

Skilled artisans are able to identify and use a suitable database of known allergens for this purpose. In some embodiments the database is made by selecting proteins from more than one database source. In some embodiments the custom database comprises pooled allergen lists collected by the Food Allergy Research and Resource Program (http://www.allergenonline.org/), UNIPROT annotations (http://www.uniprot.org/docs/allergen), and the Structural Database of Allergenic Proteins (SDAP, http://fermi.utmb.edu/SDAP/sdap_lnk.html). This database includes all currently recognized allergens by the International Union of Immunological Societies (IUIS, http://www.allergen.org/) as well as a large number of additional allergens not yet officially named.

In some embodiments all (or a selected subset) contiguous amino acid windows of different lengths (e.g., 70, 60, 50, 40, 30, 20, 10, 8 or 6 amino acid windows) of an engineered protein are tested against an allergen database and peptide sequences that have 100% identity, 95% or higher identity, 90% or higher identity, 85% or higher identity, 80% or higher identity, 75% or higher identity, 70% or higher identity, 65% or higher identity, 60% or higher identity, 55% or higher identity, or 50% or higher identity matches are identified for further examination of potential allergenicity.

One feature that can enhance the utility of a engineered protein is its charge (or per amino acid charge). Engineered proteins with higher charge can in some embodiments exhibit desirable characteristics such as increased solubility, increased stability, resistance to aggregation, and desirable taste profiles. For example, a charged engineered protein that exhibits enhanced solubility can be formulated into a beverage or liquid formulation that includes a high concentration of engineered protein in a relatively low volume of solution, thus delivering a large dose of protein nutrition per unit volume. A charged engineered protein that exhibits enhanced solubility can be useful in sports drinks or recovery drinks wherein a user (e.g., an athlete) wants to ingest protein before, during or after physical activity. A charged engineered protein that exhibits enhanced solubility can also be particularly useful in a clinical setting wherein a subject (e.g., a patient or an elderly person) is in need of protein nutrition but is unable to ingest solid foods or large volumes of liquids.

Certain free amino acids and mixtures of free amino acids are known to have a bitter or otherwise unpleasant taste. In addition, hydrolysates of common proteins (e.g., whey and soy) often have a bitter or unpleasant taste. In some embodiments, an engineered protein disclosed and described herein does not have a bitter or otherwise unpleasant taste. In some embodiments, an engineered protein disclosed and described herein has a more acceptable taste as compared to at least one of free amino acids, mixtures of free amino acids, and/or protein hydrolysates. In some embodiments, an engineered protein disclosed and described herein has a taste that is equal to or exceeds at least one of whey protein and whey protein hydrolysates.

Proteins are known to have tastes covering the five established taste modalities: sweet, sour, bitter, salty and umami. The taste of a particular protein (or its lack thereof) can be attributed to several factors, including the primary structure, the presence of charged side chains, and the electronic and conformational features of the protein. In some embodiments, an engineered protein disclosed and described herein is designed to have a desired taste (e.g., sweet, salty, umami) and/or not to have an undesired taste (e.g., bitter, sour). In this context “design” includes, for example, selecting naturally occurring proteins embodying features that achieve the desired taste property, as well as creating muteins of naturally-occurring proteins that have desired taste properties. For example, an engineered protein can be designed to interact with specific taste receptors, such as sweet receptors (T1R2-T1R3 heterodimer) or umami receptors (T1R1-T1R3 heterodimer, mGluR4, and/or mGluR1). Further, an engineered protein may be designed not to interact, or to have diminished interaction, with other taste receptors, such as bitter receptors (T2R receptors).

An engineered protein disclosed and described herein can also elicit different physical sensations in the mouth when ingested, sometimes referred to as “mouth feel”. The mouth feel of the engineered protein may be due to one or more factors including primary structure, the presence of charged side chains, and the electronic and conformational features of the protein. In some embodiments, an engineered protein elicits a buttery or fat-like mouth feel when ingested.

In some embodiments the engineered protein comprises from 20 to 5,000 amino acids, from 20-2,000 amino acids, from 20-1,000 amino acids, from 20-500 amino acids, from 20-250 amino acids, from 20-200 amino acids, from 20-150 amino acids, from 20-100 amino acids, from 20-40 amino acids, from 30-50 amino acids, from 40-60 amino acids, from 50-70 amino acids, from 60-80 amino acids, from 70-90 amino acids, from 80-100 amino acids, at least 25 amino acids, at least 30 amino acids, at least 35 amino acids, at least 40 amino acids, at least 2455 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, at least 105 amino acids, at least 110 amino acids, at least 115 amino acids, at least 120 amino acids, at least 125 amino acids, at least 130 amino acids, at least 135 amino acids, at least 140 amino acids, at least 145 amino acids, at least 150 amino acids, at least 155 amino acids, at least 160 amino acids, at least 165 amino acids, at least 170 amino acids, at least 175 amino acids, at least 180 amino acids, at least 185 amino acids, at least 190 amino acids, at least 195 amino acids, at least 200 amino acids, at least 205 amino acids, at least 210 amino acids, at least 215 amino acids, at least 220 amino acids, at least 225 amino acids, at least 230 amino acids, at least 235 amino acids, at least 240 amino acids, at least 245 amino acids, or at least 250 amino acids. In some embodiments the engineered protein consists of from 20 to 5,000 amino acids, from 20-2,000 amino acids, from 20-1,000 amino acids, from 20-500 amino acids, from 20-250 amino acids, from 20-200 amino acids, from 20-150 amino acids, from 20-100 amino acids, from 20-40 amino acids, from 30-50 amino acids, from 40-60 amino acids, from 50-70 amino acids, from 60-80 amino acids, from 70-90 amino acids, from 80-100 amino acids, at least 25 amino acids, at least 30 amino acids, at least 35 amino acids, at least 40 amino acids, at least 2455 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, at least 105 amino acids, at least 110 amino acids, at least 115 amino acids, at least 120 amino acids, at least 125 amino acids, at least 130 amino acids, at least 135 amino acids, at least 140 amino acids, at least 145 amino acids, at least 150 amino acids, at least 155 amino acids, at least 160 amino acids, at least 165 amino acids, at least 170 amino acids, at least 175 amino acids, at least 180 amino acids, at least 185 amino acids, at least 190 amino acids, at least 195 amino acids, at least 200 amino acids, at least 205 amino acids, at least 210 amino acids, at least 215 amino acids, at least 220 amino acids, at least 225 amino acids, at least 230 amino acids, at least 235 amino acids, at least 240 amino acids, at least 245 amino acids, or at least 250 amino acids.

1. Methods of Identifying Reference Secreted Proteins

Without wishing to be bound by any theory, it is believed that modifying the amino acid sequence of reference secreted proteins to improve at least one nutritive feature of the protein is a useful way to make proteins with useful nutritive amino acid compositions. Because the reference secreted protein is naturally secreted by the organism it is possible, in some embodiments, to create proteins with useful nutritive content which are secreted using this approach. Secreted nutritive proteins may be particular useful in certain embodiments because secretion can aid in manufacture of engineered proteins in certain applications.

To this end, in some embodiments annotated databases of the proteins of organisms of interest are screened to identify those that are characterized as secreted. An alternative or additional method is to screen sequence information for the proteins of an organism of interest and identify those proteins that comprise a secretion leader sequence. An alternative or additional method is to obtain cDNAs encoding proteins of an organism of interest and to screen those cDNAs functionally to identify those that encode secreted proteins. The resulting set of proteins that are identified by one or more of these methods in or any equivalent method for an organism is teamed the secretome for that organism. In some embodiments any secreted protein is used as a reference secreted protein in the methods of this disclosure.

In some embodiments secreted proteins are screened to identify those that comprise structural domains and/or folds that have been used in previous studies to reengineer protein-protein binding interactions. The NCBI Conserved Domain Database (Marchler-Bauer A., and Bryant, S. H. “CD-Search: protein domain annotations on the fly”. Nuc. Acid. Res. (2004) 32: W327-W331) includes such protein domains. (Binz, K H, and Pluckthun, A. “Engineered proteins as specific binding reagents”. Curr. Op. Biotech. (2005) 16: 459-469; Gebauer, M. and Skerra, A. “Engineered protein scaffolds as next-generation antibody therapeutics”. Curr. Op. Chem. Biol. (2009) 13: 245-255; Lehtio, J., Teeri T. T., and Nygren P. A. “Alpha-Amylase Inhibitors Selected From a Combinatorial Library of a Cellulose Binding Domain Scaffold”. Proteins: Struct., Func., Gene., (2000) 41: 316-322; and Olson C A and Roberts R W. “Design, expression, and stability of a diverse protein library based on the human fibronectin type III domain”. Prot. Sci. (2007) 16: 476-484.) As such, the database can be used to identify protein scaffolds that are expected to contain a robust, stable fold with known variable positions or regions, wherein such variable positions or regions can be tailored to match a desired overall amino acid distribution. In some embodiments the naturally occurring protein comprising such a domain is used as a reference secreted protein. In some embodiments some or all of the remaining portions of the naturally occurring protein comprising such a domain is not included in an engineered protein comprising a derivative of the domain.

2. Methods of Identifying Amino Acid Positions in Reference Secreted Proteins to Modify in Engineered Proteins

This disclosure identifies six factors that may be used to identify amino acid positions in a reference secreted protein for substitution by another amino acid, for example, positions where the amino acid in the reference secreted protein sequence are non-Leu for substitution with a Leu amino acid. The six factors are amino acid likelihood (AALike), amino acid type likelihood (AATLike), position entropy (Sₚₒₛ), amino acid type position entropy (S_{AATpos}), relative free energy of folding (ΔΔG_{fold}), and secondary structure identity (LoopID). These factors may be combined to identify amino acid positions for substitution using the following Formula 3.

((α)AALike+(β)AATLike+(γ)Spos+(δ)SAATpos+(ε)ΔΔGfold+(ζ)LoopID)/(α+β+γ+δ+ε+ζ) Formula 3:

In Formula 3, the coefficients α, β, γ, δ, ε, and ζ are scaling coefficients chosen by a skilled artisan that indicate the relative importance of each factor when rank ordering a set of positions in a secreted protein. In some embodiments 1, 2, 3, 4, or 5 of the coefficients are set to 0.

B. Nucleic Acids

Also provided herein are nucleic acids encoding engineered proteins disclosed herein. In some embodiments the nucleic acid is isolated. In some embodiments the nucleic acid is purified. In some embodiments the nucleic acid is synthetic.

In some embodiments the nucleic acid comprises the coding sequence for an engineered protein disclosed herein. In some embodiments the nucleic acid consists of the coding sequence for an engineered protein disclosed herein. In some embodiments the nucleic acid further comprises an expression control sequence operably linked to the coding sequence.

In some embodiments of the nucleic acid, the nucleic acid comprises a nucleic acid sequence that encodes an engineered protein disclosed in Section A above. In some embodiments of the nucleic acid, the nucleic acid consists of a nucleic acid sequence that encodes an engineered protein disclosed in Section A above.

In some embodiments the nucleic acid comprises at least 10 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1,000 nucleotides. In some embodiments the nutritrive nucleic acid comprises from 10 to 100 nucleotides, from 20 to 100 nucleotides, from 10 to 50 nucleotides, or from 20 to 40 nucleotides. In some embodiments the nucleic acid comprises all or part of an open reading frame that encodes a nutritive polypeptide. In some embodiments the nucleic acid consists of an open reading frame that encodes a fragment of a naturally occurring protein, wherein the open reading frame does not encode the complete naturally occurring protein. In some embodiments the nucleic acid is a cDNA. In some embodiments nucleic acid molecules are provided that comprise a sequence that is at least 50%, 60%, 70%, 80%, 85%, 90%, 95%, 96%, 97%, 98%, 99%, or 99.9% identical to a naturally occurring nucleic acid. In some embodiments nucleic acids are provided that hybridize under stringent hybridization conditions with at least one reference nucleic acid.

C. Vectors

Also provided are vectors, including expression vectors, which comprise at least one of the nucleic acid molecules disclosed herein, as described further herein. In some embodiments, the vectors comprise at least one isolated nucleic acid molecule encoding an engineered protein as disclosed herein. In alternative embodiments, the vectors comprise such a nucleic acid molecule operably linked to one or more expression control sequence. The vectors can thus be used to express at least one recombinant protein in a recombinant microbial host cell.

Suitable vectors for expression of nucleic acids in microorganisms are well known to those of skill in the art. Suitable vectors for use in cyanobacteria are described, for example, in Heidorn et al., “Synthetic Biology in Cyanobacteria: Engineering and Analyzing Novel Functions,” Methods in Enzymology, Vol. 497, Ch. 24 (2011). Exemplary replicative vectors that can be used for engineering cyanobacteria as disclosed herein include pPMQAK1, pSL1211, pFC1, pSB2A, pSCR119/202, pSUN119/202, pRL2697, pRL25C, pRL1050, pSG111M, and pPBH201.

Other vectors such as pJB161 which are capable of receiving nucleic acid sequences disclosed herein may also be used. Vectors such as pJB161 comprise sequences which are homologous with sequences present in plasmids endogenous to certain photosynthetic microorganisms (e.g., plasmids pAQ1, pAQ3, and pAQ4 of certain Synechococcus species). Examples of such vectors and how to use them is known in the art and provided, for example, in Xu et al., “Expression of Genes in Cyanobacteria: Adaptation of Endogenous Plasmids as Platforms for High-Level Gene Expression in Synechococcus sp. PCC 7002,” Chapter 21 in Robert Carpentier (ed.), “Photosynthesis Research Protocols,” Methods in Molecular Biology, Vol. 684, 2011, which is hereby incorporated herein. Recombination between pJB161 and the endogenous plasmids in vivo yield engineered microbes expressing the genes of interest from their endogenous plasmids. Alternatively, vectors can be engineered to recombine with the host cell chromosome, or the vector can be engineered to replicate and express genes of interest independent of the host cell chromosome or any of the host cell's endogenous plasmids.

A further example of a vector suitable for recombinant protein production is the pET system (Novagen®). This system has been extensively characterized for use in E. coli and other microorganisms. In this system, target genes are cloned in pET plasmids under control of strong bacteriophage T7 transcription and (optionally) translation signals; expression is induced by providing a source of T7 RNA polymerase in the host cell. T7 RNA polymerase is so selective and active that, when fully induced, almost all of the microorganism's resources are converted to target gene expression; the desired product can comprise more than 50% of the total cell protein a few hours after induction. It is also possible to attenuate the expression level simply by lowering the concentration of inducer. Decreasing the expression level may enhance the soluble yield of some target proteins. In some embodiments this system also allows for maintenance of target genes in a transcriptionally silent un-induced state.

In some embodiments of using this system, target genes are cloned using hosts that do not contain the T7 RNA polymerase gene, thus alleviating potential problems related to plasmid instability due to the production of proteins potentially toxic to the host cell. Once established in a non-expression host, target protein expression may be initiated either by infecting the host with λCE6, a phage that carries the T7 RNA polymerase gene under the control of the λ pL and pI promoters, or by transferring the plasmid into an expression host containing a chromosomal copy of the T7 RNA polymerase gene under lacUV5 control. In the second case, expression is induced by the addition of IPTG or lactose to the bacterial culture or using an autoinduction medium. Other plasmids systems that are controlled by the lac operator, but do not require the T7 RNA polymerase gene and rely upon E. coli's native RNA polymerase include the pTrc plasmid suite (Invitrogen) or pQE plamid suite (QIAGEN).

In other embodiments it is possible to clone directly into expression hosts. Two types of T7 promoters and several hosts that differ in their stringency of suppressing basal expression levels are available, providing great flexibility and the ability to optimize the expression of a wide variety of target genes.

Promoters useful for expressing the recombinant genes described herein include both constitutive and inducible/repressible promoters. Examples of inducible/repressible promoters include nickel-inducible promoters (e.g., PnrsA, PnrsB; see, e.g., Lopez-Mauy et al., Cell (2002) v.43: 247-256) and urea repressible promoters such as PnirA (described in, e.g., Qi et al., Applied and Environmental Microbiology (2005) v.71: 5678-5684). Additional examples of inducible/repressible promoters include PnirA (promoter that drives expression of the nirA gene, induced by nitrate and repressed by urea) and Psuf (promoter that drives expression of the sufB gene, induced by iron stress).

Examples of constitutive promoters include Pcpc (promoter that drives expression of the cpc operon), Prbc (promoter that drives expression of rubisco), PpsbAII (promoter that drives expression of the D1 protein of photosystem II reaction center), Pcro (lambda phage promoter that drives expression of cro). In other embodiments, a PaphII and/or a laclq-Ptrc promoter can used to control expression. Where multiple recombinant genes are expressed in an engineered microorganism, the different genes can be controlled by different promoters or by identical promoters in separate operons, or the expression of two or more genes may be controlled by a single promoter as part of an operon.

Further non-limiting examples of inducible promoters include, but are not limited to, those induced by expression of an exogenous protein (e.g., T7 RNA polymerase, SP6 RNA polymerase), by the presence of a small molecule (e.g., IPTG, galactose, tetracycline, steroid hormone, abscisic acid), by absence or low concentration of small molecules (e.g., CO₂, iron, nitrogen), by metals or metal ions (e.g., copper, zinc, cadmium, nickel), and by environmental factors (e.g., heat, cold, stress, light, darkness), and by growth phase. In some embodiments, the inducible promoter is tightly regulated such that in the absence of induction, substantially no transcription is initiated through the promoter. In some embodiments, induction of the promoter does not substantially alter transcription through other promoters. Also, generally speaking, the compound or condition that induces an inducible promoter is not be naturally present in the organism or environment where expression is sought.

In some embodiments, the inducible promoter is induced by limitation of CO₂ supply to a cyanobacteria culture. By way of non-limiting example, the inducible promoter may be the promoter sequence of Synechocystis PCC 6803 that are up-regulated under the CO₂-limitation conditions, such as the cmp genes, ntp genes, ndh genes, sbt genes, chp genes, and rbc genes, or a variant or fragment thereof.

In some embodiments, the inducible promoter is induced by iron starvation or by entering the stationary growth phase. In some embodiments, the inducible promoter may be variant sequences of the promoter sequence of cyanobacterial genes that are up-regulated under Fe-starvation conditions such as isiA, or when the culture enters the stationary growth phase, such as isiA, phrA, sigC, sigB, and sigH genes, or a variant or fragment thereof.

In some embodiments, the inducible promoter is induced by a metal or metal ion. By way of non-limiting example, the inducible promoter may be induced by copper, zinc, cadmium, mercury, nickel, gold, silver, cobalt, and bismuth or ions thereof. In some embodiments, the inducible promoter is induced by nickel or a nickel ion. In some embodiments, the inducible promoter is induced by a nickel ion, such as Ni²⁺. In another exemplary embodiment, the inducible promoter is the nickel inducible promoter from Synechocystis PCC 6803. In another embodiment, the inducible promoter may be induced by copper or a copper ion. In yet another embodiment, the inducible promoter may be induced by zinc or a zinc ion. In still another embodiment, the inducible promoter may be induced by cadmium or a cadmium ion. In yet still another embodiment, the inducible promoter may be induced by mercury or a mercury ion. In an alternative embodiment, the inducible promoter may be induced by gold or a gold ion. In another alternative embodiment, the inducible promoter may be induced by silver or a silver ion. In yet another alternative embodiment, the inducible promoter may be induced by cobalt or a cobalt ion. In still another alternative embodiment, the inducible promoter may be induced by bismuth or a bismuth ion.

In some embodiments, the promoter is induced by exposing a cell comprising the inducible promoter to a metal or metal ion. The cell may be exposed to the metal or metal ion by adding the metal to the microbial growth media. In certain embodiments, the metal or metal ion added to the microbial growth media may be efficiently recovered from the media. In other embodiments, the metal or metal ion remaining in the media after recovery does not substantially impede downstream processing of the media or of the bacterial gene products.

Further non-limiting examples of constitutive promoters include constitutive promoters from Gram-negative bacteria or a bacteriophage propagating in a Gram-negative bacterium. For instance, promoters for genes encoding highly expressed Gram-negative gene products may be used, such as the promoter for Lpp, OmpA, rRNA, and ribosomal proteins. Alternatively, regulatable promoters may be used in a strain that lacks the regulatory protein for that promoter. For instance P_{lac}, P_{tac}, and P_{trc}, may be used as constitutive promoters in strains that lack Lac1. Similarly, P22 P_{R} and P_{L} may be used in strains that lack the lambda C2 repressor protein, and lambda P_{R} and P_{L} may be used in strains that lack the lambda C1 repressor protein. In one embodiment, the constitutive promoter is from a bacteriophage. In another embodiment, the constitutive promoter is from a Salmonella bacteriophage. In yet another embodiment, the constitutive promoter is from a cyanophage. In some embodiments, the constitutive promoter is a Synechocystis promoter. For instance, the constitutive promoter may be the PpsbAll promoter or its variant sequences, the Prbc promoter or its variant sequences, the P_{cpc} promoter or its variant sequences, and the PrnpB promoter or its variant sequences.

D. Host Microorganisms

Also provided are host cells transformed with the nucleic acid molecules or vectors disclosed herein, and descendants thereof. In some embodiments the host cells are microbial cells. In some embodiments, the host cells carry the nucleic acid sequences on vectors, which may but need not be freely replicating vectors. In other embodiments, the nucleic acids have been integrated into the genome of the host cells and/or into an endogenous plasmid of the host cells. The transformed host cells find use, e.g., in the production of recombinant engineered proteins disclosed herein.

In some embodiments the protein is an endogenous protein of the host cell used to express it. That is, the cellular genome of the host cell comprises an open reading frame that encodes the recombinant protein. In some embodiments regulatory sequences sufficient to increase expression of the protein are inserted into the host cell genome and operatively linked to the endogenous open reading frame such that the regulatory sequences drive overexpression of the recombinant protein from a recombinant nucleic acid. In some embodiments heterologous nucleic acid sequences are fused to the endogenous open reading frame of the protein and cause the protein to be synthesized comprising a heterologous amino acid sequence that changes the cellular trafficking of the recombinant protein, such as directing it to an organelle or to a secretion pathway. In some embodiments an open reading frame that encodes the endogeneous host cell protein is introduced into the host cell on a plasmid that further comprises regulatory sequences operatively linked to the open reading frame. In some embodiments the recombinant host cell expresses at least 2 times, at least 3 times, at least 4 times, at least 5 times, at least 10 times, or at least 20 times, at least 30 times, at least 40 times, at least 50 times, or at least 100 times more of the recombinant protein than the amount of the protein produced by a similar host cell grown under similar conditions.

“Microorganisms” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

A variety of host microorganisms can be transformed with a nucleic acid sequence disclosed herein and can in some embodiments produce a recombinant engineered protein disclosed herein. Suitable host microorganisms include both autotrophic and heterotrophic microbes. In some applications the autotrophic microorganisms allows for a reduction in the fossil fuel and/or electricity inputs required to make an engineered protein encoded by a recombinant nucleic acid sequence introduced into the host microorganism. This, in turn, in some applications reduces the cost and/or the environmental impact of producing the engineered protein and/or reduces the cost and/or the environmental impact in comparison to the cost and/or environmental impact of manufacturing alternative nutritive proteins, such as whey, egg, and soy. For example, the cost and/or environmental impact of making an engineered protein disclosed herein using a host microorganism as disclosed herein is in some embodiments lower that the cost and/or environmental impact of making whey protein in a form suitable for human consumption by processing of cow's milk.

Photoautotrophic microorganisms include eukaryotic algae, as well as prokaryotic cyanobacteria, green-sulfur bacteria, green non-sulfur bacteria, purple sulfur bacteria, and purple non-sulfur bacteria.

Extremophiles are also contemplated as suitable organisms. Such organisms withstand various environmental parameters such as temperature, radiation, pressure, gravity, vacuum, desiccation, salinity, pH, oxygen tension, and chemicals. They include hyperthermophiles, which grow at or above 80° C. such as Pyrolobus fumarii; thermophiles, which grow between 60-80° C. such as Synechococcus lividis; mesophiles, which grow between 15-60° C.; and psychrophiles, which grow at or below 15° C. such as Psychrobacter and some insects. Radiation tolerant organisms include Deinococcus radiodurans. Pressure-tolerant organisms include piezophiles, which tolerate pressure of 130 MPa. Weight-tolerant organisms include barophiles. Hypergravity (e.g., >1 g) hypogravity (e.g., <1 g) tolerant organisms are also contemplated. Vacuum tolerant organisms include tardigrades, insects, microbes and seeds. Dessicant tolerant and anhydrobiotic organisms include xerophiles such as Artemia salina; nematodes, microbes, fungi and lichens. Salt-tolerant organisms include halophiles (e.g., 2-5 M NaCl) Halobacteriacea and Dunaliella salina. pH-tolerant organisms include alkaliphiles such as Natronobacterium, Bacillus firmus OF4, Spirulina spp. (e.g., pH>9) and acidophiles such as Cyanidium caldarium, Ferroplasma sp. (e.g., low pH). Anaerobes, which cannot tolerate O₂ such as Methanococcus jannaschii; microaerophils, which tolerate some O₂ such as Clostridium and aerobes, which require O₂ are also contemplated. Gas-tolerant organisms, which tolerate pure CO₂ include Cyanidium caldarium and metal tolerant organisms include metalotolerants such as Ferroplasma acidarmanus (e.g., Cu, As, Cd, Zn), Ralstonia sp. CH34 (e.g., Zn, Co, Cd, Hg, Pb). Gross, Michael. Life on the Edge: Amazing Creatures Thriving in Extreme Environments. New York: Plenum (1998) and Seckbach, J. “Search for Life in the Universe with Terrestrial Microbes Which Thrive Under Extreme Conditions.” In Cristiano Batalli Cosmovici, Stuart Bowyer, and Dan Wertheimer, eds., Astronomical and Biochemical Origins and the Search for Life in the Universe, p. 511. Milan: Editrice Compositori (1997).

Algae and cyanobacteria include but are not limited to the following genera: Acanthoceras, Acanthococcus, Acaryochloris, Achnanthes, Achnanthidium, Actinastrum, Actinochloris, Actinocyclus, Actinotaenium, Amphichrysis, Amphidinium, Amphikrikos, Amphipleura, Amphiprora, Amphithrix, Amphora, Anabaena, Anabaenopsis, Aneumastus, Ankistrodesmus, Ankyra, Anomoeoneis, Apatococcus, Aphanizomenon, Aphanocapsa, Aphanochaete, Aphanothece, Apiocystis, Apistonema, Arthrodesmus, Artherospira, Ascochloris, Asterionella, Asterococcus, Audouinella, Aulacoseira, Bacillaria, Balbiania, Bambusina, Bangia, Basichlamys, Batrachospermum, Binuclearia, Bitrichia, Blidingia, Botrdiopsis, Botrydium, Botryococcus, Botryosphaerella, Brachiomonas, Brachysira, Brachytrichia, Brebissonia, Bulbochaete, Bumilleria, Bumilleriopsis, Caloneis, Calothrix, Campylodiscus, Capsosiphon, Carteria, Catena, Cavinula, Centritractus, Centronella, Ceratium, Chaetoceros, Chaetochloris, Chaetomorpha, Chaetonella, Chaetonema, Chaetopeltis, Chaetophora, Chaetosphaeridium, Chamaesiphon, Chara, Characiochloris, Characiopsis, Characium, Charales, Chilomonas, Chlainomonas, Chlamydoblepharis, Chlamydocapsa, Chlamydomonas, Chlamydomonopsis, Chlamydomyxa, Chlamydonephris, Chlorangiella, Chlorangiopsis, Chlorella, Chlorobotrys, Chlorobrachis, Chlorochytrium, Chlorococcum, Chlorogloea, Chlorogloeopsis, Chlorogonium, Chlorolobion, Chloromonas, Chlorophysema, Chlorophyta, Chlorosaccus, Chlorosarcina, Choricystis, Chromophyton, Chromulina, Chroococcidiopsis, Chroococcus, Chroodactylon, Chroomonas, Chroothece, Chrysamoeba, Chrysapsis, Chrysidiastrum, Chrysocapsa, Chrysocapsella, Chrysochaete, Chrysochromulina, Chrysococcus, Chrysocrinus, Chrysolepidomonas, Chrysolykos, Chrysonebula, Chrysophyta, Chrysopyxis, Chrysosaccus, Chrysophaerella, Chrysostephanosphaera, Clodophora, Clastidium, Closteriopsis, Closterium, Coccomyxa, Cocconeis, Coelastrella, Coelastrum, Coelosphaerium, Coenochloris, Coenococcus, Coenocystis, Colacium, Coleochaete, Collodictyon, Compsogonopsis, Compsopogon, Conjugatophyta, Conochaete, Coronastrum, Cosmarium, Cosmioneis, Cosmocladium, Crateriportula, Craticula, Crinalium, Crucigenia, Crucigeniella, Cryptoaulax, Cryptomonas, Cryptophyta, Ctenophora, Cyanodictyon, Cyanonephron, Cyanophora, Cyanophyta, Cyanothece, Cyanothomonas, Cyclonexis, Cyclostephanos, Cyclotella, Cylindrocapsa, Cylindrocystis, Cylindrospermum, Cylindrotheca, Cymatopleura, Cymbella, Cymbellonitzschia, Cystodinium Dactylococcopsis, Debarya, Denticula, Dermatochrysis, Dermocarpa, Dermocarpella, Desmatractum, Desmidium, Desmococcus, Desmonema, Desmosiphon, Diacanthos, Diacronema, Diadesmis, Diatoma, Diatomella, Dicellula, Dichothrix, Dichotomococcus, Dicranochaete, Dictyochloris, Dictyococcus, Dictyosphaerium, Didymocystis, Didymogenes, Didymosphenia, Dilabifilum, Dimorphococcus, Dinobryon, Dinococcus, Diplochloris, Diploneis, Diplostauron, Distrionella, Docidium, Draparnaldia, Dunaliella, Dysmorphococcus, Ecballocystis, Elakatothrix, Ellerbeckia, Encyonema, Enteromorpha, Entocladia, Entomoneis, Entophysalis, Epichrysis, Epipyxis, Epithemia, Eremosphaera, Euastropsis, Euastrum, Eucapsis, Eucocconeis, Eudorina, Euglena, Euglenophyta, Eunotia, Eustigmatophyta, Eutreptia, Fallacia, Fischerella, Fragilaria, Fragilariforma, Franceia, Frustulia, Curcilla, Geminella, Genicularia, Glaucocystis, Glaucophyta, Glenodiniopsis, Glenodinium, Gloeocapsa, Gloeochaete, Gloeochrysis, Gloeococcus, Gloeocystis, Gloeodendron, Gloeomonas, Gloeoplax, Gloeothece, Gloeotila, Gloeotrichia, Gloiodictyon, Golenkinia, Golenkiniopsis, Gomontia, Gomphocymbella, Gomphonema, Gomphosphaeria, Gonatozygon, Gongrosia, Gongrosira, Goniochloris, Gonium, Gonyostomum, Granulochloris, Granulocystopsis, Groenbladia, Gymnodinium, Gymnozyga, Gyrosigma, Haematococcus, Hafniomonas, Hallassia, Hammatoidea, Hannaea, Hantzschia, Hapalosiphon, Haplotaenium, Haptophyta, Haslea, Hemidinium, Hemitoma, Heribaudiella, Heteromastix, Heterothrix, Hibberdia, Hildenbrandia, Hillea, Holopedium, Homoeothrix, Hormanthonema, Hormotila, Hyalobrachion, Hyalocardium, Hyalodiscus, Hyalogonium, Hyalotheca, Hydrianum, Hydrococcus, Hydrocoleum, Hydrocoryne, Hydrodictyon, Hydrosera, Hydrurus, Hyella, Hymenomonas, Isthmochloron, Johannesbaptistia, Juranyiella, Karayevia, Kathablepharis, Katodinium, Kephyrion, Keratococcus, Kirchneriella, Klebsormidium, Kolbesia, Koliella, Komarekia, Korshikoviella, Kraskella, Lagerheimia, Lagynion, Lamprothamnium, Lemanea, Lepocinclis, Leptosira, Lobococcus, Lobocystis, Lobomonas, Luticola, Lyngbya, Malleochloris, Mallomonas, Mantoniella, Marssoniella, Martyana, Mastigocoleus, Gastogloia, Melosira, Merismopedia, Mesostigma, Mesotaenium, Micractinium, Micrasterias, Microchaete, Microcoleus, Microcystis, Microglena, Micromonas, Microspora, Microthamnion, Mischococcus, Monochrysis, Monodus, Monomastix, Monoraphidium, Monostroma, Mougeotia, Mougeotiopsis, Myochloris, Myromecia, Myxosarcina, Naegeliella, Nannochloris, Nautococcus, Navicula, Neglectella, Neidium, Nephroclamys, Nephrocytium, Nephrodiella, Nephroselmis, Netrium, Nitella, Nitellopsis, Nitzschia, Nodularia, Nostoc, Ochromonas, Oedogonium, Oligochaetophora, Onychonema, Oocardium, Oocystis, Opephora, Ophiocytium, Orthoseira, Oscillatoria, Oxyneis, Pachycladella, Palmella, Palmodictyon, Pnadorina, Pannus, Paralia, Pascherina, Paulschulzia, Pediastrum, Pedinella, Pedinomonas, Pedinopera, Pelagodictyon, Penium, Peranema, Peridiniopsis, Peridinium, Peronia, Petroneis, Phacotus, Phacus, Phaeaster, Phaeodermatium, Phaeophyta, Phaeosphaera, Phaeothamnion, Phormidium, Phycopeltis, Phyllariochloris, Phyllocardium, Phyllomitas, Pinnularia, Pitophora, Placoneis, Planctonema, Planktosphaeria, Planothidium, Plectonema, Pleodorina, Pleurastrum, Pleurocapsa, Pleurocladia, Pleurodiscus, Pleurosigma, Pleurosira, Pleurotaenium, Pocillomonas, Podohedra, Polyblepharides, Polychaetophora, Polyedriella, Polyedriopsis, Polygoniochloris, Polyepidomonas, Polytaenia, Polytoma, Polytomella, Porphyridium, Posteriochromonas, Prasinochloris, Prasinocladus, Prasinophyta, Prasiola, Prochlorphyta, Prochlorothrix, Protoderma, Protosiphon, Provasoliella, Prymnesium, Psammodictyon, Psammothidium, Pseudanabaena, Pseudenoclonium, Psuedocarteria, Pseudochate, Pseudocharacium, Pseudococcomyxa, Pseudodictyosphaerium, Pseudokephyrion, Pseudoncobyrsa, Pseudoquadrigula, Pseudosphaerocystis, Pseudostaurastrum, Pseudostaurosira, Pseudotetrastrum, Pteromonas, Punctastruata, Pyramichlamys, Pyramimonas, Pyrrophyta, Quadrichloris, Quadricoccus, Quadrigula, Radiococcus, Radiofilum, Raphidiopsis, Raphidocelis, Raphidonema, Raphidophyta, Peimeria, Rhabdoderma, Rhabdomonas, Rhizoclonium, Rhodomonas, Rhodophyta, Rhoicosphenia, Rhopalodia, Rivularia, Rosenvingiella, Rossithidium, Roya, Scenedesmus, Scherffelia, Schizochlamydella, Schizochlamys, Schizomeris, Schizothrix, Schroederia, Scolioneis, Scotiella, Scotiellopsis, Scourfieldia, Scytonema, Selenastrum, Selenochloris, Sellaphora, Semiorbis, Siderocelis, Diderocystopsis, Dimonsenia, Siphononema, Sirocladium, Sirogonium, Skeletonema, Sorastrum, Spennatozopsis, Sphaerellocystis, Sphaerellopsis, Sphaerodinium, Sphaeroplea, Sphaerozosma, Spiniferomonas, Spirogyra, Spirotaenia, Spirulina, Spondylomorum, Spondylosium, Sporotetras, Spumella, Staurastrum, Stauerodesmus, Stauroneis, Staurosira, Staurosirella, Stenopterobia, Stephanocostis, Stephanodiscus, Stephanoporos, Stephanosphaera, Stichococcus, Stichogloea, Stigeoclonium, Stigonema, Stipitococcus, Stokesiella, Strombomonas, Stylochrysalis, Stylodinium, Styloyxis, Stylosphaeridium, Surirella, Sykidion, Symploca, Synechococcus, Synechocystis, Synedra, Synochromonas, Synura, Tabellaria, Tabularia, Teilingia, Temnogametum, Tetmemorus, Tetrachlorella, Tetracyclus, Tetradesmus, Tetraedriella, Tetraedron, Tetraselmis, Tetraspora, Tetrastrum, Thalassiosira, Thamniochaete, Thorakochloris, Thorea, Tolypella, Tolypothrix, Trachelomonas, Trachydiscus, Trebouxia, Trentepholia, Treubaria, Tribonema, Trichodesmium, Trichodiscus, Trochiscia, Tryblionella, Ulothrix, Uroglena, Uronema, Urosolenia, Urospora, Uva, Vacuolaria, Vaucheria, Volvox, Volvulina, Westella, Woloszynskia, Xanthidium, Xanthophyta, Xenococcus, Zygnema, Zygnemopsis, and Zygonium.

Additional cyanobacteria include members of the genus Chamaesiphon, Chroococcus, Cyanobacterium, Cyanobium, Cyanothece, Dactylococcopsis, Gloeobacter, Gloeocapsa, Gloeothece, Microcystis, Prochlorococcus, Prochloron, Synechococcus, Synechocystis, Cyanocystis, Dermocarpella, Stanieria, Xenococcus, Chroococcidiopsis, Myxosarcina, Arthrospira, Borzia, Crinalium, Geitlerinemia, Leptolyngbya, Limnothrix, Lyngbya, Microcoleus, Oscillatoria, Planktothrix, Prochiorothrix, Pseudanabaena, Spirulina, Starria, Symploca, Trichodesmium, Tychonema, Anabaena, Anabaenopsis, Aphanizomenon, Cyanospira, Cylindrospermopsis, Cylindrospermum, Nodularia, Nostoc, Scylonema, Calothrix, Rivularia, Tolypothrix, Chlorogloeopsis, Fischerella, Geitieria, Iyengariella, Nostochopsis, Stigonema and Thermosynechococcus.

Green non-sulfur bacteria include but are not limited to the following genera: Chloroflexus, Chloronema, Oscillochloris, Heliothrix, Herpetosiphon, Roseiflexus, and Thermomicrobium.

Green sulfur bacteria include but are not limited to the following genera: Chlorobium, Clathrochloris, and Prosthecochloris.

Purple sulfur bacteria include but are not limited to the following genera: Allochromatium, Chromatium, Halochromatium, Isochromatium, Marichromatium, Rhodovulum, Thermochromatium, Thiocapsa, Thiorhodococcus, and Thiocystis.

Purple non-sulfur bacteria include but are not limited to the following genera: Phaeospirillum, Rhodobaca, Rhodobacter, Rhodomicrobium, Rhodopila, Rhodopseudomonas, Rhodothalassium, Rhodospirillum, Rodovibrio, and Roseospira.

Aerobic chemolithotrophic bacteria include but are not limited to nitrifying bacteria such as Nitrobacteraceae sp., Nitrobacter sp., Nitrospina sp., Nitrococcus sp., Nitrospira sp., Nitrosomonas sp., Nitrosococcus sp., Nitrosospira sp., Nitrosolobus sp., Nitrosovibrio sp.; colorless sulfur bacteria such as, Thiovulum sp., Thiobacillus sp., Thiomicrospira sp., Thiosphaera sp., Thermothrix sp.; obligately chemolithotrophic hydrogen bacteria such as Hydrogenobacter sp., iron and manganese-oxidizing and/or depositing bacteria such as Siderococcus sp., and magnetotactic bacteria such as Aquaspirillum sp.

Archaeobacteria include but are not limited to methanogenic archaeobacteria such as Methanobacterium sp., Methanobrevibacter sp., Methanothermus sp., Methanococcus sp., Methanomicrobium sp., Methanospirillum sp., Methanogenium sp., Methanosarcina sp., Methanolobus sp., Methanothrix sp., Methanococcoides sp., Methanoplanus sp.; extremely thermophilic S-Metabolizers such as Thermoproteus sp., Pyrodictium sp., Sulfolobus sp., Acidianus sp. and other microorganisms such as, Bacillus subtilis, Saccharomyces cerevisiae, Streptomyces sp., Ralstonia sp., Rhodococcus sp., Corynebacteria sp., Brevibacteria sp., Mycobacteria sp., and oleaginous yeast.

Yet other suitable organisms include synthetic cells or cells produced by synthetic genomes as described in Venter et al. US Pat. Pub. No. 2007/0264688, and cell-like systems or synthetic cells as described in Glass et al. US Pat. Pub. No. 2007/0269862.

Still other suitable organisms include Escherichia coli, Acetobacter aceti, Bacillus subtilis, yeast and fungi such as Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis. In some embodiments those organisms are engineered to fix carbon dioxide while in other embodiments they are not.

E. Production of Recombinant Engineered Proteins

Skilled artisans are aware of many suitable methods available for culturing recombinant cells to produce (and optionally secrete) a recombinant engineered protein as disclosed herein, as well as for purification and/or isolation of expressed engineered proteins. The methods chosen for protein purification depend on many variables, including the properties of the protein of interest, its location and form within the cell, the vector, host strain background, and the intended application for the expressed protein. Culture conditions can also have an effect on solubility and localization of a given target protein. Many approaches can be used to purify target proteins expressed in recombinant microbial cells as disclosed herein, including without limitation ion exchange and gel filtration.

It is generally recognized that nearly all secreted bacterial proteins, and those proteins from other unicellular hosts, are synthesized as pre-proteins that contain N-terminal sequences known as signal peptides. These signal peptides influence the final destination of the protein and the mechanisms by which they are transported. Most signal peptides can be placed into one of four groups based on their translocation mechanism (e.g., Sec- or Tat-mediated) and the type of signal peptidase used to cleave the signal peptide from the preprotein. Also provided are N-terminal signal peptides containing a lipoprotein signal peptide. Although proteins carrying this type of signal are transported via the Sec translocase, their peptide signals tend to be shorter than normal Sec-signals and they contain a distinct sequence motif in the C-domain known as the lipo box (L(AS)(GA)C) at the −3 to +1 position. The cysteine at the +1 position is lipid modified following translocation whereupon the signal sequence is cleaved by a type II signal peptidase. Also provided are type IV or prepilin signal peptides, wherein type IV peptidase cleavage domains are localized between the N- and H-domain rather than in the C-domain common in other signal peptides.

As provided herein, the signal peptides can be attached to a heterologous polypeptide sequence (i.e., different than the protein the signal peptide is derived or obtained from) containing a nutritive polypeptide, in order to generate a recombinant nutritive polypeptide sequence. Alternatively, if a nutritive polypeptide is naturally secreted in the host organism, it can be sufficient to use the native signal sequence or a variety of signal sequences that directs secretion. In some embodiments of the nutritive polypeptides, the heterologous nutritive polypeptide sequence attached to the carboxyl terminus of the signal peptide is a naturally occurring eukaryotic protein, a mutein or derivative thereof, or a polypeptide nutritional domain. In other embodiments of the polypeptide, the heterologous nutritive polypeptide sequence attached to the carboxyl terminus of the signal peptide is a naturally occurring intracellular protein, a mutein or derivative thereof, or a polypeptide nutritional domain.

Purification of Nutritive Polypeptides.

Also provided are methods for recovering the secreted nutritive polypeptide from the culture medium. In some embodiments the secreted nutritive polypeptide is recovered from the culture medium during the exponential growth phase or after the exponential growth phase (e.g., in pre-stationary phase or stationary phase). In some embodiments the secreted nutritive polypeptide is recovered from the culture medium during the stationary phase. In some embodiments the secreted nutritive polypeptide is recovered from the culture medium at a first time point, the culture is continued under conditions sufficient for production and secretion of the recombinant nutritive polypeptide by the microorganism, and the recombinant nutritive polypeptide is recovered from the culture medium at a second time point. In some embodiments the secreted nutritive polypeptide is recovered from the culture medium by a continuous process. In some embodiments the secreted nutritive polypeptide is recovered from the culture medium by a batch process. In some embodiments the secreted nutritive polypeptide is recovered from the culture medium by a semi-continuous process. In some embodiments the secreted nutritive polypeptide is recovered from the culture medium by a fed-batch process. Those skilled in the art are aware of many suitable methods available for culturing recombinant cells to produce (and optionally secrete) a recombinant nutritive polypeptide as disclosed herein, as well as for purification and/or isolation of expressed recombinant polypeptides. The methods chosen for polypeptide purification depend on many variables, including the properties of the polypeptide of interest. Various methods of purification are known in the art including diafilitration, precipitation, and chromatography.

In some embodiments a peptide fusion tag is added to the recombinant protein making possible a variety of affinity purification methods that take advantage of the peptide fusion tag. In some embodiments, the use of an affinity method enables the purification of the target protein to near homogeneity in one step. Purification may include cleavage of part or all of the fusion tag with enterokinase, factor Xa, thrombin, or HRV 3C proteases, for example. In some embodiments, before purification or activity measurements of an expressed target protein, preliminary analysis of expression levels, cellular localization, and solubility of the target protein is performed. The target protein may be found in any or all of the following fractions: soluble or insoluble cytoplasmic fractions, periplasm, or medium. Depending on the intended application, preferential localization to inclusion bodies, medium, or the periplasmic space can be advantageous, in some embodiments, for rapid purification by relatively simple procedures.

While Escherichia coli is widely regarded as a robust host for heterologous protein expression, it is also widely known that over-expression of many proteins in this host is prone to aggregation in the form of insoluble inclusion bodies. One of the most commonly used methods for either rescuing inclusion body formation, or to improve the titer of the protein itself, is to include an amino-terminal maltose-binding protein (MBP) [Austin B P, Nallamsetty S, Waugh D S. Hexahistidine-tagged (SEQ ID NO: 22138) maltose-binding protein as a fusion partner for the production of soluble recombinant proteins in Escherichia coli. Methods Mol Biol. 2009; 498:157-72], or small ubiquitin-related modifier (SUMO) [Saitoh H, Uwada J, Azusa K. Strategies for the expression of SUMO-modified target proteins in Escherichia coli. Methods Mol Biol. 2009; 497:211-21; Malakhov M P, Mattern M R, Malakhova O A, Drinker M, Weeks S D, Butt T R. SUMO fusions and SUMO-specific protease for efficient expression and purification of proteins. J Struct Funct Genomics. 2004; 5(1-2):75-86; Panavas T, Sanders C, Butt T R. SUMO fusion technology for enhanced protein production in prokaryotic and eukaryotic expression systems. Methods Mol Biol. 2009; 497:303-17] fusion to the protein of interest. These two proteins are expressed extremely well, and in the soluble form, in Escherichia coli such that the protein of interest is also effectively produced in the soluble form. The protein of interest can be cleaved by designing a site specific protease recognition sequence (such as the tobacco etch virus (TEV) protease) in-between the protein of interest and the fusion protein [1].

In some embodiments the recombinant engineered protein is initially not folded correctly or is insoluble. A variety of methods are well known for refolding of insoluble proteins. Most protocols comprise the isolation of insoluble inclusion bodies by centrifugation followed by solubilization under denaturing conditions. The protein is then dialyzed or diluted into a non-denaturing buffer where refolding occurs. Because every protein possesses unique folding properties, the preferred refolding protocol for any given protein can be empirically determined by a skilled artisan. Preferred refolding conditions can, for example, be rapidly determined on a small scale by a matrix approach, in which variables such as protein concentration, reducing agent, redox treatment, divalent cations, etc., are tested. Once the preferred concentrations are found, they can be applied to a larger scale solubilization and refolding of the target protein.

In some embodiments a CAPS buffer at alkaline pH in combination with N-lauroylsarcosine is used to achieve solubility of the inclusion bodies, followed by dialysis in the presence of DTT to promote refolding. Depending on the target protein, expression conditions, and intended application, proteins solubilized from washed inclusion bodies may be >90% homogeneous and may not require further purification. Purification under fully denaturing conditions (before refolding) is possible using His•Tag® fusion proteins and His•Bind® immobilized metal affinity chromatography (Novogen®). In addition, S•TagTm, T7•Tag®, and Strep•Tag® II fusion proteins solubilized from inclusion bodies using 6 M urea can be purified under partially denaturing conditions by dilution to 2 M urea (S•Tag and T7•Tag) or 1 M urea (Strep•Tag II) prior to chromatography on the appropriate resin. Refolded fusion proteins can be affinity purified under native conditions using His•Tag, S•Tag, Strep•Tag II, and other appropriate affinity tags (e.g., GST•Tag™, and T7•Tag) (Novogen®).

In some embodiments proteins of this disclosure are synthsized chemically without the use of a recombinant production system. Protein synthesis can be carried out in a liquid-phase system or in a solid-phase system using techniques known in the art (see, e.g., Atherton, E., Sheppard, R. C. (1989). Solid Phase peptide synthesis: a practical approach. Oxford, England: IRL Press; Stewart, J. M., Young, J. D. (1984). Solid phase peptide synthesis (2nd ed.). Rockford: Pierce Chemical Company. Peptide chemistry and synthetic methods are well known in the art and a protein of this disclosure can be made using any method known in the art. A non-limiting example of such a method is the synthesis of a resin-bound peptide (including methods for de-protection of amino acids, methods for cleaving the peptide from the resin, and for its purification). For example, Fmoc-protected amino acid derivatives that can be used to synthesize the peptides are the standard recommended: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(BOC)-OH, Fmoc-Met-OH, Fmoc-Phe-OH, Fmoc-Pro-OH, Fmoc-Ser(tBu)-OH, Fmoc-Thr(tBu)-OH, Fmoc-Trp(BOC)-OH, Fmoc-Tyr(tBu)-OH and Fmoc-Val-OH (supplied from, e.g., Anaspec, Bachem, Iris Biotech, or NovabioChem). Resin bound peptide synthesis is performed, for example, using Fmoc based chemistry on a Prelude Solid Phase Peptide Synthesizer from Protein Technologies (Tucson, Ariz. 85714 U.S.A.). A suitable resin for the preparation of C-terminal carboxylic acids is a pre-loaded, low-load Wang resin available from NovabioChem (e.g. low load fmoc-Thr(tBu)-Wang resin, LL, 0.27 mmol/g). A suitable resin for the synthesis of peptides with a C-terminal amide is PAL-ChemMatrix resin available from Matrix-Innovation. The N-terminal alpha amino group is protected with Boc. Fmoc-deprotection can be achieved with 20% piperidine in NMP for 2×3 min. The coupling chemistry is DIC/HOAt/collidine in NMP. Amino acid/HOAt solutions (0.3 M/0.3 M in NMP at a molar excess of 3-10 fold) are added to the resin followed by the same molar equivalent of DIC (3 M in NMP) followed by collidine (3 M in NMP). For example, the following amounts of 0.3 M amino acid/HOAt solution are used per coupling for the following scale reactions: Scale/ml, 0.05 mmol/1.5 mL, 0.10 mmol/3.0 mL, 0.25 mmol/7.5 mL. Coupling time is either 2×30 min or 1×240 min. After synthesis the resin is washed with DCM, and the peptide is cleaved from the resin by a 2-3 hour treatment with TFA/TIS/water (95/2.5/2.5) followed by precipitation with diethylether. The precipitate is washed with diethylether. The crude peptide is dissolved in a suitable mixture of water and MeCN such as water/MeCN (4:1) and purified by reversed-phase preparative HPLC (Waters Deltaprep 4000 or Gilson) on a column containing C18-silica gel. Elution is performed with an increasing gradient of MeCN in water containing 0.1% TFA. Relevant fractions are checked by analytical HPLC or UPLC. Fractions containing the pure target peptide are mixed and concentrated under reduced pressure. The resulting solution is analyzed (HPLC, LCMS) and the product is quantified using a chemiluminescent nitrogen specific HPLC detector (Antek 8060 HPLC-CLND) or by measuring UV-absorption at 280 nm. The product is dispensed into glass vials. The vials are capped with Millipore glassfibre prefilters. Freeze-drying affords the peptide trifluoroacetate as a white solid. The resulting peptides can be detected and characterized using LCMS and/or UPLC, for example, using standard methods known in the art. LCMS can be performed on a setup consisting of Waters Acquity UPLC system and LCT Premier XE mass spectrometer from Micromass. The UPLC pump is connected to two eluent reservoirs containing: A) 0.1% Formic acid in water; and B) 0.1% Formic acid in acetonitrile. The analysis is performed at RT by injecting an appropriate volume of the sample (preferably 2-100 onto the column which is eluted with a gradient of A and B. The UPLC conditions, detector settings and mass spectrometer settings are: Column: Waters Acquity UPLC BEH, C-18, 1.7 μm, 2.1 mm×50 mm. Gradient: Linear 5%-95% acetonitrile during 4.0 min (alternatively 8.0 min) at 0.4 ml/min. Detection: 214 nm (analogue output from TUV (Tunable UV detector)). MS ionisation mode: API-ES Scan: 100-2000 amu (alternatively 500-2000 amu), step 0.1 amu. UPLC methods are well known. Non-limiting examples of methods that can be used are described at pages 16-17 of US 2013/0053310 A1, published Feb. 28, 2013, for example.

F. Compositions

At least one engineered protein disclosed herein can be combined with at least one second component to form a nutritive composition. In some embodiments the only source of amino acid in the composition is the at least one engineered protein. In such embodiments the amino acid composition of the composition is the same as the amino acid composition of the at least one engineered protein. In some embodiments the composition comprises at least one engineered protein and at least one second protein. In some embodiments the at least one second protein is an engineered protein, while in other embodiments the at least one second protein is not an engineered protein. In some embodiments the composition comprises 1, 2, 3, 4, 5, 6, 7, 8, 9, 10, 11, 12, 13, 14, 15, 16, 17, 18, 19, 20 or more engineered proteins. In some embodiments the composition comprises 0, 1, 2, 3, 4, 5, 6, 7, 8, 9, 10, 11, 12, 13, 14, 15, 16, 17, 18, 19, 20 or more non-engineered proteins. In some embodiments the composition comprises 1, 2, 3, 4, 5, 6, 7, 8, 9, 10, 11, 12, 13, 14, 15, 16, 17, 18, 19, 20 or more engineered proteins and the composition comprises 0, 1, 2, 3, 4, 5, 6, 7, 8, 9, 10, 11, 12, 13, 14, 15, 16, 17, 18, 19, 20 or more non-engineered proteins.

In some embodiments the nutritive composition as described in the preceding paragraph, further comprises at least one of at least one polypeptide, at least one peptide, and at least one free amino acid. In some embodiments the nutritive composition comprises at least one polypeptide and at least one peptide. In some embodiments the nutritive composition comprises at least one polypeptide and at least one free amino acid. In some embodiments the nutritive composition comprises at least one peptide and at least one free amino acid. In some embodiments the at least one polypeptide, at least one peptide, and/or at least one free amino acid comprises amino acids selected from 1) branch chain amino acids, 2) leucine, and 3) essential amino acids. In some embodiments the at least one polypeptide, at least one peptide, and/or at least one free amino acid consists of amino acids selected from 1) branch chain amino acids, 2) leucine, and 3) essential amino acids.

By adding at least one of a polypeptide, a peptide, and a free amino acid to a nutritive composition the proportion of at least one of branch chain amino acids, leucine, and essential amino acids, to total amino acid, present in the composition can be increased.

In some embodiments the composition comprises at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

In some embodiments the composition comprises at least one lipid. As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments the composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, a coloring agent.

In some embodiments the excipient is a buffering agent. Non-limiting examples of suitable buffering agents include sodium citrate, magnesium carbonate, magnesium bicarbonate, calcium carbonate, and calcium bicarbonate.

In some embodiments the excipient comprises a preservative. Non-limiting examples of suitable preservatives include antioxidants, such as alpha-tocopherol and ascorbate, and antimicrobials, such as parabens, chlorobutanol, and phenol.

In some embodiments the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments the composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments the excipient comprises a flavoring agent. Flavoring agents can be chosen from synthetic flavor oils and flavoring aromatics; natural oils; extracts from plants, leaves, flowers, and fruits; and combinations thereof. In some embodiments the flavoring agent is selected from cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

The weight fraction of the excipient or combination of excipients in the formulation is usually about 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the protein in the composition.

The engineered proteins and nutritive compositions disclosed herein can be formulated into a variety of forms and administered by a number of different means. The compositions can be administered orally, rectally, or parenterally, in formulations containing conventionally acceptable carriers, adjuvants, and vehicles as desired. The term “parenteral” as used herein includes subcutaneous, intravenous, intramuscular, or intrasternal injection and infusion techniques. In an exemplary embodiment, the engineered protein or nutritive composition is administered orally.

Solid dosage forms for oral administration include capsules, tablets, caplets, pills, troches, lozenges, powders, and granules. A capsule typically comprises a core material comprising an engineered protein or composition and a shell wall that encapsulates the core material. In some embodiments the core material comprises at least one of a solid, a liquid, and an emulsion. In some embodiments the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer functions as taste-masking agents.

Tablets, pills, and the like can be compressed, multiply compressed, multiply layered, and/or coated. The coating can be single or multiple. In one embodiment, the coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments the coating material comprises a protein. In some embodiments the coating material comprises at least one of a fat and an oil. In some embodiments the at least one of a fat and an oil is high temperature melting. In some embodiments the at least one of a fat and an oil is hydrogenated or partially hydrogenated. In some embodiments the at least one of a fat and an oil is derived from a plant. In some embodiments the at least one of a fat and an oil comprises at least one of glycerides, free fatty acids, and fatty acid esters. In some embodiments the coating material comprises at least one edible wax. The edible wax can be derived from animals, insects, or plants. Non-limiting examples include beeswax, lanolin, bayberry wax, carnauba wax, and rice bran wax. Tablets and pills can additionally be prepared with enteric coatings.

Alternatively, powders or granules embodying the engineered proteins and nutritive compositions disclosed herein can be incorporated into a food product. In some embodiments the food product is be a drink for oral administration. Non-limiting examples of a suitable drink include fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

In some embodiments the food product is a solid foodstuff. Suitable examples of a solid foodstuff include without limitation a food bar, a snack bar, a cookie, a brownie, a muffin, a cracker, an ice cream bar, a frozen yogurt bar, and the like.

In some embodiments, the proteins and compositions disclosed herein are incorporated into a therapeutic food. In some embodiments, the therapeutic food is a ready-to-use food that optionally contains some or all essential macronutrients and micronutrients. In some embodiments, the proteins and compositions disclosed herein are incorporated into a supplementary food that is designed to be blended into an existing meal. In some embodiments, the supplemental food contains some or all essential macronutrients and micronutrients. In some embodiments, the proteins and compositions disclosed herein are blended with or added to an existing food to fortify the food's protein nutrition. Examples include food staples (grain, salt, sugar, cooking oil, margarine), beverages (coffee, tea, soda, beer, liquor, sports drinks), snacks, sweets and other foods.

The compositions disclosed herein can be utilized in methods to increase at least one of muscle mass, strength and physical function, thermogenesis, metabolic expenditure, satiety, mitochondrial biogenesis, weight or fat loss, and lean body composition for example.

A formulation can contain a nutritive polypeptide up to about 25 g per 100 kilocalories (25 g/100 kcal) in the formulation, meaning that all or essentially all of the energy present in the formulation is in the form of the nutritive polypeptide. More typically, about 99%, 98%, 97%, 96%, 95%, 90%, 85%, 80%, 75%, 70%, 65%, 60%, 55%, 50%, 45%, 40%, 35%, 30%, 25%, 20%, 15%, 10%, 5% or less than 5% of the energy present in the formulation is in the form of the nutritive polypeptide. In other formulations, the nutritive polypeptide is present in an amount sufficient to provide a nutritional benefit equivalent to or greater than at least about 0.1% of a reference daily intake value of polypeptide. Suitable reference daily intake values for protein are well known in the art. See, e.g., Dietary Reference Intakes for Energy, Carbohydrate, Fiber, Fat, Fatty Acids, Cholesterol, Protein and Amino Acids, Institute of Medicine of the National Academies, 2005, National Academies Press, Washington D.C. A reference daily intake value for protein is a range wherein 10-35% of daily calories are provided by protein and isolated amino acids. Another reference daily intake value based on age is provided as grams of protein per day: children ages 1-3: 13 g, children ages 4-8: 19 g, children ages 9-13: 34 g, girls ages 14-18: 46, boys ages 14-18: 52, women ages 19-70+: 46, and men ages 19-70+: 56. In other formulations, the nutritive polypeptide is present in an amount sufficient to provide a nutritional benefit to a human subject suffering from protein malnutrition or a disease, disorder or condition characterized by protein malnutrition. Protein malnutrition is commonly a prenatal or childhood condition. Protein malnutrition with adequate energy intake is termed kwashiorkor or hypoalbuminemic malnutrition, while inadequate energy intake in all forms, including inadequate protein intake, is termed marasmus. Adequately nourished individuals can develop sarcopenia from consumption of too little protein or consumption of proteins deficient in nutritive amino acids. Prenatal protein malnutrition can be prevented, treated or reduced by administration of the nutritive polypeptides described herein to pregnant mothers, and neonatal protein malnutrition can be prevented, treated or reduced by administration of the nutritive polypeptides described herein to the lactation mother. In adults, protein malnutrition is commonly a secondary occurrence to cancer, chronic renal disease, and in the elderly. Additionally, protein malnutrition can be chronic or acute. Examples of acute protein malnutrition occur during an acute illness or disease such as sepsis, or during recovery from a traumatic injury, such as surgery, thermal injury such as a burn, or similar events resulting in substantial tissue remodeling. Other acute illnesses treatable by the methods and compositions described herein include sarcopenia, cachexia, diabetes, insulin resistance, and obesity.

A formulation can contain a nutritive polypeptide in an amount sufficient to provide a feeling of satiety when consumed by a human subject, meaning the subject feels a reduced sense or absence of hunger, or desire to eat. Such a formulation generally has a higher satiety index than carbohydrate-rich foods on an equivalent calorie basis.

A formulation can contain a nutritive polypeptide in an amount based on the concentration of the nutritive polypeptide (e.g., on a weight-to-weight basis), such that the nutritive polypeptide accounts for up to 100% of the weight of the formulation, meaning that all or essentially all of the matter present in the formulation is in the form of the nutritive polypeptide. More typically, about 99%, 98%, 97%, 96%, 95%, 90%, 85%, 80%, 75%, 70%, 65%, 60%, 55%, 50%, 45%, 40%, 35%, 30%, 25%, 20%, 15%, 10%, 5% or less than 5% of the weight present in the formulation is in the form of the nutritive polypeptide. In some embodiments, the formulation contains 10 mg, 100 mg, 500 mg, 750 mg, 1 g, 2 g, 3 g, 4 g, 5 g, 6 g, 7 g, 8 g, 9, 10 g, 15 g, 20 g, 25 g, 30 g, 35 g, 40 g, 45 g, 50 g, 60 g, 70 g, 80 g, 90 g, 100 g or over 100 g of nutritive polypeptide.

Preferably, the formulations provided herein are substantially free of non-comestible products. Non-comestible products are often found in preparations of recombinant proteins of the prior art, produced from yeast, bacteria, algae, insect, mammalian or other expression systems. Exemplary non-comestible products include surfactant, a polyvinyl alcohol, a propylene glycol, a polyvinyl acetate, a polyvinylpyrrolidone, a non-comestible polyacid or polyol, a fatty alcohol, an alkylbenzyl sulfonate, an alkyl glucoside, or a methyl paraben.

In aspects, the provided formulations contain other materials, such as a tastant, a nutritional carbohydrate and/or a nutritional lipid. In addition, formulations may include bulking agents, texturizers, and fillers.

In preferred embodiments, the nutritive polypeptides provided herein are isolated and/or substantially purified. The nutritive polypeptides and the compositions and formulations provided herein, are substantially free of non-protein components. Such non-protein components are generally present in protein preparations such as whey, casein, egg and soy preparations, which contain substantial amounts of carbohydrates and lipids that complex with the polypeptides and result in delayed and incomplete protein digestion in the gastrointestinal tract. Such non-protein components can also include DNA. Thus, the nutritive polypeptides, compositions and formulations are characterized by improved digestability and decreased allergenicity as compared to food-derived polypeptides and polypeptide mixtures. In some embodiments, improved digestability means a faster rate of digestion when consumed or otherwise administered into the gastrointestinal tract of a human subject. In an alternative embodiment, improved digestability means a slower rate of digestion when consumed or otherwise administered into the gastrointestinal tract of a human subject, for example in situations where the human suffers from impaired protein absorption ability. Furthermore, these formulations and compositions are characterized by more reproducible digestability from a time and/or a digestion product at a given unit time basis. In certain embodiments, a nutritive polypeptide is at least 10% reduced in lipids and/or carbohydrates, and optionally one or more other materials that decreases digestibility and/or increases allergenicity, relative to a reference polypeptide or reference polypeptide mixture, e.g., is reduced by 20%, 30%, 40%, 50%, 60%, 70%, 80%, 90%, 95%, 99% or greater than 99%. In certain embodiments, the nutritive formulations contain a nutritional carbohydrate and/or nutritional lipid, which are selected for digestibility and/or reduced allegenicity.

The compositions disclosed herein can be utilized in methods to increase at least one of muscle mass, strength and physical function, thermogenesis, metabolic expenditure, satiety, mitochondrial biogenesis, weight or fat loss, and lean body composition for example.

G. Methods of Use

In some embodiments the proteins and compositions disclosed herein are administered to a patient or a user (sometimes collectively referred to as a “subject”). As used herein “administer” and “administration” encompasses embodiments in which one person directs another to consume a protein or composition in a certain manner and/or for a certain purpose, and also situations in which a user uses a protein or composition in a certain manner and/or for a certain purpose independently of or in variance to any instructions received from a second person. Non-limiting examples of embodiments in which one person directs another to consume a protein or composition in a certain manner and/or for a certain purpose include when a physician prescribes a course of conduct and/or treatment to a patient, when a trainer advises a user (such as an athlete) to follow a particular course of conduct and/or treatment, and when a manufacturer, distributer, or marketer recommends conditions of use to an end user, for example through advertisements or labeling on packaging or on other materials provided in association with the sale or marketing of a product.

In some embodiments the proteins or compositions are provided in a dosage form. In some embodiments the dosage form is designed for administration of at least one protein disclosed herein, wherein the total amount of protein administered is selected from 0.1 g to 1 g, 1 g to 5 g, from 2 g to 10 g, from 5 g to 15 g, from 10 g to 20 g, from 15 g to 25 g, from 20 g to 40 g, from 25-50 g, and from 30-60 g. In some embodiments the dosage form is designed for administration of at least one protein disclosed herein, wherein the total amount of protein administered is selected from about 0.1 g, 0.1 g-1 g, 1 g, 2 g, 3 g, 4 g, 5 g, 6 g, 7 g, 8 g, 9 g, 10 g, 15 g, 20 g, 25 g, 30 g, 35 g, 40 g, 45 g, 50 g, 55 g, 60 g, 65 g, 70 g, 75 g, 80 g, 85 g, 90 g, 95 g, and 100 g.

In some embodiments the dosage form is designed for administration of at least one protein disclosed herein, wherein the total amount of essential amino acids administered is selected from 0.1 g to 1 g, from 1 g to 5 g, from 2 g to 10 g, from 5 g to 15 g, from 10 g to 20 g, and from 1-30 g. In some embodiments the dosage form is designed for administration of at least one protein disclosed herein, wherein the total amount of protein administered is selected from about 0.1 g, 0.1-1 g, 1 g, 2 g, 3 g, 4 g, 5 g, 6 g, 7 g, 8 g, 9 g, 10 g, 15 g, 20 g, 25 g, 30 g, 35 g, 40 g, 45 g, 50 g, 55 g, 60 g, 65 g, 70 g, 75 g, 80 g, 85 g, 90 g, 95 g, and 100 g.

In some embodiments the protein or composition is consumed at a rate of from 0.1 g to 1 g a day, 1 g to 5 g a day, from 2 g to 10 g a day, from 5 g to 15 g a day, from 10 g to 20 g a day, from 15 g to 30 g a day, from 20 g to 40 g a day, from 25 g to 50 g a day, from 40 g to 80 g a day, from 50 g to 100 g a day, or more.

In some embodiments, of the total protein intake by the subject, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or about 100% of the total protein intake by the subject over a dietary period is made up of at least one protein according to this disclosure. In some embodiments, of the total protein intake by the subject, from 5% to 100% of the total protein intake by the subject, from 5% to 90% of the total protein intake by the subject, from 5% to 80% of the total protein intake by the subject, from 5% to 70% of the total protein intake by the subject, from 5% to 60% of the total protein intake by the subject, from 5% to 50% of the total protein intake by the subject, from 5% to 40% of the total protein intake by the subject, from 5% to 30% of the total protein intake by the subject, from 5% to 20% of the total protein intake by the subject, from 5% to 10% of the total protein intake by the subject, from 10% to 100% of the total protein intake by the subject, from 10% to 100% of the total protein intake by the subject, from 20% to 100% of the total protein intake by the subject, from 30% to 100% of the total protein intake by the subject, from 40% to 100% of the total protein intake by the subject, from 50% to 100% of the total protein intake by the subject, from 60% to 100% of the total protein intake by the subject, from 70% to 100% of the total protein intake by the subject, from 80% to 100% of the total protein intake by the subject, or from 90% to 100% of the total protein intake by the subject, over a dietary period, is made up of at least one protein according to this disclosure. In some embodiments the at least one protein of this disclosure accounts for at least 5%, at least 10%, at least 15%, at least 20%, at least 25%, at least 30%, at least 35%, at least 40%, at least 45%, or at least 50% of the subject's calorie intake over a dietary period.

In some embodiments the at least one protein according to this disclosure comprises at least 2 proteins of this disclosure, at least 3 proteins of this disclosure, at least 4 proteins of this disclosure, at least 5 proteins of this disclosure, at least 6 proteins of this disclosure, at least 7 proteins of this disclosure, at least 8 proteins of this disclosure, at least 9 proteins of this disclosure, at least 10 proteins of this disclosure, or more.

In some embodiments the dietary period is 1 meal, 2 meals, 3 meals, at least 1 day, at least 2 days, at least 3 days, at least 4 days, at least 5 days, at least 6 days, at least 1 week, at least 2 weeks, at least 3 weeks, at least 4 weeks, at least 1 month, at least 2 months, at least 3 months, at least 4 months, at least 5 months, at least 6 months, or at least 1 year. In some embodiments the dietary period is from 1 day to 1 week, from 1 week to 4 weeks, from 1 month, to 3 months, from 3 months to 6 months, or from 6 months to 1 year.

Clinical studies provide evidence that protein prevents muscle loss due to aging or bed rest. In particular, studies have shown that protein supplementation increases muscle fractional synthetic rate (FSR) during prolonged bed rest, maintains leg mass and strength during prolonged bed rest, increases lean body mass, improves functional measures of gait and balance, and may serve as a viable intervention for individuals at risk of sarcopenia due to immobility or prolonged bed rest. See, e.g., Paddon-Jones D, et al. J Clin Endocrinol Metab 2004, 89:4351-4358; Ferrando, A et al. Clinical Nutrition 2009 1-6; Katsanos C et al. Am J Physiol Endocrinol Metab. 2006, 291: 381-387.

Studies on increasing muscle protein anabolism in athletes have shown that protein provided following exercise promotes muscle hypertrophy to a greater extent than that achieved by exercise alone. It has also been shown that protein provided following exercise supports protein synthesis without any increase in protein breakdown, resulting in a net positive protein balance and muscle mass accretion. While muscle protein synthesis appears to respond in a dose-response fashion to essential amino acid supplementation, not all proteins are equal in building muscle. For example, the amino acid leucine is an important factor in stimulating muscle protein synthesis. See, e.g., Borscheim E et al. Am J Physiol Endocrinol Metab 2002, 283: E648-E657; Borsheim E et al. Clin Nutr. 2008, 27: 189-95; Esmarck B et al J Physiol 2001, 535: 301-311; Moore D et al. Am J Clin Nutr 2009, 89: 161-8).

In another aspect this disclosure provides methods of maintaining or increasing at least one of muscle mass, muscle strength, and functional performance in a subject. In some embodiments the methods comprise providing to the subject a sufficient amount of a protein of this disclosure, a composition of this disclosure, or a composition made by a method of this disclosure. In some embodiments the subject is at least one of elderly, critically-medically ill, and suffering from protein-energy malnutrition. In some embodiments the sufficient amount of a protein of this disclosure, a composition of this disclosure, or a composition made by a method of this disclosure is consumed by the subject in coordination with performance of exercise. In some embodiments the protein of this disclosure, composition of this disclosure, or composition made by a method of this disclosure is consumed by the subject by an oral, enteral, or parenteral route. In some embodiments the protein of this disclosure, composition of this disclosure, or composition made by a method of this disclosure is consumed by the subject by an oral route. In some embodiments the protein of this disclosure, composition of this disclosure, or composition made by a method of this disclosure is consumed by the subject by an enteral route.

In another aspect this disclosure provides methods of maintaining or achieving a desirable body mass index in a subject. In some embodiments the methods comprise providing to the subject a sufficient amount of a protein of this disclosure, a composition of this disclosure, or a composition made by a method of this disclosure. In some embodiments the subject is at least one of elderly, critically-medically ill, and suffering from protein-energy malnutrition. In some embodiments the sufficient amount of a protein of this disclosure, a composition of this disclosure, or a composition made by a method of this disclosure is consumed by the subject in coordination with performance of exercise. In some embodiments the protein of this disclosure, composition of this disclosure, or composition made by a method of this disclosure is consumed by the subject by an oral, enteral, or parenteral route.

In another aspect this disclosure provides methods of providing protein to a subject with protein-energy malnutrition. In some embodiments the methods comprise providing to the subject a sufficient amount of a protein of this disclosure, a composition of this disclosure, or a composition made by a method of this disclosure. In some embodiments the protein of this disclosure, composition of this disclosure, or composition made by a method of this disclosure is consumed by the subject by an oral, enteral, or parenteral route.

The need for essential amino acid supplementation has been suggested in cancer patients and other patients suffering from cachexia. Dietary studies in mice have shown survival and functional benefits to cachectic cancer-bearing mice through dietary intervention with essential amino acids. Beyond cancer, essential amino acid supplementation has also shown benefits, such as improved muscle function and muscle gain, in patients suffering from other diseases that have difficulty exercising and therefore suffer from muscular deterioration, such as chronic obstructive pulmonary disease, chronic heart failure, HIV, and other disease states.

Studies have shown that specific amino acids have advantages in managing cachexia. A relatively high content of BCAAs and Leu in diets are thought to have a positive effect in cachexia by promoting total protein synthesis by signaling an increase in translation, enhancing insulin release, and inhibiting protein degradation. Thus, consuming increased dietary BCAAs in general and/or Leu in particular will contribute positively to reduce or reverse the effects of cachexia. Because nitrogen balance is important in countering the underlying cause of cachexia it is thought that consuming increased dietary glutamine and/or arginine will contribute positively to reduce or reverse the effects of cachexia. See, e.g., Op den Kamp C, Langen R, Haegens A, Schols A. “Muscle atrophy in cachexia: can dietary protein tip the balance?” Current Opinion in Clinical Nutrition and Metabolic Care 2009, 12:611-616; Poon R T-P, Yu W-C, Fan S-T, et al. “Long-term oral branched chain amino acids in patients undergoing chemoembolization for hepatocellular carcinoma: a randomized trial.” Aliment Pharmacol Ther 2004; 19:779-788; Tayek J A, Bistrian B R, Hehir D J, Martin R, Moldawer L L, Blackburn G L. “Improved protein kinetics and albumin synthesis by branched chain amino acid-enriched total parenteral nutrition in cancer cachexia.” Cancer. 1986; 58:147-57; Xi P, Jiang Z, Zheng C, Lin Y, Wu G “Regulation of protein metabolism by glutamine: implications for nutrition and health.” Front Biosci. 2011 Jan. 1; 16:578-97.

Accordingly, also provided herein are methods of treating cachexia in a subject. In some embodiments a sufficient amound of a protein of this disclosure, a composition of this disclosure, or a composition made by a method of this disclosure for a subject with cachexia is an amount such that the amount of protein of this disclosure ingested by the person meets or exceeds the metabolic needs (which are often elevated). A protein intake of 1.5 g/kg of body weight per day or 15-20% of total caloric intake appears to be an appropriate target for persons with cachexia. In some embodiments all of the protein consumed by the subject is a protein according to this disclosure. In some embodiments protein according to this disclosure is combined with other sources of protein and/or free amino acids to provide the total protein intake of the subject. In some embodiments the subject is at least one of elderly, critically-medically ill, and suffering from protein-energy malnutrition. In some embodiments the subject suffers from a disease that makes exercise difficult and therefore causes muscular deterioration, such as chronic obstructive pulmonary disease, chronic heart failure, HIV, cancer, and other disease states. In some embodiments, the protein according to disclosure, the composition according to disclosure, or the composition made by a method according to disclosure is consumed by the subject in coordination with performance of exercise. In some embodiments, the protein according to this disclosure, the composition according to disclosure, or the composition made by a method according to disclosure is consumed by the subject by an oral, enteral, or parenteral route.

Sarcopenia is the degenerative loss of skeletal muscle mass (typically 0.5-1% loss per year after the age of 25), quality, and strength associated with aging. Sarcopenia is a component of the frailty syndrome. The European Working Group on Sarcopenia in Older People (EWGSOP) has developed a practical clinical definition and consensus diagnostic criteria for age-related sarcopenia. For the diagnosis of sarcopenia, the working group has proposed using the presence of both low muscle mass and low muscle function (strength or performance). Sarcopenia is characterized first by a muscle atrophy (a decrease in the size of the muscle), along with a reduction in muscle tissue “quality,” caused by such factors as replacement of muscle fibres with fat, an increase in fibrosis, changes in muscle metabolism, oxidative stress, and degeneration of the neuromuscular junction. Combined, these changes lead to progressive loss of muscle function and eventually to frailty. Frailty is a common geriatric syndrome that embodies an elevated risk of catastrophic declines in health and function among older adults. Contributors to frailty can include sarcopenia, osteoporosis, and muscle weakness. Muscle weakness, also known as muscle fatigue, (or “lack of strength”) refers to the inability to exert force with one's skeletal muscles. Weakness often follows muscle atrophy and a decrease in activity, such as after a long bout of bedrest as a result of an illness. There is also a gradual onset of muscle weakness as a result of sarcopenia.

The proteins of this disclosure are useful for treating sarcopenia or frailty once it develops in a subject or for preventing the onset of sarcopenia or frailty in a subject who is a member of an at risk groups. In some embodiments all of the protein consumed by the subject is a protein according to this disclosure. In some embodiments protein according to this disclosure is combined with other sources of protein and/or free amino acids to provide the total protein intake of the subject. In some embodiments the subject is at least one of elderly, critically-medically ill, and suffering from protein-energy malnutrition. In some embodiments, the protein according to disclosure, the composition according to disclosure, or the composition made by a method according to disclosure is consumed by the subject in coordination with performance of exercise. In some embodiments, the protein according to this disclosure, the composition according to disclosure, or the composition made by a method according to disclosure is consumed by the subject by an oral, enteral, or parenteral route.

Obesity is a multifactorial disorder associated with a host of comorbidities including hypertension, type 2 diabetes, dyslipidemia, coronary heart disease, stroke, cancer (eg, endometrial, breast, and colon), osteoarthritis, sleep apnea, and respiratory problems. The incidence of obesity, defined as a body mass index>30 kg/m2, has increased dramatically in the United States, from 15% (1976-1980) to 33% (2003-2004), and it continues to grow. Although the mechanisms contributing to obesity are complex and involve the interplay of behavioral components with hormonal, genetic, and metabolic processes, obesity is largely viewed as a lifestyle-dependent condition with 2 primary causes: excessive energy intake and insufficient physical activity. With respect to energy intake, there is evidence that modestly increasing the proportion of protein in the diet, while controlling total energy intake, may improve body composition, facilitate fat loss, and improve body weight maintenance after weight loss. Positive outcomes associated with increased dietary protein are thought to be due primarily to lower energy intake associated with increased satiety, reduced energy efficiency and/or increased thermogenesis, positive effects on body composition (specifically lean muscle mass), and enhanced glycemic control.

Dietary proteins are more effective in increasing post-prandial energy expenditure than isocaloric intakes of carbohydrates or fat (see, e.g., Dauncey M, Bingham S. “Dependence of 24 h energy expenditure in man on composition of the nutrient intake.” Br J Nutr 1983, 50: 1-13; Karst H et al. “Diet-induced thermogenesis in man: thermic effects of single proteins, carbohydrates and fats depending on their energy amount.” Ann Nutr Metab. 1984, 28: 245-52; Tappy L et al “Thermic effect of infused amino acids in healthy humans and in subjects with insulin resistance.” Am J Clin Nutr 1993, 57 (6): 912-6). This property along with other properties (satiety induction; preservation of lean body mass) make protein an attractive component of diets directed at weight management. The increase in energy expenditure caused by such diets may in part be due to the fact that the energy cost of digesting and metabolizing protein is higher than for other calorie sources. Protein turnover, including protein synthesis, is an energy consuming process. In addition, high protein diets may also up-regulate uncoupling protein in liver and brown adipose, which is positively correlated with increases in energy expenditure. It has been theorized that different proteins may have unique effects on energy expenditure.

Studies suggest that ingestion of protein, particularly proteins with high EAA and/or BCAA content, leads to distinct effects on thermogenesis and energy expenditure (see, e.g., Mikkelsen P. et al. “Effect of fat-reduced diets on 24 h energy expenditure: comparisons between animal protein, vegetable protein and carbohydrate.” Am J Clin Nutr 2000, 72:1135-41; Acheson K. et al. “Protein choices targeting thermogenesis and metabolism.” Am J Clin Nutr 2011, 93:525-34; Alfenas R. et al. “Effects of protein quality on appetite and energy metabolism in normal weight subjects” Arg Bras Endocrinol Metabol 2010, 54 (1): 45-51; Lorenzen J. et al. “The effect of milk proteins on appetite regulation and diet-induced thermogenesis.” J Clin Nutr 2012 66 (5): 622-7). Additionally, L-tyrosine has been identified as an amino acid that plays a role in thermogenesis (see, e.g., Belza A. et al. “The beta-adrenergic antagonist propranolol partly abolishes thermogenic response to bioactive food ingredients.” Metabolism 2009, 58 (8):1137-44). Further studies suggest that Leucine and Arginine supplementation appear to alter energy metabolism by directing substrate to lean body mass rather than adipose tissue (Dulloo A. “The search for compounds that stimulate thermogenesis in obesity management: from pharmaceuticals to functional food ingredients.” Obes Rev 2011 12: 866-83).

Collectively the literature suggests that different protein types leads to distinct effects on thermogenesis. Because proteins or peptides rich in EAAs, BCAA, and/or at least one of Tyr, Arg, and Leu are believed to have a stimulatory effect on thermogenesis, and because stimulation of thermogenesis is believed to lead to positive effects on weight management, this disclosure also provides products and methods useful to stimulation thermogenesis and/or to bring about positive effects on weight management in general.

More particularly, this disclosure provides methods of increasing thermogenesis in a subject. In some embodiments the methods comprise providing to the subject a sufficient amount of a protein of this disclosure, a composition of this disclosure, or a composition made by a method of this disclosure. In some embodiments the subject is obese. In some embodiments, the protein according to disclosure, the composition according to disclosure, or the composition made by a method according to disclosure is consumed by the subject in coordination with performance of exercise. In some embodiments, the protein according to disclosure, the composition according to disclosure, or the composition made by a method according to disclosure is consumed by the subject by an oral, enteral, or parenteral route.

At the basic level, the reason for the development of an overweight condition is due to an imbalance between energy intake and energy expenditure. Attempts to reduce food at any particular occasion (satiation) and across eating occasions (satiety) have been a major focus of recent research. Reduced caloric intake as a consequence of feeling satisfied during a meal and feeling full after a meal results from a complex interaction of internal and external signals. Various nutritional studies have demonstrated that variation in food properties such as energy density, content, texture and taste influence both satiation and satiety.

There are three macronutrients that deliver energy: fat, carbohydrates and proteins. A gram of protein or carbohydrate provides 4 calories while a gram of fat 9 calories. Protein generally increases satiety to a greater extent than carbohydrates or fat and therefore may facilitate a reduction in calorie intake. However, there is considerable evidence that indicates the type of protein matters in inducing satiety (see, e.g., W. L. Hall, et al. “Casein and whey exert different effects on plasma amino acid profiles, gastrointestinal hormone secretion and appetite.” Br J Nutr. 2003 February, 89(2):239-48; R. Abou-Samra, et al. “Effect of different protein sources on satiation and short-term satiety when consumed as a starter.” Nutr J. 2011 Dec. 23, 10:139; T. Akhavan, et al. “Effect of premeal consumption of whey protein and its hydrolysate on food intake and postmeal glycemia and insulin responses in young adults.” Am J Clin Nutr. 2010 April, 91(4):966-75, Epub 2010 Feb. 17; MA Veldhorst “Dose-dependent satiating effect of whey relative to casein or soy” Physiol Behav. 2009 Mar. 23, 96(4-5):675-82). Evidence indicates that protein rich in Leucine is particularly effective at inducing satiety (see, e.g., Fromentin G et al “Peripheral and central mechanisms involved in the control of food intake by dietary amino acids and proteins.” Nutr Res Rev 2012 25: 29-39).

Because of the role of dietary protein in inducing satiety, the engineered protein and nutritive compositions disclosed herein can be used to induce a satiety response in a mammal, such as a human. In some embodiments, the engineered protein comprises a ratio of branch chain amino acid residues to total amino acid residues that is equal to or greater than the ratio of branch chain amino acid residues to total amino acid residues present in at least one of whey protein, egg protein, and soy protein.

In some embodiments incorporating a least one engineered protein or nutritive composition of this disclosure into the diet of a subject has at least one effect selected from inducing postprandial satiety (including by suppressing hunger), inducing thermogenesis, reducing glycemic response, positively affecting energy expenditure and lean body mass, reducing the weight gain caused by overeating, and decreasing energy intake. In some embodiments incorporating a least one engineered protein or nutritive composition of this disclosure into the diet of a subject has at least one effect selected from greater loss of body fat, less lean tissue loss, a better lipid profile, and improved glucose tolerance and insulin sensitivity.

In some embodiments the subject consumes the engineered protein at a rate of from 0.1 g to 1 g a day, from 1 g to 5 g a day, from 2 g to 10 g a day, from 5 g to 15 g a day, from 10 g to 20 g a day, from 15 g to 30 g a day, from 20 g to 40 g a day, from 25 g to 50 g a day, from 40 g to 80 g a day, from 50 g to 100 g a day, or more. In some embodiments the engineered protein accounts for at least 5%, at least 10%, at least 15%, at least 20%, at least 25%, at least 30%, at least 35%, at least 40%, at least 45%, or at least 50% of the subjects calorie intake over a period of 1 meal, 1 day, 2 days, 3 days, 4 days, 5 days, 1 week, 2 weeks, 3 weeks, 1 month, 1-3 months, 2-6 months, 6-12 months, or longer.

### EXAMPLES

Examples of the techniques and protocols described herein can be found in Remington's Pharmaceutical Sciences, 16th edition, Osol, A. (ed), 1980.

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1

### Construction of Protein Libraries

Reference secreted proteins were identified in the annotated proteome for selected microorganisms as defined by the UniProt database. Specifically, proteins that have been observed and/or annotated as being present outside the various cellular plasma membranes, were identified. This procedure was applied to all species of the genera Acremonium, Aspergillus, Chrysosporium, Corynebacterium, Fusarium, Penicillium, Pichia pastoris, Rhizopus, Synechocystis, Synechococcus, Trametes, and Trichoderma, as well as to Bacillus subtilis, Escherichia coli, and Saccharomyces cerevisiae, to build a protein library. The selected proteins from each genus (species) are listed using their UniProt IDs in Appendix A.

Non-limiting examples of proteins and fragments of proteins are provided in the following Examples.

### Example 2

### Selection of Reference Secreted Proteins for Engineering

The NCBI Conserved Domain Database (Marchler-Bauer A., and Bryant, S. H. “CD-Search: protein domain annotations on the fly”. Nuc. Acid. Res. (2004) 32: W327-W331) includes protein domains and/or folds used in previous studies to reengineer protein-protein binding interactions. (Binz, K H, and Pluckthun, A. “Engineered proteins as specific binding reagents”. Curr. Op. Biotech. (2005) 16: 459-469; Gebauer, M. and Skerra, A. “Engineered protein scaffolds as next-generation antibody therapeutics”. Curr. Op. Chem. Biol. (2009) 13: 245-255; Lehtio, J., Teeri T. T., and Nygren P. A. “Alpha-Amylase Inhibitors Selected From a Combinatorial Library of a Cellulose Binding Domain Scaffold”. Proteins: Struct., Func., Gene., (2000) 41: 316-322; and Olson C A and Roberts R W. “Design, expression, and stability of a diverse protein library based on the human fibronectin type III domain”. Prot. Sci. (2007) 16: 476-484.) As such, the database can be used to identify protein scaffolds that are expected to contain a robust, stable fold with known variable positions or regions, wherein such variable positions or regions can be tailored to match a desired overall amino acid distribution.

In this experiment, the folds/domains selected for this analysis were ankyrin repeats, Leucine rich repeats, tetratricopeptide repeats, armadillo repeats, fibronectine type III domains, lipocalin-like domains, knottins, cellulose binding domains, carbohydrate binding domains, protein Z folds, PDZ domains, SH3 domains, SH2 domains, WW domains, thioredoxins, Leucine zipper, plant homeodomain, tudor domain, and hydrophobins.

Representative proteins that include each type of fold/domain are presented in Appendix B.

To identify candidate folds/domains in proteins believed or known to be secreted by species belonging to the Aspergillus, Trichoderma, Penicillium, Chrysosporium, Trametes, and Rhizopus fungal genera, possible conserved domains were identified using the reverse position specific blast (rpsblast) algorithm, as implemented in the NCBI Blast toolkit v2.2.26+(Marchler-Bauer 2004, Altschul 1997). The following default parameters were used to screen the secretome proteins for those genera listed in Appendix A: a gap opening penalty of −11, gap extension penalty or −1, an e-value cutoff of 1 and the BLOSUM62 scoring matrix.

Using this procedure, proteins comprising at least one of the folds/domains of interest were identified. When the database of sequences was searched using the RPSblast algorithm, hits were defined by both a fold/domain as well as a sequence range that best matches that fold/domain. It was determined that these sequence bookends often don't cover the entire range of the fold, so the protein sequences were checked and the domains expanded or reduced by reference to the crystal structure, which usually provided a clearer picture of where a fold starts and/or ends.

The four tables list identified proteins that comprise cellulose binding domains, carbohydrate binding modules, fibronectin type III domains, and hydrophobins.

Cellulose Binding Domains:

| | UniProt Accessions |
|---|---|
| Hit Name | (binding domain locations start:end) |
| Acetylxylan esterase A | Q4WBW4 (339:371), Q99034 (270:302), |
| | A1DBP9 (335:368), Q8NJP6 (349:382) |
| 1,4-beta-D-glucan | A1CU44 (507:539), B0Y8K2 (500:532), |
| cellobiohydrolase B | Q4WM08 (500:532), Q0CMT2 (509:541), |
| | Q8NK02 (494:526), A1DNL0 (498:530) |
| 1,4-beta-D-glucan | A1CCN4 (24:54), B0XWL3 (22:55), |
| cellobiohydrolase C | Q4WFK4 (22:55), A2QYR9 (21:53), |
| | Q0CFP1 (23:54), Q5B2E8 (22:55), |
| | A1DJQ7 (22:55) |
| endo-beta-1,4-glucanase | A1C4H2 (318:351), B0Y9G4 (316:347), |
| D | B8MXJ7 (332:365), Q4WBU0 (316:347), |
| | Q96WQ9 (372:405), A2R5N0 (378:409), |
| | Q2US83 (332:365), Q0CEU4 (327:358), |
| | Q5BCX8 (324:355), A1DBS6 (315:346) |
| Feruloyl esterase B | Q9HE18 (320:353) |
| Endoglucanase-4 | O14405 (311:343) |
| Exoglucanase 1 and 2 | P62694 (481:513), Q06886 (506:537), |
| | P13860 (485:516), Q9P8P3 (472:505), |
| | P62695 (481:513), P07987 (30:62) |
| Mannan endo-1,4-beta- | A1C8U0 (21:54), B0Y9E7 (21:54), |
| mannosidase F | B8NIV9 (21:54), Q4WBS1 (21:54), |
| | Q2U2I3 (21:54), Q5AR04 (20:53), |
| | A1DBV1 (21:54) |

Carbohydrate Binding Modules:

| | UniProt Accessions |
|---|---|
| Hit Name | (binding module locations start:end) |
| Alpha-galactosidase | B0YEK2 (497:646), B8N7Z0 (505:654), |
| D (CBM35) | A4DA70 (497:646), A2R2S6 (508:658), |
| | Q2UI87 (507:656), Q0CVX4 (504:653), |
| | Q5AX28 (506:657), A1D9S3 (497:646) |
| Alpha-glucuronidase | A1CC12 (698:833), B0Y2K1 (698:833), |
| A (CBM6) | Q4WW45 (698:833), Q5AQZ4 (705:840), |
| | Q99024 (709:840) |
| Chitinase 1 and 2 | P29026 (353:398), P29027 (355:400) |
| (CBD19) | |
| Glucoamylase | P69328 (547:639), P69327 (543:634), |
| (CBM20) | P36914 (513:603), P23176 (542:638), |
| | P22832 (542:638), A2QHE1 (543:639) |

Fibronectin Type III Domains:

| | UniProt Accessions |
|---|---|
| Hit Name | (domain locations start:end) |
| beta-glucosidase A | A1CR85(786:854), B0XPE1(792:860), |
| | B8NRX2 (780:848), Q4WJJ3 (792:860), |
| | P87076 (779:847), A2RAL4 (779:847), |
| | Q2UUD6 (780:848), D0VKF5 (780:848), |
| | Q0CTD7 (780:848), Q5B5S8 (782:850), |
| | A1D451 (792:860) |
| beta-glucosidase D | B8NJF4 (668:737), A2QPK4 (670:739), |
| | Q2UNR0 (668:737), Q5AUW5 (728:797) |
| beta-glucosidase F | B0Y7Q8 (781:853), B8NP65 (777:850), |
| | Q4WMU3 (781:853), Q2UN12 (777:850), |
| | Q0CI67 (778:851), Q5B6C6 (779:852), |
| | A1DMR8 (781:853) |
| beta-glucosidase G | B8NMR5 (731:801), Q2U325 (731:801), |
| | Q0CUC1 (731:801), Q5B0F4 (735:805), |
| | A1DC16 (731:801) |
| beta-glucosidase | A1CUR8 (742:809), B0XM94 (742:809), |
| H, I, J | B8NPL7 (738:807), Q4WL79 (742:809), |
| | Q2U9M7 (738:807), Q5B6C7 (742:811), |
| | A1DPG0 (742:809), A1CA51 (748:815), |
| | B0Y3M6 (748:815), B8NDE2 (749:809), |
| | Q4WU49 (748:815), A2R989 (728:795), |
| | Q2U8Y5 (749:816), Q0CAF5 (749:816), |
| | Q5BB53 (749:816), A1DFA8 (748:815), |
| | B0Y8M8 (772:844), Q4WLY1 (772:844), |
| | Q5AV15 (758:826), A1DNN8 (771:843) |
| beta-glucosidase K | Q5BA18 (746:818) |
| beta-glucosidase | B0YB65 (654:724), Q4WGT3 (654:724), |
| L, M, N, O | Q0CEF3 (655:725), Q5B9F2 (656:726), |
| | A1DCV5 (654:724), B0XPB8 (692:758), |
| | B8N5S6 (691:757), Q4WR62 (692:758), |
| | A5ABF5 (688:754), Q2UDK7 (691:757), |
| | Q0C7L4 (705:771), Q5AWD4 (695:761), |
| | A1D122 (692:758), Q5B681 (587:656), |
| | Q5BG51 (477:516) |
| exo-1,4-beta-xylosidase | A1CCL9 (674:740), Q0CB82 (666:732), |
| bxlB | Q5ATH9 (666:732) |
| exo-1,4-beta-xylosidase | Q4AEG8 (728:776), B0XP71 (695:758), |
| bxlD | B8MYV0 (700:763), Q4WRB0 (695:758), |
| | A2QA27 (728:776), O00089 (728:776), |
| | Q2UR38 (701:764), Q0CMH8 (695:758), |
| | Q5BAS1 (692:755) |
| Chitinase 1 and 2 | P29026 (34:304), P29027 (34:299) |
| 42 kDa endochitinase | P48827 (43:291) |
| Alkaline protease 1 | A1CIA7 (186:363), B0Y708 (153:363), |
| | P35211 (187:364), B8N106 (153:360), |
| | P28296 (153:363), P12547 (153:360), |
| | Q00208 (152:359), A1CWF3 (153:363) |

Hydrophobins:

| | UniProt Accessions |
|---|---|
| Hit Name | (hydrophobin location start:end) |
| Hydrophobin dewA | P52750 |
| Hydrophobin-1,2 | P52754 (29:94), P79073 (18:82) |
| Cell wall protein qid3 | P52755 (73:123) |
| Hydrophobin | P41746 (58:156) |
| Rodlet Protein | P28346 (59:154) |

### Example 3

### Identification of Amino Acid Positions in Reference Secreted Proteins for Substitution—Methods

Positions in reference secreted proteins for substitution with nutritive amino acids were identified by analyzing position amino acid likelihood, position entropy, mutation effect on relative folding free energy, and secondary structure type.

Position Amino Acid Likelihood

For a given query protein sequence, homologous proteins were identified by performing local sequence alignments of the query with NCBI's library of non-redundant proteins. The initial local alignments were performed using the blastp program from the NCBI toolkit v.2.2.26+(Altschul S. F., Gish W., Miller W., Myers E. W., and Lipman D. J. “Basic Local Alignment Search Tool”. J. Mol. Biol. (1990) 215: 403-410) with an e-value cutoff of 1, a gap opening penalty of −11, a gap extension penalty of −1, and the BLOSUM62 scoring matrix. The multiple sequence alignment of the resulting library was performed using the Align123 algorithm as implemented in Discovery Studio v3.1 (Accelrys Software Inc., Discovery Studio Modeling Environment, Release 3.1, San Diego: Accelrys Software Inc., 2012). Residue secondary structure was assigned using the DSC algorithm (King R. D., Sternberg M. J. E. “Identification and application of the concepts important for accurate and reliable protein secondary structure prediction”. Prot. Sci. (1996) 5: 2298-2310) with a weight of 1. Pairwise alignments were performed using the Smith and Waterman algorithm with a Gap opening penalty of −10 and gap extension penalty of −0.1, and the BLOSUM30 scoring matrix. Higher order alignments used the BLOSUM scoring matrix set, a gap opening penalty of −10, a gap extension penalty of −0.5, and an alignment delay identity cutoff (delay divergent parameter) of 40%.

All proteins with a local alignment expectation value less than 1 (from 75 to 1000 unique hits) were identified and aligned to generate a multiple sequence alignment (MSA). The proteins used for each MSA are presented in Appendix C.

From this MSA, the probability of observing each amino acid (or member of a group of amino acids) at each position in the protein sequence was computed using MATLAB 2012a software. For a given position, the likelihood of any given amino acid (or group of amino acids) is equal to the probability of observing that amino acid (or group of amino acids) across all sequences in the MSA. From this data, a rank ordered list of positions that are expected to be tolerant to each given amino acid substitution was generated for the protein. The rank ordered tables were then analyzed to assess the number of substitutions necessary to achieve a given increase in nutritive amino acid content.

In the examples disclosed herein, substitutions of Leu for non-Leu amino acids in reference protein sequences were examined That is, non-Leu amino acids in reference proteins were replaced with Leu amino acids. As one of skill in the art will appreciate, this approach can be broadly applied to any amino acid or group of amino acids (e.g., essential amino acids or branched chain amino acids, or the specific branch chain amino acids Ile or Val).

The rank ordered tables can be used to generate engineered versions of a reference protein in which one or more non-Leu residue that appears at a position with a Leu-likelihood score of at least a given threshold is substituted with a Leu amino acid. In the examples presented below all possible thresholds were examined and the results are presented graphically. To generate an engineered version of a reference protein corresponding to a Leu likelihood threshold of 0.6, for example, the non-Leu amino acids in the reference protein with Leu likelihood scores of at least 0.6 were identified and replaced with Leu to generate an engineered protein sequence comprising an increased number of Leu amino acids.

Without wishing to be bound to any particular theory, it is believed that positions in the reference protein that do not have a Leu amino acid but that correspond to Leu amino acids in homologous proteins are likely to tolerate replacement of the non-Leu amino acid with a Leu amino acid. Alternatively, the branched chain amino acid (BCAA) likelihood score of each amino acid position in the reference protein can be calculated as described above, then the positions in the reference protein that do not have a Leu amino acid but correspond to a particular frequency of occurrence of any BCAA in homologous proteins can be identified and replaced with Leu. Another strategy is to calculate the hydrophobic amino acid likelihood score (wherein the hydrophobic amino acids consist of Ala, Met, Ile, Leu, and Val) of each amino acid position in the reference protein as described above, then the positions in the reference protein that do not have a Leu amino acid but correspond to a particular frequency of occurrence of any hydrophobic amino acid in homologous proteins can be identified and replaced with Leu.

Position Entropy

The multiple sequence alignments were also used to compute the entropy of each amino acid position in a given reference amino acid sequence using the full amino acid alphabet, AA=[A, R, N, D, C, Q, E, G, H, I, L, K, M, F, P, S, T, W, Y, V]:

S=−Σ_{jεAA}pⱼ ln pⱼ

where pⱼ is the probability of seeing the amino acid j at that position. The entropy of each position was computed using the equation shown above using in-house code implemented in MATLAB2012a. This is a measure of the spread of the amino acid distribution. Highly variable positions will have large entropies (the maximum entropy at a position corresponds to each amino acid being equally likely, which yields an entropy of 2.996) and highly conserved positions will have an entropy close to 0.

Each amino acid residue in the protein was then rank ordered based on the calculated entropy to find positions that were likely tolerant to a variety of substitutions. For a desired amino acid enrichment, the number of mutations needed was determined as well as the probability of the least likely mutation to achieve a given amino acid fraction or nutritive content (e.g., essential amino acid content or branched chain amino acid content) by weight.

In a variation on this method, the same analysis was repeated, but instead of using the full amino acid alphabet to calculate the position entropy, amino acids were grouped based on physiochemical properties as follows: hydrophobic [A, V, I, L, M], aromatic [F, Y, W], polar [S, T, N, Q], charged [R, H, K, D, E], and non-classified [G, P, C]. As described above, each amino acid residue in the protein was then rank ordered based on the calculated entropy to find positions that were likely tolerant to a variety of substitutions. For a desired amino acid enrichment, the number of mutations needed was determined as well as the probability of the least likely mutation to achieve a given amino acid fraction or nutritive content (e.g., essential amino acid content or branched chain amino acid content) by weight. Using this physiochemical alphabet, pⱼ now corresponds to the probability of seeing each amino acid type (hydrophobic, aromatic, polar, charged, or non-classified) at position j. These amino acid type (AAType) probabilities are the sum of the probabilities of seeing each amino acid of that type. The equation for the position entropy stays the same, although the theoretical maximum is now ln(5)≈1.6.

Relative Folding Free Energy

Without wishing to be bound to any particular theory, it is believed that a given secreted protein that is engineered according to the methods described herein will continue to be secreted as long as it has a functional secretion leader sequence and maintains a stable, similar structural fold upon mutation. Accordingly, to analyze the effect on relative folding free energy of making amino acid substitutions to a reference secreted protein to modify its nutritive properties, an all atom structural model of the protein was constructed based on the structure of known structural homologues. All structural models and free energy calculations were performed using Discovery Studio v3.1 (Accelrys Software Inc., Discovery Studio Modeling Environment, Release 3.1, San Diego: Accelrys Software Inc., 2012). When possible, protein structural models were obtained from the protein databank (H. M. Berman, K. Henrick, H. Nakamura. “Announcing the worldwide Protein Data Bank Nature Structural Biology”. Nat. Struct. Biol. (2003) 10: 98). If a protein model was not available in the protein database, a model was constructed using the closest available structural homologue using the homology modeling software MODELLER (Eswar, N.; Eramian, D.; Webb, B.; Shen, M. Y.; Sali, A. “Protein structure modeling with MODELLER”. Methods Mol. Biol. (2008) 426: 145-159) as implemented in Discovery Studio v3.1 (Accelrys Software Inc., Discovery Studio Modeling Environment, Release 3.1, San Diego: Accelrys Software Inc., 2012). All energies were computed with the CHARMM software package (Brooks, B. R.; Brooks, C. L. 3rd; Mackerell, A. D. Jr.; Nilsson, L.; Petrella, R. J.; Roux, B.; Won, Y.; Archontis, G.; Bartels, C.; Boresch, S.; Caflisch, A.; Caves, L.; Cui, Q.; Dinner, A. R.; Feig, M.; Fischer, S.; Gao, J.; Hodoscek, M.; Im, W.; Kuczera, K.; Lazaridis, T.; Ma, J.; Ovchinnikov, V.; Paci, E.; Pastor, R. W.; Post, C. B.; Pu, J. Z.; Schaefer, M.; Tidor, B.; Venable, R. M.; Woodcock, H. L.; Wu, X.; Yang, W.; York, D. M.; Karplus, M. “CHARMM: the biomolecular simulation program”. J. Comput. Chem. (2009) 30: 1545-1614) and CHARMm polar hydrogen forcefield, as implemented in Discovery Studio v3.1 (Accelrys 2012), using a generalized born electrostatic model (Spassov V. Z., Yan L., and Szalma S. “Introducing an Implicit Membrane in Generalized Born/Solvent Accessibility Continuum Solvent Models”. J. Phys. Chem. B. (2002) 106: 8726-8738.) and empirical configurational entropy model (Abagyan R. and Totrov M. “Biased Probability Monte Carlo Conformational Searches and Electrostatic Calculations for Peptides and Proteins”. J. Mol. Biol. (1994) 235: 983-1002).

For each position, the free energy of folding (ΔΔG_{fold}) for all possible single amino acid mutations relative to the wild type folding free energy was computed. Each amino acid substitution was then rank ordered based on its predicted effect on folding stability. We also decomposed each ΔΔG_{fold} into contributions from van der Waals, electrostatic, and thermodynamic entropic free energy changes. In the absence of having the ΔΔG_{fold} for all possible mutation combinations, knowing how each mutation affects each free energy component offers a way of reducing possible errors with using in silico predictions. When selecting a number of mutations to make to a single protein, one consideration is minimizing ΔΔG_{fold}, however in some cases multiple mutations with comparable changes to ΔΔG_{fold} are available. Given possible imperfections in the in silico model, it is possible that for a given protein, one component of the calculated energy change is more predictive than another. As such, the likelihood of finding a successful combination of mutants is increased by selecting a combination that affects the free energy change in different ways.

Secondary Structure Type

Given a structural model for a given protein, loop residues were identified using the DSC algorithm (King R. D., Sternberg M. J. E. “Identification and application of the concepts important for accurate and reliable protein secondary structure prediction”. Prot. Sci. (1996) 5: 2298-2310), as these residues are not a part of any specific backbone hydrogen bonding pattern (i.e. devoid of secondary structure) and often show significant structural variability (Shehu, A.; Kavraki, L. E. Modeling Structures and Motions of Loops in Protein Molecules. Entropy 2012, 14, 252-290.). Additionally, these sites are often the source of functional variability in protein-protein or protein-ligand interactions (Lehtio, J., Teeri T. T., and Nygren P. A. “Alpha-Amylase Inhibitors Selected From a Combinatorial Library of a Cellulose Binding Domain Scaffold”. Proteins: Struct., Func., Gene., (2000) 41: 316-322; Bloom L. and Calabro V. “FN3: a new protein scaffold reaches the clinic”. Drug Disc. Today (14): 949:955; Hackel B. J., Kapila A., and Wittrup K. D. “Picomolar Affinity Fibronectin Domains Engineered Utilizing Loop Length Diversity, Recursive Mutagenesis, and Loop Shuffling”. J. Mol. Biol. (2008) 381: 1238-1252; and Olson C A and Roberts R W. “Design, expression, and stability of a diverse protein library based on the human fibronectin type III domain”. Prot. Sci. (2007) 16: 476-484), and site directed mutagenesis of these residues can cause changes in binding specificity without significantly affecting stability. As such, the primary and tertiary structural plasticity of these positions make them high priority positions for sequence variation to improve nutritional content.

### Example 4

### Identification of Amino Acid Positions for Substitution in the A. niger Glucoamylase Protein (SEQ ID NO: 1)

Glucoamylase from A. niger (SEQ ID NO: 1) contains 7.4% by weight Leu, 17.4% by weight branch chain amino acids, and 42.2% by weight essential amino acids.

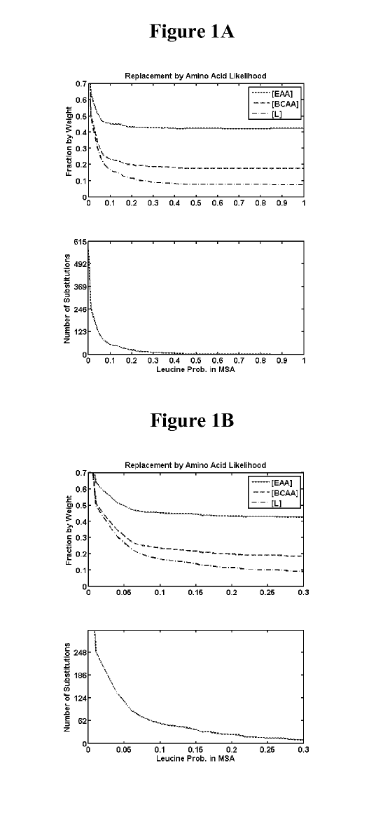

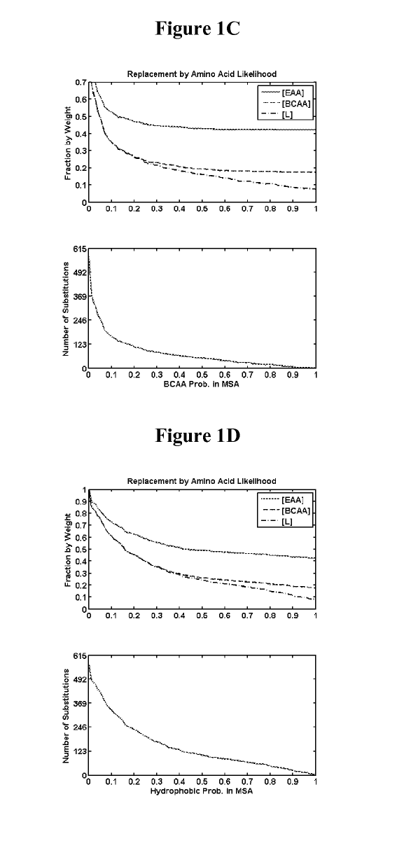

FIG. 1A analyzes the amino acid content (by weight) of engineered proteins generated by replacing all non-Leu amino acids that occur at amino acid positions identified using different Leu likelihood thresholds from 0 to 1. Specifically, the weight fraction of Leu, BCAAs, and EAAs in SEQ ID NO: 1 are shown. In the top panel, the likelihood threshold for making amino acid replacements is presented on the X-axis. Thus the value 0.6 on the X-axis, for example, represents an engineered protein sequence created by identifying every amino acid position in SEQ ID NO: 1 having a Leu-likelihood score of at least 0.6 and replacing all non-Leu amino acids appearing at one of those positions with a Leu amino acid. In the top panel, the fraction by weight of Leu, BCAA, and EAA in the protein following any necessary Leu replacements is shown on the Y-axis. In the bottom panel the Y-axis indicates the total of number of Leu replacements made to a protein when every amino acid position that has a given Leu likelihood score on the X-axis is occupied by a Leu amino acid in the engineered protein. The top and bottom panels of FIG. 1B present a close-up view of the data for Leu likelihood scores of 0 to 0.3 (i.e., the left portion of the graphs shown in FIG. 1A).

This analysis was repeated, but instead of calculating Leu likelihood to identify amino acid positions for mutation, BCAA likelihood (FIG. 1C) and hydrophobic amino acid likelihood (FIG. 1D) were calculated and every non-Leu amino acid at the identified positions was replaced with a Leu amino acid. As expected, these two less stringent screens lead to replacement of more non-Leu amino acids with Leu at each likelihood cut off.

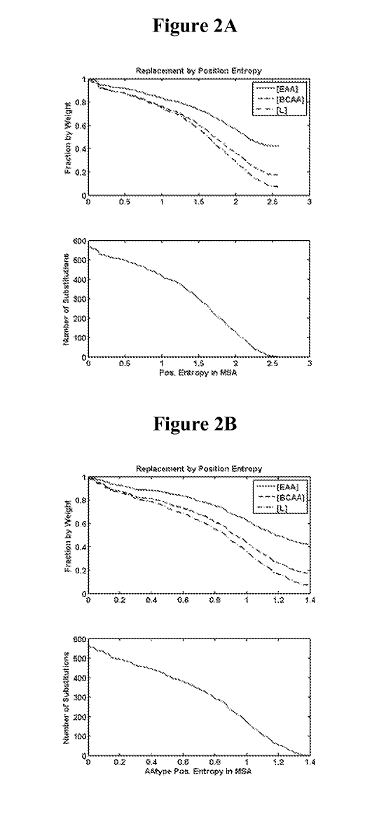

The analysis was then repeated using position entropy instead of amino acid likelihood to rank amino acid positions for substitution by Leu. Results obtained when position entropy was calculated using the frequency of each amino acid at each position are presented in FIG. 2A and when position entropy was calculated using the frequency of amino acid type at each position in FIG. 2B.

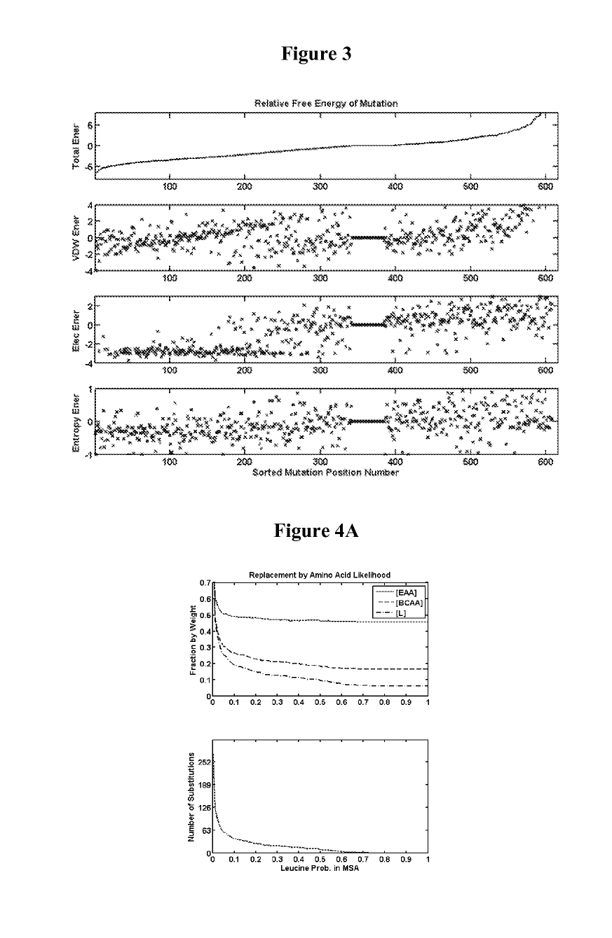

The free energy of folding (ΔΔG_{fold}) for all possible single amino acid mutations of non-Leu amino acids in SEQ ID NO: 1 to Leu, relative to the wild type free energy of folding in SEQ ID NO: 1, was calculatede. Each amino acid substitution was then rank ordered based on its predicted effect on folding stability. The results are shown in FIG. 3A, top panel. We also decomposed each ΔΔG_{fold} into contributions from van der Waals, electrostatic, and thermodynamic entropic free energy changes. (FIG. 3A, lower three panels.) Given all mutations that are predicted to result in a favorable free energy of folding (ΔΔG_{fold}<0), 21% are driven by VDW energy changes, 76% by electrostatic energy changes, and 3% by entropic free energy changes. As such, while the majority of mutations are predicted to improve stability through favorable vdw changes, many are driven by electrostatic changes, and a balanced, hedged approach would involve selecting mutants that improve both vdw as well as electrostatics.

For each amino acid in SEQ ID NO: 1, the loop ID (1=loop; 0=non-loop), Leu likelihood, BCAA likelihood, EAA likelihood, hydrophobic amino acid likelihood, amino acid position entropy, amino acid type position entropy, total free energy of folding (ΔΔG_{fold}) (for substitution by Leu), the van der Waals free energy change contribution to total free energy of folding (ΔΔGfold VDW) (for substitution by Leu), the electrostatic free energy change contribution to total free energy of folding (ΔΔGfold Elec) (for substitution by Leu), and the thermodynamic entropic free energy change contribution to total free energy of folding (ΔΔGfold Entropy) (for substitution by Leu) was calculated. The results are shown in Table 1 in Appendix D.

### Example 5

### Identification of Amino Acid Positions for Substitution in the A. niger Endo-Beta-1,4-Glucanase Protein (SEQ ID NO: 2)

Endo-beta-1,4-glucanase from A. niger (SEQ ID NO: 2) contains 6.2% by weight Leu, 16.5% by weight branch chain amino acids, and 45.6% by weight essential amino acids.

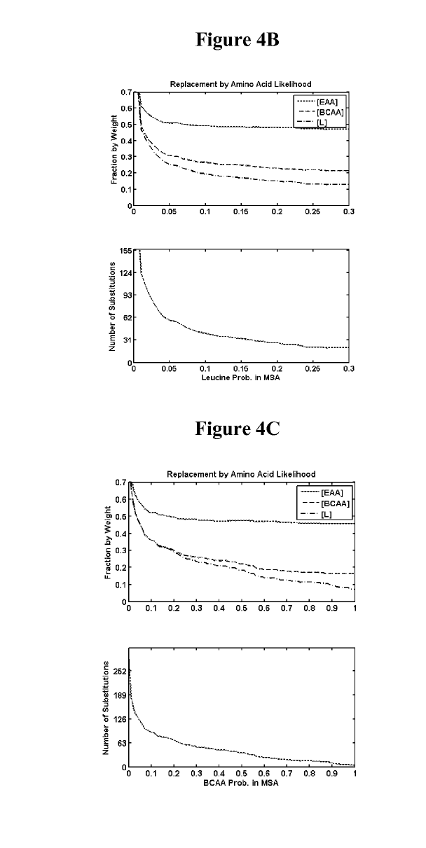

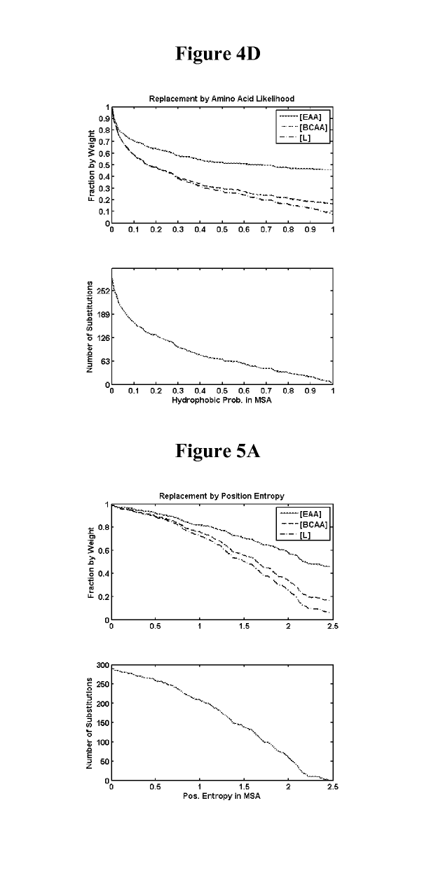

FIG. 4A analyzes the amino acid content (by weight) of engineered proteins generated by replacing all non-Leu amino acids that occur at amino acid positions identified using different Leu likelihood thresholds from 0 to 1. Specifically, the weight fraction of Leu, BCAAs, and EAAs in SEQ ID NO: 2 are shown. In the top panel, the likelihood threshold for making amino acid replacements is presented on the X-axis. So the value 0.6 on the X-axis, for example, represents an engineered protein sequence created by identifying every amino acid position in SEQ ID NO: 2 having a Leu-likelihood score of at least 0.6 and replacing all non-Leu amino acids appearing at one of those positions with a Leu amino acid. In the top panel, the fraction by weight of Leu, BCAA, and EAA in the protein following the making of any necessary Leu replacements is shown on the Y-axis. In the bottom panel the Y-axis indicates the total of number of Leu replacements made to a protein when every amino acid position that has a given Leu likelihood score on the X-axis is occupied by a Leu amino acid in the engineered protein. The top and bottom panels of FIG. 4B present a close-up view of the left end of the graphs (for Leu likelihood scores of 0 to 0.3) shown in FIG. 4A.

This analysis was repeated, but instead of assessing Leu likelihood to identify amino acid positions for mutation the BCAA likelihood and hydrophobic amino acid likelihood in the MSA data was used to identify amino acid positions, and then replaced every non-Leu amino acid at an identified position replaced with a Leu amino acid. The results are presented in FIG. 4C (BCAA probability) and FIG. 4D (position entropy). As expected, these two less stringent screens lead to replacement of more non-Leu amino acids with Leu at each likelihood cut off.

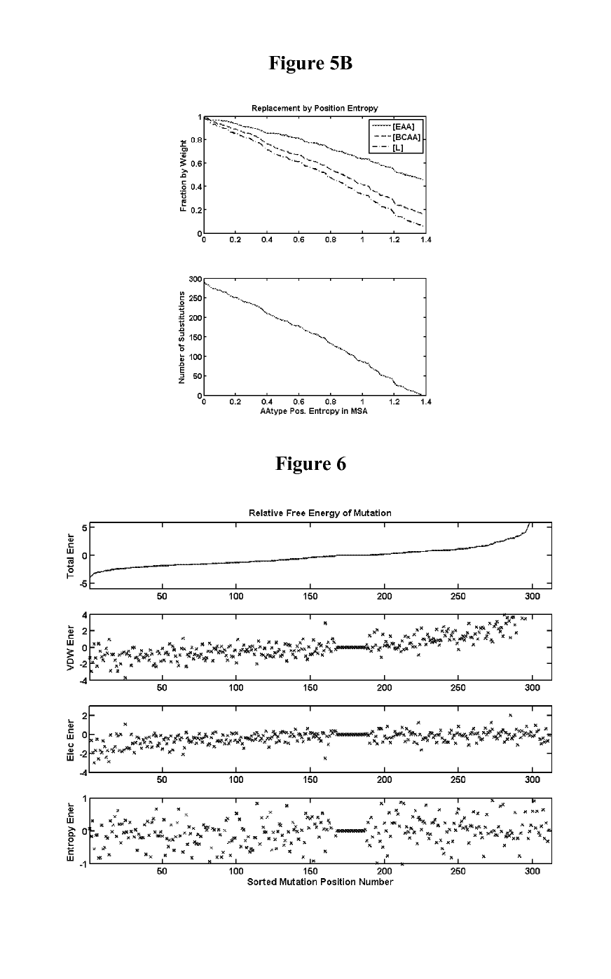

The same analysis was repeated, using position entropy instead of raw amino acid likelihood to rank amino acid positions for substitution by Leu. Results obtained when position entropy was calculated using the frequency of each amino acid at each position are presented in FIG. 5A and when position entropy was calculated using the frequency of amino acid type at each position in FIG. 5B.

The free energy of folding (ΔΔG_{fold}) was also calculated for all possible single amino acid mutations of non-Leu amino acids in SEQ ID NO: 2 to Leu, relative to the wild type free energy of folding in SEQ ID NO: 2. For each amino acid substitution, the positions were then rank ordered based on their predicted effect on folding stability. The results are shown in FIG. 6.

For each amino acid in SEQ ID NO: 2, the loop ID (1=loop; 0=non-loop), Leu likelihood, BCAA likelihood, EAA likelihood, hydrophobic amino acid likelihood, amino acid position entropy, amino acid type position entropy, total free energy of folding (ΔΔG_{fold}) (for substitution by Leu), the van der Waals free energy change contribution to total free energy of folding (ΔΔGfold VDW) (for substitution by Leu), the electrostatic free energy change contribution to total free energy of folding (ΔΔGfold Elec) (for substitution by Leu), and the thermodynamic entropic free energy change contribution to total free energy of folding (ΔΔGfold Entropy) (for substitution by Leu) was calculated. The results are shown in Table 2 in Appendix D.

### Example 5

### Identification of Amino Acid Positions for Substitution in the A. niger 1,4-beta-D-glucan cellobiohydrolase Protein (SEQ ID NO: 3)

1,4-beta-D-glucan cellobiohydrolase from A. niger (SEQ ID NO: 3) contains 5.5% by weight Leu, 13.1% by weight branch chain amino acids, and 37.7% by weight essential amino acids.

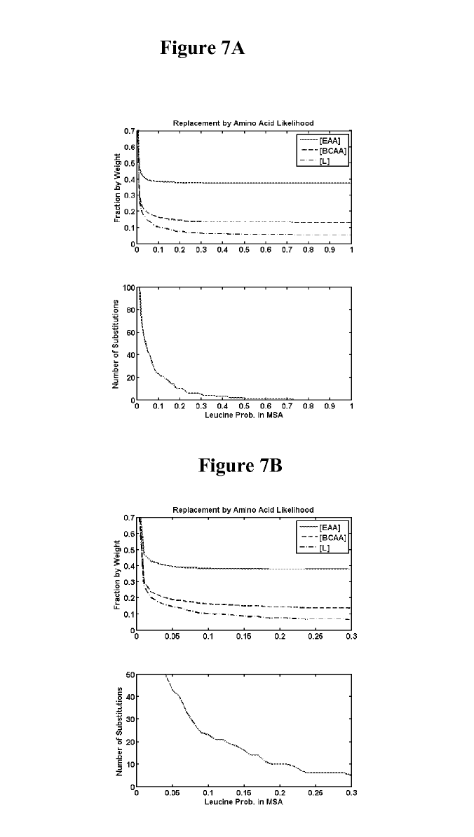

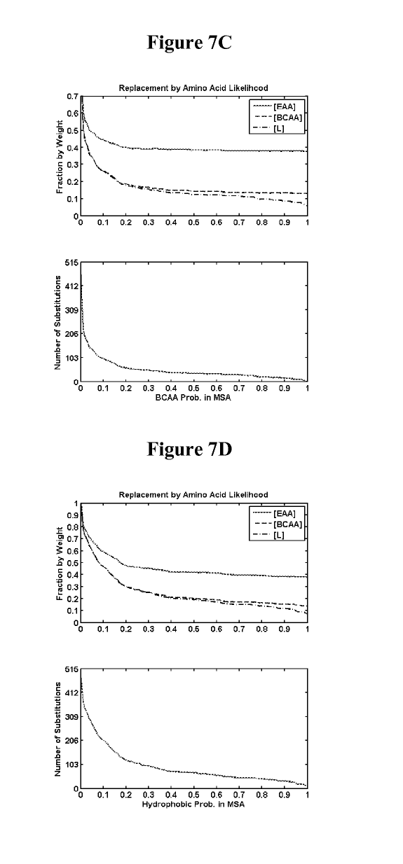

FIG. 7A analyzes the amino acid content (by weight) of engineered proteins generated by replacing all non-Leu amino acids that occur at amino acid positions identified using different Leu likelihood thresholds from 0 to 1. Specifically, the weight fraction of Leu, BCAAs, and EAAs in SEQ ID NO: 3 are shown. In the top panel, the likelihood threshold for making amino acid replacements is presented on the X-axis. So the value 0.6 on the X-axis, for example, represents an engineered protein sequence created by identifying every amino acid position in SEQ ID NO: 3 having a Leu-likelihood score of at least 0.6 and replacing all non-Leu amino acids appearing at one of those positions with a Leu amino acid. In the top panel, the fraction by weight of Leu, BCAA, and EAA in the protein following the making of any necessary Leu replacements is shown on the Y-axis. In the bottom panel the Y-axis indicates the total of number of Leu replacements made to a protein when every amino acid position that has a given Leu likelihood score on the X-axis is occupied by a Leu amino acid in the engineered protein. The top and bottom panels of FIG. 7B present a close-up view of the left end of the graphs (for Leu likelihood scores of 0 to 0.3) shown in FIG. 7A.

This analysis was repeated, but instead of assessing Leu likelihood to identify amino acid positions for mutation the BCAA likelihood and hydrophobic amino acid likelihood in the MSA data was measured to identify amino acid positions, and then every non-Leu amino acid at identified positions was replaced with a Leu amino acid. The results are presented in FIG. 7C (BCAA likelihood) and FIG. 7D (hydrophobic amino acid likelihood). As expected, these two less stringent screens lead to replacement of more non-Leu amino acids with Leu at each likelihood cut off.

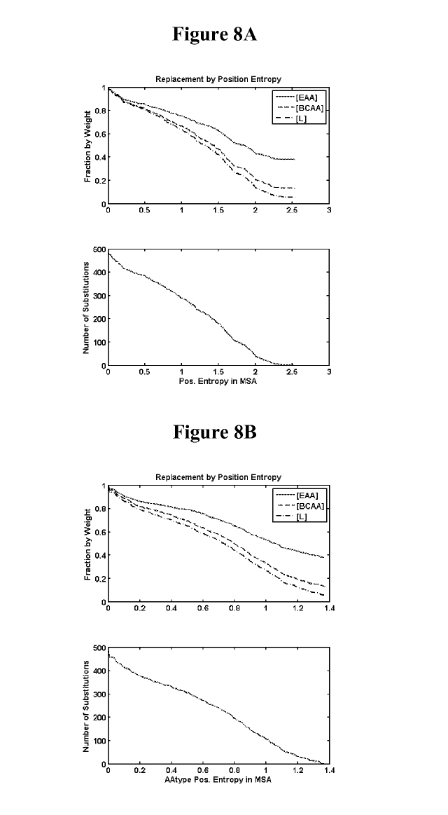

The same analysis was repeated, using position entropy instead of raw amino acid likelihood to rank amino acid positions for substitution by Leu. Results obtained when position entropy was calculated using the frequency of each amino acid at each position are presented in FIG. 8A and when position entropy was calculated using the frequency of amino acid type at each position in FIG. 8B.

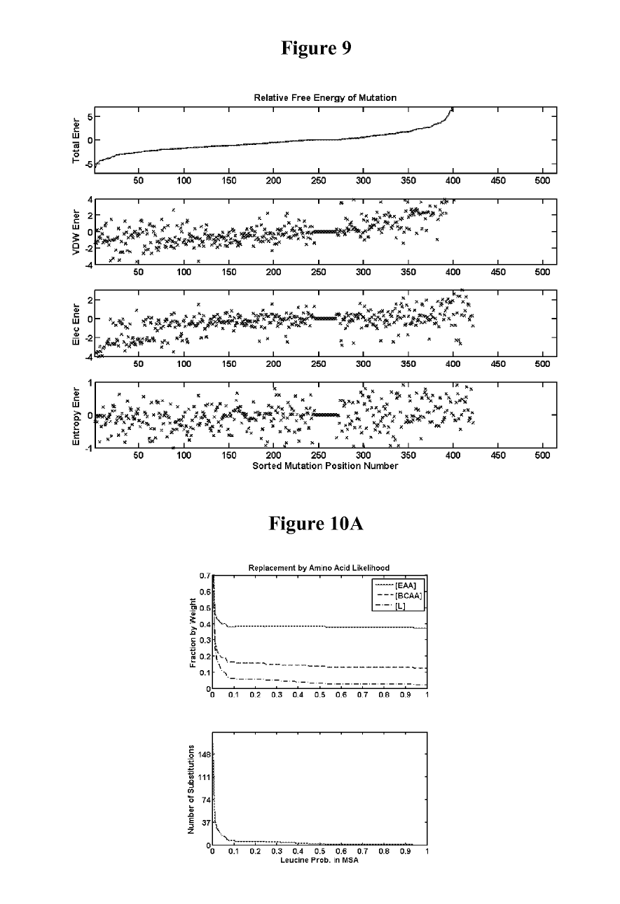

The free energy of folding (ΔΔG_{fold}) for all possible single amino acid mutations of non-Leu amino acids in SEQ ID NO: 3 to Leu, relative to the wild type free energy of folding in SEQ ID NO: 3, was also calculated. For each amino acid substitution, the positions were rank ordered based on their predicted effect on folding stability. The results are shown in FIG. 9.

For each amino acid in SEQ ID NO: 3, the loop ID (1=loop; 0=non-loop), Leu likelihood, BCAA likelihood, EAA likelihood, hydrophobic amino acid likelihood, amino acid position entropy, amino acid type position entropy, total free energy of folding (ΔΔG_{fold}) (for substitution by Leu), the van der Waals free energy change contribution to total free energy of folding (ΔΔGfold VDW) (for substitution by Leu), the electrostatic free energy change contribution to total free energy of folding (ΔΔGfold Elec) (for substitution by Leu), and the thermodynamic entropic free energy change contribution to total free energy of folding (ΔΔGfold Entropy) (for substitution by Leu) were calculated. The results are shown in Table 3 in Appendix D.

### Example 6

### Identification of Amino Acid Positions for Substitution in the A. niger Endo-1,4-beta-xylanase Protein (SEQ ID NO: 4)

Endo-1,4-beta-xylanase from A. niger (SEQ ID NO: 4) contains 2.2% by weight Leu, 12.6% by weight branch chain amino acids, and 37.4% by weight essential amino acids.

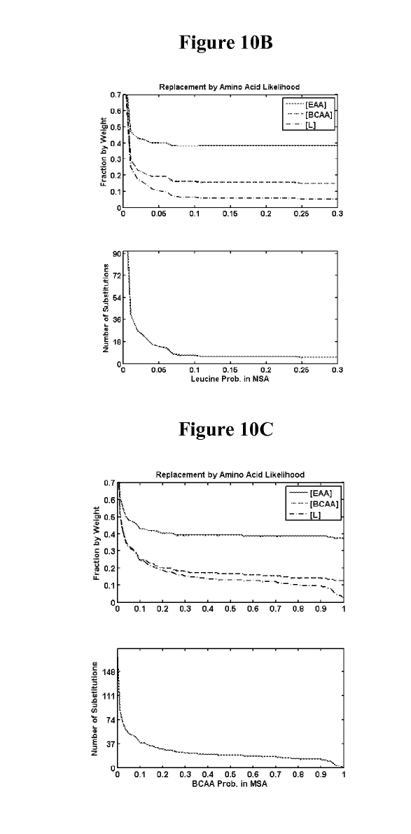

FIG. 10A analyzes the amino acid content (by weight) of engineered proteins generated by replacing all non-Leu amino acids that occur at amino acid positions identified using different Leu likelihood thresholds from 0 to 1. Specifically, the weight fraction of Leu, BCAAs, and EAAs in SEQ ID NO: 4 are shown. In the top panel, the likelihood threshold for making amino acid replacements is presented on the X-axis. So the value 0.6 on the X-axis, for example, represents an engineered protein sequence created by identifying every amino acid position in SEQ ID NO: 4 having a Leu-likelihood score of at least 0.6 and replacing all non-Leu amino acids appearing at one of those positions with a Leu amino acid. In the top panel, the fraction by weight of Leu, BCAA, and EAA in the protein following the making of any necessary Leu replacements is shown on the Y-axis. In the bottom panel the Y-axis indicates the total of number of Leu replacements made to a protein when every amino acid position that has a given Leu likelihood score on the X-axis is occupied by a Leu amino acid in the engineered protein. The top and bottom panels of FIG. 10B present a close-up view of the left end of the graphs (for Leu likelihood scores of 0 to 0.3) shown in FIG. 10A.

This analysis was repeated, but instead of assessing Leu likelihood to identify amino acid positions for mutation BCAA likelihood and hydrophobic amino acid likelihood was measured in the MSA data to identify amino acid positions, and then very non-Leu amino acid at identified positions was replaced with a Leu amino acid. The results are presented in FIG. 10C (BCAA likelihood) and FIG. 10D (hydrophobic amino acid likelihood). As expected, these two less stringent screens lead to replacement of more non-Leu amino acids with Leu at each likelihood cut off.

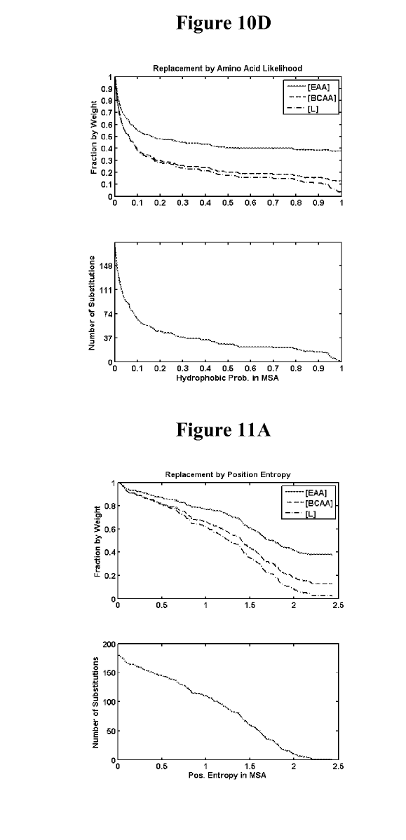

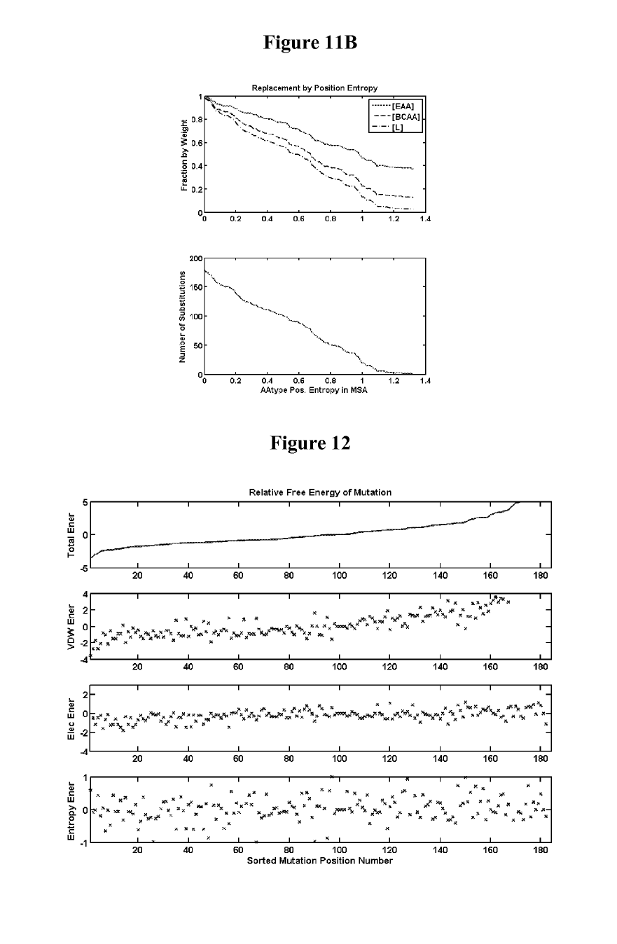

The same analysis was repeated, using position entropy instead of raw amino acid likelihood to rank amino acid positions for substitution by Leu. Results obtained when position entropy was calculated using the frequency of each amino acid at each position are presented in FIG. 11A and when position entropy was calculated using the frequency of amino acid type at each position in FIG. 11B.

The free energy of folding (ΔΔG_{fold}) for all possible single amino acid mutations of non-Leu amino acids in SEQ ID NO: 4 to Leu, relative to the wild type free energy of folding in SEQ ID NO: 4 was also calculated. For each amino acid substitution, the positions were rank ordered based on their predicted effect on folding stability. The results are shown in FIG. 12.

For each amino acid in SEQ ID NO: 4, the loop ID (1=loop; 0=non-loop), Leu likelihood, BCAA likelihood, EAA likelihood, hydrophobic amino acid likelihood, amino acid position entropy, amino acid type position entropy, total free energy of folding (ΔΔG_{fold}) (for substitution by Leu), the van der Waals free energy change contribution to total free energy of folding (ΔΔGfold VDW) (for substitution by Leu), the electrostatic free energy change contribution to total free energy of folding (ΔΔGfold Elec) (for substitution by Leu), and the thermodynamic entropic free energy change contribution to total free energy of folding (ΔΔGfold Entropy) (for substitution by Leu) were calculated. The results are shown in Table 4 in Appendix D.

### Example 7

### Identification of Amino Acid Positions for Substitution in the A. niger Cellulose Binding Domain 1 Protein (SEQ ID NO: 5)

Cellulose binding domain 1 from A. niger (SEQ ID NO: 5) contains 3.0% by weight Leu, 5.6% by weight branch chain amino acids, and 23.8% by weight essential amino acids.

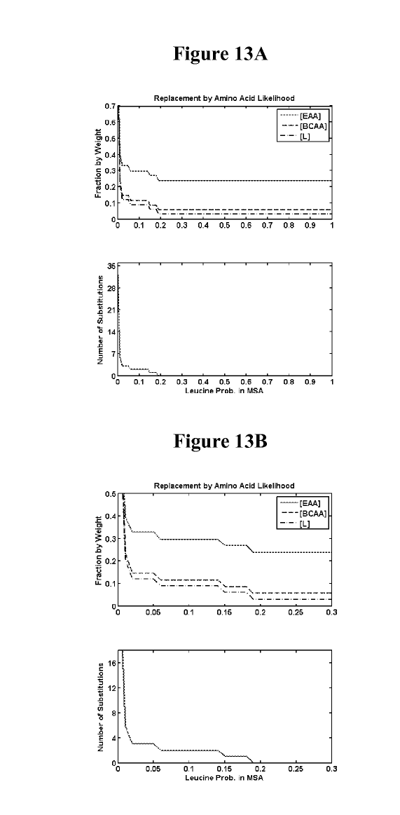

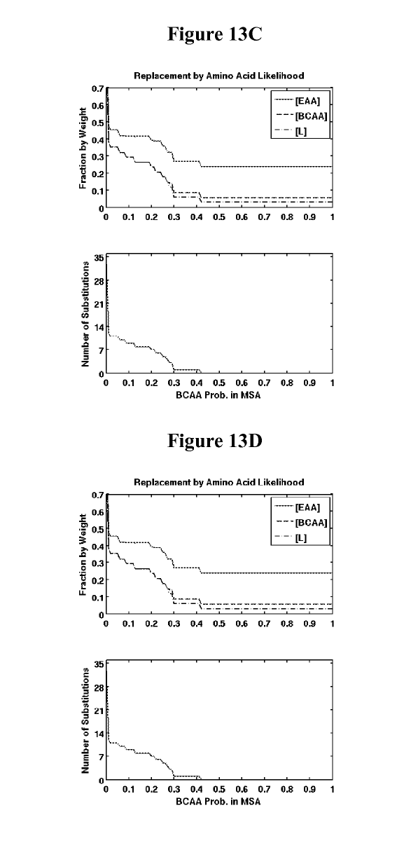

FIG. 13A analyzes the amino acid content (by weight) of engineered proteins generated by replacing all non-Leu amino acids that occur at amino acid positions identified using different Leu likelihood thresholds from 0 to 1. Specifically, the weight fraction of Leu, BCAAs, and EAAs in SEQ ID NO: 5 are shown. In the top panel, the likelihood threshold for making amino acid replacements is presented on the X-axis. So the value 0.6 on the X-axis, for example, represents an engineered protein sequence created by identifying every amino acid position in SEQ ID NO: 5 having a Leu-likelihood score of at least 0.6 and replacing all non-Leu amino acids appearing at one of those positions with a Leu amino acid. In the top panel, the fraction by weight of Leu, BCAA, and EAA in the protein following the making of any necessary Leu replacements is shown on the Y-axis. In the bottom panel the Y-axis indicates the total of number of Leu replacements made to a protein when every amino acid position that has a given Leu likelihood score on the X-axis is occupied by a Leu amino acid in the engineered protein. The top and bottom panels of FIG. 13B present a close-up view of the left end of the graphs (for Leu likelihood scores of 0 to 0.3) shown in FIG. 13A.

This analysis was repeated, but instead of assessing Leu likelihood to identify amino acid positions for mutation BCAA likelihood and hydrophobic amino acid likelihood were measured in the MSA data to identify amino acid positions, and every non-Leu amino acid at identified positions was replaced with a Leu amino acid. The results are presented in FIG. 13C (BCAA likelihood) and FIG. 13D (hydrophobic amino acid likelihood). As expected, these two less stringent screens lead to replacement of more non-Leu amino acids with Leu at each likelihood cut off.

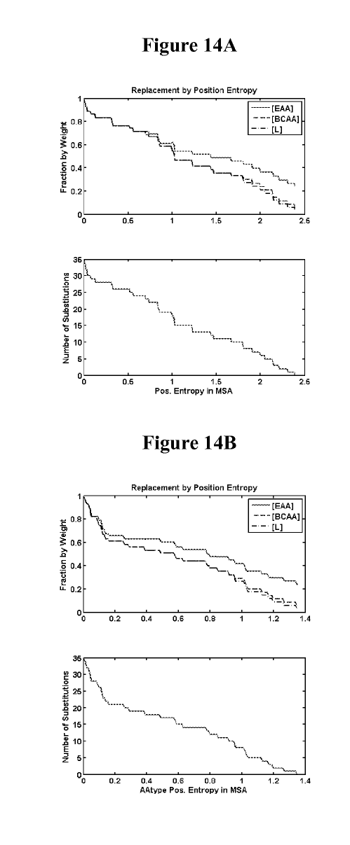

The same analysis was repeated, using position entropy instead of raw amino acid likelihood to rank amino acid positions for substitution by Leu. Results obtained when position entropy was calculated using the frequency of each amino acid at each position are presented in FIG. 14A and when position entropy was calculated using the frequency of amino acid type at each position in FIG. 14B.

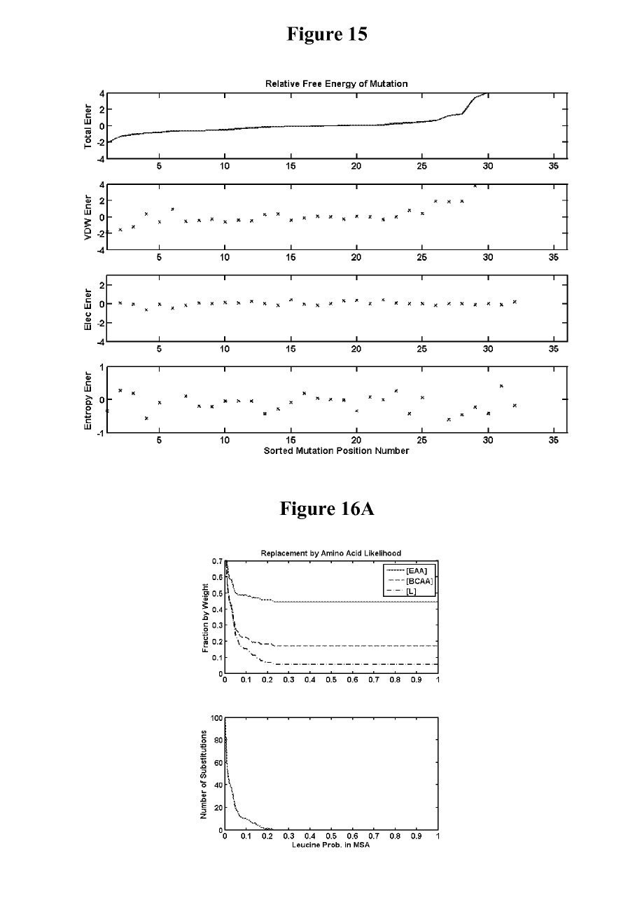

The free energy of folding (ΔΔG_{fold}) for all possible single amino acid mutations of non-Leu amino acids in SEQ ID NO: 5 to Leu, relative to the wild type free energy of folding in SEQ ID NO: 5 was also computed. For each amino acid substitution, the positions were rank ordered based on their predicted effect on folding stability. The results are shown in FIG. 15.

For each amino acid in SEQ ID NO: 5, the loop ID (1=loop; 0=non-loop), Leu likelihood, BCAA likelihood, EAA likelihood, hydrophobic amino acid likelihood, amino acid position entropy, amino acid type position entropy, total free energy of folding (ΔΔG_{fold}) (for substitution by Leu), the van der Waals free energy change contribution to total free energy of folding (ΔΔGfold VDW) (for substitution by Leu), the electrostatic free energy change contribution to total free energy of folding (ΔΔGfold Elec) (for substitution by Leu), and the thermodynamic entropic free energy change contribution to total free energy of folding (ΔΔGfold Entropy) (for substitution by Leu) were calculated. The results are shown in Table 5 in Appendix D.

### Example 8

### Identification of Amino Acid Positions for Substitution in the A. niger Carbohydrate Binding Module 20 Protein (SEQ ID NO: 6)

Carbohydrate binding module 20 protein from A. niger (SEQ ID NO: 6) contains 5.7% by weight Leu, 17.2% by weight branch chain amino acids, and 44.6% by weight essential amino acids.

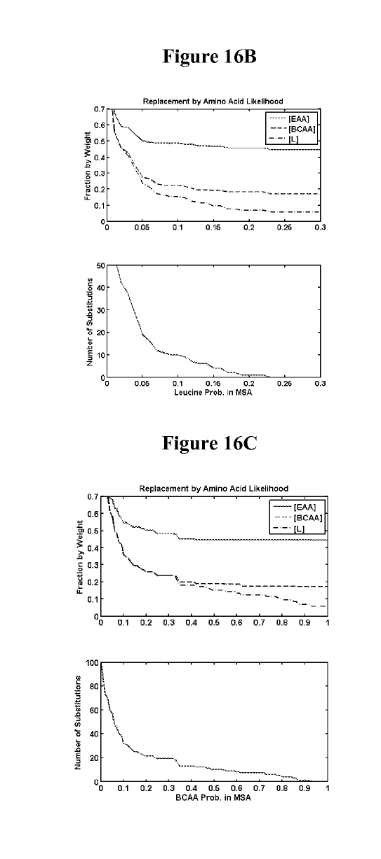

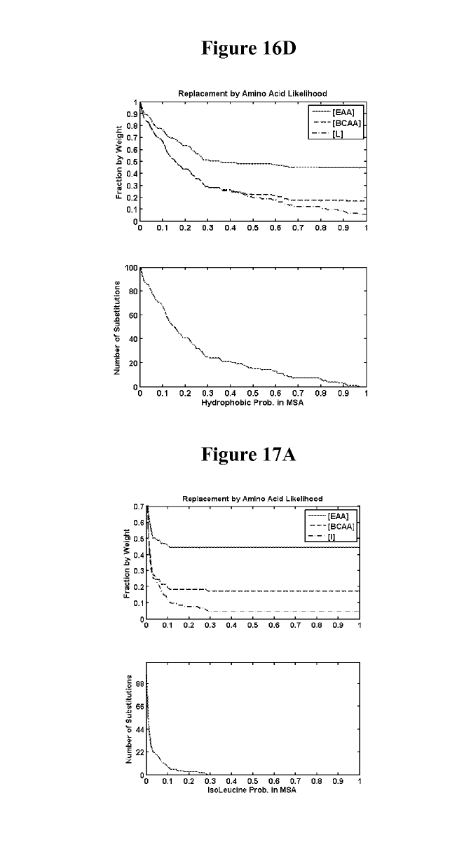

FIG. 16A analyzes the amino acid content (by weight) of engineered proteins generated by replacing all non-Leu amino acids that occur at amino acid positions identified using different Leu likelihood thresholds from 0 to 1. Specifically, the weight fraction of Leu, BCAAs, and EAAs in SEQ ID NO: 6 are shown. In the top panel, the likelihood threshold for making amino acid replacements is presented on the X-axis. So the value 0.6 on the X-axis, for example, represents an engineered protein sequence created by identifying every amino acid position in SEQ ID NO: 6 having a Leu-likelihood score of at least 0.6 and replacing all non-Leu amino acids appearing at one of those positions with a Leu amino acid. In the top panel, the fraction by weight of Leu, BCAA, and EAA in the protein following the making of any necessary Leu replacements is shown on the Y-axis. In the bottom panel the Y-axis indicates the total of number of Leu replacements made to a protein when every amino acid position that has a given Leu likelihood score on the X-axis is occupied by a Leu amino acid in the engineered protein. The top and bottom panels of FIG. 16B present a close-up view of the left end of the graphs (for Leu likelihood scores of 0 to 0.3) shown in FIG. 16A.

This analysis was repeated, but instead of assessing Leu likelihood to identify amino acid positions for mutation BCAA likelihood and hydrophobic amino acid likelihood were measured in the MSA data to identify amino acid positions, and every non-Leu amino acid at identified positions was replaced with a Leu amino acid. The results are presented in FIG. 16C (BCAA likelihood) and FIG. 16D (hydrophobic amino acid likelihood). As expected, these two less stringent screens lead to replacement of more non-Leu amino acids with Leu at each likelihood cut off.

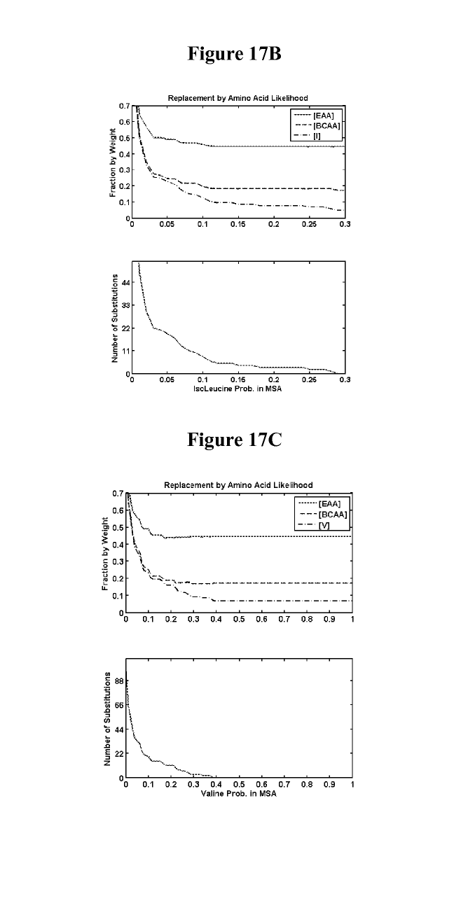

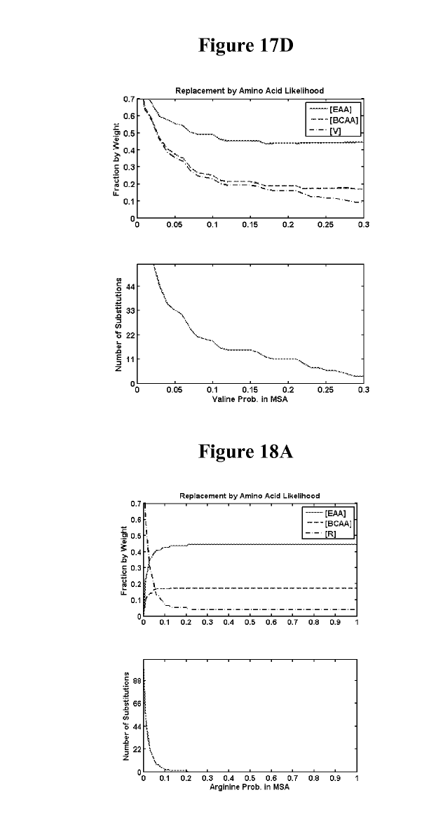

Replacing non-Leu residues with Leu residues increases the Leu content of a reference secreted protein, as well as the BCAA and EAA content. An alternative way to increase the BCAA and EAA content simultaneously is to increase the Val or Ile content of the reference secreted protein. To generate engineered proteins comprising increased Val or Ile content amino acid positions in Carbohydrate binding module 20 protein were identified based on Val likelihood or Ile likelihood. FIG. 17A analyzes the amino acid content (by weight) of engineered proteins generated by replacing all non-Ile amino acids that occur at amino acid positions identified using different Ile likelihood thresholds from 0 to 1. Specifically, the weight fraction of Ile, BCAAs, and EAAs in SEQ ID NO: 6 are shown. In the top panel, the likelihood threshold for making amino acid replacements is presented on the X-axis. So the value 0.6 on the X-axis, for example, represents an engineered protein sequence created by identifying every amino acid position in SEQ ID NO: 6 having an Ile-likelihood score of at least 0.6 and replacing all non-Ile amino acids appearing at one of those positions with an Ile amino acid. In the top panel, the fraction by weight of Ile, BCAA, and EAA in the protein following the making of any necessary Ile replacements is shown on the Y-axis. In the bottom panel the Y-axis indicates the total of number of Ile replacements made to a protein when every amino acid position that has a given Ile likelihood score on the X-axis is occupied by an Ile amino acid in the engineered protein. The top and bottom panels of FIG. 17B present a close-up view of the left end of the graphs (for Ile likelihood scores of 0 to 0.3) shown in FIG. 17A.

FIGS. 17C and 17D present a corresponding analysis for Val replacement. FIG. 17C analyzes the amino acid content (by weight) of engineered proteins generated by replacing all non-Val amino acids that occur at amino acid positions identified using different Val likelihood thresholds from 0 to 1. Specifically, the weight fraction of Val, BCAAs, and EAAs in SEQ ID NO: 6 are shown. In the top panel, the likelihood threshold for making amino acid replacements is presented on the X-axis. So the value 0.6 on the X-axis, for example, represents an engineered protein sequence created by identifying every amino acid position in SEQ ID NO: X having a Val-likelihood score of at least 0.6 and replacing all non-Val amino acids appearing at one of those positions with a Val amino acid. In the top panel, the fraction by weight of Val, BCAA, and EAA in the protein following the making of any necessary Val replacements is shown on the Y-axis. In the bottom panel the Y-axis indicates the total of number of Val replacements made to a protein when every amino acid position that has a given Val likelihood score on the X-axis is occupied by a Val amino acid in the engineered protein. The top and bottom panels of FIG. 17D present a close-up view of the left end of the graphs (for Ile likelihood scores of 0 to 0.3) shown in FIG. 17C.

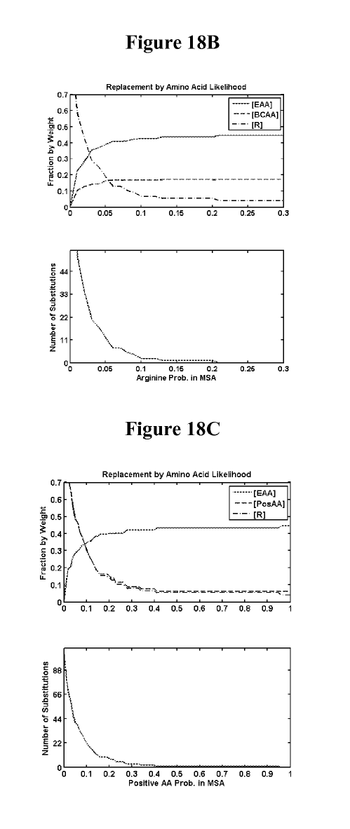

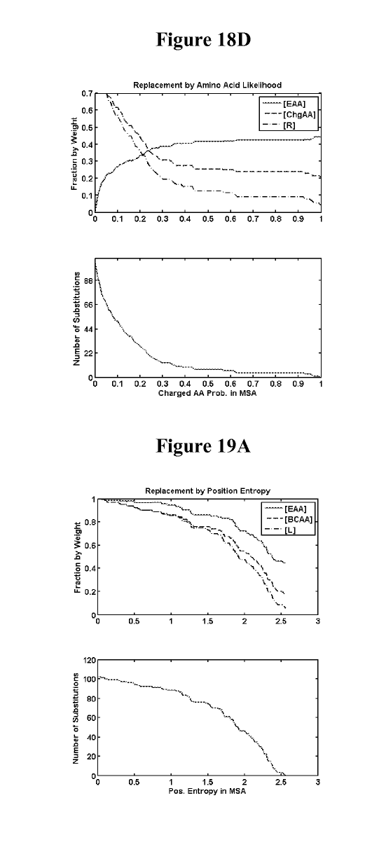

For some uses it may be desirable to increase the proportion of a non-BCAA and in some cases of a non-EAA in an engineered secreted protein. Arginine is a conditionally nonessential amino acid, meaning most of the time it can be manufactured by the human body, and does not need to be obtained directly through the diet. The amino acid arginine is known to have a large number of health benefits. See Wu et al. “Arginine metabolism and nutrition in growth health, and disease”. Amino Acids (2009) 37:153-168. AND Wu, G. “Functional Amino Acids in Growth, Reproduction, and Health” Adv. Nutr. (2010) 1: 31-37. A similar approach was applied to increasing the Arg content of Carbohydrate binding module 20 protein. FIG. 18A analyzes the amino acid content (by weight) of engineered proteins generated by replacing all non-Arg amino acids that occur at amino acid positions identified using different Arg likelihood thresholds from 0 to 1. Specifically, the weight fraction of Arg, BCAAs, and EAAs in SEQ ID NO: 6 are shown. In the top panel, the likelihood threshold for making amino acid replacements is presented on the X-axis. So the value 0.6 on the X-axis, for example, represents an engineered protein sequence created by identifying every amino acid position in SEQ ID NO: 6 having an Arg-likelihood score of at least 0.6 and replacing all non-Arg amino acids appearing at one of those positions with an Arg amino acid. In the top panel, the fraction by weight of Arg, BCAA, and EAA in the protein following the making of any necessary Arg replacements is shown on the Y-axis. In the bottom panel the Y-axis indicates the total of number of Arg replacements made to a protein when every amino acid position that has a given Arg likelihood score on the X-axis is occupied by an Arg amino acid in the engineered protein. The top and bottom panels of FIG. 18B present a close-up view of the left end of the graphs (for Arg likelihood scores of 0 to 0.3) shown in FIG. 18A.

This analysis was repeated, but instead of assessing Arg likelihood to identify amino acid positions for mutation positive amino acid (R, K, H) likelihood and charged amino acid (R, K, H, D, E) likelihood were measured in the MSA data to identify amino acid positions, and then every non-Arg amino acid at identified positions was replaced with an Arg amino acid. The results are presented in FIG. 18C (positive amino acid likelihood) and FIG. 18D (charged amino acid likelihood). As expected, these two less stringent screens lead to replacement of more non-Arg amino acids with Arg at each likelihood cut off.

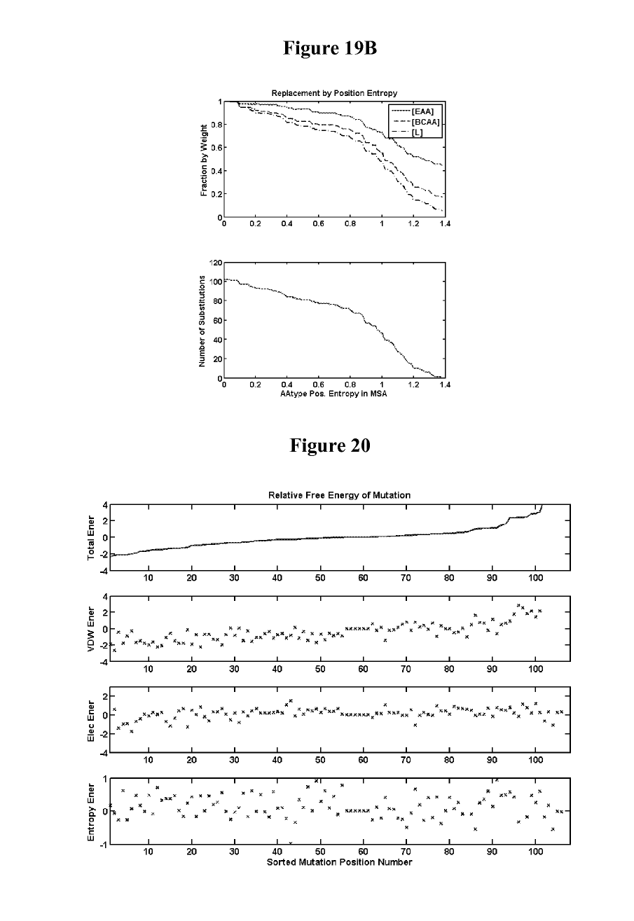

The Leu replacement analysis was repeated, using position entropy instead of raw amino acid likelihood to rank amino acid positions for substitution by Leu. Results obtained when position entropy was calculated using the frequency of each amino acid at each position are presented in FIG. 19A and when position entropy was calculated using the frequency of amino acid type at each position in FIG. 19B.

The free energy of folding (ΔΔG_{fold}) for all possible single amino acid mutations of non-Leu amino acids in SEQ ID NO: 6 to Leu, relative to the wild type free energy of folding in SEQ ID NO: 6 was also computed. For each amino acid substitution, the positions were rank ordered based on their predicted effect on folding stability. The results are shown in FIG. 20.

The following list presents the percentage of the mutations to Leucine, Valine, Isoleucine, or Arginine that are predicted to have a favorable effect on the relative free energy of folding, that are driven by van der Waals (vdw), electrostatic (elec), and entropic free energy changes:

Leu: 75.0% vdw, 11.3% elec, 13.7% entropic free energy;
Val: 45.1% vdw, 21.0% elec, 33.9% entropic free energy;
Ile: 72.0% vdw, 12.0% elec, 16.0% entropic free energy; and
Arg: 86.3% vdw, 10.0% elec, 3.7% entropic free energy.

It is interesting to note that there is a significant degree of variability in the major free energy component for any given amino acid mutation, as well as between mutations to different amino acids. Most mutations are favorable due to improved vdw folding energies, but a nontrivial number of mutations are predicted to be favorable due to favorable changes in electrostatic and entropic free energies, especially in the case of Valine. This suggests that there are more strategies available when trying to optimize for higher valine concentrations by differentially affecting the relative free energy of folding components. In this case, the importance of the entropic free energy contributions to the overall relative free energy of folding is consistant with what is expected given the number of rotatable bonds present in each amino acid side chain (Leu=2, Val=1, Ile=2, Arg=4). Replacement of a highly flexible amino acid with a less flexible one results in a favorable relative change in the entropic free energy of folding.

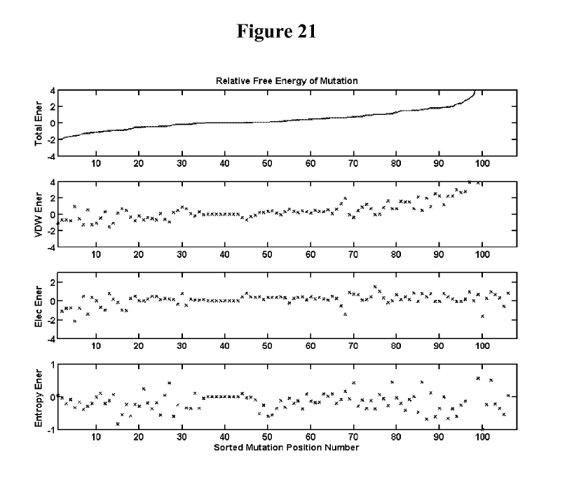

The free energy of folding (ΔΔG_{fold}) for all possible single amino acid mutations of non-Ile amino acids in SEQ ID NO: 6 to Ile, relative to the wild type free energy of folding in SEQ ID NO: 6 was also computed. For each amino acid substitution, we then rank ordered the positions based on their predicted effect on folding stability. The results are shown in FIG. 21.

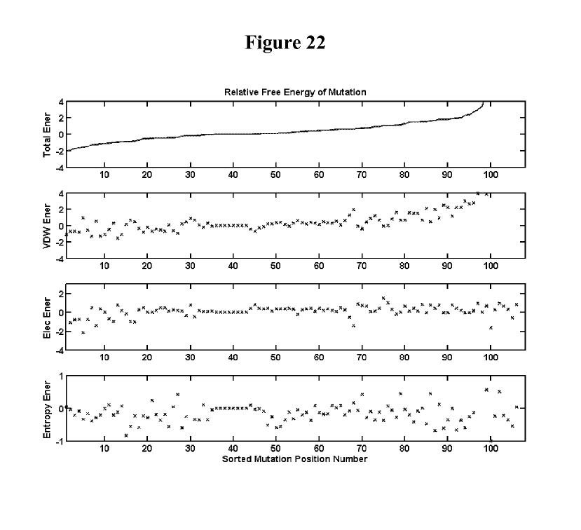

The free energy of folding (ΔΔG_{fold}) for all possible single amino acid mutations of non-Val amino acids in SEQ ID NO: 6 to Val, relative to the wild type free energy of folding in SEQ ID NO: 6 was also computed. For each amino acid substitution, we then rank ordered the positions based on their predicted effect on folding stability. The results are shown in FIG. 22.

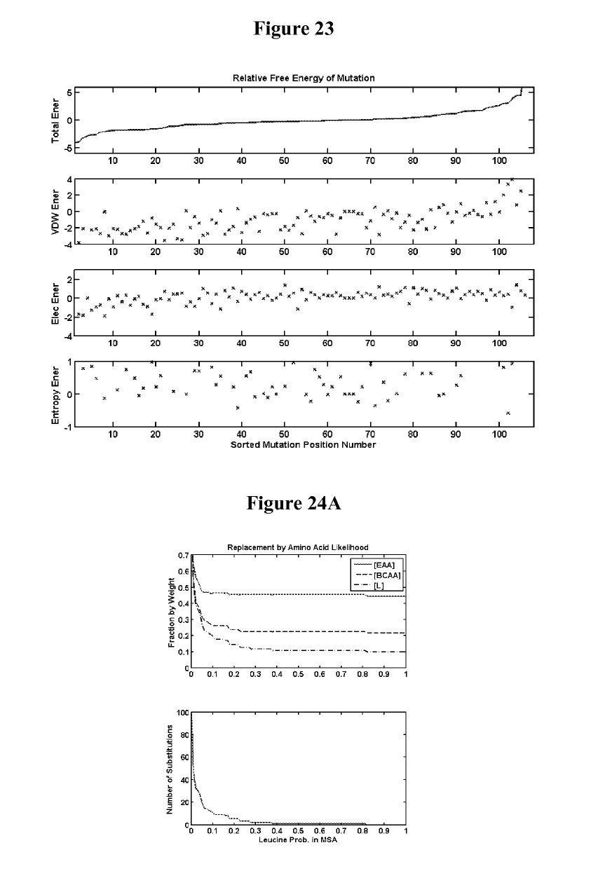

The free energy of folding (ΔΔG_{fold}) for all possible single amino acid mutations of non-Arg amino acids in SEQ ID NO: 6 to Arg, relative to the wild type free energy of folding in SEQ ID NO: 6 was also computed. For each amino acid substitution, we then rank ordered the positions based on their predicted effect on folding stability. The results are shown in FIG. 23.

For each amino acid in SEQ ID NO: 6, the loop ID (1=loop; 0=non-loop), Leu likelihood, BCAA likelihood, EAA likelihood, hydrophobic amino acid likelihood, amino acid position entropy, amino acid type position entropy, total free energy of folding (ΔΔG_{fold}) (for substitution by Leu), the van der Waals free energy change contribution to total free energy of folding (ΔΔGfold VDW) (for substitution by Leu), the electrostatic free energy change contribution to total free energy of folding (ΔΔGfold Elec) (for substitution by Leu), and the thermodynamic entropic free energy change contribution to total free energy of folding (ΔΔGfold Entropy) (for substitution by Leu) was calculated. The results are shown in Table 6A in Appendix D.

For each amino acid in SEQ ID NO: 6, the loop ID (1=loop; 0=non-loop), Ile likelihood, BCAA likelihood, EAA likelihood, hydrophobic amino acid likelihood, amino acid position entropy, amino acid type position entropy, total free energy of folding (ΔΔG_{fold}) (for substitution by Ile), the van der Waals free energy change contribution to total free energy of folding (ΔΔGfold VDW) (for substitution by Ile), the electrostatic free energy change contribution to total free energy of folding (ΔΔGfold Elec) (for substitution by Ile), and the thermodynamic entropic free energy change contribution to total free energy of folding (ΔΔGfold Entropy) (for substitution by Ile) was calculated. The results are shown in Table 6B in Appendix D.

For each amino acid in SEQ ID NO: 6, the loop ID (1=loop; 0=non-loop), Val likelihood, BCAA likelihood, EAA likelihood, hydrophobic amino acid likelihood, amino acid position entropy, amino acid type position entropy, total free energy of folding (ΔΔG_{fold}) (for substitution by Val), the van der Waals free energy change contribution to total free energy of folding (ΔΔGfold VDW) (for substitution by Val), the electrostatic free energy change contribution to total free energy of folding (ΔΔGfold Elec) (for substitution by Val), and the thermodynamic entropic free energy change contribution to total free energy of folding (ΔΔGfold Entropy) (for substitution by Val) was calculated. The results are shown in Table 6C in Appendix D.

For each amino acid in SEQ ID NO: 6, the loop ID (1=loop; 0=non-loop), Arg likelihood, positive AA likelihood, charged AA likelihood, amino acid position entropy, amino acid type position entropy, total free energy of folding (ΔΔG_{fold}) (for substitution by Arg), the van der Waals free energy change contribution to total free energy of folding (ΔΔGfold VDW) (for substitution by Arg), the electrostatic free energy change contribution to total free energy of folding (ΔΔGfold Elec) (for substitution by Arg), and the thermodynamic entropic free energy change contribution to total free energy of folding (ΔΔGfold Entropy) (for substitution by Arg) was calculated. The results are shown in Table 6D in Appendix D.

### Example 9

### Identification of Amino Acid Positions for Substitution in the A. niger Glucosidase Fibronectin Type III Domain Protein (SEQ ID NO: 7)

Glucosidase fibronectin type III domain from A. niger (SEQ ID NO: 7) contains 9.9% by weight Leu, 21.5% by weight branch chain amino acids, and 44.5% by weight essential amino acids.

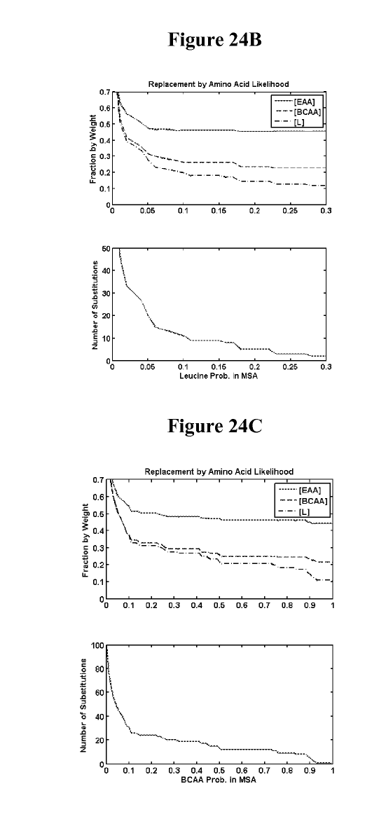

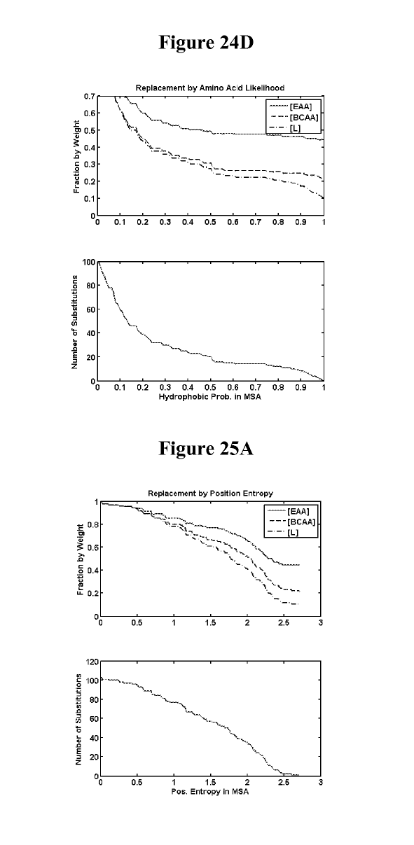

FIG. 24A analyzes the amino acid content (by weight) of engineered proteins generated by replacing all non-Leu amino acids that occur at amino acid positions identified using different Leu likelihood thresholds from 0 to 1. Specifically, the weight fraction of Leu, BCAAs, and EAAs in SEQ ID NO: 7 are shown. In the top panel, the likelihood threshold for making amino acid replacements is presented on the X-axis. So the value 0.6 on the X-axis, for example, represents an engineered protein sequence created by identifying every amino acid position in SEQ ID NO: 7 having a Leu-likelihood score of at least 0.6 and replacing all non-Leu amino acids appearing at one of those positions with a Leu amino acid. In the top panel, the fraction by weight of Leu, BCAA, and EAA in the protein following the making of any necessary Leu replacements is shown on the Y-axis. In the bottom panel the Y-axis indicates the total of number of Leu replacements made to a protein when every amino acid position that has a given Leu likelihood score on the X-axis is occupied by a Leu amino acid in the engineered protein. The top and bottom panels of FIG. 24B present a close-up view of the left end of the graphs (for Leu likelihood scores of 0 to 0.3) shown in FIG. 24A.

This analysis was repeated, but instead of assessing Leu likelihood to identify amino acid positions for mutation BCAA likelihood and hydrophobic amino acid likelihood was measured in the MSA data to identify amino acid positions, and every non-Leu amino acid at identified positions was replaced with a Leu amino acid. The results are presented in FIG. 24C (BCAA likelihood) and FIG. 24D (hydrophobic amino acid likelihood). As expected, these two less stringent screens lead to replacement of more non-Leu amino acids with Leu at each likelihood cut off.

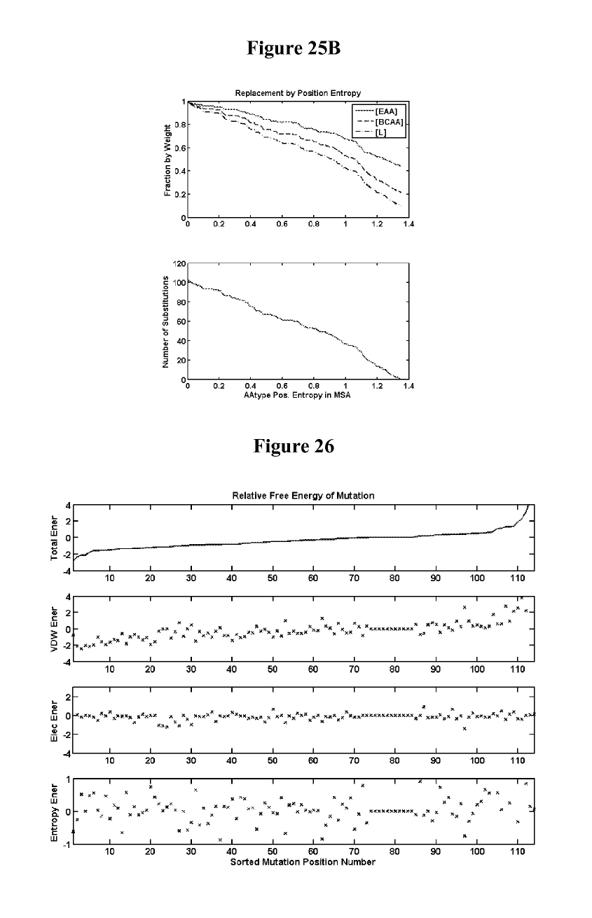

The same analysis was repeated, using position entropy instead of raw amino acid likelihood to rank amino acid positions for substitution by Leu. Results obtained when position entropy was calculated using the frequency of each amino acid at each position are presented in FIG. 25A and when position entropy was calculated using the frequency of amino acid type at each position in FIG. 25B.

The free energy of folding (ΔΔG_{fold}) for all possible single amino acid mutations of non-Leu amino acids in SEQ ID NO: 7 to Leu, relative to the wild type free energy of folding in SEQ ID NO: 7 was also calculated. For each amino acid substitution, the positions were rank ordered based on their predicted effect on folding stability. The results are shown in FIG. 26.

For each amino acid in SEQ ID NO: 7, the loop ID (1=loop; 0=non-loop), Leu likelihood, BCAA likelihood, EAA likelihood, hydrophobic amino acid likelihood, amino acid position entropy, amino acid type position entropy, total free energy of folding (ΔΔG_{fold}) (for substitution by Leu), the van der Waals free energy change contribution to total free energy of folding (ΔΔGfold VDW) (for substitution by Leu), the electrostatic free energy change contribution to total free energy of folding (ΔΔGfold Elec) (for substitution by Leu), and the thermodynamic entropic free energy change contribution to total free energy of folding (ΔΔGfold Entropy) (for substitution by Leu) was calculated. The results are shown in Table 7 in Appendix D.

### Example 10

### Identification of Amino Acid Positions for Substitution in the T. Reesei Hydrophobin I Protein (SEQ ID NO: 8)

Hydrophobin I protein from T. Reesei (SEQ ID NO: 8) contains 10.5% by weight Leu, 22.5% by weight branch chain amino acids, and 35.2% by weight essential amino acids.

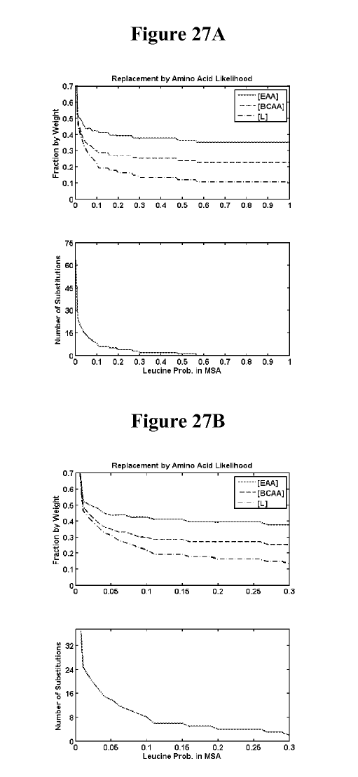

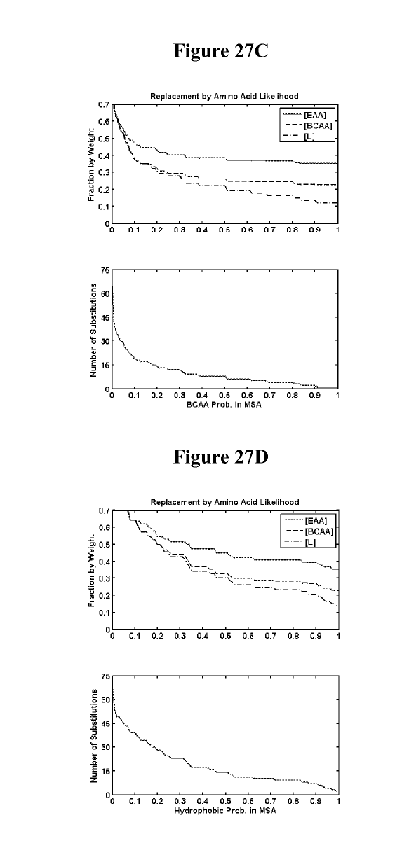

FIG. 27A analyzes the amino acid content (by weight) of engineered proteins generated by replacing all non-Leu amino acids that occur at amino acid positions identified using different Leu likelihood thresholds from 0 to 1. Specifically, the weight fraction of Leu, BCAAs, and EAAs in SEQ ID NO: 8 are shown. In the top panel, the likelihood threshold for making amino acid replacements is presented on the X-axis. So the value 0.6 on the X-axis, for example, represents an engineered protein sequence created by identifying every amino acid position in SEQ ID NO: 8 having a Leu-likelihood score of at least 0.6 and replacing all non-Leu amino acids appearing at one of those positions with a Leu amino acid. In the top panel, the fraction by weight of Leu, BCAA, and EAA in the protein following the making of any necessary Leu replacements is shown on the Y-axis. In the bottom panel the Y-axis indicates the total of number of Leu replacements made to a protein when every amino acid position that has a given Leu likelihood score on the X-axis is occupied by a Leu amino acid in the engineered protein. The top and bottom panels of FIG. 27B present a close-up view of the left end of the graphs (for Leu likelihood scores of 0 to 0.3) shown in FIG. 27A.

This analysis was repeated, but instead of assessing Leu likelihood to identify amino acid positions for mutation BCAA likelihood and hydrophobic amino acid likelihood was measured in the MSA data to identify amino acid positions, and then every non-Leu amino acid at identified positions was replaced with a Leu amino acid. The results are presented in FIG. 27C (BCAA likelihood) and FIG. 27D (hydrophobic amino acid likelihood). As expected, these two less stringent screens lead to replacement of more non-Leu amino acids with Leu at each likelihood cut off.

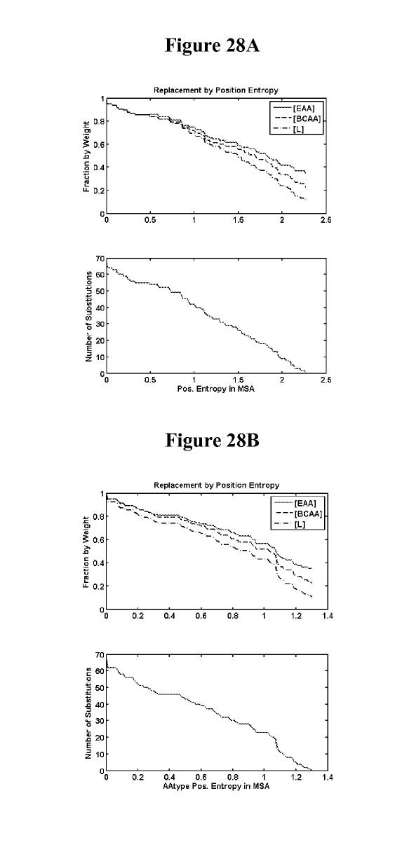

The same analysis was repeated, using position entropy instead of raw amino acid likelihood to rank amino acid positions for substitution by Leu. Results obtained when position entropy was calculated using the frequency of each amino acid at each position are presented in FIG. 28A and when position entropy was calculated using the frequency of amino acid type at each position in FIG. 28B.

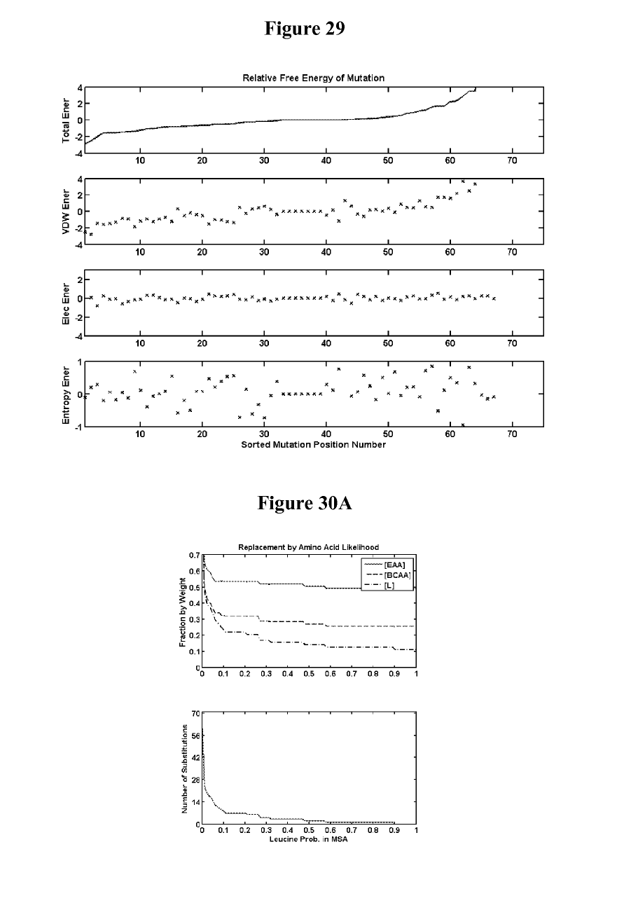

The free energy of folding (ΔΔG_{fold}) for all possible single amino acid mutations of non-Leu amino acids in SEQ ID NO: X to Leu, relative to the wild type free energy of folding in SEQ ID NO: 8 was also computed. For each amino acid substitution, we then rank ordered the positions based on their predicted effect on folding stability. The results are shown in FIG. 29.

For each amino acid in SEQ ID NO: 8, the loop ID (1=loop; 0=non-loop), Leu likelihood, BCAA likelihood, EAA likelihood, hydrophobic amino acid likelihood, amino acid position entropy, amino acid type position entropy, total free energy of folding (ΔΔG_{fold}) (for substitution by Leu), the van der Waals free energy change contribution to total free energy of folding (ΔΔGfold VDW) (for substitution by Leu), the electrostatic free energy change contribution to total free energy of folding (ΔΔGfold Elec) (for substitution by Leu), and the thermodynamic entropic free energy change contribution to total free energy of folding (ΔΔGfold Entropy) (for substitution by Leu) were calculated. The results are shown in Table 8 in Appendix D.

### Example 11

### Identification of Amino Acid Positions for Substitution in the T. Reesei Hydrophobin II Protein (SEQ ID NO: 9)

Hydrophobin II protein from T. Reesei (SEQ ID NO: 9) contains 11.0% by weight Leu, 25.6% by weight branch chain amino acids, and 49.2% by weight essential amino acids.

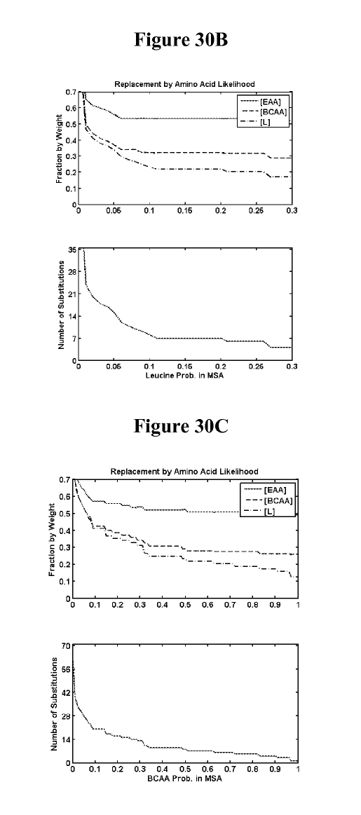

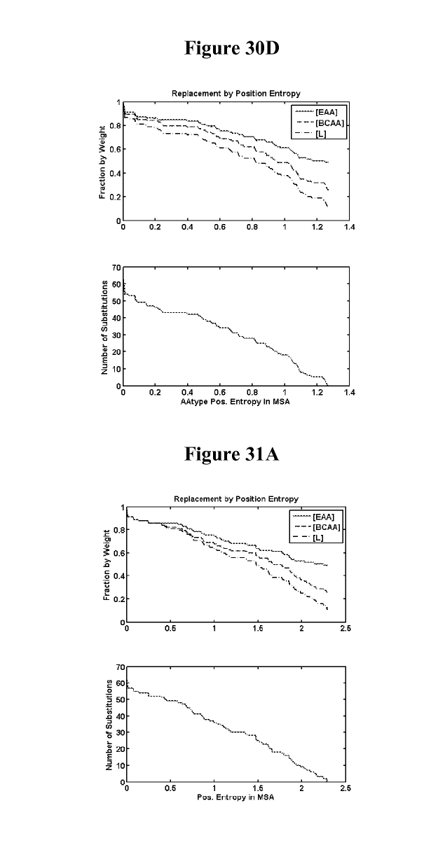

FIG. 30A analyzes the amino acid content (by weight) of engineered proteins generated by replacing all non-Leu amino acids that occur at amino acid positions identified using different Leu likelihood thresholds from 0 to 1. Specifically, the weight fraction of Leu, BCAAs, and EAAs in SEQ ID NO: 9 are shown. In the top panel, the likelihood threshold for making amino acid replacements is presented on the X-axis. So the value 0.6 on the X-axis, for example, represents an engineered protein sequence created by identifying every amino acid position in SEQ ID NO: 9 having a Leu-likelihood score of at least 0.6 and replacing all non-Leu amino acids appearing at one of those positions with a Leu amino acid. In the top panel, the fraction by weight of Leu, BCAA, and EAA in the protein following the making of any necessary Leu replacements is shown on the Y-axis. In the bottom panel the Y-axis indicates the total of number of Leu replacements made to a protein when every amino acid position that has a given Leu likelihood score on the X-axis is occupied by a Leu amino acid in the engineered protein. The top and bottom panels of FIG. 30B present a close-up view of the left end of the graphs (for Leu likelihood scores of 0 to 0.3) shown in FIG. 30A.

This analysis was repeated, but instead of assessing Leu likelihood to identify amino acid positions for mutation BCAA likelihood and hydrophobic amino acid likelihood were measured in the MSA data to identify amino acid positions, and then every non-Leu amino acid at identified positions was replaced with a Leu amino acid. The results are presented in FIG. 30C (BCAA likelihood) and FIG. 30D (position entropy). As expected, these two less stringent screens lead to replacement of more non-Leu amino acids with Leu at each likelihood cut off.

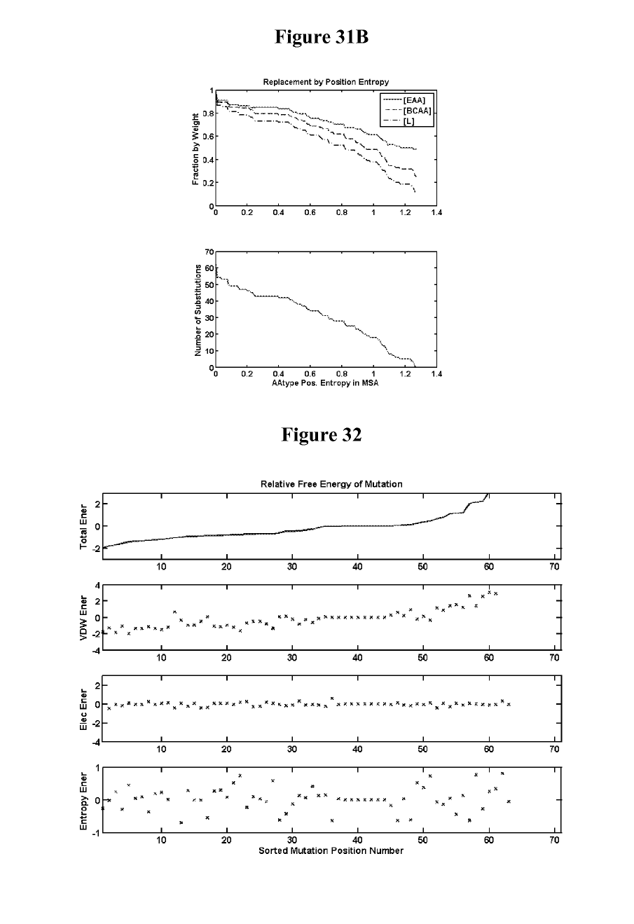

The same analysis was repeated, using position entropy instead of raw amino acid likelihood to rank amino acid positions for substitution by Leu. Results obtained when position entropy was calculated using the frequency of each amino acid at each position are presented in FIG. 31A and when position entropy was calculated using the frequency of amino acid type at each position in FIG. 31B.

The free energy of folding (ΔΔG_{fold}) for all possible single amino acid mutations of non-Leu amino acids in SEQ ID NO: 9 to Leu, relative to the wild type free energy of folding in SEQ ID NO: 9 was also computed. For each amino acid substitution, the positions were rank ordered based on their predicted effect on folding stability. The results are shown in FIG. 32.

For each amino acid in SEQ ID NO: 9, the loop ID (1=loop; 0=non-loop), Leu likelihood, BCAA likelihood, EAA likelihood, hydrophobic amino acid likelihood, amino acid position entropy, amino acid type position entropy, total free energy of folding (ΔΔG_{fold}) (for substitution by Leu), the van der Waals free energy change contribution to total free energy of folding (ΔΔGfold VDW) (for substitution by Leu), the electrostatic free energy change contribution to total free energy of folding (ΔΔGfold Elec) (for substitution by Leu), and the thermodynamic entropic free energy change contribution to total free energy of folding (ΔΔGfold Entropy) (for substitution by Leu) was calculated. The results are shown in Table 9 in Appendix D.

### Example 12

### Amino Acid Selection Algorithm

The analyses of position amino acid likelihood, position entropy, mutation effect on relative folding free energy, and secondary structure type can be combined to screen for and identify amino acids in reference secreted proteins to mutate to more nutritive amino acid types, such as Leu. In effect, the selection and ranking procedure is a multiobjective optimization problem. Multiple different objectives can be attained by designing engineered proteins using these factors: high amino acid likelihood (AALike), high amino acid type likelihood (AATLike), high position entropy (Sₚₒₛ), high amino acid type position entropy (S_{AATpos}), low relative free energy of folding (ΔΔG_{fold}), and secondary structure identity (LoopID). It is also possible to select positions that maximize all or a subset of objectives simultaneously. To this end, aggregate objective functions that score each mutation based on their individual objective scores were constructed. When ranking possible mutation sites for a given protein, in order to directly compare two objective functions and/or add them together with controlled weighting, the distribution of values was mapped onto the range [0-1] by shifting the minimum value to 0 and normalizing all values by the maximum value. Note that in the case of ΔΔG_{fold}, the minimum value was mapped onto 1 (as negative values are favorable) and the maximum value defined to be 1, as a cutoff to limit consideration to positions with ΔΔG_{fold}<1. In addition to all the single objective functions, eleven exemplary aggregate objective functions are:

$$\begin{matrix}\frac{1}{2} AAlike + \frac{1}{2} ΔΔ {G}_{fold} & ( 1 ) \\ \frac{1}{2} AATlike + \frac{1}{2} ΔΔ {G}_{fold} & ( 2 ) \\ \frac{1}{3} AAlike + \frac{1}{3} AATlike + \frac{1}{3} ΔΔ {G}_{fold} & ( 3 ) \\ \frac{1}{2} {S}_{pos} + \frac{1}{2} ΔΔ {G}_{fold} & ( 4 ) \\ \frac{1}{2} {S}_{AATpos} + \frac{1}{2} ΔΔ {G}_{fold} & ( 5 ) \\ \frac{1}{2} LoopID + \frac{1}{2} ΔΔ {G}_{fold} & ( 6 ) \\ \frac{1}{3} AAlike + \frac{1}{3} ΔΔ {G}_{fold} + \frac{1}{3} LoopID & ( 7 ) \\ \frac{1}{4} AAlike + \frac{1}{4} AATlike + \frac{1}{4} ΔΔ {G}_{fold} + \frac{1}{4} LoopID & ( 8 ) \\ \frac{1}{3} AATlike + \frac{1}{3} ΔΔ {G}_{fold} + \frac{1}{3} LoopID & ( 9 ) \\ \frac{1}{3} {S}_{pos} + \frac{1}{3} ΔΔ {G}_{fold} + \frac{1}{3} LoopID & ( 10 ) \\ \frac{1}{3} {S}_{AATpos} + \frac{1}{3} ΔΔ {G}_{fold} + \frac{1}{3} LoopID & ( 11 )\end{matrix}$$

The first six functions select for positions that have favorable effects on folding stability and either high amino acid likelihoods [(1), (2), and (3)], high position entropies [(4) and (5)], or are structurally plastic loop positions (6). The seventh through eleventh objective functions select for loop positions with favorable, relative folding energies and either high amino acid likelihoods [(7), (8), and (9)] or position entropies [(10) and (11)]. In order to enrich a secreted protein for a particular amino acid, the top set of positions that rank highly according to the desired objective function 1-11 are selected and those amino acids mutated to generate an engineered protein.

Below are example sequences for cellulose binding domain 1 (CBD1) (SEQ ID NO: 5) mutations ranking all 36 positions according to objective function 3 using Leucine as the target amino acid and branched chain amino acids as the amino acid type:

$$\frac{1}{3} Leulike + \frac{1}{3} BCAAlike + \frac{1}{3} ΔΔ {G}_{fold} .$$

Remapping all values onto a common range, [0, 1], as described above, and ranking according to the aggregate objective function gives the rank ordered list (excluding all those positions with ΔΔG_{fold}>1 kcal/mol or cysteine residues involved in intramolecular disulfide bonds) presented in Table 10.

TABLE 10

| Position | Score 3 |
|---|---|
| 36 | 0.773 |
| 28 | 0.443 |
| 4 | 0.421 |
| 6 | 0.333 |
| 20 | 0.268 |
| 30 | 0.223 |
| 27 | 0.221 |
| 26 | 0.202 |
| 29 | 0.194 |
| 24 | 0.192 |
| 18 | 0.186 |
| 7 | 0.176 |
| 1 | 0.174 |
| 31 | 0.173 |
| 32 | 0.168 |
| 11 | 0.161 |
| 5 | 0.147 |
| 17 | 0.118 |
| 3 | 0.118 |
| 16 | 0.117 |
| 14 | 0.107 |
| 23 | 0.107 |
| 33 | 0.102 |
| 9 | 0.101 |
| 21 | 0.101 |
| 34 | 0.042 |

Note that the top hit, position 36, is already a Leucine, so no mutation need be performed at this site. Thus, in order to increase the Leucine concentration to ˜11% (the native sequence is approximately 3% Leucine) 3 mutations are required, and this analysis suggests positions E28, A4, and G6. The resulting engineered protein has the sequence of SEQ ID NO: 10.

To increase the Leucine concentration to ˜22%, 7 mutations are required, and this analysis suggests positions E28, A4, G6, V20, A30, Y27, and T26. The resulting engineered protein has the sequence of SEQ ID NO: 11.

To increase the Leucine concentration to ˜31%, 10 mutations are required, and this analysis suggests positions E28, A4, G6, V20, A30, Y27, T26, N29, T24, and T18. The resulting engineered protein has the sequence of SEQ ID NO: 12.

Finally, to increase the Leucine concentration to ˜42%, 14 mutations are required, and this analysis suggests positions E28, A4, G6, V20, A30, Y27, T26, N29, T24, T18, Q7, A1, Y31, and Y32. The resulting engineered protein has the sequence of SEQ ID NO: 13.

Tables 11, 12, and 13 show the equivalent rank ordered lists found when using Leucine as the target amino acid, branched chain amino acids as the amino acid type, and objective functions 1 through 11, as defined above. To increase the Leucine concentration to ˜11%, the top 3 positions from the position lists in Tables 11, 12, and 13, that are not already Leucine in CBD1 (SEQ ID NO: 5), may be selected. Thus, to select for positions with favorable relative free energies of folding and high amino acid likelihoods, using the objective function 1, 2, or 3 rankings is appropriate. To select for positions with favorable relative free energies of folding and high position entropies or loop positions, using objective function 4, 5, or 6 rankings would be appropriate. To select for positions with favorable, relative folding energies and either high amino acid likelihoods or position entropies, using objective function 7, 8, or 9 rankings or objective function 10 or 11 rankings, respectively, would be appropriate.

TABLE 11
Additional Objective Function Rankings (1-4) for CBD1

| Posi- | Score | Posi- | Score | Posi- | Score | Posi- | Score |
|---|---|---|---|---|---|---|---|
| tion | 1 | tion | 2 | tion | 3 | tion | 4 |
| 36 | 0.660 | 36 | 0.660 | 36 | 0.773 | 4 | 0.866 |
| 6 | 0.500 | 4 | 0.513 | 28 | 0.443 | 28 | 0.788 |
| 4 | 0.485 | 28 | 0.512 | 4 | 0.421 | 6 | 0.712 |
| 28 | 0.456 | 6 | 0.500 | 6 | 0.333 | 30 | 0.711 |
| 30 | 0.331 | 20 | 0.402 | 20 | 0.268 | 18 | 0.654 |
| 29 | 0.291 | 30 | 0.335 | 30 | 0.223 | 27 | 0.647 |
| 7 | 0.265 | 27 | 0.319 | 27 | 0.221 | 20 | 0.637 |
| 20 | 0.261 | 26 | 0.301 | 26 | 0.202 | 26 | 0.620 |
| 31 | 0.259 | 29 | 0.291 | 29 | 0.194 | 3 | 0.588 |
| 32 | 0.249 | 24 | 0.287 | 24 | 0.192 | 11 | 0.553 |
| 24 | 0.246 | 18 | 0.269 | 18 | 0.186 | 24 | 0.552 |
| 5 | 0.220 | 7 | 0.265 | 7 | 0.176 | 16 | 0.521 |
| 18 | 0.215 | 1 | 0.262 | 1 | 0.174 | 23 | 0.487 |
| 27 | 0.196 | 31 | 0.260 | 31 | 0.173 | 31 | 0.475 |
| 26 | 0.194 | 32 | 0.250 | 32 | 0.168 | 21 | 0.447 |
| 17 | 0.176 | 11 | 0.225 | 11 | 0.161 | 14 | 0.415 |
| 16 | 0.174 | 5 | 0.221 | 5 | 0.147 | 29 | 0.407 |
| 3 | 0.172 | 17 | 0.177 | 17 | 0.118 | 5 | 0.400 |
| 14 | 0.158 | 3 | 0.174 | 3 | 0.118 | 1 | 0.370 |
| 33 | 0.152 | 16 | 0.173 | 16 | 0.117 | 36 | 0.361 |
| 9 | 0.152 | 14 | 0.160 | 14 | 0.107 | 33 | 0.328 |
| 21 | 0.148 | 33 | 0.152 | 23 | 0.107 | 17 | 0.322 |
| 23 | 0.132 | 9 | 0.152 | 33 | 0.102 | 32 | 0.316 |
| 11 | 0.119 | 21 | 0.150 | 9 | 0.101 | 7 | 0.291 |
| 1 | 0.113 | 23 | 0.114 | 21 | 0.101 | 9 | 0.159 |
| 34 | 0.062 | 34 | 0.061 | 34 | 0.042 | 12 | 0.141 |

TABLE 12
Additional Objective Function Rankings (5-8) for CBD1

| Posi- | Score | Posi- | Score | Posi- | Score | Posi- | Score |
|---|---|---|---|---|---|---|---|
| tion | 5 | tion | 6 | tion | 7 | tion | 8 |
| 30 | 0.803 | 6 | 1.000 | 36 | 0.773 | 36 | 0.830 |
| 6 | 0.797 | 4 | 0.866 | 6 | 0.667 | 28 | 0.582 |
| 4 | 0.784 | 30 | 0.831 | 4 | 0.657 | 4 | 0.566 |
| 3 | 0.669 | 28 | 0.804 | 28 | 0.637 | 6 | 0.500 |
| 18 | 0.651 | 7 | 0.765 | 30 | 0.554 | 20 | 0.451 |
| 28 | 0.646 | 20 | 0.761 | 7 | 0.510 | 30 | 0.417 |
| 20 | 0.618 | 31 | 0.759 | 20 | 0.507 | 27 | 0.416 |
| 16 | 0.605 | 32 | 0.748 | 31 | 0.506 | 26 | 0.401 |
| 26 | 0.578 | 24 | 0.744 | 32 | 0.500 | 24 | 0.394 |
| 27 | 0.565 | 5 | 0.720 | 24 | 0.497 | 18 | 0.390 |
| 24 | 0.560 | 18 | 0.706 | 5 | 0.480 | 7 | 0.382 |
| 21 | 0.502 | 26 | 0.691 | 18 | 0.477 | 1 | 0.381 |
| 11 | 0.483 | 27 | 0.684 | 27 | 0.464 | 31 | 0.380 |
| 29 | 0.385 | 3 | 0.669 | 26 | 0.462 | 32 | 0.376 |
| 23 | 0.376 | 36 | 0.660 | 3 | 0.448 | 5 | 0.361 |
| 33 | 0.365 | 33 | 0.652 | 33 | 0.435 | 3 | 0.338 |
| 1 | 0.330 | 21 | 0.647 | 21 | 0.432 | 23 | 0.330 |
| 17 | 0.321 | 1 | 0.613 | 23 | 0.421 | 33 | 0.326 |
| 31 | 0.311 | 23 | 0.586 | 1 | 0.409 | 21 | 0.325 |
| 7 | 0.308 | 34 | 0.560 | 34 | 0.375 | 34 | 0.282 |
| 32 | 0.283 | 22 | 0.436 | 22 | 0.292 | 22 | 0.220 |
| 14 | 0.263 | 29 | 0.291 | 29 | 0.194 | 29 | 0.146 |
| 5 | 0.237 | 17 | 0.176 | 17 | 0.117 | 11 | 0.120 |
| 12 | 0.200 | 16 | 0.171 | 16 | 0.116 | 17 | 0.089 |
| 9 | 0.164 | 14 | 0.158 | 14 | 0.105 | 16 | 0.087 |
| 36 | 0.161 | 9 | 0.152 | 9 | 0.101 | 14 | 0.080 |

TABLE 13
Additional Objective Function Rankings (9-11) for CBD1

| Position | Score 9 | Position | Score 10 | Position | Score 11 |
|---|---|---|---|---|---|
| 36 | 0.773 | 4 | 0.911 | 30 | 0.868 |
| 4 | 0.675 | 28 | 0.859 | 6 | 0.865 |
| 28 | 0.675 | 6 | 0.808 | 4 | 0.856 |
| 6 | 0.667 | 30 | 0.807 | 3 | 0.779 |
| 20 | 0.601 | 18 | 0.770 | 18 | 0.767 |
| 30 | 0.556 | 27 | 0.765 | 28 | 0.764 |
| 27 | 0.546 | 20 | 0.758 | 20 | 0.746 |
| 26 | 0.534 | 26 | 0.746 | 26 | 0.718 |
| 24 | 0.525 | 3 | 0.725 | 27 | 0.710 |
| 18 | 0.513 | 24 | 0.701 | 24 | 0.706 |
| 7 | 0.510 | 23 | 0.658 | 21 | 0.668 |
| 1 | 0.508 | 31 | 0.650 | 23 | 0.584 |
| 31 | 0.506 | 21 | 0.631 | 33 | 0.576 |
| 32 | 0.500 | 5 | 0.600 | 1 | 0.553 |
| 5 | 0.481 | 1 | 0.580 | 31 | 0.541 |
| 3 | 0.449 | 36 | 0.574 | 7 | 0.538 |
| 33 | 0.435 | 33 | 0.552 | 32 | 0.522 |
| 21 | 0.433 | 32 | 0.544 | 5 | 0.491 |
| 23 | 0.410 | 7 | 0.527 | 36 | 0.441 |
| 34 | 0.374 | 34 | 0.417 | 16 | 0.403 |
| 22 | 0.291 | 22 | 0.393 | 34 | 0.402 |
| 29 | 0.194 | 11 | 0.369 | 22 | 0.328 |
| 11 | 0.150 | 16 | 0.348 | 11 | 0.322 |
| 17 | 0.118 | 14 | 0.277 | 29 | 0.257 |
| 16 | 0.115 | 29 | 0.271 | 17 | 0.214 |
| 14 | 0.107 | 17 | 0.215 | 14 | 0.175 |

### Example 13

### Selection and Design of Engineered Secreted Protein from Bacillus subtilis

To demonstrate the engineering of secreted polypeptides for enriched amino acid content, we chose a microorganism known to secrete protein at high levels Bacillus subtilis. SEQID-45001 was identified a major secreted protein in Bacillus subtilis. Using sequence conservation and crystal structure data for SEQID-45001, we identified contiguous regions within each protein that were predicted to be tolerant to mutations without negatively affecting the structural stability of the protein and/or the ability of the host organism to secrete the protein.

We analyzed the secondary structure of SEQID-45001 reported in the structural protein databank entry 1UA7. We identified 19 loop regions within the sequence of the protein that are not part of an α-helix or β-sheet. These loop regions are defined by the following amino acid residues: 73-76, 130-133, 147-152, 157-161, 189-192, 222-227, 239-244, 283-286, 291-298, 305-308, 318-323, 336-340, 356-360, 365-368, 387-392, 417-421, 428-432, 437-442, and 464-466. Loop regions less than 4 amino acids in length were not considered for mutation.

Conservation of sequence over evolutionary space was also considered for identifying positions amenable for engineering while maintaining structural stability and secretion competency. Positions that are less conserved within a family of homologous sequences are inherently variable and likely more amenable to mutation without affecting activity, which is intrinsically tied to structure. To find positions that are less conserved, we downloaded the alignment of the pfam00128 from the NCBI Conserved Domain Database, which contains 31 protein sequences including the SEQID-45001 catalytic domain (Marchler-Bauer A., Zheng C., Chitsaz F., Derbyshire M. K., Geer L. Y., Geer R. C., Gonzales N. R., Gwadz M., Hurwitz D. I., Lanczycki C. J., Lu F., Lu S., Marchler G. H., Song J. S., Thanki N., Yamashita R. A., Zhang D., and S. H. Bryant. Nucleic Acids Res. (2013) 41:D348-52). We also performed a PSI-BLAST search of the NCBI protein reference sequence database (Pruitt K. D., Tatusova T., and D. R. Maglott. Nucleic Acids Res. (2005) 33:D501-504) using SEQID-45001 and obtained 500 sequences homologous to SEQID-45001. In both cases, a single iteration was performed using the BLOSUM62 position specific scoring matrix, a gap penalty of −11, a gap extension penalty of −1, and an alignment inclusion e-value cutoff of 0.005 (Altschul S. F., Nucleic Acids Res. (1997) 25:3389-3402). All protein sequence alignments were used to generate position-specific scoring matrices (PSSM) specific to each query sequence as part of the PSI-BLAST search. From the PSSMs, we identified regions hypothesized to be tolerant to mutation by counting the number of different amino acids associated with a positive PSSM score at each position within each loop as well as the sum and average of the PSSM scores for essential amino acid substitutions at each position Furthermore, from the multiple sequence alignments obtained from each PSI-BLAST search, we calculated the amino acid entropy at each position, as defined by

$${S}_{j} = - {\sum }_{i ∈ AA} {p}_{i} ln {p}_{i}$$

where Sⱼ is the entropy at position j and pⱼ is the probability of observing amino acid i at position j.

Using these measures of mutation tolerance, we identified four loop regions expected to be tolerant to mutations into essential amino acids. To enrich the identified regions in essential amino acids we used a combinatorial codon library where any selected position could be either a F, I, L, V, or M (denoted Z) or a R, K, T, I, or M (denoted X). In each of the loop regions selected for mutation into an essential amino acid, each variable position was assigned as a Z or X depending upon its relative tolerance of hydrophobic residues (based upon their respective PSSM values). Positions that were tolerant of hydrophobic residues were assigned as Z and genetically encoded using the codon NTN. Positions more tolerant of hydrophilic residues were assigned as an X and genetically encoded using the codon ANR. We note that in one of the identified variable regions of SEQID-45001 (147-153), a glycine residue was inserted into the center of the loop in an attempt to enhance the conformational flexibility of this region. For SEQID-45001 the sequences of the identified regions are summarized in the following table:

| Start residue # | original | degenerate |
|---|---|---|
| 148 | YAAI | XXGXX |
| | (SEQ ID NO: 22139) | |
| 240 | NTSA | ZXXZ |
| | (SEQ ID NO: 22140) | |
| 291 | SHYASD | XZYXXZ |
| | (SEQ ID NO: 22141) | |
| 389 | QPEE | XPZZ |
| | (SEQ ID NO: 22142) | |

X = NTN, codes for F, L, I, M, V
Z = ANR, codes for I, M, T, K, R

### Library Design and Construction

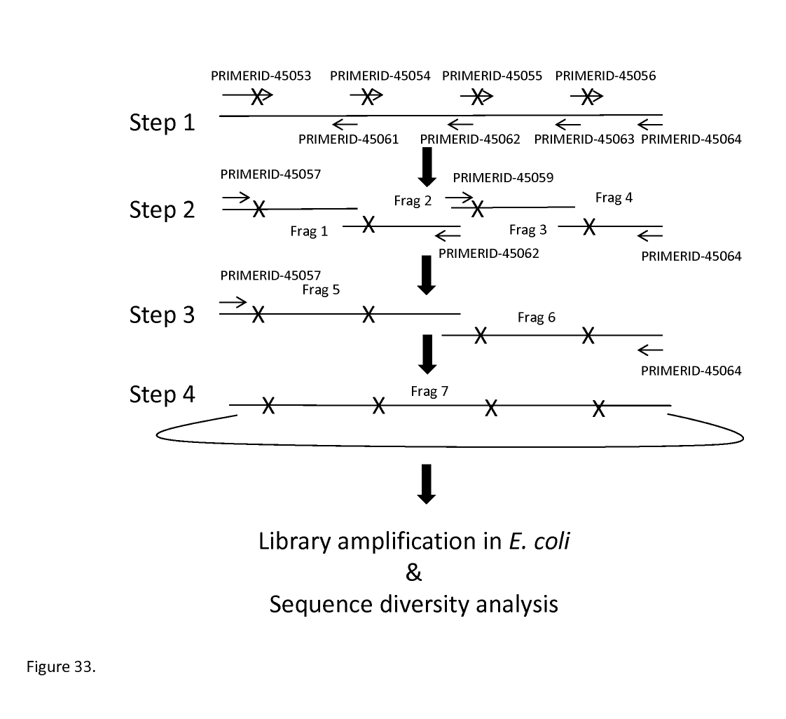

Based on identification of variable regions, we designed primers that can amplify each variable region as explained in FIG. 33. For example if there are four variable regions, we need four pair of primers to generate four variable fragments. In step 1 we used pES1205 as the template which contains SEQID-45001 fused with N-terminal AmyQ signal peptide and downstream of pGrac promoter. pES1205 is a derivative of the vector, pHT43 (MoBiTec), containing a 1905-bp DNA fragment encoding the amyE gene from B. subtilis (minus the initial 93-bp encoding the AmyE signal peptide) plus a C-terminal 1×FLAG tag. The amyE::1×FL:AG sequence is cloned, in-frame with the SamyQ sequence encoded on pHT43. For fragment 1,2,3,4, the forward PRIMERID-45053, PRIMERID-45054, PRIMERID-45055, and PRIMERID-45056 contain 25 bases of constant sequence before the variable region followed by degenerate sequences to represent the variable region and 25 bases of constant sequence downstream of the variable region. For fragment 1, 2, 3, the reverse primers PRIMERID-45061, PRIMERID-45062, and PRIMERID-45063 contain 25 bases of reverse complementary sequence upstream of next variable region respectively. For fragment 4, the reverse primer PRIMERID-45064 contains 25 bases of reverse complementary sequence at an arbitrary distance from variable region 4. Four separate PCR amplifications were run using Phusion DNA polymerase (New England Biolabs, Beverly, Mass.) and reaction parameters recommended by the manufacturer. As separate reactions, four wild type fragments, WT-frag-1, WT-frag-2, WT-frag-3 and WT-frag-4 were generated using PES1205 as template and primer pairs PRIMERID-45057 & PRIMERID-45061, PRIMERID-45058 & PRIMERID-45062, PRIMERID-45059 & PRIMERID-45063, and PRIMERID-45060 & PRIMERID-45064, respectively. All PCR fragments were gel purified. In step 2, two separate PCR reactions were set. The first PCR reaction contain fragment 1 and 2 in equimolar ratio as template and PRIMERID-45057 and PRIMERID-45062 as primers. The second PCR reaction contain fragment 3 and 4 in equimolar ratio and PRIMERID-45059 and PRIMERID-45064 as primers. In both the reactions, respective wild type fragments were added in a molar ratio of library members present in each variable fragments. Fragment 5 and 6 are gel purified and used as templates in equimolar ratio in step 3. The primers used in the PCR reaction include PRIMERID-45057 and PRIMERID-45064. The vector PCR product was generated using pES1205 and primer pairs, PRIMERID-45065 and PRIMERID-45066. Both fragment 7 and vector PCR product were gel purified and cloned together using the Gibson Assembly Master Mix (New England Biolabs, Beverly, Mass.) and transformed into the cloning host E. coli Turbo (New England Biolabs) according to manufacturer's instructions. 50 colonies were sequenced to determine the diversity of the library. The colonies on the agar plate were then suspended in LB media and harvested for plasmid purification. In a similar fashion, we generated 9 specific variants of SEQID-45001 which were altered with 9 specific amino acids, F, L, I, M, V, T, K, R, W at every variable position identified in the mutant design. Specific variant primers are denoted by the single letter amino acid abbreviation in the name. All primers are listed in table PRIMERID below.

| TABLE PRIMERID | Primer sequence (SEQ ID NOS 45053-45102, respectively, in order of appearance) |
|---|---|
| PRIMERID-45053 | GGTCATCAATCATACCACCAGTGATNTNNTNGGCNTNNTNTCCAATGAGGTTAAGAGTATTCCAAACTGG |
| PRIMERID-45054 | CAGTCAATTTTGGCCGAATATCACAANRNTNNTNANRGAGTTCCAATACGGAGAAATCCTGC |
| PRIMERID-45055 | TCGTAATCTGGGCGTGTCGAATATCNTNANRTATNTNNTNANRGTGTCTGCGGACAAGCTAGTGAC |
| PRIMERID-45056 | GATTTCACAATGTGATGGCTGGANTNCCTANRANRCTCTCGAACCCGAATGGAAAC |
| PRIMERID-45057 | GGTCATCAATCATACCACCAGTG |
| PRIMERID-45058 | CAGTCAATTTTGGCCGAATATCAC |
| PRIMERID-45059 | TCGTAATCTGGGCGTGTCG |
| PRIMERID-45060 | GATTTCACAATGTGATGGCTGG |
| PRIMERID-45061 | TGTGATATTCGGCCAAAATTGACTG |
| PRIMERID-45062 | GATATTCGACACGCCCAGATTACG |
| PRIMERID-45063 | TCCAGCCATCACATTGTGAAATC |
| PRIMERID-45064 | ATCTGCACGCAAGGTAATCGTCAG |
| PRIMERID-45065 | CTGACGATTACCTTGCGTGCAG |
| PRIMERID-45066 | CACTGGTGGTATGATTGATGACC |
| PRIMERID-45067 | GGTCATCAATCATACCACCAGTGATCTTCTGGGCCTTCTGTCCAATGAGGTTAAGAGTATTCCAAACTGG |
| PRIMERID-45068 | GGTCATCAATCATACCACCAGTGATATTATCGGCATTATCTCCAATGAGGTTAAGAGTATTCCAAACTGG |
| PRIMERID-45069 | GGTCATCAATCATACCACCAGTGATGTTGTGGGCGTTGTGTCCAATGAGGTTAAGAGTATTCCAAACTGG |
| PRIMERID-45070 | GGTCATCAATCATACCACCAGTGATTTTTTCGGCTTTTTCTCCAATGAGGTTAAGAGTATTCCAAACTGG |
| PRIMERID-45071 | GGTCATCAATCATACCACCAGTGATTGGTGGGGATGGTGGTCCAATGAGGTTAAGAGTATTCCAAACTGG |
| PRIMERID-45072 | GGTCATCAATCATACCACCAGTGATATGATGGGCATGATGTCCAATGAGGTTAAGAGTATTCCAAACTGG |
| PRIMERID-45073 | GGTCATCAATCATACCACCAGTGATACAACGGGCACAACGTCCAATGAGGTTAAGAGTATTCCAAACTGG |
| PRIMERID-45074 | GGTCATCAATCATACCACCAGTGATTATAAGAAAGGCAAGAAAAATGAGGTTAAGAGTATTCCAAACTGG |
| PRIMERID-45075 | GGTCATCAATCATACCACCAGTGATTATCATCATGGCCATCACAATGAGGTTAAGAGTATTCCAAACTGG |
| PRIMERID-45076 | CAGTCAATTTTGGCCGAATATCACAAAGCTTCTGGGCGAGTTCCAATACGGAGAAATCCTGC |
| PRIMERID-45077 | CAGTCAATTTTGGCCGAATATCACAAAGATTATCGGCGAGTTCCAATACGGAGAAATCCTGC |
| PRIMERID-45078 | CAGTCAATTTTGGCCGAATATCACAAAGGTTGTGGGCGAGTTCCAATACGGAGAAATCCTGC |
| PRIMERID-45079 | CAGTCAATTTTGGCCGAATATCACAAAGTTCTTTGGCGAGTTCCAATACGGAGAAATCCTGC |
| PRIMERID-45080 | CAGTCAATTTTGGCCGAATATCACAAAGTGGTGGGGCGAGTTCCAATACGGAGAAATCCTGC |
| PRIMERID-45081 | CAGTCAATTTTGGCCGAATATCACAAAGATGATGGGCGAGTTCCAATACGGAGAAATCCTGC |
| PRIMERID-45082 | CAGTCAATTTTGGCCGAATATCACAAAGACGACAGGCGAGTTCCAATACGGAGAAATCCTGC |
| PRIMERID-45083 | CAGTCAATTTTGGCCGAATATCACAAAGAAAGGAGCAGAGTTCCAATACGGAGAAATCCTGC |
| PRIMERID-45084 | CAGTCAATTTTGGCCGAATATCACACATCATGGAGCAGAGTTCCAATACGGAGAAATCCTGC |
| PRIMERID-45085 | TCGTAATCTGGGCGTGTCGAATATCCTTCACTATCTTCTGGATGTGTCTGCGGACAAGCTAGTGAC |
| PRIMERID-45086 | TCGTAATCTGGGCGTGTCGAATATCATTCACTATATCATTGATGTGTCTGCGGACAAGCTAGTGAC |
| PRIMERID-45087 | TCGTAATCTGGGCGTGTCGAATATCGTTCACTATGTTGTGGATGTGTCTGCGGACAAGCTAGTGAC |
| PRIMERID-45088 | TCGTAATCTGGGCGTGTCGAATATCTTCCACTATTTCTTTGATGTGTCTGCGGACAAGCTAGTGAC |
| PRIMERID-45089 | TCGTAATCTGGGCGTGTCGAATATCTGGCACTATTGGTGGGATGTGTCTGCGGACAAGCTAGTGAC |
| PRIMERID-45090 | TCGTAATCTGGGCGTGTCGAATATCATGCACTATATGATGGATGTGTCTGCGGACAAGCTAGTGAC |
| PRIMERID-45091 | TCGTAATCTGGGCGTGTCGAATATCACACACTATACAACGGATGTGTCTGCGGACAAGCTAGTGAC |
| PRIMERID-45092 | TCGTAATCTGGGCGTGTCGAATATCTCCAAGTATAAAGCAAAGGTGTCTGCGGACAAGCTAGTGAC |
| PRIMERID-45093 | TCGTAATCTGGGCGTGTCGAATATCTCCCATTATCACGCACATGTGTCTGCGGACAAGCTAGTGAC |
| PRIMERID-45094 | GATTTCACAATGTGATGGCTGGACTTCCTGAGGAACTCTCGAACCCGAATGGAAAC |
| PRIMERID-45095 | GATTTCACAATGTGATGGCTGGAATTCCTGAGGAACTCTCGAACCCGAATGGAAAC |
| PRIMERID-45096 | GATTTCACAATGTGATGGCTGGAGTTCCTGAGGAACTCTCGAACCCGAATGGAAAC |
| PRIMERID-45097 | GATTTCACAATGTGATGGCTGGATTCCCTGAGGAACTCTCGAACCCGAATGGAAAC |
| PRIMERID-45098 | GATTTCACAATGTGATGGCTGGATGGCCTGAGGAACTCTCGAACCCGAATGGAAAC |
| PRIMERID-45099 | GATTTCACAATGTGATGGCTGGAATGCCTGAGGAACTCTCGAACCCGAATGGAAAC |
| PRIMERID-45100 | GATTTCACAATGTGATGGCTGGAACACCTGAGGAACTCTCGAACCCGAATGGAAAC |
| PRIMERID-45101 | GATTTCACAATGTGATGGCTGGAAAGCCTGAGGAACTCTCGAACCCGAATGGAAAC |
| PRIMERID-45102 | GATTTCACAATGTGATGGCTGGACATCCTGAGGAACTCTCGAACCCGAATGGAAAC |

Bacillus subtilis Strain Construction

B. subtilis strain WB800N (MoBiTec, Gottingen, Germany) and used as the expression host for this study. WB800N is a derivative of a well-studied strain (B. subtilis 168) and it has been engineered to reduce protease degradation of secreted proteins by deletion of genes encoding 8 extracellular proteases (nprE, aprE, epr, bpr, mpr, nprB, vpr and wprA). B. subtilis transformations were performed according to the manufacturer's instructions. Approximately 5 μg of library for SEQID-45001 variant constructs was transformed into WB800N and single colonies were selected at 37° C. by plating on LB agar containing 5.0 μg/ml chloramphenicol (Cm5). For 9 specific variants, 1 μg of specific SEQID-45001 variant was transformed into WB800N and single colonies were selected at 37° C. by plating on LB agar containing 5.0 μg/ml chloramphenicol (Cm5).

Bacillus subtilis Library Screening

˜800 individual transformants of the B. subtilis SEQID-45001 library were used to inoculate individual, 1-ml cultures of 2×-MAL medium (20 g/l NaCl, 20 g/l tryptone, and 10 g/l yeast extract, 75 g/l maltose) with Cm5, in deep well blocks (96-square wells). In addition to the library strains, a strain containing plasmid with AmyE and the SamyQ leader peptide was inoculated as a positive control and a strain containing plasmid with no gene of interest was inoculated as negative control. Culture blocks were covered with porous adhesive plate seals and incubated overnight in a micro-expression chamber (Glas-Col, Terre Haute, Ind.) at 37° C. and 880 rpm. Overnight cultures were used to inoculate fresh, 2×-MAL, Cm5 cultures, in deep well blocks, to a starting OD600=0.1.

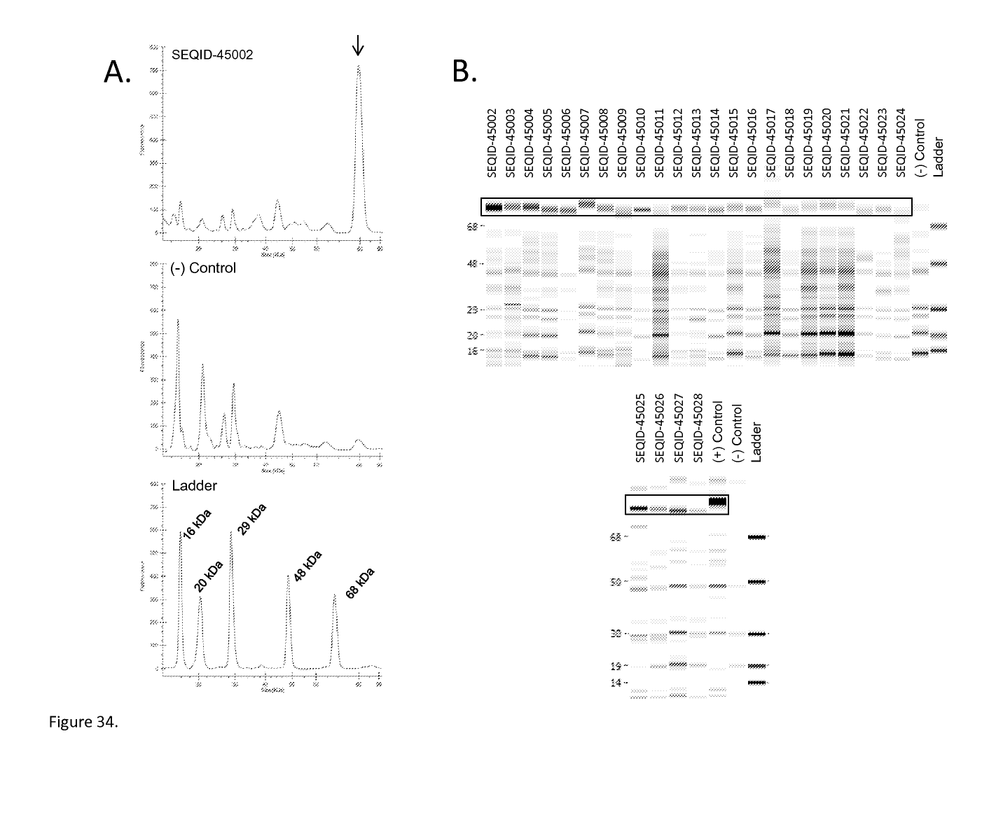

Expression cultures were incubated at 37° C., 880 rpm until the OD600=1.0 (approx. 4 hrs) at which time they were induced by adding isopropyl β-D-1-thiogalactopyranoside (IPTG) at a final concentration of 1 mM and continuing incubation for 4 hrs. After 4 hrs, the cell densities of each culture was measured (OD600) and cells were harvested by centrifugation (3000 rpm, 10 min, RT). After centrifugation, culture supernatant was carefully removed and transferred to a new block and cell pellets were frozen at −80° C. To determine the levels of secreted protein, 0.5-ml aliquots of the culture supernatants were filtered first through a 0.45-μm filter followed by a 0.22 μm filter. The filtrates were then assayed to determine the levels of secreted protein of interest (POI) by chip electrophoresis system and compared with the level of secretion of base construct. Briefly, samples were prepared by adding 2 μl of sample to 7 μl sample buffer, heating at 95 C for 5 minutes, and then adding 35 μl of water. Analysis was completed using HT Low MW Protein Express LabChip® Kit or HT Protein Express LabChip® Kit (following the manufacturer's protocol). A protein ladder ran every 12 samples for molecular weight determination (kDa) and quantification (ng/μl). An example of electropherogram demonstrating hit#3 secretion is shown in FIG. 34(A) along with negative control and ladder. An example of secretion of 23 different variants of SEQID-45001 screened from the library using this method is shown in FIG. 34(B).

Hit number 11 and 27 were confirmed by LC/MS/MS of the gel band of interest. Selected hits were mixed with Invitrogen LDS Sample Buffer containing 5% β-mercaptoethanol, boiled and loaded on a Novex® NuPAGE® 10% Bis-Tris gel (Life Technologies). After running, the gels were stained using SimplyBlue™ SafeStain (Life Technologies) and desired bands were excised and submitted for analysis. Gel bands were washed, reduced and alkylated, and then digested with Trypsin for 4 hours followed by quenching with formic acid. Digests were then analyzed by nano LC/MS/MS with a Waters NanoAcquity HPLC system interfaced to a ThermoFisher Q Exactive. Peptides were loaded on a trapping column and eluted over a 75 μm analytical column at 350 nL/min; both columns were packed with Jupiter Proteo resin (Phenomenex). The mass spectrometer was operated in data-dependent mode, with MS and MS/MS performed in the Orbitrap at 70,000 FWHM resolution and 17,500 FWHM resolution, respectively. The fifteen most abundant ions were selected for MS/MS. The resulting peptide data were searched using Mascot against the relevant host database with relevant variant protein sequence appended.

Diluted overnight cultures were used as inoculum for LB broth cultures containing Cm5. These cultures were grown at 37 C until they reached log phase. Aliquots of these cultures were mixed with glycerol (20% final concentration) and frozen at −80° C. The top 30 hits are then purified using Instagene matrix (Biorad, USA) and amplified using PRIMERID-45103 CTTGAAATTGGAAGGGAGATTC (SEQ ID NO: 45103) and PRIMERID-45104 GTATAAACTTTTCAGTTGCAGAC (SEQ ID NO: 45104), and sequenced using the same primers to identify the SEQID-45001 variant sequence.

Bacillus subtilis Secretion Library Analysis

All the secreted variants of SEQID-45001 (SEQIDs 45002-45028) were analyzed to determine if there were any position specific biases in the amino acids present in the secreted variants, relative to the expected position specific biases present in the initial genetic library. To this end, an exact binomial test was performed for each amino acid at each position to determine the likelihood that the observed number of each amino acid was significantly (p<0.05) more or less than expected by chance. Table 13a shows the p-values of this single tailed test, where those highlighted elements have p values<0.05. Note that aside from wild type values, which were all significantly higher than expected, all other significant different amino acid frequencies were less than expected. The expected position specific amino acid biases are shown in Table 13b, and were found by sequencing 47 randomly selected variants after the library had been constructed and transformed into e. coli. It was assumed that all positions designed to be an X effectively sampled from the same distribution of L, I, V, F, and M codons (i.e., for all X positions, there were no position specific amino acid biases). As such, the observed counts of each amino acid were aggregated across positions to determine the expected amino acid likelihoods for all X positions. A similar assumption was made for all positions designed to be a Z. As can be seen in Table 13a, in addition to the strong bias toward the wild type sequence at each position, there are a number of different amino acids that were observed significantly less than expected, indicating a bias away from those amino acids at that position in the secreted library. This data provides additional information for the design of specific, rationally designed variants with specific mutations at each position. As an example, to enrich a secreted variant in leucine, positions 241 and 291 may be less desired choices. Alternatively, to enrich a secreted variant in valine, positions 149, 241, 242, 291, 294, 295, and 389 may be less desired choices.

a: Single Tailed Binomial Test p-Values Assessing Position Specific Amino Acid Biases in Secreted Variants of SEQID-450001

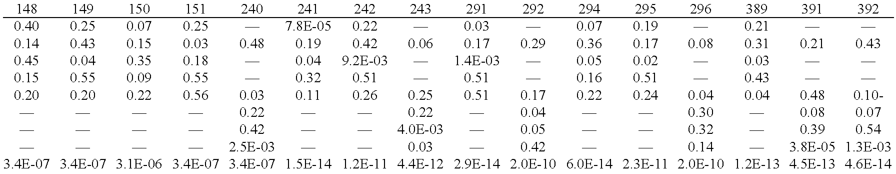

### b: Position Specific, Expected Amino Acid Likelihoods in the Constructed SEQID-450001 Library

| X | Z |
|---|---|
| 30.2% | — |
| 12.3% | 9.7% |
| 36.4% | — |
| 6.7% | — |
| 10.7% | 12.2% |
| — | 18.3% |
| — | 17.9% |
| — | 36.2% |
| 3.7% | 5.7% |

Bacillus subtilis Expression Testing of Specific Variants

Three separate colonies of B. subtilis expression strains were used to inoculate 1-ml of 2×-MAL medium (20 g/l NaCl, 20 g/l tryptone, and 10 g/l yeast extract, 75 g/l maltose) with Cm5, in deep well blocks (96-square wells). Culture blocks were covered with porous adhesive plate seals and incubated overnight in a micro-expression chamber (Glas-Col, Terre Haute, Ind.) at 37° C. and 880 rpm. Overnight cultures were used to inoculate fresh, 2×-MAL, Cm5 cultures, in deep well blocks, to a starting OD600=0.1. These expression cultures were incubated at 37° C., 880 rpm until the OD600=1.0 (approx. 4 hrs) at which time they were induced by adding isopropyl β-D-1-thiogalactopyranoside (IPTG) at a final concentration of 0.1 M and continuing incubation for 4 hrs. After 4 hrs, the cell densities of each culture was measured (0D600) and cells were harvested by centrifugation (3000 rpm, 10 min, RT). After centrifugation, culture supernatant was carefully removed and transferred to a new block and cell pellets were frozen at −80° C. To determine the levels of secreted protein, 0.5-ml aliquots of the culture supernatants were filtered first through a 0.45-μm filter followed by a 0.22 μm filter. The filtrates were then assayed to determine the levels of secreted protein of interest (POI) by chip electrophoresis. Briefly, samples were prepared by adding 2 μl of sample to 7 μl sample buffer, heating at 95 C for 5 minutes, and then adding 35 μl of water. Analysis was completed using HT Low MW Protein Express LabChip® Kit or HT Protein Express LabChip® Kit (following the manufacturer's protocol). A protein ladder ran every 12 samples for molecular weight determination (kDa) and quantification (ng/μl). An example of secretion of variants of SEQID-45001 that were enriched with methionine, threonine, lysine and histidine respectively were shown in FIG. 34(B).

SEQID-45025, SEQID-45026, SEQID-45027, and SEQID-45028 were confirmed by LC/MS/MS of the gel band of interest. Selected hits were mixed with Invitrogen LDS Sample Buffer containing 5% β-mercaptoethanol, boiled and loaded on a Novex® NuPAGE® 10% Bis-Tris gel (Life Technologies). After running, the gels were stained using SimplyBlue™ SafeStain (Life Technologies) and desired bands were excised and submitted for analysis. Gel bands were washed, reduced and alkylated, and then digested with Trypsin for 4 hours followed by quenching with formic acid. Digests were then analyzed by nano LC/MS/MS with a Waters NanoAcquity HPLC system interfaced to a ThermoFisher Q Exactive. Peptides were loaded on a trapping column and eluted over a 75 μm analytical column at 350 nL/min; both columns were packed with Jupiter Proteo resin (Phenomenex). The mass spectrometer was operated in data-dependent mode, with MS and MS/MS performed in the Orbitrap at 70,000 FWHM resolution and 17,500 FWHM resolution, respectively. The fifteen most abundant ions were selected for MS/MS. The resulting peptide data were searched using Mascot against the relevant host database with relevant variant protein sequence appended.

### Example 14

### Selection and Design of Engineered Secreted Protein from Aspergillus niger

To demonstrate the engineering of secreted polypeptides for enriched amino acid content, we chose a microorganism known to secrete protein at industrial scales, namely Aspergillus niger. The secreted polypeptide SEQID-45029 was identified as the major secreted protein in wild-type Aspergillus niger. Using sequence conservation and crystal structure data for SEQID-45029, we identified contiguous regions within each protein that were predicted to be amenable to mutation without negatively affecting the structural stability of the protein and/or the ability of the host organism to secrete the protein.

We analyzed the secondary structure of SEQID-45029 as reported in the structural protein databank 3EQA. We identified 13 loop regions within the sequence of the protein that are not part of an α-helix or a β-sheet. These loop regions are defined by the following amino acid residues: 48-76, 114-125, 131-148, 195-209, 253-268, 280-286, 309-312, 318-333, 364-370, 380-390, 417-438, 455-461, 467-486. Loop regions less than 4 amino acids in length were not considered for mutation.

Conservation of sequence over evolutionary space was also considered for identifying positions amenable for engineering while maintaining secretion competency. Positions that are less conserved within a family of homologous sequences are inherently variable and likely more amenable to mutation without affecting activity, which is intrinsically tied to structure. To find positions that are less conserved, we performed a PSI-BLAST search of the NCBI protein reference sequence database (Pruitt K. D., Tatusova T., and D. R. Maglott. Nucleic Acids Res. (2005) 33:D501-504) using SEQID-45029 and obtained 500 sequences homologous to SEQID-45029. In both cases, a single iteration was performed using the BLOSUM62 position specific scoring matrix, a gap penalty of −11, a gap extension penalty of −1, and an alignment inclusion e-value cutoff of 0.005 (Altschul S. F., Gish W., Miller W., Myers E. W., and D. J. Lipman. J. Mol. Biol. (1990) 215:403-410; Madden T L., Tatusov R. L., and Zhang, J., Meth. Enzymol. (1996) 266:131-141; Altschul S. F., Madden T. L., Schäffer A. A., Zhang J., Zhang Z., Miller W., and Lipman D. J. Nucleic Acids Res. (1997) 25:3389-3402). All protein sequence alignments were used to generate position-specific scoring matrices (PSSM) specific to each query sequence as part of the PSI-BLAST search. From the PSSMs, we identified regions hypothesized to be tolerant to mutation by counting the number of different amino acids associated with a positive PSSM score at each position within each loop as well as the sum and average of the PSSM scores for essential amino acid substitutions at each position Furthermore, from the multiple sequence alignments obtained from each PSI-BLAST search, we calculated the amino acid entropy at each position, as defined by

$${S}_{j} = - {\sum }_{i ∈ AA} {p}_{i} ln {p}_{i}$$

where Sⱼ is the entropy at position j and pⱼ is the probability of observing amino acid i at position j.

Using these measures of mutation tolerance we identified four loop regions expected to be tolerant to mutations into essential amino acids. To enrich the identified regions in essential amino acids we used a combinatorial codon library where any selected position could be either a F, I, L, V, or M (denoted Z) or a R, K, T, I, or M (denoted X). In each of the loop regions selected for mutation into an essential amino acid, each variable position was assigned as a Z or X depending upon its relative tolerance of hydrophobic residues (based upon their respective PSSM values). Positions that were tolerant of hydrophobic residues were assigned as Z and genetically encoded using the codon NTN. Positions more tolerant of hydrophilic residues were assigned as an X and genetically encoded using the codon ANR. For SEQID-45029 the sequences of the identified regions are summarized in the following table.

| Start residue # | original | degenerate |
|---|---|---|
| 121 | DLSSGA | ZLZZGZ |
| | (SEQ ID NO: 22143) | |
| 320 | SDSE | ZZXZ |
| | (SEQ ID NO: 22144) | |
| 429 | SDGEQ | XZGXX |
| | (SEQ ID NO: 22145) | |
| 474 | AATSA | XXTSX |
| | (SEQ ID NO: 22146) | |

X = NTN, codes for F, L, I, M, V
Z = ANR, codes for I, M, T, K, R

### Library Design and Construction

Based on identification of variable regions, we designed primers that can amplify each variable region as explained in FIG. 33. For example if there are four variable regions, we need four pair of primers to generate four variable fragments. In step 1 we used pES1962 (a derivative of LMBP2236 obtained from BCCM/LMBP, Ghent University with HIL6 replaced with a 3×FLAG tag) as the template which contains SEQID-45029 under glaA promoter with a C terminal 3×FLAG tag followed by the Aspergillus nidulans TrpC terminator. For fragment 1,2,3,4, the forward PRIMERID-45105, PRIMERID-45106, PRIMERID-45107, and PRIMERID-45108 contain 25 bases of constant sequence before the variable region followed by degenerate sequences to represent the variable region and 25 bases of constant sequence downstream of the variable region. For fragment 1, 2, 3, the reverse primers PRIMERID-45113, PRIMERID-45114, and PRIMERID-45115 contain 25 bases of reverse complementary sequence upstream of next variable region respectively. For fragment 4, the reverse primer PRIMERID-45116 contains 25 bases of reverse complementary sequence at an arbitrary distance from variable region 4. Four separate PCR amplifications were done using Phusion DNA polymerase (New England Biolabs, Beverly, Mass.) using the recommended manufacturer's protocol. As separate reactions, four wild type fragments, WT-frag-1, WT-frag-2, WT-frag-3 and WT-frag-4 were generated using PES1205 PES1962 as template and primer pairs PRIMERID-45109 & PRIMERID-45113, PRIMERID-45110 & PRIMERID-45114, PRIMERID-45111 & PRIMERID-45115, and PRIMERID-45112 & PRIMERID-45116, respectively. All fragments were gel purified. In step 2, two separate PCR reactions were set. The first PCR reaction contain fragment 1 and 2 in equimolar ratio as template and PRIMERID-45109 and PRIMERID-45114 as primers. The second PCR reaction contain fragment 3 and 4 in equimolar ratio and PRIMERID-45111 and PRIMERID-45116 as primers. In both the reactions, respective wild type fragments were added in a molar ratio of library members present in each variable fragments. Fragment 5 and 6 are gel purified and used as templates in equimolar ratio in step 3. The primers used in the PCR reaction include PRIMERID-45109 and PRIMERID-45116. The vector PCR product i was generated using pES1205 pES1962 and primer pairs, PRIMERID-45117 and PRIMERID-45118. Both fragment 7 and vector PCR product were gel purified and cloned together using the Gibson Assembly Master Mix (New England Biolabs, Beverly, Mass.) and transformed into the cloning host E. coli Turbo (New England Biolabs) according to manufacturer's instructions. 50 colonies were sequenced to determine the diversity of the library. The colonies on the agar plate were then suspended in LB media and harvested for plasmid purification. In a similar fashion, we generated 9 specific variants of SEQID-45029 which were altered with 9 specific amino acids, F, L, I, M, V, T, K, R, W at every variable position identified in the mutant design. Specific variant primers are denoted by the single letter amino acid abbreviation in the name. All primers are listed in table PRIMERID1 below.

| TABLE PRIMERID1 | Primer Sequence (SEQ ID NOS 45105-45154, respectively, in order of appearance) |
|---|---|
| PRIMERID-45105 | CCAGGGTATCAGTAACCCCTCTGGTANRCTGANRANRGGCANRGGTCTCGGTGAACCCAAGTTC |
| PRIMERID-45106 | CTCAATCTATACCCTCAACGATGGTCTCANRANRNTNANRGCTGTTGCGGTGGGTCGG |
| PRIMERID-45107 | CTCCATGTCCGAGCAATACGACAAGNTNANRGGCNTNNTNCTTTCCGCTCGCGACCTG |
| PRIMERID-45108 | TGCCAGCAGCGTGCCCGGCACCTGTNTNNTNACATCTNTNATTGGTACCTACAGCAGTGTGACTGTCAC |
| PRIMERID-45109 | CCAGGGTATCAGTAACCCCTCTGG |
| PRIMERID-45110 | CTCAATCTATACCCTCAACGATGGTCTC |
| PRIMERID-45111 | CTCCATGTCCGAGCAATACGAC |
| PRIMERID-45112 | TGCCAGCAGCGTGCCCG |
| PRIMERID-45113 | GAGACCATCGTTGAGGGTATAGATTGAG |
| PRIMERID-45114 | CTTGTCGTATTGCTCGGACATGG |
| PRIMERID-45115 | ACAGGTGCCGGGCACGC |
| PRIMERID-45116 | GATCGATCCGACCAGGTAGATGTTC |
| PRIMERID-45117 | GAACATCTACCTGGTCGGATCGATC |
| PRIMERID-45118 | ACCAGAGGGGTTACTGATACCCTG |
| PRIMERID-45119 | CCAGGGTATCAGTAACCCCTCTGGTCTCCTGCTCCTGGGCCTCGGTCTCGGTGAACCCAAGTTC |
| PRIMERID-45120 | CCAGGGTATCAGTAACCCCTCTGGTateCTGattatcGGCattGGTCTCGGTGAACCCAAGTTC |
| PRIMERID-45121 | CCAGGGTATCAGTAACCCCTCTGGTgtcCTGgtggtcGGCgtgGGTCTCGGTGAACCCAAGTTC |
| PRIMERID-45122 | CCAGGGTATCAGTAACCCCTCTGGTttcCTGttcttcGGCttcGGTCTCGGTGAACCCAAGTTC |
| PRIMERID-45123 | CCAGGGTATCAGTAACCCCTCTGGTtggCTGtggtggGGCtggGGTCTCGGTGAACCCAAGTTC |
| PRIMERID-45124 | CCAGGGTATCAGTAACCCCTCTGGTatgCTGatgatgGGCatgGGTCTCGGTGAACCCAAGTTC |
| PRIMERID-45125 | CCAGGGTATCAGTAACCCCTCTGGTaccCTGactaccGGCactGGTCTCGGTGAACCCAAGTTC |
| PRIMERID-45126 | CCAGGGTATCAGTAACCCCTCTGGTaagCTGaagaagGGCaagGGTCTCGGTGAACCCAAGTTC |
| PRIMERID-45127 | CCAGGGTATCAGTAACCCCTCTGGTcacCTGcaccacGGCcacGGTCTCGGTGAACCCAAGTTC |
| PRIMERID-45128 | CTCAATCTATACCCTCAACGATGGTCTCCTCCTGCTCCTGGCTGTTGCGGTGGGTCGG |
| PRIMERID-45129 | CTCAATCTATACCCTCAACGATGGTCTCatcattatcattGCTGTTGCGGTGGGTCGG |
| PRIMERID-45130 | CTCAATCTATACCCTCAACGATGGTCTCgtcgtggtcgtgGCTGTTGCGGTGGGTCGG |
| PRIMERID-45131 | CTCAATCTATACCCTCAACGATGGTCTCttcttcttcttcGCTGTTGCGGTGGGTCGG |
| PRIMERID-45132 | CTCAATCTATACCCTCAACGATGGTCTCtggtggtggtggGCTGTTGCGGTGGGTCGG |
| PRIMERID-45133 | CTCAATCTATACCCTCAACGATGGTCTCatgatgatgatgGCTGTTGCGGTGGGTCGG |
| PRIMERID-45134 | CTCAATCTATACCCTCAACGATGGTCTCaccactaccactGCTGTTGCGGTGGGTCGG |
| PRIMERID-45135 | CTCAATCTATACCCTCAACGATGGTCTCaagaagaagaagGCTGTTGCGGTGGGTCGG |
| PRIMERID-45136 | CTCAATCTATACCCTCAACGATGGTCTCcaccaccaccacGCTGTTGCGGTGGGTCGG |
| PRIMERID-45137 | CTCCATGTCCGAGCAATACGACAAGctcctgGGCctcctgCTTTCCGCTCGCGACCTG |
| PRIMERID-45138 | CTCCATGTCCGAGCAATACGACAAGatcattGGCatcattCTTTCCGCTCGCGACCTG |
| PRIMERID-45139 | CTCCATGTCCGAGCAATACGACAAGgtcgtgGGCgtcgtgCTTTCCGCTCGCGACCTG |
| PRIMERID-45140 | CTCCATGTCCGAGCAATACGACAAGttcttcGGCttcttcCTTTCCGCTCGCGACCTG |
| PRIMERID-45141 | CTCCATGTCCGAGCAATACGACAAGtggtggGGCtggtggCTTTCCGCTCGCGACCTG |
| PRIMERID-45142 | CTCCATGTCCGAGCAATACGACAAGatgatgGGCatgatgCTTTCCGCTCGCGACCTG |
| PRIMERID-45143 | CTCCATGTCCGAGCAATACGACAAGaccactGGCaccactCTTTCCGCTCGCGACCTG |
| PRIMERID-45144 | CTCCATGTCCGAGCAATACGACAAGaagaagGGCaagaagCTTTCCGCTCGCGACCTG |
| PRIMERID-45145 | CTCCATGTCCGAGCAATACGACAAGcaccacGGCcaccacCTTTCCGCTCGCGACCTG |
| PRIMERID-45146 | TGCCAGCAGCGTGCCCGGCACCTGTCTCCTGACATCTCTCATTGGTACCTACAGCAGTGTGACTGTCAC |
| PRIMERID-45147 | TGCCAGCAGCGTGCCCGGCACCTGTatcattACATCTatcATTGGTACCTACAGCAGTGTGACTGTCAC |
| PRIMERID-45148 | TGCCAGCAGCGTGCCCGGCACCTGTgtcgtgACATCTgtcATTGGTACCTACAGCAGTGTGACTGTCAC |
| PRIMERID-45149 | TGCCAGCAGCGTGCCCGGCACCTGTttettcACATCTttcATTGGTACCTACAGCAGTGTGACTGTCAC |
| PRIMERID-45150 | TGCCAGCAGCGTGCCCGGCACCTGTtggtggACATCTtggATTGGTACCTACAGCAGTGTGACTGTCAC |
| PRIMERID-45151 | TGCCAGCAGCGTGCCCGGCACCTGTatgatgACATCTatgATTGGTACCTACAGCAGTGTGACTGTCAC |
| PRIMERID-45152 | TGCCAGCAGCGTGCCCGGCACCTGTaccactACATCTaccATTGGTACCTACAGCAGTGTGACTGTCAC |
| PRIMERID-45153 | TGCCAGCAGCGTGCCCGGCACCTGTaagaagACATCTaagATTGGTACCTACAGCAGTGTGACTGTCAC |
| PRIMERID-45154 | TGCCAGCAGCGTGCCCGGCACCTGTcaccacACATCTcacATTGGTACCTACAGCAGTGTGACTGTCAC |

Aspergillus niger Strain Construction

An ΔaamA, pyrE derivative of Aspergillus niger MGG029 (Conesa et al., Applied and Environmental Microbiology, 2000) was used in the study. Expression vectors were co-transformed with a vector encoding the marker pyrE from Aspergillus niger using a standard protoplast transformation method (Punt et al., Methods in Enzymology, 1992). Protoplasts were transformed with 5 μg of expression vector and 1 μg vector encoding pyrE. Transformants were selected on minimal media supplemented with 1.2 M sorbitol (1.5% bacto agar, 10 g/l glucose, 4 g/l sodium nitrate, 20 ml/l salts solution (containing 26.2 g/l potassium chloride and 74.8 g/l Potassium phosphate monobasic at pH 5.5), and 1 ml/l of metals solution (containing 20 g/l Zinc sulfate heptahydrate (ZnSO4-7H2O), 11 g/l Boric acid (H3B03), 5 g/l Manganese (II) chloride tetrahydrate (MnCl2-4H2O), 5 g/l Iron (II) sulfate heptahydrate (FeSO4-7H2O), 1.7 g/l Cobalt(II) chloride hexahydrate (CoCl2-6H2O), 1.6 g/l Copper(II) sulfate pentahydrate (CuSO4-5H2O), 1.5 g/l Sodium molybdate dihydrate (NaMoO4-2H2O), and 5.0 g/l EDTA disodium salt dihydrate (Na2EDTA-2H2O) at pH 6.5). Plates were incubated at 30 C for 4 days until the majority of colonies had visible conidiophores.

Aspergillus niger Expression Testing

Conidia were picked from individual colonies using a sterile toothpick and inoculated directly in 800 μL of complete media (5.0 g/l yeast extract, 2.0 g/l casamino acids, 10 g/l maltose, 4 g/l sodium nitrate, 20 ml/l salts solution (containing 26.2 g/l potassium chloride and 74.8 g/l Potassium phosphate monobasic at pH 5.5), and 1 ml/l of metals solution (containing 20 g/l Zinc sulfate heptahydrate (ZnSO4-7H2O), 11 g/l Boric acid (H3BO3), 5 g/l Manganese (II) chloride tetrahydrate (MnCl2-4H2O), 5 g/l Iron (II) sulfate heptahydrate (FeSO4-7H2O), 1.7 g/l Cobalt(II) chloride hexahydrate (CoCl2-6H2O), 1.6 g/l Copper(II) sulfate pentahydrate (CuSO4-5H2O), 1.5 g/l Sodium moly′bdate dihydrate (NaMoO4-2H2O), and 5.0 g/l EDTA disodium salt dihydrate (Na2EDTA-2H2O) at pH 6.51), 1 ml/l vitamin solution (containing 100 mg/l Pyridoxine hydrochloride, 150 mg/l Thiamine hydrochloride, 750 mg/l 4-Aminobenzoic acid, 2.5 g/l Nicotinic acid, 2.5 g/l riboflavin, 20 g/l choline chloride, and 30 mg/l biotin), adjusted to pH 7 with 40 mM MES and supplemented with SigmaFast Protease Inhibitor Cocktail EDTA-Free (1 tab/100 mL, SigmaAldrich) in 96 well square bottom deep well blocks. Culture blocks were covered with porous adhesive plate seals and incubated for 48 hrs in a micro-expression chamber (Glas-Col, Terre Haute, Ind.) at 30° C. and shaking at 1000 rpm. After the growth period, 500 μL aliquots of the culture supernatants were filtered first through a 25 μm/0.45-μm dual stage filter followed by a 0.22 μm filter. The filtrates were then assayed to determine the levels of secreted protein of interest.

Aspergillus niger Sequencing Analysis

Fungal tissues were harvested from individual wells of the 96 deep well block and remaining supernatant was aspirated with a fine-tipped gel loading pipette tip. DNA was extracted using the ZRFungal/Bacterial DNA Miniprep kit (Zymo Research). Approximately 5 ng of genomic DNA was used as a template for PCR with PRIMERID-45155 (GAGAGCCTGAGCTTCATC) (SEQ ID NO: 45155) and PRIMERID-45156 (CACCAACGATCTTATATCCAGATTC) (SEQ ID NO: 45156) to amplify the entire expression cassette. The PCR reaction was purified using a Zymoclean Gel DNA recovery kit (Zymo Research) and sequenced PRIMERID-45155, PRIMERID-45156, and PRIMERID-45157 (AGCAGAGCTAACCCGC) (SEQ ID NO: 45157). Genomic DNA preps exhibiting polymorphisms at randomized loci were subcloned into pCRBluntII TOPO (Life Technologies) and 15 colonies were sequenced with PRIMERID-45155, PRIMERID-45156, and PRIMERID-45157.

### Anti-FLAG Dot Blot Analysis

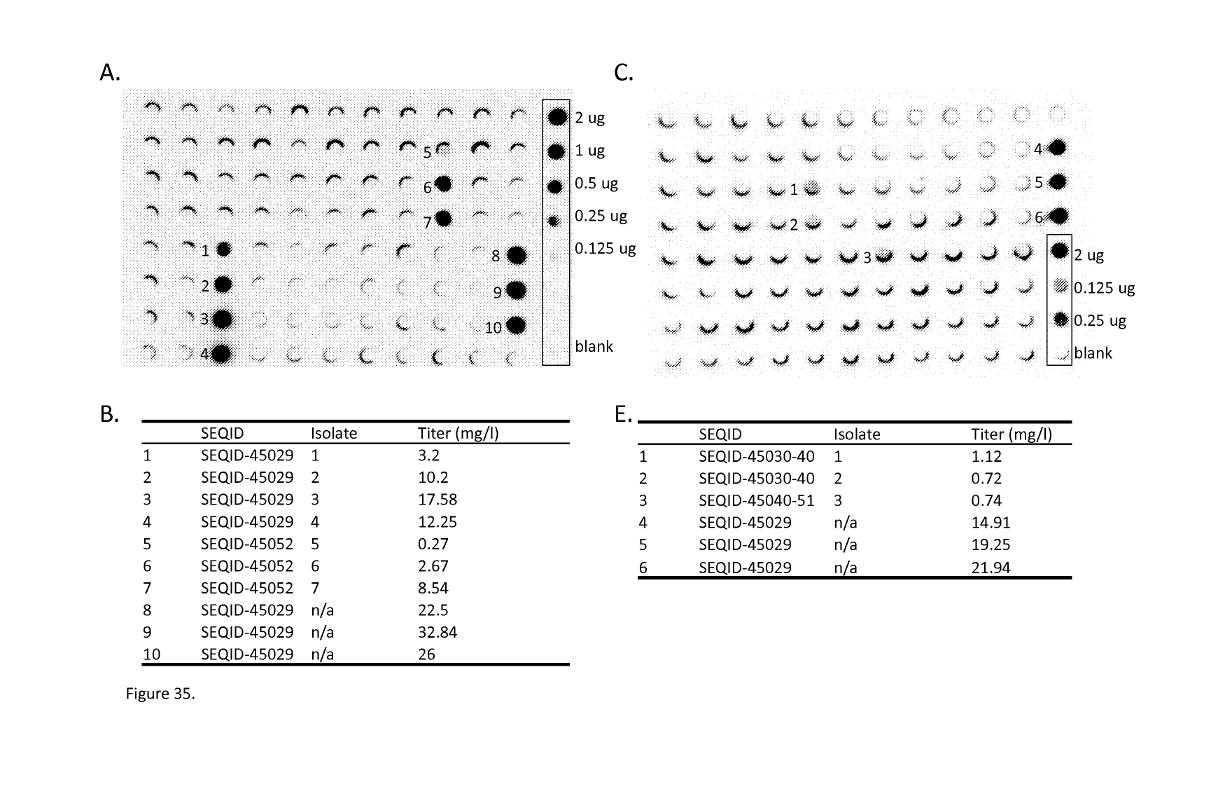

Extracellular protein was quantified using a dot blot method. 110 μl of 0.2 μm filtered sample was mixed with 110 μl 8.0M Guanidine Hydrochloride, 0.1M Sodium Phosphate (Denaturing Buffer) to allow for normalized protein binding and to ensure exposure of the tag. A standard curve of Amino-terminal FLAG-BAP™ Fusion Protein (Sigma) was prepared in the same matrix as the samples, starting at 2 μg, diluting 2× serially to 0.0313 μg. Invitrogen 0.45 μm nitrocellulose membrane was pre-wet in 1×PBS buffer for 5 minutes and then loaded onto Bio-Rad Dot Blot Apparatus. 300 μl of PBS was vacuumed through to further wet the membrane. Next, 200 μl the 1:1 Sample:Denaturing Buffer mixture was loaded into each well and allowed to drain through the dot blot apparatus by gravity for 30 minutes. Next, a 300 μl PBS wash was performed on all wells by vacuum followed by loading 300 μl of Millipore Blok CH Noise Cancelling reagent and incubating for 60 minutes. After blocking, the membrane was washed with 300 μl of 1×PBS+0.1% Tween 20. Next, antibody solution was prepared by adding 2.4 μl of Sigma Monoclonal ANTI-FLAG® M2-Peroxidase (HRP) antibody to 12 ml of Millipore Blok CH Noise Cancelling reagent (1:5000 dilution). 100 μl of the resulting antibody solution was added to each well and allowed to incubate for 30 minutes by gravity. After antibody incubation, three final washes are performed with 300 μl 1×PBS+0.1% Tween 20 by vacuum. After washes, the nitrocellulose membrane was removed and placed into a reagent tray. 20 ml of Millipore Luminata Classico Western HRP substrate was added and allowed to incubate for 1 minute. After incubation, membrane was placed into imaging tray of Gel Doc™ XR+System (Bio-rad) and imaged using a chemiluminescent protocol. FIG. 35 shows an example of an anti-FLAG dot-blot demonstrating secretion of SEQID-45029 variants in Aspergillus niger.

### Protein Identification by LC/MS/MS

The protein sequence of secreted variants is further confirmed by LC/MS/MS of the gel band of interest. Selected hits are mixed with Invitrogen LDS Sample Buffer containing 5% β-mercaptoethanol, boiled and loaded on a Novex® NuPAGE® 10% Bis-Tris gel (Life Technologies). After running, the gels are stained using SimplyBlue™ SafeStain (Life Technologies) and desired bands are excised and submitted for analysis. Gel bands are washed, reduced and alkylated, and then digested with Trypsin for 4 hours followed by quenching with formic acid. Digests will then analyzed by nano LC/MS/MS with a Waters NanoAcquity HPLC system interfaced to a ThermoFisher Q Exactive. Peptides are loaded on a trapping column and eluted over a 75 μm analytical column at 350 nL/min; both columns are packed with Jupiter Proteo resin (Phenomenex). The mass spectrometer is operated in data-dependent mode, with MS and MS/MS performed in the Orbitrap at 70,000 FWHM resolution and 17,500 FWHM resolution, respectively. The fifteen most abundant ions are selected for MS/MS. The resulting peptide data are searched using Mascot against the relevant host database with relevant variant protein sequence appended.

### Results

An Aspergillus niger strain was transformed with eight specific SEQID-45029 variants (pES2009, pES2010, pES2012, pES2013, pES2014, pES2015, pES2016, pES2017, pES1962). Primary transformants were selected on minimal media plates and conidia from approximately ten individual colonies were inoculated into 96 deep well blocks containing complete media. Cultures were incubated for 48 hrs after which period supernatants were assayed for the protein of interest with anti-FLAG dotblot analysis. For specific variants, only transformation with pES1962 encoding wild type SEQID-45029 and pES2016 encoding a poly-lysine substituted SEQID-45029 sequence gave FLAG signal in the supernatant (FIG. 35A, B).

An Aspergillus niger strain (see methods) was transformed with the SEQID-45029 expression vector library (see Table 1). Primary transformants were selected on minimal media plates and conidia from 43 individual colonies were inoculated (in duplicate) into a 96 deep well block containing complete media. Cultures were incubated for 48 hrs after which period supernatants were assayed for the protein of interest with anti-FLAG dotblot analysis. Supernatant analysis of solates 18 and 27 gave above background FLAG signal in the supernatant (FIG. 35C,D).

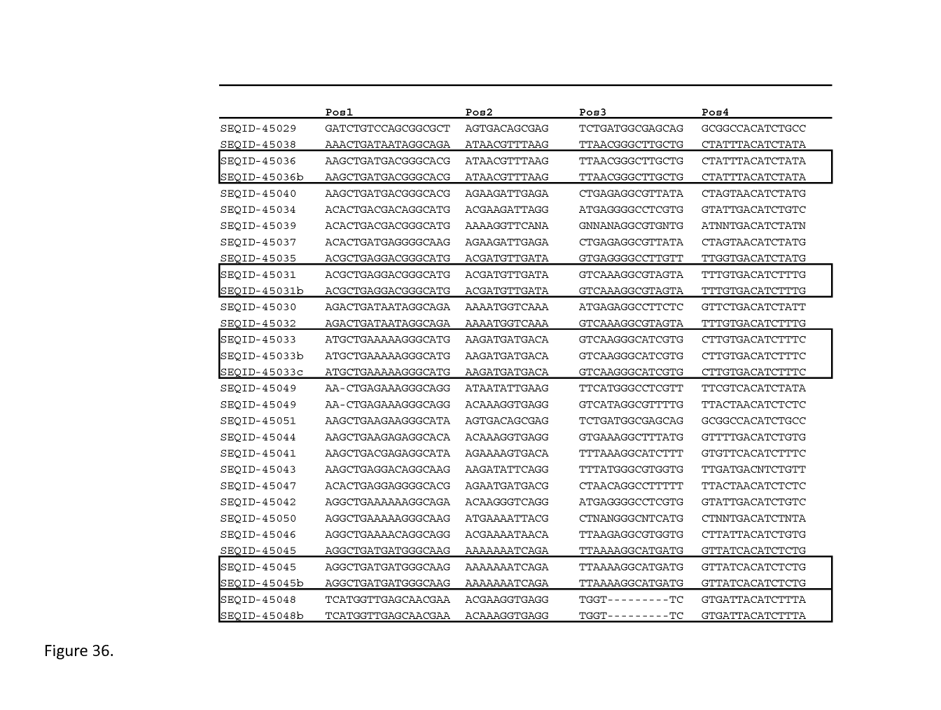

We isolated DNA form isolates 18 and 27 and amplified the SEQID-45029 expression cassette. The PCR product was fully sequenced to identify the specific DNA sequences found in the cells. The DNA sequences of isolates 18 and 27 showed polymorphisms at all four variable positions indicating that each isolate harbored multiple, distinct expression vectors. The PCR product were subcloned into pCRBlutII TOPO vector, transformed into E. coli, and 15 subclones were sequenced to determine the diversity of expression vectors (FIG. 36). For isolate 18, we identified 11 unique expression cassettes with no variable region identical to the wild-type sequence. Isolates 18-1 and 18-3 contained identical 247 base pair deletions spanning exon 3 and exon 4. For isolate 27, we identified 12 unique expression cassettes, one of which, 27-14 which was identical to the wild-type sequence in variable position 2, 3, and 4 but different than the wild-type sequence in variable position 1. The large number of unique expression cassettes among 15 isolates suggests that each primary isolate had multiple (e.g., more than 1, such as 2, 3, 4, 5, 6, 7, 8, 9, 10, 11, 12, 13, 14, 15 or more than 15) integration events or that each isolate was not a clonal colony and contained multiple primary transformants. It is possible that one or more of these expression cassettes contributes to the positive FLAG signal on the dot-blot. Mass spectrometric analyses of the supernatant or retransformation of all identified expression cassettes are used to identify which amino acid enhanced mutants are readily secreted.

### Example 15

### 15A. Secreted Protein from Bacillus Engineered for Increased Digestive Protease Cleavage Sites

The engineered secreted variants SEQID-45009, SEQID-45014, and SEQID-45027 are all enriched in a recognition site for a digestive protease. By adding sites for proteolysis the polypeptide will breakdown further into more small peptides for continued proteolysis until they are absorbed by the intestine. The three key proteases in protein digestion are Pepsin, Trypsin, and Chymotrypsin. Pepsin recognition sites are any site in a polypeptide sequence after (i.e., downstream of) an amino acid residue selected from Phe, Trp, Tyr, Leu, Ala, Glu, and Gln, provided that the following residue is not an amino acid residue selected from Ala, Gly, and Val. Trypsin recognition sites are any site in a polypeptide sequence after an amino acid residue selected from Lys or Arg, provided that the following residue is not a proline. Chymotrypsin recognition sites are any site in a polypeptide sequence after an amino acid residue selected from Phe, Trp, Tyr, and Leu.

SEQID-45009 is enriched in Arginine from 4.7% to 5.3%, a 13.8% increase in arginine content, thus enriching the polypeptide in cleavage sites for trypsin. SEQID-45014 is enriched in Leucine from 5.5% to 6.3%, a 14.3% increase in Leucine content, thus enriching the polypeptide in cleavage sites for both pepsin and chymotrypsin. SEQID-45027, is enriched in Lysine from 6.2% to 8.0%, a 28.9% increase in Lysine content, thus enriching the polypeptide in cleavage sites for trypsin. The amino acid content and PDCAAS score of the wild-type SEQID-45001 and variants are listed in Table 15A.

The digestibility of engineered secreted variants can be measure via an in vitro simulated digestion assay combined with analysis by electrophoresis, HPLC, and LC-MS/MS. In vitro digestion systems have history of being used to simulate the breakdown of polypeptides into bioaccessible peptides and amino acids while passing through the stomach and intestine (Kopf-Bolanz, K. A. et al., The Journal of nutrition 2012; 142: 245-250, Hur, S. J. et al., Food Chemistry 2011; 125: 1-12). Digestibility is also predictive of potentially allergenic sequences since polypeptide resistance to digestive proteases can lead to intestinal absorption and sensitization (Astwood et al., Nature Biotechnology 1996; 14: 1269-1273).

To measure digestibility the polypeptide is first treated at a concentration of 2 g/L with simulated gastric fluid (0.03 M NaCl, titrated with HCl to pH 1.5 with a final pepsin:polypeptide ratio of 1:20 w/w) at 37° C. Time points are sampled from the reaction and quenched by addition of 0.2 M Na2CO3. After 120 mins in simulated gastric fluid the remaining reaction is mixed 50:50 with simulated intestinal fluid (15 mM sodium glycodeoxycholate, 15 mM taurocholic acid, 18.4 mM CaCl2, 50 mM MES pH 6.5 with a final trypsin:chymotrypsin:substrate ratio of 1:4:400 w/w) and neutralized with NaOH to pH 6.5. Time points are sampled from the reaction and quenched by addition of Trypsin/Chymotrypsin Inhibitor solution until 120 mins. Sampled time points can then be analyzed by chip electrophoresis, reverse phase HPLC, and LC-MS/MS.

Chip electrophoresis (Labchip GX II) is used to evaluate the digestion rate (half-life) of intact protein. Samples are analyzed using a HT Low MW Protein Express LabChip® Kit (following the manufacturer's protocol) a protein ladder is loaded every 12 samples for molecular weight determination (kDa) and quantification. The concentration of the polypeptide at each time point (if detected) is plotted to calculate the half-life of digestion and represents the speed of protein digestion. By increasing protease recognition sites the intact protein is more likely to have an exposed cleavage sequence to increase the initial steps in proteolysis of intact protein.

To analyze digestions by reverse phase HPLC the samples are automatically derivatized with o-phthalaldehyde (OPA) and in-line analyzed by RP-HPLC with UV-Vis and fluorescence detection according to an Agilent application note, Henderson et al. “Rapid, Accurate, Sensitive, and Reproducible HPLC Analysis of Amino Acids” Agilent (2000) Amino acids and small peptides are detected and quantified by comparing to standard amino acids and peptide mixtures. The quantity of amino acids in digest samples represents the efficiency of a protein to digest into small bioavailable components. By adding more protease cleavage sites more amide bonds are broken and the efficiency of a protein to break down into amino acids is increased.

To analyze digest peptides by LC-MS/MS the sample pH is adjusted to pH3 with trifluoroacetic acid (TFA) and peptides are extracted using HLB solid phase extraction cartridges (Waters). The eluted peptides are then loaded on-column and analyzed by nano LC/MS/MS. Data are searched against an appropriate database using Mascot to identify peptides. Using this method large peptides that are resistant to digestion can be detected. By adding protease recognition sites to sequence space that is resistant to digestion the polypeptide can breakdown more thoroughly into small peptides and amino acids.

Secreted Protein from Bacillus Engineered for Increased Essential Amino Acid Content

Essential amino acids include Histidine, Isoleucine, Leucine, Lysine, Methionine, Phenylalanine, Threonine, Tryptophan, and Valine. Because their carbon skeletons are not synthesized de novo by the body to meet metabolic requirements, they must be taken as food. The engineered secreted polypeptides SEQID-45009, SEQID-45010, SEQID-45014, SEQID-45024, SEQID-45025, SEQID-45026, SEQID-45028, and SEQID-45027 have increased essential amino acid content by 1.1-2.5% as compared to wild-type. Particularly, SEQID-45014 increased the essential amino acid content of wild type from 42.1% to 43.7%, a 3.8% increase. Also, all of these variants contain a complete set of all essential amino acids. The administration of these nutritive polypeptides can provide the essential amino acids absent or present in insufficient amounts in a subject's diet to treat or prevent essential amino acid deficiency. The amino acid content and PDCAAS score of the wild-type SEQID-45001 and variants are listed in Table 15A.

Secreted Protein from Bacillus Engineered for Increased PDCAAS (Protein Digestibility Corrected Amino Acid Score)

PDCAAS is required by the United States Food and Drug Administration (US-FDA) labeling regulations, which were promulgated out of the Nutrition Labeling and Education Act of 1990 (NLEA), when making claims about the quality of protein content. The method was described and recommended for use by the Food and Agriculture Organization/World Health Organization (FAO/WHO) in 1991 (FAO/WHO. Protein Quality Evaluation; Report of a Joint FAO/WHO Expert Consultation, United Nations; Rome, Italy, 1991). PDCAAS is a measure for protein quality based on the preferred amino acid requirements of humans and their ability to digest it by evaluating the ratio of the limiting amino acid with respect to reference protein normalized by a true fecal digestibility percentage. Mutant variants SEQID-45009, SEQID-45010, SEQID-45024, and SEQID-45026 have elevated PDCAAS scores as compared to wild-type, especially for SEQID-45009 which increased the PDCAAS score from 0.92 to 1.04 a 13% increase. Polypeptides with higher PDCAAS score are able to provide a superior ratio of important amino acids delivered to the body. The amino acid content and PDCAAS score of the wild-type SEQID-45001 and variants are listed in Table 15A.

Secreted Protein from Bacillus Engineered for Increased Lysine Content

The engineered secreted variant SEQID-45027 has enriched lysine content when compared to wild-type protein. In SEQID-45027 lysine was increased from 6.2% to 8.0%, a 28.9% increase in the lysine content. By enriching secreted proteins in lysine, the content of an essential amino acid which cannot be synthesized has been increased and an important amino acid has been added that has additional utility to growth and health. The amino acid content and PDCAAS score of the wild-type SEQID-45001 and variants are listed in Table 15A.

Secreted Protein from Bacillus Engineered for Increased Methionine Content

The engineered secreted variants SEQID-45010 and SEQID-45026 have enriched methionine content when compared to wild-type protein. In SEQID-45010 methionine was increased from 1.9% to 2.4%, a 29.3% increase in the methionine content. In SEQID-45026 methionine was increased from 1.9% to 3.5%, an 89.0% increase in the methionine content. By enriching secreted proteins in methionine, the content of an essential amino acid which cannot be synthesized has been increased and an important amino acid has been added that has additional utility to growth and health. The amino acid content and PDCAAS score of the wild-type SEQID-45001 and variants are listed in Table 15A.

Secreted Protein from Bacillus Engineered for Increased Histidine Content

Mutant variant SEQID-45028 has increased histidine amino acid content to 4.9% from 3.1%, a 55% increase in histidine as compared to wild-type. By enriching secreted proteins in histidine, the content of an essential amino acid which cannot be synthesized has been increased and an important amino acid has been added that has additional utility to growth and health. The amino acid content and PDCAAS score of the wild-type SEQID-45001 and variants are listed in Table 15A.

Secreted Protein from Bacillus Engineered for Increased Arginine Content

The engineered secreted variants SEQID-45009, and SEQID-45010 have enriched arginine content when compared to wild-type protein. In SEQID-45009 arginine was increased from 4.7% to 5.3%, a 13.8% increase in the arginine content. In SEQID-45010 was increased from 4.7% to 5.3%, a 13.7% increase in the arginine content. By enriching secreted proteins in arginine an important non-essential amino acid has been added that has utility to growth and health. The amino acid content and PDCAAS score of the wild-type SEQID-45001 and variants are listed in Table 15A.

Secreted Protein from Bacillus Engineered for Increased Threonine Content

The engineered secreted variant SEQID-45025 has enriched threonine content when compared to wild-type protein. In SEQID-45025 threonine was increased from 6.9% to 8.2%, a 18.6% increase in the threonine content. By enriching secreted proteins in threonine, the content of an essential amino acid which cannot be synthesized has been increased and an important amino acid has been added that has additional utility to growth and health. The amino acid content and PDCAAS score of the wild-type SEQID-45001 and variants are listed in Table 15A.

Secreted Protein from Bacillus Engineered for Increased BCAA Content

We demonstrated SEQID-45009, SEQID-45010, SEQID-45014, SEQID-45024 variants are readily secreted and contain increased branched chain amino acids relative to wild-type SEQID-45001. SEQID-45009, SEQID-45010, SEQID-45014, SEQID-45024 contains 7.2%, 6.4%, 9.7%, and 8.1% increased branched chain amino acids relative to wild-type SEQID-45001. By enriching secreted proteins in BCAAs, the content of essential amino acids and an important family of amino acids have been increased. The amino acid content and PDCAAS score of the wild-type SEQID-45001 and variants are listed in Table 15A.

Branched Chain Amino Acids have been shown to have anabolic effects on protein metabolism by increasing the rate of protein synthesis and decreasing the rate of protein degradation in resting human muscle. Additionally, BCAAs are shown to have anabolic effects in human muscle during post endurance exercise recovery. These effects are mediated through the phosphorylation of mTOR and sequential activation of 70-kD S6 protein kinase (p70-kD S6), and eukaryotic initiation factor 4E-binding protein 1. P70-kD S6 is known for its role in modulating cell-cycle progression, cell size, and cell survival. P70-kD S6 activation in response to mitogen stimulation up-regulates ribosomal biosynthesis and enhances the translational capacity of the cell (W-L An, et al., Am J Pathol. 2003 August; 163(2): 591-607; E. Blomstrand, et al., J. Nutr. January 2006 136: 269S-273S). Eukaryotic initiation factor 4E-binding protein 1 is a limiting component of the multi-subunit complex that recruits 40S ribosomal subunits to the 5′ end of mRNAs. Activation of p70 S6 kinase, and subsequent phosphorylation of the ribosomal protein S6, is associated with enhanced translation of specific mRNAs.

BCAAs given to subjects during and after one session of quadriceps muscle resistance exercise show an increase in mTOR, p70 S6 kinase, and S6 phosphorylation was found in the recovery period after the exercise. However, there was no such effect of BCAAs on Akt or glycogen synthase kinase 3 (GSK-3). Exercise without BCAA intake leads to a partial phosphorylation of p70 S6 kinase without activating the enzyme, a decrease in Akt phosphorylation, and no change in GSK-3. BCAA infusion also increases p70 S6 kinase phosphorylation in an Akt-independent manner in resting subjects. Leucine is furthermore known to be the primary signaling molecule for stimulating mTOR1 phosphorylation in a cell-specific manner. This regulates cellular protein turnover (autophagy) and integrates insulin-like growth signals to protein synthesis initiation across tissues. This biology has been directly linked to biogenesis of lean tissue mass in skeletal muscle, metabolic shifts in disease states of obesity and insulin resistance, and aging.

Secreted Protein from Bacillus Engineered for Increased Leucine Content

The engineered secreted variants SEQID-45009, SEQID-45010, SEQID-45014, and SEQID-45024 have enriched leucine content when compared to wild-type protein. In SEQID-45009 leucine was increased from 5.5% to 6.1%, a 11.3% increase in the leucine content. In SEQID-45010 leucine was increased from 5.5% to 6.0%, a 8.3% increase in the leucine content. In SEQID-45014 leucine was increased from 5.5% to 6.3%, a 14.3% increase in the leucine content. In SEQID-45024 leucine was increased from 5.5% to 5.8%, a 5.6% increase in the leucine content. The amino acid content and PDCAAS score of the wild-type SEQID-45001 and variants are listed in Table 15A.

Secreted Protein from Bacillus Engineered for Increased Isoleucine Content

The engineered secreted variants SEQID-45009, SEQID-45010, and SEQID-45014 have enriched leucine content when compared to wild-type protein. In SEQID-45009 leucine was increased from 5.5% to 6.1%, a 11.3% increase in the leucine content. In SEQID-45010 leucine was increased from 5.5% to 6.0%, a 8.3% increase in the leucine content. In SEQID-45014 leucine was increased from 5.5% to 6.3%, a 14.3% increase in the leucine content. In SEQID-45024 leucine was increased from 5.5% to 5.8%, a 5.6% increase in the leucine content. The amino acid content and PDCAAS score of the wild-type SEQID-45001 and variants are listed in Table 15A.

Secreted Protein from Bacillus Engineered for Increased Valine Content

We demonstrated SEQID-45009, SEQID-45010, SEQID-45014, SEQID-45024 variants are readily secreted and contain increased in valine relative to wild-type SEQID-45001. SEQID-45009, SEQID-45010, SEQID-45014, SEQID-45024 contains 15.6%, 9.1%, 9.2% and 25.5% increased valine relative to wild-type SEQID-45001. The amino acid content and PDCAAS score of the wild-type SEQID-45001 and variants are listed in Table 15A.

Secreted Protein from Bacillus Engineered for Decreased Activity

In some cases the engineered secreted protein is an enzyme or has enzymatic activity. Since activity is not necessarily important for nutritional quality, it can be desirable to inactivate or reduce the enzymatic activity. The active sites of SEQID-45001 are predicted to be residues D217 and E249, which are acidic residues lying in the center of the catalytic domain. To produce a polypeptide free of enzymatic activity and enriched in amino acids important to nutrition and health, we can mutate those two sites to disrupt the catalytic activity of SEQID-45001. Because D217 and E249 in SEQID-45001 may act as nucleophiles and proton donors or acceptors to form hydrogen bonds with their ligands, we can mutate both residues into alanine or an essential amino acid to disrupt the activity. Alanine, phenylalanine, leucine, isoleucine, valine, and methionine are lack of oxygen or nitrogen atoms in their side chain and cannot act as nucleophiles or proton donors with the ligand. Threonine, lysine, and arginine are different from glutamic acid and aspartic acid in their charges under physiological pH and their sizes and shapes.

What is claimed is:

1. A formulation comprising an isolated nutritive polypeptide, wherein the nutritive polypeptide comprises a ratio of one or more essential amino acids to total amino acids that is higher than the ratio of one or more essential amino acids to total amino acids in a reference secreted protein at least 50 amino acids in length, wherein the nutritive polypeptide is present in the formulation in a nutritional amount, and wherein the formulation is substantially free of non-comestible products.

2. The formulation of claim 1, wherein the one or more essential amino acids are present in the formulation in a nutritional amount.

3. The formulation of claim 1, wherein the nutritive polypeptide comprises a ratio of total essential amino acids to total amino acids that is higher than the ratio of total essential amino acids to total amino acids in the reference secreted protein.

4. The formulation of claim 1, wherein the nutritive polypeptide comprises a ratio of a single essential amino acid to total amino acids that is higher than the ratio of a single essential amino acid to total amino acids in the reference secreted protein.

5. The formulation of claim 1, wherein the nutritive polypeptide comprises a ratio of two essential amino acids to total amino acids that is higher than the ratio of two essential amino acids to total amino acids in the reference secreted protein.

6. The formulation of claim 1, wherein the reference secreted protein comprises a secreted enzyme polypeptide.

7. The formulation of claim 6, wherein the isolated nutritive polypeptide is capable of a decreased level of the primary enzymatic activity of the secreted enzyme polypeptide.

8. The formulation of claim 1, wherein the isolated nutritive polypeptide is substantially purified from a host cell.

9. The formulation of claim 1, wherein the solubility of the nutritive polypeptide exceeds about 10 g/l at pH 7.

10. The formulation of claim 1, wherein the solubility of the nutritive polypeptide exceeds the solubility of the reference secreted protein.

11. The formulation of claim 1, wherein the digestibility of the nutritive polypeptide has a simulated gastric digestion half-life of less than sixty minutes.

12. The formulation of claim 1, wherein the digestibility of the nutritive polypeptide exceeds the digestibility of the reference secreted protein.

13. The formulation of claim 1, wherein the thermostability of the nutritive polypeptide exceeds the thermostability of the reference secreted protein.

14. The formulation of claim 1, wherein the nutritive polypeptide has a calculated solvation score of −20 or less.

15. The formulation of claim 1, wherein the nutritive polypeptide as a calculated aggregation score of 0.75 or less.

16. The formulation of claim 1, wherein the solubility and digestibility of the nutritive polypeptide exceeds the solubility and digestibility of the reference secreted protein.

17. The formulation of claim 1, wherein the nutritive polypeptide has less than about 50% homology to a known allergen.

18. The formulation of claim 1, wherein the reference secreted protein is i) a protein selected from proteins identified by UniProt Accession Numbers Q4WBW4, Q99034, A1DBP9, Q8NJP6, A1CU44, B0Y8K2, Q4WM08, Q0CMT2, Q8NK02, A1DNL0, A1CCN4, B0XWL3, Q4WFK4, A2QYR9, Q0CFP1, Q5B2E8, A1DJQ7, A1C4H2, B0Y9G4, B8MXJ7, Q4WBU0, Q96WQ9, A2R5N0, Q2US83, Q0CEU4, Q5BCX8, A1DBS6, Q9HE18, O14405, P62694, Q06886, P13860, Q9P8P3, P62695, P07987, A1C8U0, B0Y9E7, B8N1V9, Q4WBS1, Q2U2I3, Q5AR04, A1DBV1, B0YEK2, B8N7Z0, A4DA70, A2R2S6, Q2UI87, Q0CVX4, Q5AX28, A1D9S3, A1CC12, B0Y2K1, Q4WW45, Q5AQZ4, Q99024, P29026, P29027, P69328, P69327, P36914, P23176, P22832, A2QHE1, A1CR85, B0XPE1, B8NRX2, Q4WJJ3, P87076, A2RAL4, Q2UUD6, D0VKF5, Q0CTD7, Q5B5S8, A1D451, B8NJF4, A2QPK4, Q2UNR0, Q5AUW5, B0Y7Q8, B8NP65, Q4WMU3, Q2UN12, Q0CI67, Q5B6C6, A1DMR8, B8NMR5, Q2U325, Q0CUC1, Q5B0F4, A1DC16, A1CUR8, B0XM94, B8NPL7, Q4WL79, Q2U9M7, Q5B6C7, A1DPG0, A1CA51, B0Y3M6, B8NDE2, Q4WU49, A2R989, Q2U8Y5, Q0CAF5, Q5BB53, A1DFA8, B0Y8M8, Q4WLY1, Q5AV15, A1DNN8, Q5BA18, B0YB65, Q4WGT3, Q0CEF3, Q5B9F2, A1DCV5, B0XPB8, B8N5S6, Q4WR62, A5ABF5, Q2UDK7, Q0C7L4, Q5AWD4, A1D122, Q5B681, Q5BG51, A1CCL9, Q0CB82, Q5ATH9, Q4AEG8, B0XP71, B8MYV0, Q4WRB0, A2QA27, O00089, Q2UR38, Q0CMH8, Q5BAS1, P29026, P29027, P48827, A1CIA7, B0Y708, P35211, B8N106, P28296, P12547, Q00208, A1CWF3, P52750, P52754, P79073, P52755, P41746, or P28346, ii) SEQID-45001, iii) SEQID-45029, or iv) a fragment of i), ii) iii) at least 50 amino acids in length.

19. The formulation of any one of claims 1-18, comprising at least 1.0 g of nutritive polypeptide at a concentration of at least 100 g per 1 kg of formulation.

20. The formulation of any one of claims 1-18, wherein the formulation is present as a liquid, semi-liquid or gel in a volume not greater than about 500 ml or as a solid or semi-solid in a mass not greater than about 200 g.

21. The formulation of claim 1, wherein the nutritive polypeptide is produced in a recombinant organism.

22. The formulation of claim 1, wherein the nutritive polypeptide is produced by a unicellular organism comprising a recombinant nucleic acid sequence encoding the nutritive polypeptide.

23. The formulation of claim 1, wherein the formulation provides a nutritional benefit of at least about 2% of a reference daily intake value of protein or is otherwise present in an amount sufficient to provide a feeling of satiety when consumed by a human subject.

24. The formulation of claim 1, wherein the formulation provides a nutritional benefit of at least about 2% of a reference daily intake value of one or more essential amino acids.

25. The formulation of claim 1, wherein the formulation provides a nutritional benefit of at least about 2% of a reference daily intake value of total essential amino acids.

26. The formulation of claim 1, wherein the formulation provides at least 10 grams of nutritive polypeptide.

27. The formulation of claim 1, formulated for enteral administration.

28. The formulation of claim 1, wherein i) the nutritive polypeptide comprises at least about 98%, or 99%, or 99.5% or 99.9% overall sequence identity′ to the reference secreted protein over the full-length of the nutritive poly/peptide or the reference secreted protein, or ii) the nutritive polypeptide comprises an ortholog of the reference secreted protein, wherein the ortholog comprises at least about 70% overall sequence identity to the reference secreted protein over the full-length of the nutritive polypeptide or the reference secreted protein.

29. A food product comprising at least about 1 gram of the formulation of claim 1.

30. The formulation of claim 1, wherein the formulation provides a nutritional benefit per 100 g equivalent to or greater than at least about 2% of a reference daily intake value of protein.

31. The formulation of claim 1, wherein the effective amount of the nutritive polypeptide is lower than the effective amount of the reference secreted protein when administered to a human subject.

32. The formulation of claim 1, substantially free of a surfactant, a polyvinyl alcohol, a propylene glycol, a polyvinyl acetate, a polyvinylpyrrolidone, a non-comestible polyacid or polyol a fatty alcohol, an alkylbenzyl sulfonate, an alkyl glucoside, or a methyl paraben.

33. The formulation of claim 1, further comprising a tastant, a vitamin, a mineral, or a combination thereof.

34. The formulation of claim 1, further comprising a flavorant or non-nutritive polyol.

35. The formulation of claim 1, further comprising a nutritive carbohydrate and/or a nutritive lipid.

36. A recombinant unicellular organism comprising a recombinant nucleic acid sequence encoding an isolated nutritive polypeptide, wherein the nutritive polypeptide comprises a ratio of one or more essential amino acids to total amino acids that is higher than the ratio of one or more essential amino acids to total amino acids in a reference secreted protein at least 50 amino acids in length.

37. The recombinant unicellular organism of claim 36, wherein nutritive polypeptide is secreted from the unicellular organism.

38. A method of formulating a nutritive product, comprising the steps of providing a composition comprising an effective amount of an isolated nutritive polypeptide, wherein the nutritive polypeptide comprises a ratio of one or more essential amino acids to total amino acids that is higher than the ratio of one or more essential amino acids to total amino acids in a reference secreted protein at least 50 amino acids in length, wherein the nutritive polypeptide is present in the composition at a concentration of at least 1 mg of nutritive polypeptide per gram of the composition, and combining the composition with at least one food component, thereby formulating the nutritive product.

39. The method of claim 38, wherein the food component comprises a flavorant, a tastant, an agriculturally-derived food product, a vitamin, a mineral, a nutritive carbohydrate, a nutritive lipid, a binder, a filler or a combination thereof, wherein the nutritive product is comestible, and wherein the nutritive product comprises at least 1.0 g of nutritive polypeptide at a concentration of at least 100 g per 1 kg of nutritive product, and wherein the nutritive product is present as a liquid, semi-liquid or gel in a volume not greater than about 500 ml or as a solid or semi-solid in a mass not greater than about 200 g.

40. A method of selecting a nutritive composition for administration to a human subject who can benefit from same, the method comprising: identifying a minimal essential amino acid nutritive need in the subject; calculating an essential amino acid content score required to meet the minimal essential amino acid nutritive need; and providing a nutritive composition comprising an effective amount of a nutritive polypeptide, wherein the nutritive composition has at least the required essential amino acid content score.

41. A method of selecting a nutritive composition for administration to a human subject who can benefit from same, the method comprising: identifying a maximal essential amino acid nutritive need in the subject; calculating an essential amino acid content score required to not exceed the maximal essential amino acid nutritive need; and providing a nutritive composition comprising an effective amount of a nutritive polypeptide, wherein the nutritive composition has no greater than the required essential amino acid content score.

42. A method of treating a disease, disorder or condition characterized or exacerbated by protein malnourishment in a human subject in need thereof, comprising the step of administering to the human subject a nutritive formulation in an amount sufficient to treat such disease, disorder or condition, wherein the nutritive formulation comprises a nutritive polypeptide and an agriculturally-derived food product, wherein the nutritive polypeptide comprises a ratio of one or more essential amino acids to total amino acids that is higher than the ratio of one or more essential amino acids to total amino acids in a reference secreted protein at least 50 amino acids in length.

43. The method of claim 42, wherein the human subject is an elderly subject.

44. The method of claim 42, wherein the human subject is a child under 18 years old.

45. The method of claim 42, wherein the human subject is a pregnant subject or lactating female subject.

46. The method of claim 42, wherein the human subject is an adult between 18 years old and about 65 years old.

47. The method of claim 42, wherein the human subject is an adult suffering from or at risk of developing obesity, diabetes, or cardiovascular disease.

48. A method of improving the nutritional status of a human subject, comprising administering to the subject an effective amount of a nutritive formulation comprising an agriculturally-derived food product and an isolated nutritive polypeptide, wherein the nutritive polypeptide comprises a ratio of one or more essential amino acids to total amino acids that is higher than the ratio of one or more essential amino acids to total amino acids in a reference secreted protein at least 50 amino acids in length.

49. An engineered protein comprising: a sequence of at least 20 amino acids that comprise an altered amino acid sequence compared to the amino acid sequence of a reference secreted protein and a ratio of essential amino acids to total amino acids present in the engineered protein higher than the ratio of essential amino acids to total amino acids present in the reference secreted protein.

50. The engineered protein of claim 49, comprising at least one essential amino acid residue substitution of a non-essential amino acid residue in the reference secreted protein.

51. The engineered protein of claim 49, comprising i) at least one Arginine (Arg) or Glutamine (Glu) amino acid residue substitution of a non-Arginine (Arg) or non-Glutamine (Glu) amino acid residue in the reference secreted protein, ii) at least one phenylalanine (Phe) amino acid residue substitution of a non-Phe amino acid residue in the reference secreted protein, or iii) a combination thereof.

52. The engineered protein of claim 49, comprising i) at least one leucine (Leu) amino acid residue substitution of a non-Leu amino acid residue in the reference secreted protein, ii) at least one isoleucine (Ile) amino acid residue substitution of a non-Ile amino acid residue in the reference secreted protein, or iii) a combination thereof.

53. The engineered protein of claim 49, comprising at least one valine (Val) amino acid residue substitution of a non-Val amino acid residue in the reference secreted protein.

54. The engineered protein of claim 49, comprising at least one threonine (Thr) amino acid residue substitution of a non-Thr amino acid residue in the reference secreted protein.

55. The engineered protein of claim 49, comprising at least one lysine (Lys) amino acid residue substitution of a non-Lys amino acid residue in the reference secreted protein.

56. The engineered protein of claim 49, comprising at least one methionine (Met) amino acid residue substitution of a non-Met amino acid residue in the reference secreted protein.

57. The engineered protein of claim 49, comprising at least one histidine (His) amino acid residue substitution of a non-His amino acid residue in the reference secreted protein.

58. The engineered protein of claim 49, wherein the amino acid residue substitution is at an amino acid position with a per amino acid position entropy of at least 1.5.

59. The engineered protein of claim 49, wherein the difference in total folding free energy between the reference secreted protein and the engineered protein is less than or equal to 0.5.

60. An engineered protein comprising at least one essential amino acid residue substitution of a non-essential amino acid residue in a reference secreted protein at a position with a position entropy of at least 1.5.

61. The engineered protein of claim 49, wherein the reference secreted protein is a naturally occurring protein.

62. The engineered protein of claim 49, wherein the engineered protein is secreted from a compatible microorganism when expressed therein.

63. The engineered protein of claim 62, wherein the microorganism is the same genus as the microorganism in which the reference secreted protein naturally occurs.

64. The engineered protein of claim 62, wherein the microorganism is a heterotroph.

65. The engineered protein of claim 62, wherein the microorganism is photosynthetic.

66. The engineered protein of claim 65, wherein the photosynthetic microorganism is a cyanobacterium.

67. An isolated engineered protein comprising a sequence of at least 20 amino acids that comprise an altered amino acid sequence compared to the amino acid sequence of a reference secreted protein and a ratio of essential amino acids to total amino acids present in the engineered protein higher than the ratio of essential amino acids to total amino acids present in the reference secreted protein.

68. A formulation comprising a nutritional amount of the isolated engineered protein of claim 67.

69. The formulation of claim 68, wherein the formulation is substantially free of non-comestible products.

70. The formulation of claim 68, wherein the amino acid sequence of the engineered protein is at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.5% homologous to the reference secreted protein.

71. The formulation of claim 68, wherein the amino acid sequence of the engineered protein is at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 99.5% identical to the reference secreted protein.

72. The formulation of claim 68, wherein at least 2 non-essential amino acid residues in the reference secreted protein are substituted by essential amino acid residues.

73. The formulation of claim 68, wherein from about 5 to about 50 non-essential amino acid residues in the reference secreted protein are substituted by essential amino acid residues.

74. The formulation of claim 68, wherein at least about 1% of one or more non-essential amino acid residues in the reference secreted protein are substituted by one or more essential amino acid residues.

75. The formulation of claim 68, wherein at least about 1.5% of one or more non-essential amino acid residues in the reference secreted protein are substituted by one or more essential amino acid residues.

76. The formulation of claim 68, wherein at least about 2% of one or more non-essential amino acid residues in the reference secreted protein are substituted by one or more essential amino acid residues.

77. The formulation of claim 68, wherein at least about 3% of one or more non-essential amino acid residues in the reference secreted protein are substituted by one or more essential amino acid residues.

78. The formulation of claim 68, wherein at least about 4% of one or more non-essential amino acid residues in the reference secreted protein are substituted by one or more essential amino acid residues.

79. The formulation of claim 68, wherein the reference secreted protein is native to an organism of a genus selected from Aspergillus, Trichoderma, Penicillium, Thermomyces, Kluyveromyces, Chrysosporium, Myceliopthora, Acremonium, Fusarium, Trametes, and Rhizopus.

80. The formulation of claim 68, wherein the reference secreted protein is native to a microorganism selected from Escherichia coil, Bacillus subtilis, Saccharomyces cerevisiae, Pichia pastoris, Corynebacterium species, Bacillus amyloliquefaciens, Bacillus licheniformis, Synechocystis species, and Synechococcus species.

81. The formulation of claim 68, wherein the reference secreted protein is a protein selected from the proteins listed in Appendix A.

82. The formulation of claim 68, wherein the reference secreted protein is selected from SEQ ID NOS: 1-9.

83. The formulation of claim 68, wherein the reference secreted protein comprises a consensus sequence for a fold selected from cellulose binding domain, carbohydrate binding module, fibronectin type III domain, and hydrophobin.

84. The formulation of claim 68, wherein the reference secreted protein is a protein selected from proteins identified by UniProt Accession Numbers Q4WBW4, Q99034, A1DBP9, Q8NJP6, A1CU44, B0Y8K2, Q4WM08, Q0CMT2, Q8NK02, A1DNL0, A1CCN4, B0XWL3, Q4WFK4, A2QYR9, Q0CFP1, Q5B2E8, A1DJQ7, A1C4H2, B0Y9G4, B8MXJ7, Q4WBU0, Q96WQ9, A2R5N0, Q2US83, Q0CEU4, Q5BCX8, A1DBS6, Q9HE18, O14405, P62694, Q06886, P13860, Q9P8P3, P62695, P07987, A1C8U0, B0Y9E7, B8N1V9, Q4WBS1, Q2U2I3, Q5AR04, A1DBV1, B0YEK2, B8N7Z0, A4DA70, A2R2S6, Q2UI87, Q0CVX4, Q5AX28, A1D9S3, A1CC12, B0Y2K1, Q4WW45, Q5AQZ4, Q99024, P29026, P29027, P69328, P69327, P36914, P23176, P22832, A2QHE1, A1CR85, B0XPE1, B8NRX2, Q4WJJ3, P87076, A2RAL4, Q2UUD6, D0VKF5, Q0CTD7, Q5B5S8, A1D451, B8NJF4, A2QPK4, Q2UNR0, Q5AUW5, B0Y7Q8, B8NP65, Q4WMU3, Q2UN12, Q0CI67, Q5B6C6, A1DMR8, B8NMR5, Q2U325, Q0CUC1, Q5B0F4, A1DC16, A1CUR8, B0XM94, B8NPL7, Q4WL79, Q2U9M7, Q5B6C7, A1DPG0, A1CA51, B0Y3M6, B8NDE2, Q4WU49, A2R989, Q2U8Y5, Q0CAF5, Q5BB53, A1DFA8, B0Y8M8, Q4WLY1, Q5AV15, A1DNN8, Q5BA18, B0YB65, Q4WGT3, Q0CEF3, Q5B9F2, A1DCV5, B0XPB8, B8N5S6, Q4WR62, A5ABF5, Q2UDK7, Q0C7L4, Q5AWD4, A1D122, Q5B681, Q5BG51, A1CCL9, Q0CB82, Q5ATH9, Q4AEG8, B0XP71, B8MYV0, Q4WRB0 A2QA27, O00089, Q2UR38, Q0CMH8, Q5BAS1, P29026, P29027, P48827, A1CIA7, B0Y708, P35211, B8N106, P28296, P12547, Q00208, A1CWF3, P52750, P52754, P79073, P52755, P41746, and P28346.

85. The engineered protein of claim 49, wherein the engineered protein further comprises a polypeptide tag for affinity purification.

86. The engineered protein of claim 85, wherein the tag for affinity purification comprises a polyhistidine-tag.

87. The formulation of claim 68, wherein the engineered protein has a net absolute per amino acid charge of at least 0.05 at pH 7.

88. The formulation of claim 68, wherein the engineered protein has a net absolute per amino acid charge of at least 0.10 at pH 7.

89. The formulation of claim 68, wherein the engineered protein has a net absolute per amino acid charge of at least 0.15 at pH 7.

90. The formulation of claim 68, wherein the engineered protein has a net absolute per amino acid charge of at least 0.20 at pH 7.

91. The formulation of claim 68, wherein the engineered protein has a net absolute per amino acid charge of at least 0.25 at pH 7.

92. The formulation of claim 68, wherein the engineered protein has a net positive charge at pH 7.

93. The formulation of claim 68, wherein the engineered protein has a net negative charge at pH 7.

94. The formulation of claim 68, wherein the engineered protein is digestible.

95. The formulation of claim 68, wherein the engineered protein comprises a protease recognition site selected from a pepsin recognition site, a trypsin recognition site, and a chymotrypsin recognition site, or wherein the engineered protein comprises an increased ratio of a protease recognition site selected from a pepsin recognition site, a trypsin recognition site, and a chymotrypsin recognition site relative to a reference secreted protein.

96. An isolated nucleic acid comprising a nucleic acid sequence that encodes the engineered protein of claim 49.

97. The isolated nucleic acid according to claim 96, further comprising an expression control sequence operatively linked to the nucleic acid sequence that encodes an engineered protein.

98. A vector comprising a nucleic acid sequence that encodes the engineered protein of claim 49.

99. The vector of claim 98, further comprising an expression control sequence operatively linked to the nucleic acid sequence that encodes an engineered protein

100. A recombinant microorganism comprising at least one of a) a nucleic acid according to any one of claims 96 and 97 and b) a vector according to any one of claims 98 and 99.

101. A method of making an engineered protein comprising culturing the recombinant microorganism of claim 100 under conditions sufficient for production of the engineered protein by the recombinant microorganism.

102. The method of claim 101, further comprising isolating the engineered protein from the culture.

103. The method of claim 101, wherein the engineered protein is soluble.

104. The method of claim 101, wherein the engineered protein is secreted by the cultured recombinant microorganism and the engineered protein is isolated from the culture medium.

105. A nutritive composition comprising the engineered protein of claim 49 and at least one second component.

106. The nutritive composition of claim 105, wherein the second component is selected from a protein, a polypeptide, a peptide, a free amino acid, a carbohydrate, a fat, a mineral or mineral source, a vitamin, and an excipient.

107. The nutritive composition according to claim 105, wherein the second component is a protein.

108. The nutritive composition according to claim 107, wherein the protein is an engineered protein.

109. The nutritive composition according to claim 105 wherein the second component is one or more free amino acids selected from the essential amino acids.

110. The nutritive composition according to claim 105, wherein the second component is one or more free amino acids selected from branch chain amino acids.

111. The nutritive composition according to claim 110, wherein the second component is Leu.

112. The nutritive composition according to claim 105, wherein the second component is an excipient.

113. The nutritive composition according to claim 112, wherein the excipient is selected from the group consisting of a buffering agent, a preservative, a stabilizer, a binder, a compaction agent, a lubricant, a dispersion enhancer, a disintegration agent, a flavoring agent, a sweetener, and a coloring agent.

114. A nutritive composition according to claim 105, wherein the nutritive composition is formulated as a liquid solution, slurry, suspension, gel, paste, powder, or solid.

115. A method of making a nutritive composition, comprising providing the engineered protein according to claim 49 and combining the engineered protein with second component.

116. The method according to claim 115, wherein the second component is selected from a protein, a polypeptide, a peptide, a free amino acid, a carbohydrate, a fat, a mineral or mineral source, a vitamin, and an excipient.

117. The method according to claim 115, wherein the second component is a protein.

118. The method according to claim 117, wherein the protein is an engineered protein.

119. A method of maintaining or increasing of muscle mass, muscle strength, and functional performance in a subject, the method comprising providing to the subject a sufficient amount of the engineered protein of claim 49, a nutritive composition according to claim 105, or a nutritive composition made by a method according to claim 115.

120. A method of maintaining or achieving a desirable body mass index in a subject, the method comprising providing to the subject a sufficient amount of the engineered protein of claim 49, a nutritive composition according to claim 105, or a nutritive composition made by a method according to claim 115.

121. The method of claim 119 or 120, wherein the subject is elderly, critically-medically ill, or suffering from protein-energy malnutrition.

122. The method of claim 119 or 120, wherein the engineered protein of claim 49, the nutritive composition according to claim 105, or the nutritive composition made by a method according to claim 115 is consumed by the subject in coordination with performance of exercise.

123. A method of providing protein to a subject with protein-energy malnutrition, the method comprising providing to the subject a sufficient amount of engineered protein of claim 49, a nutritive composition according to claim 105, or a nutritive composition made by a method according to claim 115.

124. The method of claim 123, wherein the engineered protein of claim 49, the nutritive composition of claim 105, or the nutritive composition made by the method of claim 115 is consumed by the subject by an oral, enteral, or parenteral route.

125. A method of making an engineered protein, comprising:
a) providing a reference secreted protein, b) identifying a set of amino acid positions of the reference secreted protein to mutate to improve the nutritive content of the protein, and c) synthesizing the engineered protein comprising the target amino acid substitutions.

126. The method of claim 125, wherein the amino acid substitutions are encoded by a degenerate codon capable of i) encoding a plurality of desired amino acids, or ii) not encoding one or more undesired amino acids.

127. The method of claim 126, wherein the plurality of desired amino acids are enriched for one or more amino acids.

128. The method of claim 127, further comprising d) selecting an engineered protein comprising the amino acid substitutions.

129. The method of claim 125, wherein the reference secreted protein is i) native to a member of a genus selected from Aspergillus, Trichoderma, Penicillium, Chrysosporium, Myceliopthora, Acremonium, Fusarium, Trametes, and Rhizopus, ii) native to a microorganism selected from Escherichia coli, Bacillus subtilis, Saccharomyces cerevisiae, Pichia pastoris, Corynebacterium species, Synechocystis species, and Synechococcus species, or iii) is a protein listed in Appendix A.

130. The method of claim 125, wherein the reference secreted protein comprises a consensus sequence for a fold selected from a cellulose binding domain, carbohydrate binding module, fibronectin type III domain, and hydrophobin.

131. The method of claim 125, wherein identifying the set of amino acid positions of the reference secreted protein to mutate to improve the nutritive content of the protein comprises determining at least one parameter selected from amino acid likelihood (AALike), amino acid type likelihood (AATLike), position entropy (Sₚₒₛ), amino acid type position entropy (S_{AATpos}), relative free energy of folding (ΔΔG_{fold}), and secondary structure identity (LoopID) for a plurality of amino acid positions of the reference secreted protein.

132. The method of claim 125, wherein a combination of parameters selected from: (A) AAlike and ΔΔG_{fold}, (B) AATlike and ΔΔG_{fold}, (C) AATlike, and ΔΔG_{fold}, (D) Sₚₒₛ and ΔΔG_{fold}, (E) S_{AATpos} and ΔΔG_{fold}, (F) LoopID and ΔΔG_{fold}, (G) AAlike, ΔΔG_{fold}, and LoopID, (H) AAlike, AATlike, ΔΔG_{fold}, and LoopID, (I) AATlike, ΔΔG_{fold}, and LoopID, (J) Sₚₒₛ, ΔΔG_{fold}, and LoopID, and (K) S_{AATpos}, ΔΔG_{fold}, and LoopID is determined for a plurality of amino acid positions of the reference secreted protein.

133. The method of claim 125, further comprising ranking the plurality of amino acid positions of the reference secreted protein on the basis of the parameter and mutating the amino acids at positions having at least a threshold parameter value.

134. The method of claim 125, wherein the engineered protein is synthesized in vivo.

135. A library comprising a plurality of recombinant nucleic acid sequences encoding nutritive polypeptide variants, wherein each nutritive polypeptide variant comprises a ratio of one or more essential amino acids to total amino acids that is higher than the ratio of one or more essential amino acids to total amino acids in a reference secreted protein at least 50 amino acids in length.

136. A population of recombinant unicellular organisms comprising the library of claim 135.

137. The isolated nutritive polypeptide variants secreted from the population of claim 136.

138. Isolated fragments of the nutritive polypeptide variants of claim 137, wherein the fragments are suitable for analysis by mass spectrometry.

139. A device comprising the population of claim 136, wherein two or more individual recombinant unicellular organisms containing unique polypeptide variants are spatially separated.

140. The device of claim 139, wherein secreted nutritive polypeptide variants are capable of being identified.
